(19) United States
(12) Patent Application Publication
(10) Pub. No.: US 2026/0209222 A1
(43) Pub. Date: Jul. 23, 2026

(54) CBL-B MODULATORS AND USES THEREOF

(71) Applicant: Nimbus Clio, Inc., Boston, MA (US)

(72) Inventors: Sebastien Campos, Hoddesdon (GB); Alan Graves, Boston, MA (US); Silvana Marcel Leit de Moradei, Burlington, MA (US); Angela V. West, Franklin, MA (US); Salma B. Rafi, Cambridge, MA (US); Eric René Pierre Feyfant, Cambridge, MA (US); Jeremy Robert Greenwood, Brooklyn, NY (US)

(21) Appl. No.: 19/140,313

(22) PCT Filed: Dec. 19, 2023
(86) PCT No.: PCT/US2023/084897

§ 371 Date: Jun. 17, 2025

Related U.S. Application Data
(60) Provisional application No. 63/476,027, filed on Dec. 19, 2022.
(60) Provisional application No. 63/600,976, filed on Nov. 20, 2023.

(51) Int. Cl. C07D 471/04; A61K 31/423; A61K 31/4439; A61K 31/444; A61K 31/454; A61K 31/4545; C07D 413/10; C07D 413/14; C07D 491/107; C07D 519/00

(52) U.S. Cl. CPC C07D 471/04; A61K 31/423; A61K 31/4439; A61K 31/444; A61K 31/454; A61K 31/4545; C07D 413/10; C07D 413/14; C07D 491/107; C07D 519/00

(57) ABSTRACT

The present invention provides compounds of formula I-III, compositions thereof, and methods of using the same for the inhibition of Cbl-b, and the treatment of Cbl-b-mediated disorders.

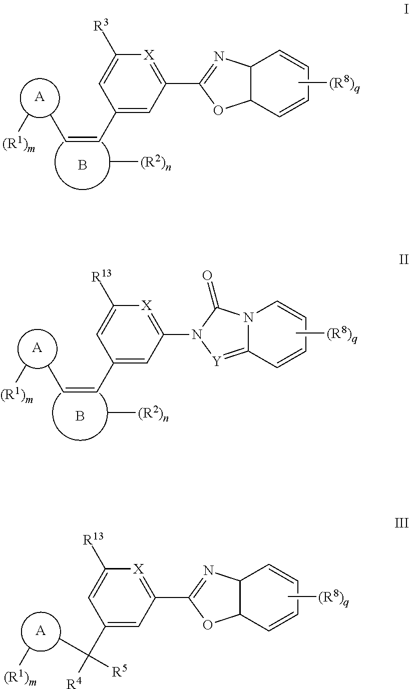

### CROSS-REFERENCE TO RELATED APPLICATIONS

This application claims the benefit of priority to U.S. Provisional Appl. 63/476,027, filed Dec. 19, 2022, and U.S. Provisional Appl. 63/600,976, filed Nov. 20, 2023, the content of each of which is herein incorporated by reference.

### TECHNICAL FIELD OF THE INVENTION

The present invention relates to compounds and methods useful for inhibiting E3 ligase Casitas B-lineage lymphoma b (Cbl-b). The invention also provides pharmaceutically acceptable compositions comprising compounds of the present invention and methods of using said compositions in the treatment of various disorders.

### BACKGROUND OF THE INVENTION

Ubiquitination is a post-translational modification that regulates the function and fate of proteins involved with physiological processes. The addition of ubiquitin to target proteins occurs via a three-step enzymatic process that involves three enzymes. The first enzyme, E1, catalyzes ubiquitin activation. Activated ubiquitin is then transferred from E1 to the ubiquitin-conjugating enzyme. E2. The third enzyme, or E3 ligase, confers substrate specificity and directly catalyzes the transfer of ubiquitin from the E2 into the protein substrate. The addition of poly-ubiquitin chains to proteins serves as a signal leading to degradation into peptides of the ubiquitin-conjugated protein by the proteasome. Additionally, poly- and mono-ubiquitination can also alter cellular localization, function, and interactions of the protein substrate with proteins required for downstream activity and signaling events.

Ubiquitination controls multiple biological processes that are often dysregulated in disease, including cell cycle, DNA repair, differentiation, and innate and adaptive immunity. Therefore, the discovery of molecules that modulate components of the ubiquitin proteasome system represents an attractive therapeutic opportunity for a wide range of conditions, including cancer and auto-immune disease.

The compounds and compositions described herein are generally useful for the inhibition of the E3 ligase Casitas B-lineage lymphoma b (Cbl-b).

Cbl-b is a RING finger E3 ligase and a member of a highly conserved family of Cbl proteins, which in mammals consists of three Cbl genes: Cbl, Cbl-b, and Cbl-c. Cbl proteins interact with target proteins via their protein-protein interaction domains, allowing regulation of multiple signaling pathways, including tyrosine kinase (TK) signaling in multiple cell types. The structure of Cbl proteins consists of an amino-terminal tyrosine kinase binding domain (TKBD), a linker helix region (LHR) and a really interesting new gene (RING) domain, followed by a carboxy-terminal region containing binding sites for Src homology 2 (SH2) and Src homology 3 (SH3) domains. Cbl TKBD is composed of a four-helix bundle (4H), an EF-hand, and a variant SH2 domain, which binds substrates, such as activated TKs, in a phospho-tyrosine dependent manner.

Ubiquitination of activated receptor TKs by Cbl-b regulates the assembly of endocytic proteins both at the membrane and at sorting endosomes to promote lysosome targeting, degradation and signal termination. Cbl-b is also important for down regulation of signaling from antigen and cytokine receptors through ubiquitination of receptor chains and associated cytosolic TKs, leading to inactivation and/or proteasomal degradation.

Cbl-b is expressed in immune cell lineages and acts as a major regulator of immune cell activation and maintenance of peripheral tolerance. Cbl-b negatively regulates adaptive immune system signaling by establishing the threshold for the activation of antigen receptors. In T cells, Cbl-b imposes a requirement for a co-stimulatory signal to mount a productive immune response upon T cell receptor (TCR) engagement. Mice deficient in Cbl-b, and more specifically in the RING Zn-finger ligase domain of Cbl-b, showed tumor rejection that is mediated by CD8+ T cells.

Additionally, Cbl-b regulates the activity of multiple cell lineages involved in innate immunity, including NK cells, antigen-presenting dendritic cells (DC) and monocytes. Therefore, due to the complexity and diversity of the protein targets of Cbl-b in a variety of immune cells, it is possible that the functions of Cbl-b are cell-type dependent.

Novel therapeutic approaches aimed at removing inhibitory signals in immune cells to boost a productive immune system have gained recent attention. Given the central role that Cbl-b plays in regulating multiples signaling mechanisms in both innate and adaptive immunity, inhibition of Cbl-b provides therapeutic opportunities, including cancer immunotherapies.

Cbl-b inhibitors may strengthen the activity of cancer vaccines. For example, it was reported that the adoptive transfer of Cbl-b−/− CD8+ T cells combined with DC vaccines delays tumor growth. Additionally, Cbl-b−/− T cells are resistant to inhibition by PDL-1/PD-1 in vitro and in vivo, which supports the rationale combination of Cbl-b inhibitors with anti-PD-1/PD-L1 checkpoint blockade.

Enhanced expression of Cbl-b associates with better prognosis in lung adenocarcinoma. Moreover, mutations in the RING finger domain of Cbl proteins and Cbl-b linker sequence are found in a variety of disorders and cancers, including Juvenile myelomonocytic leukemia (JMML), preleukemic chronic myelomonocytic leukemia (CMML), Myeloproliferative Neoplasms (MPN), and Acute myeloid leukemia (AML). These observations suggest that that degradation impairment of activated TKs represents an important cancer mechanism that involves Cbl proteins. In agreement, multiple reports have demonstrated the ubiquitination of the epidermal growth factor receptor (EGFR) and the platelet derived growth factor receptor alpha (PDGFRa) by Cbl-b. The ubiquitination of these receptors promotes their proteasomal-dependent degradation in a variety of cancer lineages. The degradation of EGFR by Cbl-b leads to lung and gastric cancer cell proliferation and mediates epithelial to mesenchymal transition (EMT) in metastatic breast and gastric cancers. Additionally, amplifications and mutations of both EGFR and PDGFR are major drivers of oncogenic transformation and are commonly found in multiple types of cancer. Thus, Cbl-b inhibition represents an opportunity for both tumor intrinsic and tumor extrinsic therapies.

PCT/US2022/071633 describes Cbl-b inhibitor compounds that also show activity for c-Cbl. Accordingly, there is an unmet medical need to provide compounds that have high Cbl-b selectivity to avoid adverse affects associated with interactions with non-efficiency conferring targets, such as c-Cbl.

### SUMMARY OF THE INVENTION

It has now been found that compounds of this invention, and pharmaceutically acceptable compositions thereof, are effective as inhibitors of Cbl-b. In certain embodiments, the invention provides for compounds of the formulae presented herein.

Compounds of the present invention, and pharmaceutically acceptable compositions thereof, are useful for treating a variety of diseases, disorders or conditions, associated with modulating the immune system implicating Cbl-b. Such diseases, disorders, or conditions include those described herein.

Compounds provided by this invention are also useful for the study of Cbl-b enzymes in biological and pathological phenomena; the study of ubiquitination occurring in bodily tissues; and the comparative evaluation of new Cbl-b inhibitors or other regulators of cell cycle, DNA repair, differentiation, and innate and adaptive immunity in vitro or in vivo.

### DETAILED DESCRIPTION OF CERTAIN EMBODIMENTS

### 1. General Description of Certain Embodiments of the Invention

In certain aspects, the present invention provides a compound of formula I:

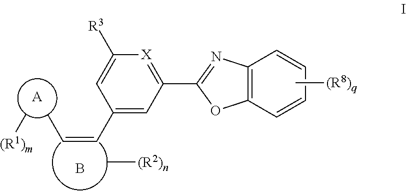

or a pharmaceutically acceptable salt thereof, wherein each of Ring A, Ring B, R¹, R², R³, R⁸, X, m, n, and q is as defined below and described in embodiments herein, both singly and in combination.

In certain aspects, the present invention provides a compound of formula II:

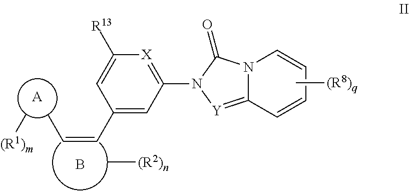

or a pharmaceutically acceptable salt thereof, wherein each of Ring A, Ring B, R¹, R², R⁸, R¹³, X, Y, n, n, and q is as defined below and described in embodiments herein, both singly and in combination.

In certain aspects, the present invention provides a compound of formula III:

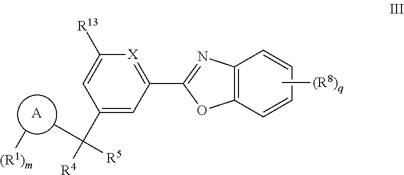

or a pharmaceutically acceptable salt thereof, wherein each of Ring A, R¹, R⁴, R⁵, R⁸, R¹³, X, m, n, and q is as defined below and described in embodiments herein, both singly and in combination.

In some embodiments, the present invention provides a pharmaceutical composition comprising a compound of formula I-III and a pharmaceutically acceptable carrier, adjuvant, or diluent.

In some embodiments, the present invention provides a method of treating a Cbl-b-mediated disease, disorder, or condition comprising administering to a patient in need thereof, a compound of formula I-III, or a pharmaceutically acceptable salt thereof.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics. 75^{th} Ed. Additionally, general principles of organic chemistry are described in “Organic Chemistry”. Thomas Sorrell, University Science Books, Sausalito: 1999, and “March's Advanced Organic Chemistry”, 5^{th} Ed. Ed.: Smith, M. B, and March, J., John Wiley & Sons, New York: 2001.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments. “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl. (cycloalkenyl)alkyl or (cycloalkyl)alkenyl.

As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur Unless otherwise specified, a bridged bicyclic group is optionally substituted with one or more substituents as set forth for aliphatic groups. Additionally or alternatively, any substitutable nitrogen of a bridged bicyclic group is optionally substituted. The term “alkyl” refers to a C₁₋₁₂ straight or branched saturated aliphatic group. In certain instances, alkyl refers to a C₁₋₈ straight or branched saturated aliphatic group or a C₁₋₆ straight or branched saturated aliphatic group. The term “lower alkyl” refers to a C₁₋₄ straight or branched alkyl group.

Exemplary lower alkyl groups are methyl, ethyl, propyl, isopropyl (also referred to interchangeably herein as 2-propyl, iPr, ⁱPr and i-Pr), butyl, isobutyl (also referred to interchangeably herein as 2-butyl, iBu, ⁱBu and i-Bu) and tert-butyl (also referred to interchangeably herein as 2-methyl-2-butyl, tBu, ^{t}Bu and t-Bu).

The term “alkenyl” refers to a C₂₋₁₂ straight or branched partially unsaturated aliphatic group comprising at least one unsaturated carbon carbon double bond. In certain instances, alkenyl refers to a C₂₋₈ or a C₁₋₆ straight or branched partially unsaturated aliphatic group comprising at least one unsaturated carbon carbon double bond. The term “lower alkenyl” refers to a C₂₋₄ straight or branched partially unsaturated aliphatic group comprising at least one unsaturated carbon carbon double bond. Alkenyl groups include both cis (Z) and trans (E) regioisomers. Exemplary lower alkenyl groups are vinyl, allyl, 2-propenyl, and butenyl isomers (—CH₂CH₂CH═CH₂, —CH₂CH═CHCH₃ and —CH═CH═CH₂CH₃).

The term “alkynyl” refers to a C₂₋₁₂ straight or branched partially unsaturated aliphatic group comprising at least one unsaturated carbon carbon triple bond. In certain instances, alkynyl refers to a C₂₋₈ or a C₁₋₆ straight or branched partially unsaturated aliphatic group comprising at least one unsaturated carbon carbon triple bond. The term “lower alknyl” refers to a C₂₋₄ straight or branched partially unsaturated aliphatic group comprising at least one unsaturated carbon carbon triple bond. Exemplary lower alkynyl groups are ethynyl, 1-propynyl, 2-propynyl, 1-butynyl, 2-butynyl, and 3-butynyl.

The term “haloalkyl” refers to a straight or branched alkyl group that is substituted with one or more halogen atoms. The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon: the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy.” refer to groups having 5 to 10 ring atoms, preferably 5, 6, 9 or 10 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, pyrazinyl, pyrazolyl, pyridazinyl, pyridyl, pyrimidinyl, triazinyl, thienyl, furanyl, pyrrolyl, imidazolyl, pyrazolyl, triazolyl (i.e., 1,2,3-triazolyl), 1,2,4-triazolyl, 1,2,5-triazolyl, 1,3,4-triazolyl, tetrazolyl, oxazolyl, isoxazolyl, oxadiazolyl, 1,2,3-oxadiazolyl, 1,2,4-oxadiazolyl, 1,2,5-oxadiazolyl, 1,3,4-oxadiazolyl, thiazolyl, isothiazolyl, thiadiazolyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, indolizinyl, isoindolin-1-only, 1,2-dihydro-3H-pyrrolo[3,4-c]pyridin-3-onyl, 2,3-dihydro-TH-pyrrolo[3,4-c]pyridin-1-only, imidazo[1,2-a]pyridyl, imidazo[1,5-a]pyridyl, pyrazolo[1,5-a]pyridyl, pyrrolo[1,2-b]pyridazinyl, pyrrolo[1,2-a]pyrimidinyl, imidazo[1,2-b]pyridazinyl, imidazo[1,2-a]pyrimidinyl, benzimidazolyl, benzthiazolyl, quinolyl, isoquinolyl, cinnolinyl, phthalazinyl, quinazolinyl, quinoxalinyl. 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, oxetanyl, azetidinyl, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{o}: —(CH₂)₀₋₄OR^{o}; —O(CH₂)₀₋₄R^{o}, —O—(CH₂)₀₋₄C(O)OR^{o}; —(CH₂)₀₋₄CH(OR^{o})₂: —(CH₂)₀₋₄SR^{o}; —(CH₂)₀₋₄Ph, which may be substituted with R^{o}; —(CH₂)₀₋₄O(CH₂)₀₋₄Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —N(R^{o})C(NR^{o})N(R^{o})₂—(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄ OC(O)R^{o}; —OC(O)(CH₂)₀₋₄SR^{o}; —(CH₂)₀₋₄SC(O)R^{o}: —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄ S(O)₂R^{o}; —(CH₂)₀₋₄S(O)₂OR^{o}: —(CH₂)₀₋₄ OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —(CH₂)₀₋₄P(O)₂R^{o}; —(CH₂)₀₋₄P(O)R^{o}₂; —(CH₂)₀₋₄OP(O)R^{o}₂; —(CH₂)₀₋₄OP(O)(OR^{o})₂; —SiR^{o}₃; —(C₁₋₄ straight or branched alkylene)O—N(R^{o})₂; or —(C₁₋₄ straight or branched alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₃, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR^{*}₂, ═NNHC(O)R^{*}, ═NNHC(O)OR^{*}, ═NNHS(O)₂R^{*}, ═NR^{*}, ═NOR^{*}, —O(C(R^{*}₂))₂₋₃O—, or —S(C(R^{*}₂))₂₋₃S—, wherein each independent occurrence of R^{*} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR^{*}₂)₂₋₃O—, wherein each independent occurrence of R^{*} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{*} include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen. C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Unless otherwise stated, structures depicted herein are also meant to include all isomeric (e.g., enantiomeric, diastereomeric, and geometric (or conformational)) forms of the structure: for example, the R and S configurations for each asymmetric center, Z and E double bond isomers, Z and E conformational isomers and Rₐ (or M) and Sₐ (or P) atropisomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention. In certain embodiments, a warhead moiety, R¹, of a provided compound comprises one or more deuterium atoms. In certain embodiments, Ring B of a provided compound may be substituted with one or more deuterium atoms.

The structures as drawn represent relative configurations, unless labeled as absolute configurations. The invention contemplates individual enantiomers and racemic mixtures.

As used herein, a “Cbl-b inhibitor” is a molecule that reduces, inhibits, or otherwise diminishes one or more of the biological activities of Cbl-b (e.g., ubiquitination, regulation of tyrosine kinase signaling, or regulation of immune cell activation and maintenance of peripheral tolerance). Inhibition using the Cbl-b inhibitor does not necessarily indicate a total elimination of the Cbl-b activity. Instead, the activity could decrease by a statistically significant amount including, for example, a decrease of at least about 5%, 10%, 15%, 20%, 25%, 30%, 35%, 40%, 45%, 50%, 55%, 60%, 65%, 70%, 75%, 80%, 85%, 95% or 100% of the activity of Cbl-b compared to an appropriate control. In some embodiments, the Cbl-b inhibitor reduces, inhibits, or otherwise diminishes the ubiquitination activity of Cbl-b. In some of these embodiments, the Cbl-b inhibitor reduces, inhibits, or otherwise diminishes the Cbl-b-mediated ubiquitination of tyrosine kinases. The presently disclosed compounds bind directly to Cbl-b and inhibit its ubiquitinating activity.

“Selectivity” and “selective” as used herein is a relative measure of the tendency for a compound to preferentially (e.g., in a statistically significant manner) associate with one target as opposed to another target (or group of targets). In some embodiments, the presently disclosed compounds reduce, inhibit, or otherwise diminishes the activity of Cbl-b greater than that of another target. For example, a selective Cbl-b inhibitor reduces at least one biological activity of Cbl-b by an amount that is statistically greater than the inhibitory effect of any other protein (e.g., other E3 ligases such as c-Cbl). In some embodiments, the activity of a selective inhibitor is reported as EC₅₀, IC₅₀, K_{D} or Kᵢ. In some embodiments, the activity of a selective inhibitor (measured as any one of EC₅₀, IC₅₀, K_{D} or Kᵢ) for Cbl-b is about 10 fold greater than the corresponding inhibitory activity for another target (e.g., other E3 ligase such as c-Cbl). In other embodiments the activity of the selective inhibitor for Cbl-b is at least about 15 fold greater, 20 fold greater, 25 fold greater, 30 fold greater, 40 fold greater or 50 fold greater than the corresponding inhibitory activity for another target (e.g., other E3 ligases such as c-Cbl). The presently disclosed compounds may or may not be a selective Cbl-b inhibitor.

A compound of the present invention may be tethered to a detectable moiety. It will be appreciated that such compounds are useful as imaging agents. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002, 41:2596-99 and Sun et al., Bioconjugate Chem., 2006, 17:52-57.

As used herein, the term “detectable moiety” is used interchangeably with the term “label” and relates to any moiety capable of being detected, e.g., primary labels and secondary labels. Primary labels, such as radioisotopes (e.g., tritium, ³²P, ³³P, ³⁵S, or ¹⁴C), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665). Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodanine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red. Rhodol Green. 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The terms “measurable affinity” and “measurably inhibit,” as used herein, means a measurable change in a Cbl-b ubiquitination activity between a sample comprising a compound of the present invention, or composition thereof, and a Cbl-b E3 ligase, and an equivalent sample comprising an Cbl-b E3 ligase, in the absence of said compound, or composition thereof.

### 3. Description of Exemplary Embodiments

### Formula I

As described above, in certain embodiments, the present invention provides a compound of formula I:

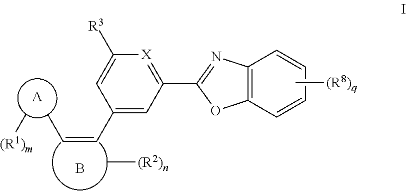

or a pharmaceutically acceptable salt thereof, wherein:
Ring A is a 5 membered heteroaryl ring having 1-3 nitrogen and 0-1 oxygen or sulfur;
R¹ is hydrogen, halogen, —CN, —OR, or an optionally substituted C₁₋₆ aliphatic;
Ring B is a divalent phenyl, or a divalent 5-6 membered heteroaryl ring having 1-4 heteroatoms independently selected from nitrogen, oxygen, and sulfur;
each R² is independently hydrogen, halogen, —CN, —CH₂OR, —CH(OR)R, —CRF₂, —CF₃, —OR, —SR, —NR₂, —SO₂R, —SO₂NR₂, —S(O)R, —C(O)R, —C(O)OR, —C(O)NR₂, —OC(O)R, —OC(O)NR₂, —NRC(O)OR, —NRC(O)R, —NRSO₂R; or an optionally substituted C₁₋₆ aliphatic or C₄₋₆ heterocycloalkyl;
X is CH or N;
R³ is fluoro, bromo, iodo, —CN, —OR^{3D}, —SR^{3A}, —N(R^{3A})(R^{3E}), —S(O)₂R^{3A}, —S(O)₂N(R^{3A})₂, —S(O)R^{3A}, —S(O)N(R^{3A})₂, —C(O)R^{3A}, —C(O)OR^{3A}, —C(O)N(R^{3A})₂, —C(O)N(R^{3A})OR^{3A}, —OC(O)R^{3A}, —OC(O)N(R^{3A})₂, —N(R^{3A})C(O)OR^{3A}, —N(R^{3A})C(O)R^{3A}, —N(R^{3A})C(O)N(R^{3A})₂, —N(R^{3A})C(NR^{3A})R^{3A}, —N(R^{3A})C(NR^{3A})N(R^{3A})₂, —N(R^{3A})N(R^{3A})₂, —N(R^{3A})S(O)₂N(R^{3A})₂, —N(R^{3A})S(O)₂R^{3A}, —N═S(O)(R^{3A})₂, —S(NR^{3A})(O)R^{3A}, —N(R^{3A})S(O)R³, —N(R^{3A})CN, —P(O)(OR^{3A})₂, —P(O)(R^{3A})₂, or an optionally substituted group selected from C₁₋₆ alkyl, alkenyl, or alkynyl; phenyl; a 4-7 membered saturated or partially unsaturated monocyclic carbocyclic ring; a 5-6 membered monocyclic heteroaryl ring having 1-4 heteroatoms independently selected from nitrogen, oxygen, and sulfur; a 4-8 membered saturated or partially unsaturated monocyclic heterocyclic ring having 1-2 heteroatoms independently selected from nitrogen, oxygen, and sulfur; and a 6-10 membered saturated or partially unsaturated spirocyclic ring having 0-3 heteroatoms independently selected from nitrogen, oxygen, and sulfur;
each R^{3A} are independently hydrogen, —CN, halogen, or an optionally substituted group selected from C₁₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic carbocyclic ring having 1-2 heteroatoms independently selected from nitrogen, oxygen, and sulfur; or:
two R^{3A} groups on the same atom are optionally taken together with the atom to form an optionally substituted ring selected from a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 7-10 membered saturated or partially unsaturated bicyclic heterocyclic ring having 1-4 heteroatoms independently selected from nitrogen, oxygen, phosphorus, and sulfur; a 5-8 membered saturated or partially unsaturated bridged bicyclic ring having 0-3 heteroatoms independently selected from nitrogen, oxygen, phosphorus, and sulfur; and a 6-10 membered saturated or partially unsaturated spirocyclic ring having 0-3 heteroatoms independently selected from nitrogen, oxygen, phosphorus, and sulfur;
R^{3B} is hydrogen, halogen, —CN, —OR^{3C}, —SR, —N(R^{3A})(R^{3A}), —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —NRC(O)OR, —NRC(O)R, —NRC(O)NR₂, —NRC(NR)NR₂, —NRNR₂, —NRS(O)₂NR₂, —NRS(O)₂R, —N═S(O)R₂, —S(NR)(O)R, —NRS(O)R, —NRCN, —P(O)R₂, —P(O)(OR)₂; or an optionally substituted group selected from C₁₋₆ aliphatic; a phenyl ring; a 4-7 membered saturated or partially unsaturated monocyclic carbocyclic ring; a 5-6 membered monocyclic heteroaryl ring having 1-4 heteroatoms independently selected from nitrogen, oxygen, and sulfur; a 4-8 membered saturated or partially unsaturated monocyclic heterocyclic ring having 1-2 heteroatoms independently selected from nitrogen, oxygen, and sulfur; and a 7-10 membered partially unsaturated or heteroaromatic bicyclic heterocyclic ring having 1-4 heteroatoms independently selected from nitrogen, oxygen, and sulfur;
each R^{3C} are independently an optionally substituted group selected from C₁₋₆ aliphatic; phenyl: naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic carbocyclic ring having 1-2 heteroatoms independently selected from nitrogen, oxygen, and sulfur;
R^{3D} is hydrogen, a substituted ethyl, or an optionally substituted group selected from methyl or C₃₋₆ aliphatic; phenyl: naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic carbocyclic ring having 1-2 heteroatoms independently selected from nitrogen, oxygen, and sulfur;
R^{3E} is an C₂ aliphatic substituted with v instances of R^{3B}, a substituted 2-hydroxyethyl, or an optionally substituted group selected from methyl or C₃₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; and a 6-10 membered saturated or partially unsaturated spirocyclic ring having 0-3 heteroatoms independently selected from nitrogen, oxygen, and sulfur; a 6-11 membered saturated or partially unsaturated bicyclic carbocyclic ring having 1-2 heteroatoms independently selected from nitrogen, oxygen, and sulfur; or:
R^{3A} and R^{3E} groups on the same nitrogen are optionally taken together with the nitrogen to form an optionally substituted 3-7 membered saturated or partially unsaturated monocyclic heterocyclic ring having 0-2 heteroatoms, in addition to the nitrogen from which R^{3A} and R^{3E} are attached, independently selected from nitrogen, oxygen, and sulfur; a 5-8 membered saturated or partially unsaturated bridged bicyclic ring having 0-2 heteroatoms, in addition to the nitrogen from which R^{3A} and R^{3E} are attached, independently selected from nitrogen, oxygen, and sulfur; and a 6-10 membered saturated or partially unsaturated spirocyclic ring having 0-2 heteroatoms, in addition to the nitrogen from which R^{3A} and R^{3E} are attached, independently selected from nitrogen, oxygen, and sulfur;
each R⁸ is independently hydrogen, oxo, halogen, —CN, —NO₂, —CHF₂, —CF₃, —OR, —(OCH₂CH₂)₁₋₁₀NR₂, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —NRC(O)OR, —NRC(O)R, —NRC(O)NR₂, —NRC(NR)NR₂, —NRNR₂, —NRS(O)₂NR₂, —NRS(O)₂R, —N═S(O)R₂, —S(NR)(O)R, —NRS(O)R, —NRCN, —P(O)R₂, —P(O)(OR)₂—CH₂NR(CH₂CH₂O)₁₋₁₀CH₂CH₂NR₂; or an optionally substituted C₁₋₆ aliphatic;
each R is independently hydrogen, or an optionally substituted group selected from C₁₋₆ aliphatic: phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic carbocyclic ring having 1-2 heteroatoms independently selected from nitrogen, oxygen, and sulfur; or:
   two R groups on the same atom are optionally taken together with the atom to form an optionally substituted 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorus, and sulfur;
m is 0, 1, or 2;
n is 0, 1, 2, or 3;
q is 0, 1, 2, or 3; and
each instance of v is independently 0, 1, 2, 3, 4, or 5.

### Formula II

As described above, in certain embodiments, the present invention provides a compound of formula II:

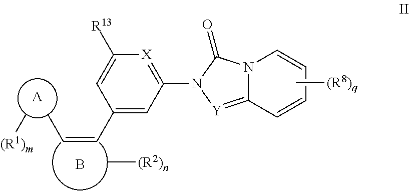

or a pharmaceutically acceptable salt thereof, wherein:
Ring A is a 5 membered heteroaryl ring having 1-3 nitrogen and 0-1 oxygen or sulfur;
R¹ is hydrogen, halogen, —CN, —OR, or an optionally substituted C₁₋₆ aliphatic;
Ring B is a divalent phenyl, or a divalent 5-6 membered heteroaryl ring having 1-4 heteroatoms independently selected from nitrogen, oxygen, and sulfur;
each R² is independently hydrogen, halogen, —CN, —CH₂OR, —CH(OR)R, —CRF₂, —CF₃, —OR, —SR, —NR₂, —SO₂R, —SO₂NR₂, —S(O)R, —C(O)R, —C(O)OR, —C(O)NR₂, —OC(O)R, —OC(O)NR₂, —NRC(O)OR, —NRC(O)R, —NRSO₂R; or an optionally substituted C₁₋₆ aliphatic or C₄₋₅ heterocycloalkyl;
X is CH or N;
Y is CH or N;
R¹³ is hydrogen, halogen, —CN, —OR^{3A}, —SR³, —N(R^{3A})(R^{3A}), —S(O)₂R^{3A}, —S(O)₂N(R^{3A})₂, —S(O)R^{3A}, —S(O)N(R^{3A})₂, —C(O)R^{3A}, —C(O)OR^{3A}, —C(O)N(R^{3A})₂, —C(O)N(R^{3A})OR^{3A}, —OC(O)R^{3A}, —OC(O)N(R^{3A})₂, —N(R^{3A})C(O)OR^{3A}, —N(R^{3A})C(O)R^{3A}, —N(R^{3A})C(O)N(R^{3A})₂, —N(R^{3A})C(NR^{3A})R^{3A}, —N(R^{3A})C(NR^{3A})N(R^{3A})₂, —N(R^{3A})N(R^{3A})₂, —N(R^{3A})S(O)₂N(R^{3A})₂, —N(R^{3A})S(O)₂R^{3A}, —N═S(O)(R^{3A})₂, —S(NR^{3A})(O)R^{3A}, —N(R^{3A})S(O)R^{3A}, —N(R^{3A})CN, —P(O)(R^{3A})OR^{3A}, —P(O)(R^{3A})₂, or an optionally substituted group selected from C₁₋₆ aliphatic; a phenyl ring; a 4-7 membered saturated or partially unsaturated monocyclic carbocyclic ring; a 5-6 membered monocyclic heteroaryl ring having 1-4 heteroatoms independently selected from nitrogen, oxygen, and sulfur; a 4-8 membered saturated or partially unsaturated monocyclic heterocyclic ring having 1-2 heteroatoms independently selected from nitrogen, oxygen, and sulfur; and a 6-10 membered saturated or partially unsaturated spirocyclic ring having 0-3 heteroatoms independently selected from nitrogen, oxygen, and sulfur;
each R^{3A} are independently hydrogen, —CN, halogen, or an optionally substituted group selected from C₁₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic carbocyclic ring having 1-2 heteroatoms independently selected from nitrogen, oxygen, and sulfur; or:
two R^{3A} groups on the same atom are optionally taken together with the atom to form an optionally substituted ring selected from a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 7-10 membered saturated or partially unsaturated bicyclic heterocyclic ring having 1-4 heteroatoms independently selected from nitrogen, oxygen, phosphorus, and sulfur; a 5-8 membered saturated or partially unsaturated bridged bicyclic ring having 0-3 heteroatoms independently selected from nitrogen, oxygen, phosphorus, and sulfur; and a 6-10 membered saturated or partially unsaturated spirocyclic ring having 0-3 heteroatoms independently selected from nitrogen, oxygen, phosphorus, and sulfur;
each R⁸ is independently hydrogen, oxo, halogen, —CN, —NO₂, —CHF₂, —CF₃, —OR, —(OCH₂CH₂)₁₋₁₀NR₂, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —NRC(O)OR, —NRC(O)R, —NRC(O)NR₂, —NRC(NR)NR₂, —NRNR₂, —NRS(O)₂NR₂, —NRS(O)₂R, —N═S(O)R₂, —S(NR)(O)R, —NRS(O)R, —NRCN, —P(O)R₂, —P(O)(OR)₂—CH₂NR(CH₂CH₂O)₁₋₁₀CH₂CH₂NR₂; or an optionally substituted C₁₋₆aliphatic;
each R is independently hydrogen, or an optionally substituted group selected from C₁₋₆ aliphatic: phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic carbocyclic ring having 1-2 heteroatoms independently selected from nitrogen, oxygen, and sulfur; or:
   two R groups on the same atom are optionally taken together with the atom to form an optionally substituted 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorus, and sulfur;
m is 0, 1, or 2;
n is 0, 1, 2, or 3; and
q is 0, 1, 2, or 3.

### Formula III

As described above, in certain embodiments, the present invention provides a compound of formula III:

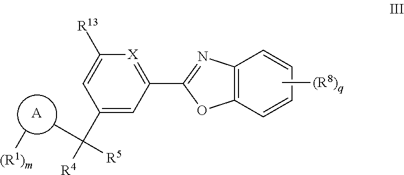

or a pharmaceutically acceptable salt thereof, wherein:
Ring A is a 5 membered heteroaryl ring having 1-3 nitrogen and 0-1 oxygen or sulfur;
R¹ is hydrogen, halogen, —CN, —OR, or an optionally substituted C₁₋₆ aliphatic;
R⁴ and R⁵ are each independently hydrogen or an optionally substituted group selected from C₁₋₆ aliphatic, 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; or:
R⁴ and R⁵ are optionally taken together with the carbon they are attached to form

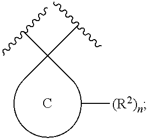

Ring C is a divalent spiro-fused 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur;
each R² is independently hydrogen, halogen, —CN, —CH₂OR, —CH(OR)R, —CRF₂, —CF₃, —OR, —SR, —NR₂, —SO₂R, —SO₂NR₂, —S(O)R, —C(O)R, —C(O)OR, —C(O)NR₂, —OC(O)R, —OC(O)NR₂, —NRC(O)OR, —NRC(O)R, —NRSO₂R; or an optionally substituted C₁₋₆ aliphatic or C₄₋₆ heterocycloalkyl;
X is CH or N;
R¹³ is hydrogen, halogen, —CN, —OR^{3A}, —SR^{3A}, —N(R^{3A})(R^{3A}), —S(O)₂R^{3A}, —S(O)₂N(R^{3A})₂, —S(O)R^{3A}, —S(O)N(R^{3A})₂, —C(O)R^{3A}, —C(O)OR^{3A}, —C(O)N(R^{3A})₂, —C(O)N(R^{3A})OR^{3A}, —OC(O)R^{3A}, —OC(O)N(R^{3A})₂, —N(R^{3A})C(O)OR^{3A}, —N(R^{3A})C(O)R^{3A}, —N(R^{3A})C(O)N(R^{3A})₂, —N(R^{3A})C(NR^{3A})R^{3A}, —N(R^{3A})C(NR^{3A})N(R^{3A})₂, —N(R^{3A})N(R^{3A})₂, —N(R^{3A})S(O)₂N(R^{3A})₂, —N(R^{3A})S(O)₂R^{3A}, —N═S(O)(R^{3A}), —S(NR^{3A})(O)R^{3A}, —N(R^{3A})S(O)R^{3A}, —N(R^{3A})CN, —P(O)(R^{3A})OR^{3A}, —P(O)(R^{3A})₂, or an optionally substituted group selected from C₁₋₆ aliphatic: a phenyl ring; a 4-7 membered saturated or partially unsaturated monocyclic carbocyclic ring; a 5-6 membered monocyclic heteroaryl ring having 1-4 heteroatoms independently selected from nitrogen, oxygen, and sulfur; a 4-8 membered saturated or partially unsaturated monocyclic heterocyclic ring having 1-2 heteroatoms independently selected from nitrogen, oxygen, and sulfur; and a 6-10 membered saturated or partially unsaturated spirocyclic ring having 0-3 heteroatoms independently selected from nitrogen, oxygen, and sulfur;
each R^{3A} are independently hydrogen, —CN, halogen, or an optionally substituted group selected from C₁₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic carbocyclic ring having 1-2 heteroatoms independently selected from nitrogen, oxygen, and sulfur; or:
two R^{3A} groups on the same atom are optionally taken together with the atom to form an optionally substituted ring selected from a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 7-10 membered saturated or partially unsaturated bicyclic heterocyclic ring having 1-4 heteroatoms independently selected from nitrogen, oxygen, phosphorus, and sulfur; a 5-8 membered saturated or partially unsaturated bridged bicyclic ring having 0-3 heteroatoms independently selected from nitrogen, oxygen, phosphorus, and sulfur; and a 6-10 membered saturated or partially unsaturated spirocyclic ring having 0-3 heteroatoms independently selected from nitrogen, oxygen, phosphorus, and sulfur;
each R⁸ is independently hydrogen, oxo, halogen, —CN, —NO₂, —CHF₂, —CF₃, —OR, —(OCH₂CH₂)₁₋₁₀NR₂, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —NRC(O)OR, —NRC(O)R, —NRC(O)NR₂, —NRC(NR)NR₂, —NRNR₂, —NRS(O)₂NR₂, —NRS(O)₂R, —N═S(O)R₂, —S(NR)(O)R, —NRS(O)R, —NRCN, —P(O)R₂, —P(O)(OR)₂—CH₂NR(CH₂CH₂O)₁₋₁₀CH₂CH₂NR₂; or an optionally substituted C₁₋₆aliphatic;
each R is independently hydrogen, or an optionally substituted group selected from C₁₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic carbocyclic ring having 1-2 heteroatoms independently selected from nitrogen, oxygen, and sulfur; or:
   two R groups on the same atom are optionally taken together with the atom to form an optionally substituted 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorus, and sulfur;
m is 0, 1, or 2;
n is 0, 1, 2, or 3; and
q is 0, 1, 2, or 3,
wherein the compound of formula III is not:

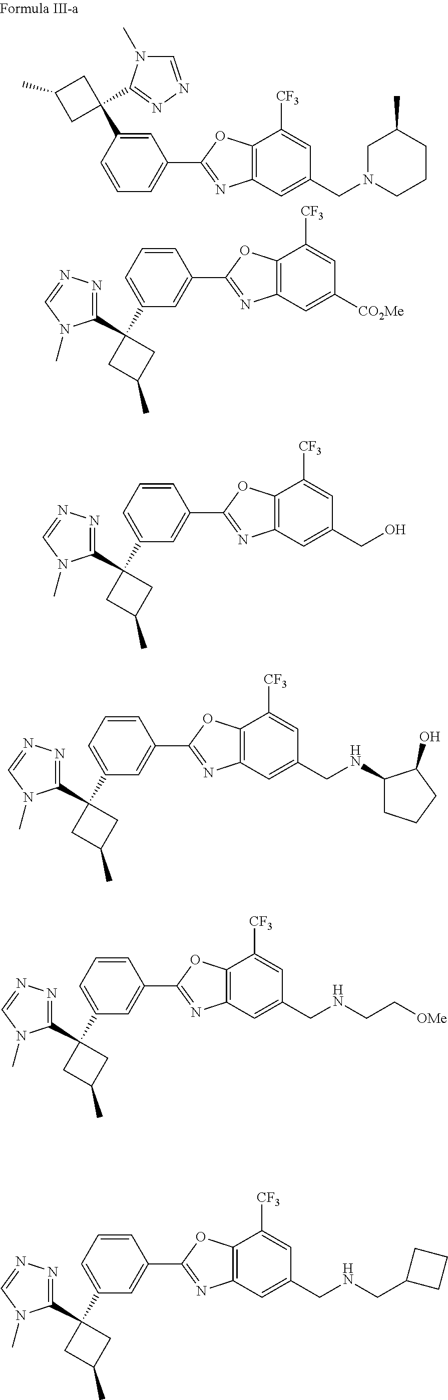

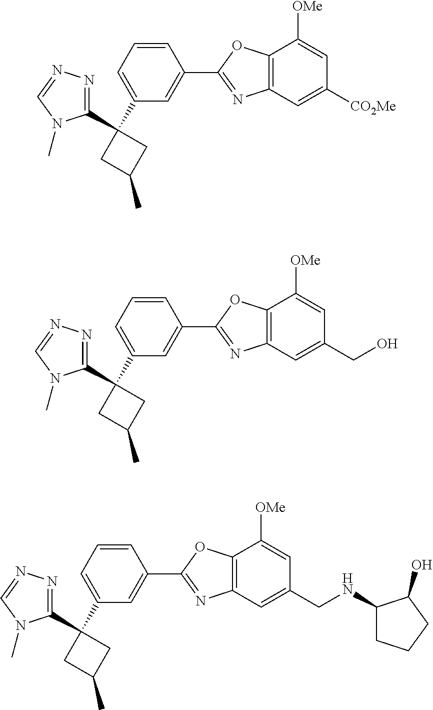 [SMILES: COC(=O)c1cc(OC)c2oc(-c3cccc([C@]4(c5nncn5C)C[C@@H](C)C4)c3)nc2c1 ; COc1cc(CO)cc2nc(-c3cccc([C@]4(c5nncn5C)C[C@@H](C)C4)c3)oc12 ; COc1cc(CN[C@@H]2CCC[C@@H]2O)cc2nc(-c3cccc([C@]4(c5nncn5C)C[C@@H](C)C4)c3)oc12]

As described above, in certain embodiments, the present invention provides a compound of formula III-a:

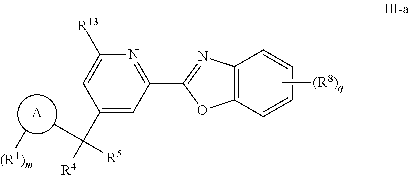

or a pharmaceutically acceptable salt thereof, wherein:
Ring A is a 5 membered heteroaryl ring having 1-3 nitrogen and 0-1 oxygen or sulfur;
R¹ is hydrogen, halogen, —CN, —OR, or an optionally substituted C₁₋₆ aliphatic;
R⁴ and R⁵ are each independently hydrogen or an optionally substituted group selected from C₁₋₆ aliphatic, 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; or:
R⁴ and R⁵ are optionally taken together with the carbon they are attached to form

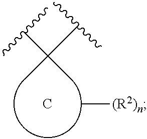

Ring C is a divalent spiro-fused 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur;
each R² is independently hydrogen, halogen, —CN, —CH₂OR, —CH(OR)R, —CRF₂, —CF₃, —OR, —SR, —NR₂, —SO₂R, —SO₂NR₂, —S(O)R, —C(O)R, —C(O)OR, —C(O)NR₂, —OC(O)R, —OC(O)NR₂, —NRC(O)OR, —NRC(O)R, —NRSO₂R; or an optionally substituted C₁₋₆ aliphatic or C₄₋₆ heterocycloalkyl; R¹³ is hydrogen, halogen, —CN, —OR^{3A}, —SR^{3A}, —N(R^{3A})(R^{3A}), —S(O)₂R^{3A}, —S(O)₂N(R^{3A})₂, —S(O)R^{3A}—S(O)N(R^{3A})₂, —C(O)R^{3A}, —C(O)OR^{3A}, —C(O)N(R^{3A})₂, —C(O)N(R^{3A})OR^{3A}, —OC(O)R^{3A}, —OC(O)N(R^{3A})₂, —N(R^{3A})C(O)OR^{3A}, —N(R^{3A})C(O)R^{3A}, —N(R^{3A})C(O)N(R^{3A})₂, —N(R^{3A})C(NR^{3A})R^{3A}, —N(R^{3A})C(NR^{3A})N(R^{3A})₂, —N(R^{3A})N(R^{3A})₂, —N(R^{3A})S(O)₂N(R^{3A})₂, —N(R^{3A})S(O)₂R^{3A}, —N═S(O)(R^{3A})₂, —S(NR^{3A})(O)R^{3A}, —N(R^{3A})S(O)R^{3A}, —N(R^{3A})CN, —P(O)(R^{3A})OR^{3A}, —P(O)(R^{3A})₂, or an optionally substituted group selected from C₁₋₆ aliphatic; a phenyl ring; a 4-7 membered saturated or partially unsaturated monocyclic carbocyclic ring; a 5-6 membered monocyclic heteroaryl ring having 1-4 heteroatoms independently selected from nitrogen, oxygen, and sulfur; a 4-8 membered saturated or partially unsaturated monocyclic heterocyclic ring having 1-2 heteroatoms independently selected from nitrogen, oxygen, and sulfur; and a 6-10 membered saturated or partially unsaturated spirocyclic ring having 0-3 heteroatoms independently selected from nitrogen, oxygen, and sulfur;
each R^{3A} are independently hydrogen, —CN, halogen, or an optionally substituted group selected from C₁₋₆ aliphatic; phenyl: naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic carbocyclic ring having 1-2 heteroatoms independently selected from nitrogen, oxygen, and sulfur; or:
two R^{3A} groups on the same atom are optionally taken together with the atom to form an optionally substituted ring selected from a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 7-10 membered saturated or partially unsaturated bicyclic heterocyclic ring having 1-4 heteroatoms independently selected from nitrogen, oxygen, phosphorus, and sulfur; a 5-8 membered saturated or partially unsaturated bridged bicyclic ring having 0-3 heteroatoms independently selected from nitrogen, oxygen, phosphorus, and sulfur; and a 6-10 membered saturated or partially unsaturated spirocyclic ring having 0-3 heteroatoms independently selected from nitrogen, oxygen, phosphorus, and sulfur;
each R⁸ is independently hydrogen, oxo, halogen, —CN, —NO₂, —CHF₂, —CF₃, —OR, —(OCH₂CH₂)₁₋₁₀NR₂, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —NRC(O)OR, —NRC(O)R, —NRC(O)NR₂, —NRC(NR)NR₂, —NRNR₂, —NRS(O)₂NR₂, —NRS(O)₂R, —N═S(O)R₂, —S(NR)(O)R, —NRS(O)R, —NRCN, —P(O)R₂, —P(O)(OR)₂—CH₂NR(CH₂CH₂O)₁₋₁₀CH₂CH₂NR₂; or an optionally substituted C₁₋₆aliphatic;
each R is independently hydrogen, or an optionally substituted group selected from C₁₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic carbocyclic ring having 1-2 heteroatoms independently selected from nitrogen, oxygen, and sulfur; or:
   two R groups on the same atom are optionally taken together with the atom to form an optionally substituted 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorus, and sulfur;
m is 0, 1, or 2;
n is 0, 1, 2, or 3; and
q is 0, 1, 2, or 3.

As defined generally above, Ring A is a 5 membered heteroaryl ring having 1-3 nitrogen and 0-1 oxygen or sulfur.

In some embodiments, Ring A is a 5 membered heteroaryl ring having 1-3 nitrogen and 0-1 oxygen or sulfur.

In some embodiments, Ring A is a furanyl, imidazolyl, isothiazolyl, isoxazolyl, oxadiazolyl, oxazolyl, pyrazolyl, pyrrolyl, thiazolyl, thienyl, triazinyl, or triazolyl.

In some embodiments, Ring A is selected from

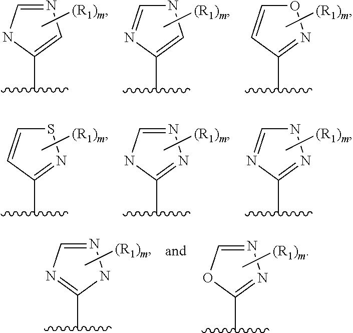

In some embodiments, Ring A is

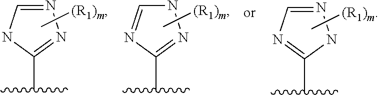

In certain embodiments, Ring A is

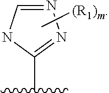

In some embodiments, Ring A is selected from those depicted in Table 1, below.

As defined generally above, R¹ is hydrogen, halogen, —CN, —OR, or an optionally substituted C₁₋₆ aliphatic.

In some embodiments, R¹ is hydrogen. In some embodiments, R¹ is halogen. In some embodiments, R¹ is —CN. In some embodiments. R¹ is —OR. In some embodiments, R¹ is an optionally substituted C₁₋₆ aliphatic.

In some embodiments, R¹ is a C₁₋₆ aliphatic.

In some embodiments, R¹ is a methyl, ethyl, n-propyl, or isopropyl.

In certain embodiments, R¹ is methyl.

In some embodiments, R¹ is selected from those depicted in Table 1, below.

As defined generally above, m is 0, 1, 2, or 3.

In some embodiments, m is 0, 1, 2, or 3.

In some embodiments, m is 0. In some embodiments, m is 1. In some embodiments, m is 2. In some embodiments, m is 3.

In some embodiments, m is 0 or 1. In some embodiments, m is 0, 1, or 2. In some embodiments, m is 1 or 2. In some embodiments, in is 1, 2, or 3. In some embodiments, m is 2 or 3.

In some embodiments, in is selected from the values represented in the compounds depicted in Table 1, below.

In certain embodiments, Ring A and its R¹ substituent is

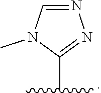 [SMILES: *c1nncn1C]

In some embodiments, Ring A together with its R¹ substituents is selected from those depicted in Table 1, below.

As defined generally above. Ring B is a divalent phenyl, or a divalent 5-6 membered heteroaryl ring having 1-4 heteroatoms independently selected from nitrogen, oxygen, and sulfur.

In some embodiments, Ring B is a divalent phenyl. In some embodiments, Ring B is a divalent 5-6 membered heteroaryl ring having 1-4 heteroatoms independently selected from nitrogen, oxygen, and sulfur.

In some embodiments, Ring B is a divalent 5-membered heteroaryl ring having 1-2 heteroatoms independently selected from nitrogen, oxygen, and sulfur. In some embodiments. Ring B is a divalent 6-membered heteroaryl ring having 1-2 nitrogen.

In some embodiments, Ring B is selected from

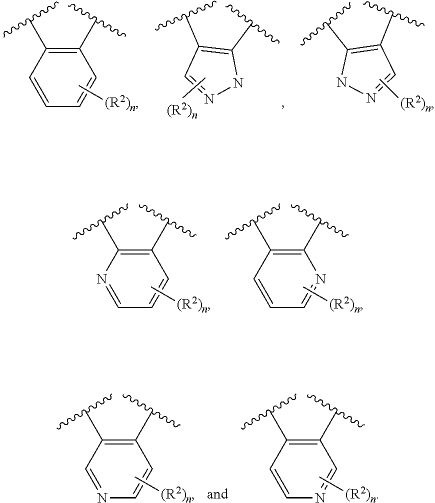

In some embodiments, Ring B is selected from

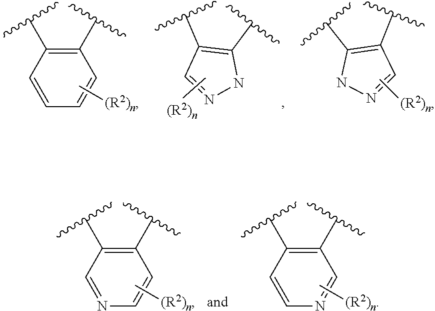

In some embodiments, Ring B is selected from those depicted in Table 1, below.

As defined generally above, R⁴ and R⁵ are each independently hydrogen or an optionally substituted group selected from C₁₋₆ aliphatic, 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur, or R⁴ and R⁵ are optionally taken together with the carbon they are attached to form

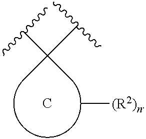

In some embodiments, R⁴ is an optionally substituted group selected from C₁₋₆ aliphatic. 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur.

In some embodiments, R⁴ is hydrogen. In some embodiments, R⁴ is an optionally substituted C₁₋₆ aliphatic. In some embodiments. R⁴ is an optionally substituted 3-7 membered saturated or partially unsaturated monocyclic carbocyclic ring. In some embodiments, R⁴ is an optionally substituted 3-7 membered saturated or partially unsaturated monocyclic heterocyclic ring having 1-2 heteroatoms independently selected from nitrogen, oxygen, and sulfur.

In some embodiments, R⁵ is an optionally substituted group selected from C₁₋₆ aliphatic, 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur.

In some embodiments, R⁵ is hydrogen. In some embodiments, R⁵ is an optionally substituted C₁₋₆ aliphatic. In some embodiments, R⁵ is an optionally substituted 3-7 membered saturated or partially unsaturated monocyclic carbocyclic ring. In some embodiments, R⁵ is an optionally substituted 3-7 membered saturated or partially unsaturated monocyclic heterocyclic ring having 1-2 heteroatoms independently selected from nitrogen, oxygen, and sulfur.

In some embodiments, R⁴ is hydrogen and R is an optionally substituted C₁₋₆ aliphatic.

In some embodiments, R⁴ is hydrogen and R is a C₁₋₆ alkyl.

In some embodiments, R⁴ is hydrogen and R⁵ is an optionally substituted 3-7 membered saturated or partially unsaturated monocyclic heterocyclic ring having 1-2 heteroatoms independently selected from nitrogen, oxygen, and sulfur.

In some embodiments, R⁴ is hydrogen and R⁵ is an optionally substituted 3-7 membered saturated or partially unsaturated monocyclic carbocyclic ring.

In some embodiments, R⁴ is hydrogen and R⁵ is cyclopropyl, cyclobutyl, oxetanyl, cyclopentyl, or tetrahydrofuryl.

In some embodiments, R⁴ is hydrogen and R is ethyl,

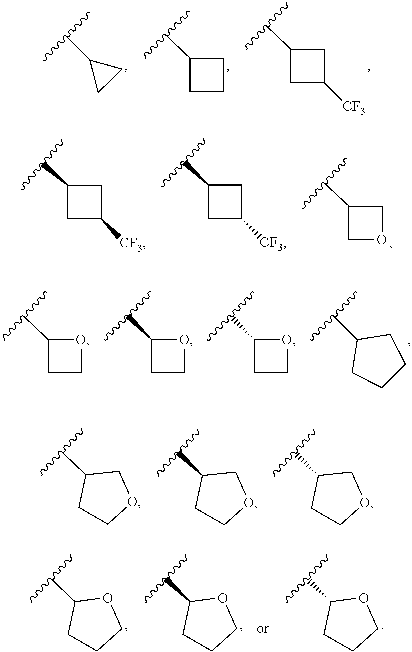

In some embodiments, R⁴ and R⁵ are taken together with the carbon they are attached to form

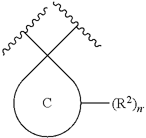

In some embodiments, R⁴ and R⁵ are selected from those depicted in Table 1, below.

As defined generally above, Ring C is a divalent spiro-fused 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur.

In some embodiments, Ring C is a divalent spiro-fused 3-7 membered saturated or partially unsaturated monocyclic carbocyclic ring. In some embodiments, Ring C is a 3-7 membered saturated or partially unsaturated monocyclic heterocyclic ring having 1-2 heteroatoms independently selected from nitrogen, oxygen, and sulfur.

In some embodiments, Ring C is

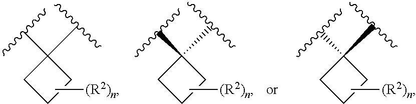

In some embodiments, Ring C is selected from those depicted in Table 1, below.

As defined generally above, each R² is independently hydrogen, halogen, —CN, —CH₂OR, —CH(OR)R, —CRF₂, —CF₃, —OR, —SR, —NR₂, —SO₂R, —SO₂NR₂, —S(O)R, —C(O)R, —C(O)OR, —C(O)NR₂, —OC(O)R, —OC(O)NR₂, —NRC(O)OR, —NRC(O)R, —NRSO₂R; or an optionally substituted C₁₋₆ aliphatic or C₄₋₆heterocycloalkyl.

In some embodiments, R² is hydrogen. In some embodiments, R² is halogen. In some embodiments. R² is —CN. In some embodiments, R² is —CH₂OR. In some embodiments. R² is —CH(OR)R. In some embodiments, R² is —CRF₂. In some embodiments, R² is —CF₃. In some embodiments, R² is —OR. In some embodiments, R² is —SR. In some embodiments, R² is —NR₂. In some embodiments, R² is —SO₂R. In some embodiments, R² is —SO₂NR₂. In some embodiments, R² is —S(O)R. In some embodiments, R² is —C(O)R. In some embodiments, R² is —C(O)OR. In some embodiments, R² is —C(O)NR₂. In some embodiments, R² is —OC(O)R. In some embodiments. R² is —OC(O)NR₂. In some embodiments, R² is —NRC(O)OR. In some embodiments, R² is —NRC(O)R. In some embodiments, R² is —NRSO₂R. In some embodiments, R² is an optionally substituted C₁₋₆ aliphatic. In some embodiments, R² is an optionally substituted C₄₋₆ heterocycloalkyl.

In some embodiments each R² is independently selected from halogen, —CN, —CH₂OR, —CH(OR)R, —OR, —NR₂, —C(O)R, —C(O)OR, —C(O)NR₂, —NRSO₂R; or an optionally substituted C₁₋₆ aliphatic.

In certain embodiments, R² is fluoro, choro, —CN, methyl, —CH₂OH, —CH₂OMe, —OMe, —CONH₂, —C(O)Me, —CH(OH)Me, —CO₂Me, or —NHSO₂Me.

In some embodiments, R² is selected from those depicted in Table 1, below.

As defined generally above, n is 0, 1, 2, or 3.

In some embodiments, n is 0, 1, 2, or 3.

In some embodiments, n is 0. In some embodiments, n is 1. In some embodiments, n is 2.

In some embodiments, n is 0 or 1. In some embodiments, n is 0, 1, or 2. In some embodiments, n is 1 or 2. In some embodiments, n is 1, 2, or 3. In some embodiments, n is 2 or 3.

In some embodiments, n is selected from the values represented in the compounds depicted in Table 1, below.

In certain embodiments, Ring B and its R² substituents are selected from

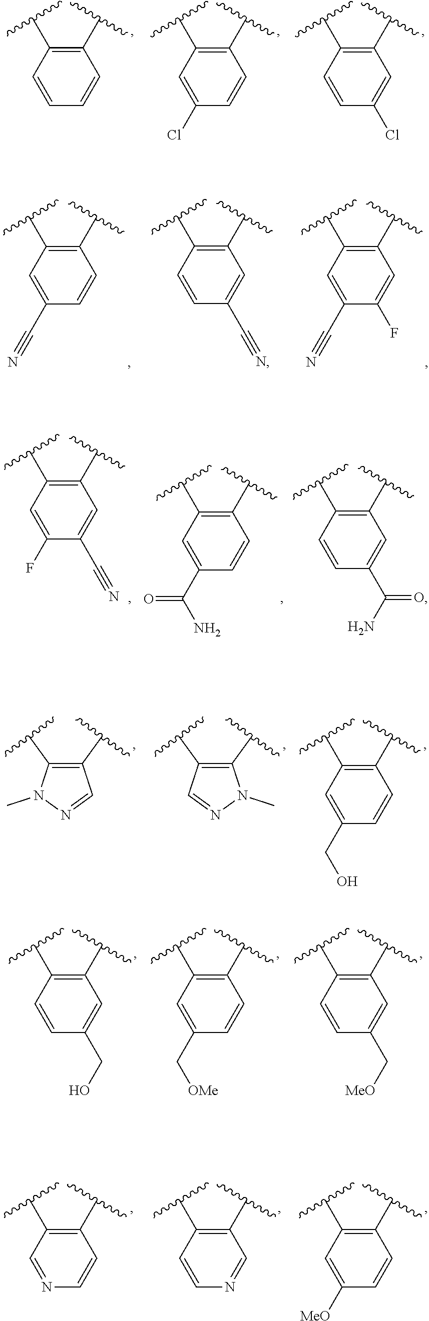

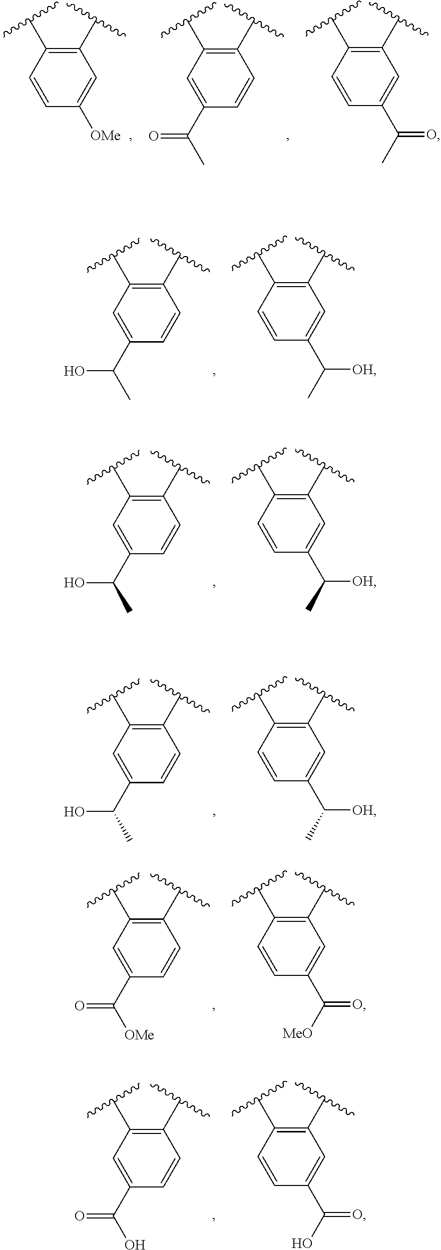

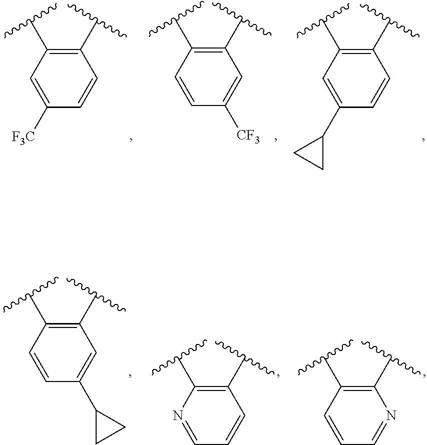 [SMILES: *c1ccc(C(F)(F)F)cc1* ; *c1ccc(C(F)(F)F)cc1* ; *c1ccc(C2CC2)cc1* ; *c1ccc(C2CC2)cc1* ; *c1cccnc1* ; *c1cccnc1*]

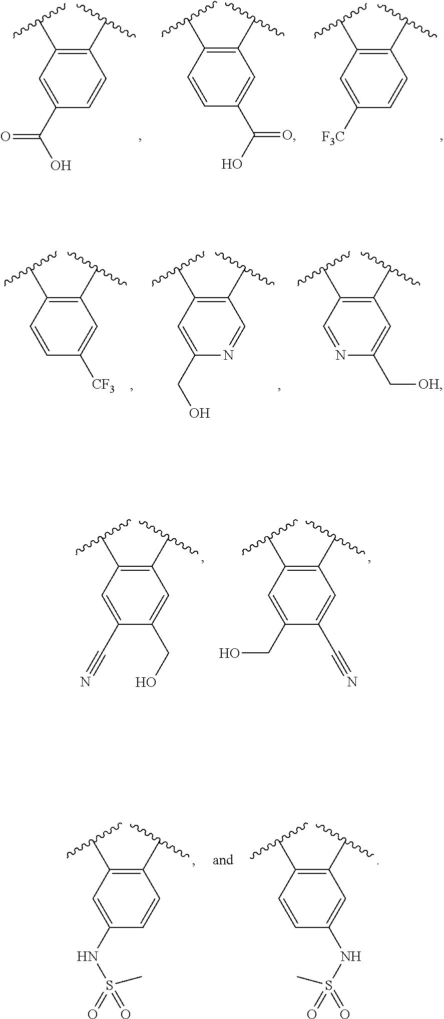

In certain embodiments. Ring B and its R² substituents are selected from N

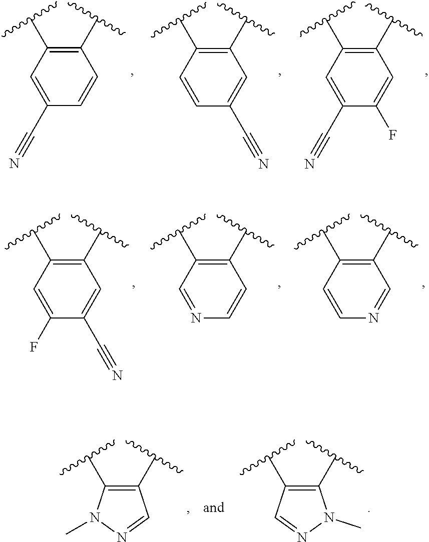

In some embodiments, Ring B together with its R² substituents is selected from those depicted in Table 1, below.

In some embodiments, Ring C and its R² substituents are selected from

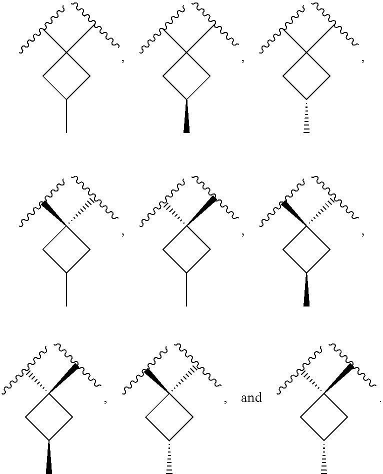

As defined generally above, X is CH or N.

In some embodiments, X is CH. In some embodiments, X is N.

In embodiments, X is selected from those depicted in Table 1, below.

As defined generally above. Y is CH or N.

In some embodiments, Y is CH. In some embodiments, Y is N.

In embodiments, Y is selected from those depicted in Table 1, below.

As defined generally above, R³ is fluoro, bromo, iodo, —CN, —ORD, —SR^{3A}, —N(R^{3A})(R^{3E}) S(O)₂R^{3A}, —S(O)₂N(R^{3A})₂, —S(O)R^{3A}, —S(O)N(R^{3A})₂, —C(O)R^{3A}, —C(O)OR^{3A}, —C(O)N(R^{3A})₂, —C(O)N(R^{3A})OR^{3A}, —OC(O)R^{3A}, —OC(O)N(R^{3A})₂, —N(R^{3A})C(O)OR^{3A}, —N(R^{3A})C(O)R^{3A}, —N(R^{3A})C(O)N(R^{3A})₂, —N(R^{3A})C(NR^{3A})R^{3A}, —N(R^{3A})C(NR^{3A})N(R^{3A})₂, —N(R^{3A})N(R^{3A})₂, —N(R^{3A})S(O)₂N(R^{3A})₂, —N(R^{3A})S(O)₂R^{3A}, —N═S(O)(R^{3A})₂, —S(NR^{3A})(O)R³, —N(R^{3A})S(O)R^{3A}, —N(R^{3A})CN, —P(O)(OR^{3A})₂, —P(O)(R^{3A})₂, or an optionally substituted group selected from C₁₋₆ alkyl, alkenyl, or alkynyl; phenyl; a 4-7 membered saturated or partially unsaturated monocyclic carbocyclic ring; a 5-6 membered monocyclic heteroaryl ring having 1-4 heteroatoms independently selected from nitrogen, oxygen, and sulfur; a 4 or 6-8 membered saturated or partially unsaturated monocyclic heterocyclic ring having 1-2 heteroatoms independently selected from nitrogen, oxygen, and sulfur; and a 6-10 membered saturated or partially unsaturated spirocyclic ring having 0-3 heteroatoms independently selected from nitrogen, oxygen, and sulfur.

In some embodiments, R³ is fluoro. In some embodiments, R³ is bromo. In some embodiments, R³ is iodo. In some embodiments, R³ is —CN. In some embodiments, R³ is —OR^{3D}. In some embodiments, R³ is —SR^{3A}. In some embodiments, R³ is —N(R^{3A})(R^{3E}). In some embodiments, R³ is —S(O)₂R^{3A}. In some embodiments, R³ is —S(O)₂N(R^{3A})₂. In some embodiments, R³ is —S(O)R^{3A}. In some embodiments, R³ is —S(O)N(R^{3A})₂. In some embodiments, R³ is —C(O)R^{3A}. In some embodiments, R³ is —C(O)OR^{3A}. In some embodiments, R³ is —C(O)N(R^{3A})₂. In some embodiments, R³ is —C(O)N(R^{3A})OR^{3A}. In some embodiments, R³ is —OC(O)R^{3A}. In some embodiments, R³ is —OC(O)N(R^{3A})₂. In some embodiments, R³ is —N(R^{3A})C(O)OR^{3A}. In some embodiments, R³ is —N(R^{3A})C(O)R^{3A}. In some embodiments, R³ is —N(R^{3A})C(O)N(R^{3A})₂. In some embodiments, R³ is —N(R^{3A})C(NR^{A})R^{3A}. In some embodiments. R³ is —N(R^{3A})C(NR^{3A})N(R^{3A})₂. In some embodiments, R³ is —N(R^{3A})N(R^{3A})₂. In some embodiments. R is —N(R^{3A})S(O)₂N(R^{3A})₂. In some embodiments. R³ is —N(R^{3A})S(O)₂R^{3A}. In some embodiments. R³ is —N═S(O)(R^{3A})₂. In some embodiments, R³ is —S(NR^{3A})(O)R^{3A}. In some embodiments, R³ is —N(R^{3A})S(O)R^{3A}. In some embodiments, R³ is —N(R^{3A})CN. In some embodiments, R³ is —P(O)(R^{3A})₂. In some embodiments, R³ is —P(O)(OR^{3A})₂. In some embodiments, R³ is an optionally substituted C₁₋₆ alkyl, alkenyl, or alkynyl. In some embodiments, R³ is an optionally substituted phenyl. In some embodiments, R³ is an optionally substituted 4-7 membered saturated or partially unsaturated monocyclic carbocyclic ring. In some embodiments, R³ is an optionally substituted 5-6 membered monocyclic heteroaryl ring having 1-4 heteroatoms independently selected from nitrogen, oxygen, and sulfur. In some embodiments, R³ is an optionally substituted 4-8 membered saturated or partially unsaturated monocyclic heterocyclic ring having 1-2 heteroatoms independently selected from nitrogen, oxygen, and sulfur. In some embodiments, R³ is an optionally substituted 6-10 membered saturated or partially unsaturated spirocyclic ring having 0-3 heteroatoms independently selected from nitrogen, oxygen, and sulfur.

In some embodiments, R³ is —CN, —OR^{3D}, —N(R^{3A})(R^{3E}), —C(O)OR^{3A}, —C(O)N(R^{3A})OR^{3A}, —N(R^{3A})C(O)OR^{3A}, —N(R^{3A})C(O)R^{3A}, —N(R^{3A})C(O)N(R^{3A})₂, —N(R^{3A})C(NR^{3A})R^{3A}, —N(R^{3A})S(O)₂R^{3A}, or an optionally substituted group selected from C₁₋₆ alkyl, alkenyl, or alkynyl; a 4-8 membered saturated or partially unsaturated monocyclic heterocyclic ring having 1-2 heteroatoms independently selected from nitrogen, oxygen, and sulfur; and a 6-10 membered saturated or partially unsaturated spirocyclic ring having 0-3 heteroatoms independently selected from nitrogen, oxygen, and sulfur.

As defined generally above, each R^{3A} are independently hydrogen, —CN, halogen, or an optionally substituted group selected from C₁₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic carbocyclic ring having 1-2 heteroatoms independently selected from nitrogen, oxygen, and sulfur; or two R^{3A} groups on the same atom are optionally taken together with the atom to form an optionally substituted ring selected from a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 7-10 membered saturated or partially unsaturated bicyclic heterocyclic ring having 1-4 heteroatoms independently selected from nitrogen, oxygen, phosphorus, and sulfur; a 5-8 membered saturated or partially unsaturated bridged bicyclic ring having 0-3 heteroatoms independently selected from nitrogen, oxygen, phosphorus, and sulfur; and a 6-10 membered saturated or partially unsaturated spirocyclic ring having 0-3 heteroatoms independently selected from nitrogen, oxygen, phosphorus, and sulfur.

In some embodiments, R^{3A} is hydrogen. In some embodiments, R^{3A} is —CN. In some embodiments, R^{3A} is halogen. In some embodiments, R^{3A} is optionally substituted C₁₋₆aliphatic. In some embodiments, R^{3A} is optionally substituted phenyl. In some embodiments, R^{3A} is optionally substituted naphthyl. In some embodiments, R^{3A} is optionally substituted 3-7 membered saturated or partially unsaturated monocyclic carbocyclic ring. In some embodiments, R^{3A} is optionally substituted 3-7 membered saturated or partially unsaturated monocyclic heterocyclic ring having 1-2 heteroatoms independently selected from nitrogen, oxygen, and sulfur. In some embodiments, R^{3A} is optionally substituted 5-6 membered monocyclic heteroaryl ring having 1-4 heteroatoms independently selected from nitrogen, oxygen, and sulfur. In some embodiments, R^{3A} is optionally substituted 8-10 membered bicyclic heteroaryl ring having 1-4 heteroatoms independently selected from nitrogen, oxygen, and sulfur. In some embodiments, R^{3A} is optionally substituted 7-12 membered saturated or partially unsaturated bicyclic heterocyclic ring having 1-4 heteroatoms independently selected from nitrogen, oxygen, and sulfur. In some embodiments, R^{3A} is optionally substituted 5-8 membered saturated or partially unsaturated bridged bicyclic ring having 0-3 heteroatoms independently selected from nitrogen, oxygen, and sulfur. In some embodiments, R^{3A} is optionally substituted 6-10 membered saturated or partially unsaturated spirocyclic ring having 0-3 heteroatoms independently selected from nitrogen, oxygen, and sulfur. In some embodiments, R^{3A} is optionally substituted 6-11 membered saturated or partially unsaturated bicyclic carbocyclic ring having 1-2 heteroatoms independently selected from nitrogen, oxygen, and sulfur.

In some embodiments, two R^{3A} groups on the same atom are optionally taken together with the atom to form an optionally substituted 3-7 membered saturated or partially unsaturated monocyclic carbocyclic ring. In some embodiments, two R^{3A} groups on the same atom are optionally taken together with the atom to form an optionally substituted 3-7 membered saturated or partially unsaturated monocyclic heterocyclic ring having 1-2 heteroatoms independently selected from nitrogen, oxygen, and sulfur. In some embodiments, two R^{3A} groups on the same atom are optionally taken together with the atom to form an optionally substituted 7-10 membered saturated or partially unsaturated bicyclic heterocyclic ring having 1-4 heteroatoms independently selected from nitrogen, oxygen, phosphorus, and sulfur. In some embodiments, two R^{3A} groups on the same atom are optionally taken together with the atom to form an optionally substituted 5-8 membered saturated or partially unsaturated bridged bicyclic ring having 0-3 heteroatoms independently selected from nitrogen, oxygen, phosphorus, and sulfur. In some embodiments, two R^{3A} groups on the same atom are optionally taken together with the atom to form an optionally substituted 6-10 membered saturated or partially unsaturated spirocyclic ring having 0-3 heteroatoms independently selected from nitrogen, oxygen, phosphorus, and sulfur.

As defined generally above, R^{3B} is hydrogen, halogen, —CN, —OR^{3C}, —SR, —N(R^{3A})(R^{3A}), —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —NRC(O)OR, —NRC(O)R, —NRC(O)NR₂, —NRC(NR)NR₂, —NRNR₂, —NRS(O)₂NR₂, —NRS(O)₂R, —N═S(O)R₂, —S(NR)(O)R, —NRS(O)R, —NRCN, —P(O)R₂, —P(O)(OR)₂; or an optionally substituted group selected from C₁₋₆ aliphatic; a phenyl ring; a 4-7 membered saturated or partially unsaturated monocyclic carbocyclic ring; a 5-6 membered monocyclic heteroaryl ring having 1-4 heteroatoms independently selected from nitrogen, oxygen, and sulfur; a 4-8 membered saturated or partially unsaturated monocyclic heterocyclic ring having 1-2 heteroatoms independently selected from nitrogen, oxygen, and sulfur; and a 7-10 membered partially unsaturated or heteroaromatic bicyclic heterocyclic ring having 1-4 heteroatoms independently selected from nitrogen, oxygen, and sulfur.

In some embodiments, R^{3B} is hydrogen. In some embodiments, R^{3B} is halogen. In some embodiments, R^{3B} is —CN. In some embodiments, R^{3B} is —OR³c. In some embodiments, R^{3B} is —SR. In some embodiments, R^{3B} is —N(R^{3A})(R^{3A}). In some embodiments, R^{3B} is —S(O)₂R. In some embodiments, R^{3B} is —S(O)₂NR₂. In some embodiments, R^{3B} is —S(O)R. In some embodiments, R^{3B} is —S(O)NR₂. In some embodiments, R^{3B} is —C(O)R. In some embodiments, R^{3B} is —C(O)OR. In some embodiments. R^{3B} is —C(O)NR₂. In some embodiments, R^{3B} is —C(O)N(R)OR. In some embodiments, R^{3B} is —OC(O)R. In some embodiments, R^{3B} is —OC(O)NR₂. In some embodiments, R^{3B} is —NRC(O)OR. In some embodiments, R^{3B} is —NRC(O)R. In some embodiments, R^{3B} is —NRC(O)NR₂. In some embodiments, R^{3B} is —NRC(NR)NR₂, —NRNR₂. In some embodiments, R^{3B} is —NRS(O)₂NR₂. In some embodiments, R^{3B} is —NRS(O)₂R. In some embodiments, R^{3B} is —N═S(O)R₂. In some embodiments, R^{3B} is —S(NR)(O)R. In some embodiments, R^{3B} is —NRS(O)R. In some embodiments, R^{3B} is —NRCN. In some embodiments, R^{3B} is —P(O)(OR)₂. In some embodiments, R^{3B} is —P(O)R₂. In some embodiments. R^{3B} is an optionally substituted C₁₋₆ aliphatic. In some embodiments, R^{3B} is an optionally substituted phenyl. In some embodiments, R^{3B} is an optionally substituted 4-7 membered saturated or partially unsaturated monocyclic carbocyclic ring. In some embodiments, R^{3B} is an optionally substituted 5-6 membered monocyclic heteroaryl ring having 1-4 heteroatoms independently selected from nitrogen, oxygen, and sulfur. In some embodiments, R^{3B} is an optionally substituted 4-8 membered saturated or partially unsaturated monocyclic heterocyclic ring having 1-2 heteroatoms independently selected from nitrogen, oxygen, and sulfur. In some embodiments. R^{3B} is an optionally substituted 7-10 membered partially unsaturated or heteroaromatic bicyclic heterocyclic ring having 1-4 heteroatoms independently selected from nitrogen, oxygen, and sulfur.

As defined generally above, each R^{3C} are independently an optionally substituted group selected from C₁₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic carbocyclic ring having 1-2 heteroatoms independently selected from nitrogen, oxygen, and sulfur.

In some embodiments, R^{3C} is an optionally substituted C₁₋₆ aliphatic. In some embodiments, R^{3C} is an optionally substituted phenyl. In some embodiments, R^{3C} is an optionally substituted naphthyl. In some embodiments, R^{3C} is an optionally substituted 3-7 membered saturated or partially unsaturated monocyclic carbocyclic ring. In some embodiments, R^{3C} is an optionally substituted 3-7 membered saturated or partially unsaturated monocyclic heterocyclic ring having 1-2 heteroatoms independently selected from nitrogen, oxygen, and sulfur. In some embodiments, R^{3C} is an optionally substituted 5-6 membered monocyclic heteroaryl ring having 1-4 heteroatoms independently selected from nitrogen, oxygen, and sulfur. In some embodiments, R^{3C} is an optionally substituted 8-10 membered bicyclic heteroaryl ring having 1-4 heteroatoms independently selected from nitrogen, oxygen, and sulfur. In some embodiments, R^{3C} is an optionally substituted 7-12 membered saturated or partially unsaturated bicyclic heterocyclic ring having 1-4 heteroatoms independently selected from nitrogen, oxygen, and sulfur. In some embodiments, R^{3C} is an optionally substituted 5-8 membered saturated or partially unsaturated bridged bicyclic ring having 0-3 heteroatoms independently selected from nitrogen, oxygen, and sulfur. In some embodiments, R^{3C} is an optionally substituted 6-10 membered saturated or partially unsaturated spirocyclic ring having 0-3 heteroatoms independently selected from nitrogen, oxygen, and sulfur. In some embodiments, R^{3C} is an optionally substituted 6-11 membered saturated or partially unsaturated bicyclic carbocyclic ring having 1-2 heteroatoms independently selected from nitrogen, oxygen, and sulfur.

As defined generally above, R^{3D} is hydrogen, a substituted ethyl or an optionally substituted group selected from methyl or C₃₋₆ aliphatic: phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic carbocyclic ring having 1-2 heteroatoms independently selected from nitrogen, oxygen, and sulfur.

In some embodiments, R^{3D} is hydrogen. In some embodiments, R^{3D} is a substituted ethyl. In some embodiments, R^{3D} is an optionally substituted methyl. In some embodiments, R^{3D} is an optionally substituted C₃₋₆ aliphatic. In some embodiments, R^{3D} is an optionally substituted phenyl. In some embodiments. R^{3D} is an optionally substituted naphthyl. In some embodiments, R^{3D} is an optionally substituted 3-7 membered saturated or partially unsaturated monocyclic carbocyclic ring. In some embodiments, R^{3D} is an optionally substituted 3-7 membered saturated or partially unsaturated monocyclic heterocyclic ring having 1-2 heteroatoms independently selected from nitrogen, oxygen, and sulfur. In some embodiments, R^{3D} is an optionally substituted 5-6 membered monocyclic heteroaryl ring having 1-4 heteroatoms independently selected from nitrogen, oxygen, and sulfur. In some embodiments. R^{3D} is an optionally substituted 8-10 membered bicyclic heteroaryl ring having 1-4 heteroatoms independently selected from nitrogen, oxygen, and sulfur. In some embodiments, R^{3D} is an optionally substituted 7-12 membered saturated or partially unsaturated bicyclic heterocyclic ring having 1-4 heteroatoms independently selected from nitrogen, oxygen, and sulfur. In some embodiments, R^{3D} is an optionally substituted 5-8 membered saturated or partially unsaturated bridged bicyclic ring having 0-3 heteroatoms independently selected from nitrogen, oxygen, and sulfur. In some embodiments, R^{3D} is an optionally substituted 6-10 membered saturated or partially unsaturated spirocyclic ring having 0-3 heteroatoms independently selected from nitrogen, oxygen, and sulfur. In some embodiments, R^{3D} is an optionally substituted 6-11 membered saturated or partially unsaturated bicyclic carbocyclic ring having 1-2 heteroatoms independently selected from nitrogen, oxygen, and sulfur.

As defined generally above, R³ is a C₂ aliphatic substituted with v instances of R^{3B} a substituted 2-hydroxyethyl, or an optionally substituted group selected from methyl or C₃_aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; and a 6-10 membered saturated or partially unsaturated spirocyclic ring having 0-3 heteroatoms independently selected from nitrogen, oxygen, and sulfur; a 6-11 membered saturated or partially unsaturated bicyclic carbocyclic ring having 1-2 heteroatoms independently selected from nitrogen, oxygen, and sulfur; or R^{3A} and R^{3E} groups on the same nitrogen are optionally taken together with the nitrogen to form an optionally substituted 3-7 membered saturated or partially unsaturated monocyclic heterocyclic ring having 0-2 heteroatoms, in addition to the nitrogen from which R^{3A} and R^{3E} are attached, independently selected from nitrogen, oxygen, and sulfur; a 5-8 membered saturated or partially unsaturated bridged bicyclic ring having 0-2 heteroatoms, in addition to the nitrogen from which R^{3A} and R^{3E} are attached, independently selected from nitrogen, oxygen, and sulfur; and a 6-10 membered saturated or partially unsaturated spirocyclic ring having 0-2 heteroatoms, in addition to the nitrogen from which R^{3A} and R^{3E} are attached, independently selected from nitrogen, oxygen, and sulfur.

In some embodiments, R^{3E} is a substituted methyl. In some embodiments, R^{3E} is a substituted C₃ aliphatic. In some embodiments, R^{3E} is a C₂ aliphatic substituted with v instances of R^{3B}. In some embodiments, R^{3E} is a substituted 2-hydroxethyl. In some embodiments, R^{3E} is an optionally substituted methyl or C₃₋₆ aliphatic. In some embodiments, R^{3E} is an optionally substituted phenyl. In some embodiments, R^{3E} is an optionally substituted naphthyl. In some embodiments, R^{E} is an optionally substituted 3-7 membered saturated or partially unsaturated monocyclic carbocyclic ring. In some embodiments, R^{3E} is an optionally substituted 3-7 membered saturated or partially unsaturated monocyclic heterocyclic ring having 1-2 heteroatoms independently selected from nitrogen, oxygen, and sulfur. In some embodiments, R^{3E} is an optionally substituted 5-6 membered monocyclic heteroaryl ring having 1-4 heteroatoms independently selected from nitrogen, oxygen, and sulfur. In some embodiments, R^{3E} is an optionally substituted 8-10 membered bicyclic heteroaryl ring having 1-4 heteroatoms independently selected from nitrogen, oxygen, and sulfur. In some embodiments, R^{3E} is an optionally substituted 7-12 membered saturated or partially unsaturated bicyclic heterocyclic ring having 1-4 heteroatoms independently selected from nitrogen, oxygen, and sulfur. In some embodiments. R^{3E} is an optionally substituted 5-8 membered saturated or partially unsaturated bridged bicyclic ring having 0-3 heteroatoms independently selected from nitrogen, oxygen, and sulfur; and a 6-10 membered saturated or partially unsaturated spirocyclic ring having 0-3 heteroatoms independently selected from nitrogen, oxygen, and sulfur. In some embodiments, R^{3E} is an optionally substituted 6-11 membered saturated or partially unsaturated bicyclic carbocyclic ring having 1-2 heteroatoms independently selected from nitrogen, oxygen, and sulfur.

In some embodiments, R^{3A} and R^{3E} groups on the same nitrogen are optionally taken together with the nitrogen to form an optionally substituted 3-7 membered saturated or partially unsaturated monocyclic heterocyclic ring having 0-2 heteroatoms, in addition to the nitrogen from which R^{3A} and R^{3E} are attached, independently selected from nitrogen, oxygen, and sulfur. In some embodiments, R^{3A} and R^{3E} groups on the same nitrogen are optionally taken together with the nitrogen to form an optionally substituted 5-8 membered saturated or partially unsaturated bridged bicyclic ring having 0-2 heteroatoms, in addition to the nitrogen from which R^{3A} and R^{3E} are attached, independently selected from nitrogen, oxygen, and sulfur. In some embodiments, R^{3A} and R^{3E} groups on the same nitrogen are optionally taken together with the nitrogen to form an optionally substituted 6-10 membered saturated or partially unsaturated spirocyclic ring having 0-2 heteroatoms, in addition to the nitrogen from which R^{3A} and R^{3E} are attached, independently selected from nitrogen, oxygen, and sulfur.

As defined generally above, each instance of v is independently 0, 1, 2, 3, 4, or 5.

In some embodiments, v is 0, 1, 2, 3, 4, or 5.

In some embodiments, v is 0. In some embodiments, v is 1. In some embodiments, v is 2. In some embodiments, v is 3. In some embodiments, v is 4. In some embodiments, v is 5.

In some embodiments, v is 0 or 1. In some embodiments, v is 0, 1, or 2. In some embodiments, v is 0, 1, 2, or 3. In some embodiments, v is 1 or 2. In some embodiments, v is 1, 2, or 3. In some embodiments, v is 1, 2, 3, or 4. In some embodiments, v is 2 or 3. In some embodiments, v is 2, 3, or 4. In some embodiments, v is 3 or 4. In some embodiments, v is 3, 4, or 5.

In some embodiments, R³ is —CN, —OR³D, —N(R^{3A})(R^{3E}), —C(O)OR^{3A}, —C(O)N(R^{3A})OR^{3A}, —N(R^{3A})C(O)OR^{3A}, —N(R^{3A})C(O)R^{3A}, —N(R^{3A})C(O)N(R^{3A})₂, —NR^{3A}, —C(NR^{3A})R^{3A}, —N(R^{3A})S(O)₂R^{3A}, or an optionally substituted group selected from C₁₋₆ aliphatic; a 4-8 membered saturated or partially unsaturated monocyclic heterocyclic ring having 1-2 heteroatoms independently selected from nitrogen, oxygen, and sulfur; and a 6-10 membered saturated or partially unsaturated spirocyclic ring having 0-3 heteroatoms independently selected from nitrogen, oxygen, and sulfur.

In some embodiments, R³ is selected from —CN, isopentyl,

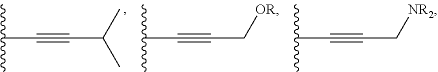

—CH₂O(R^{3A}), —CH₂N(R^{3A})COR, —CH₂N(R^{3A})SO₂R,

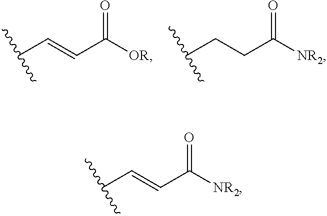

—C(O)OR^{3A}, —C(O)N(R^{3A})OMe, —C(O)N(Me)OR^{3A},

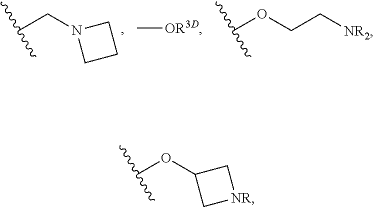

—N(R^{3A})nPr, —N(R^{3A})iBu, —N(R^{3A})COiPr, —N(R^{3A})C(NR^{3A})iPr, —NHC(NR^{3A})R^{3A}, —N(R^{3A})C(NH)R^{3A}, —N(R^{3A})C(O)OMe, —NHC(O)OR^{3A}, —N(R^{3A})CON(R^{3A})Me, —NHCON(R^{3A})₂, —N(R^{3A})CONMe₂, —N(R^{3A})SO₂Et,

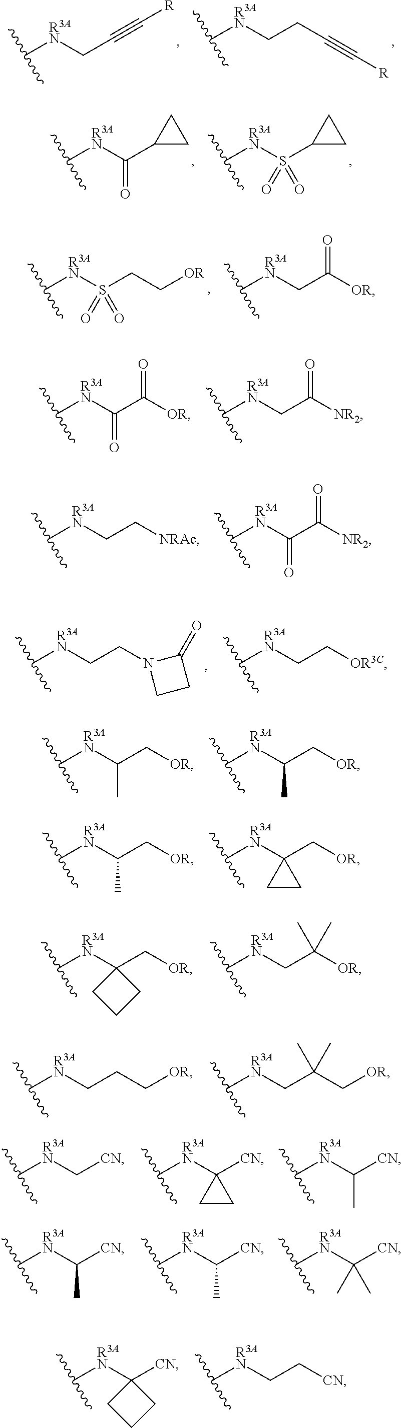

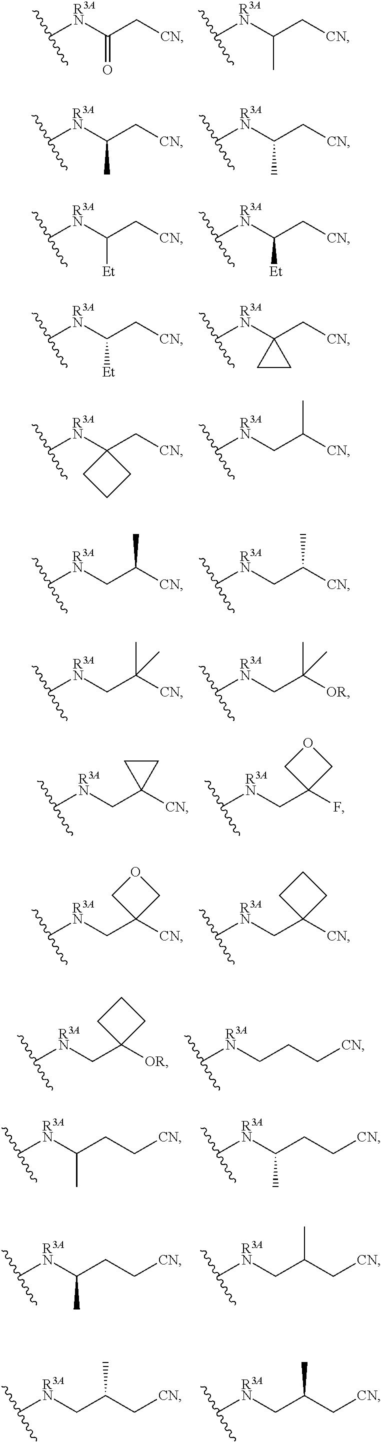

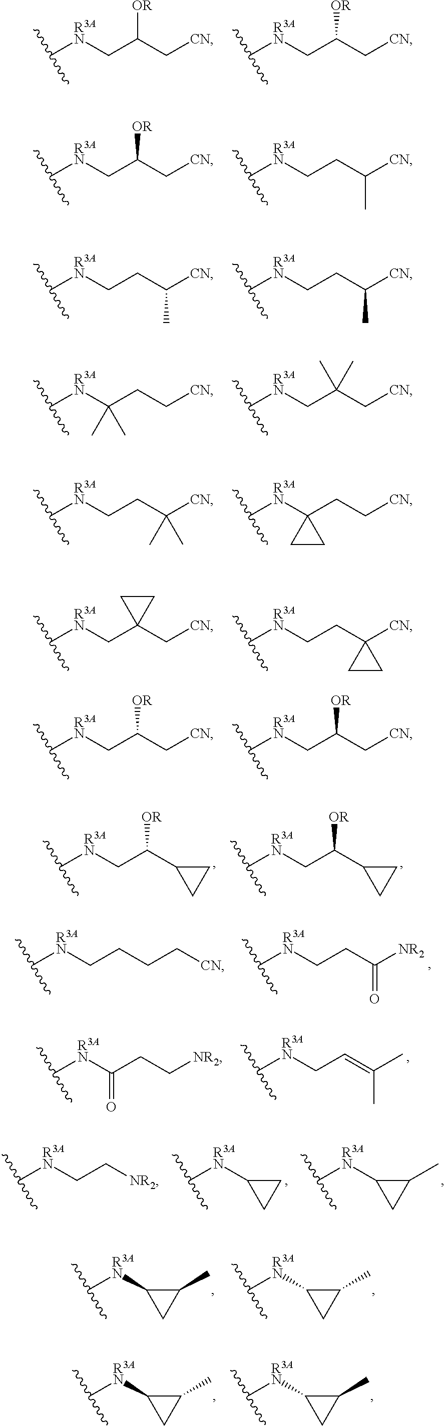

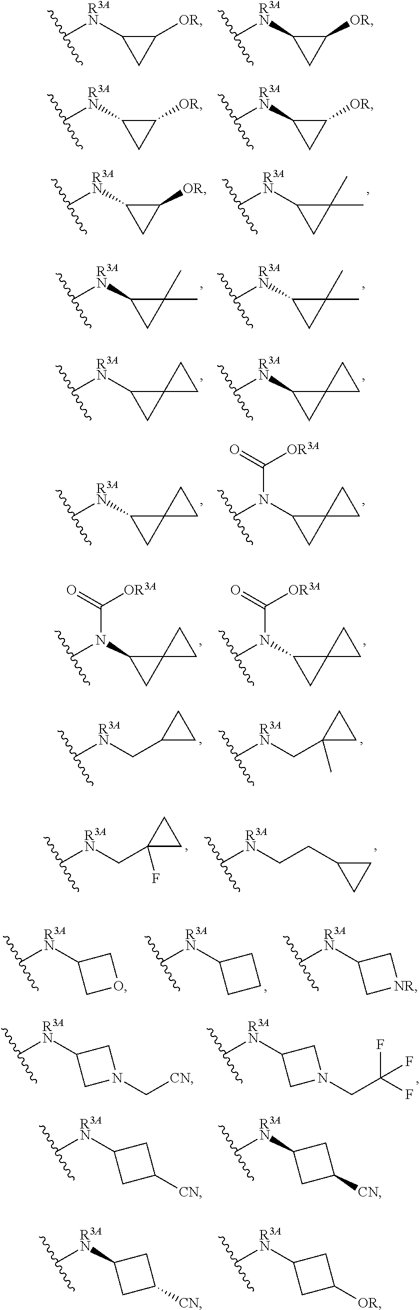

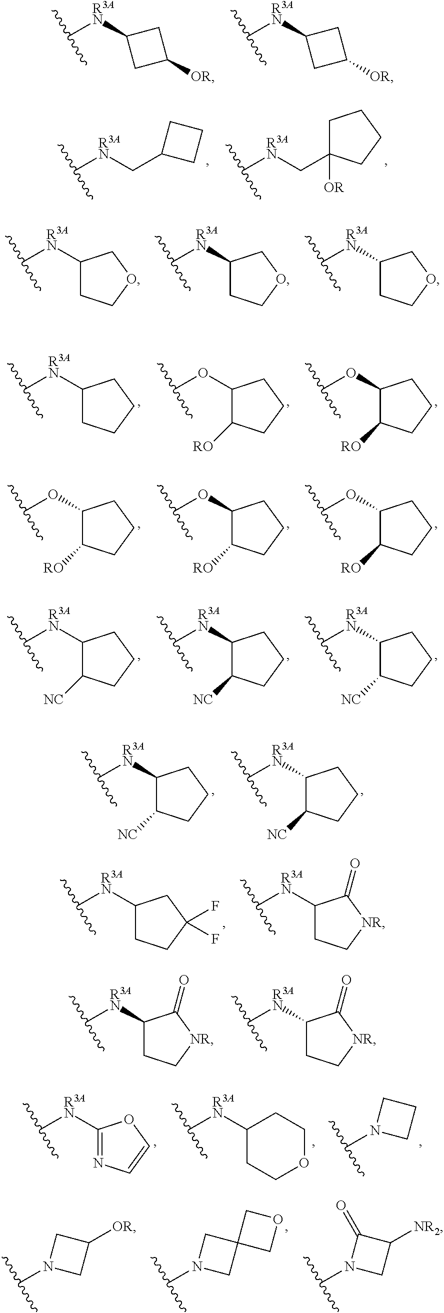

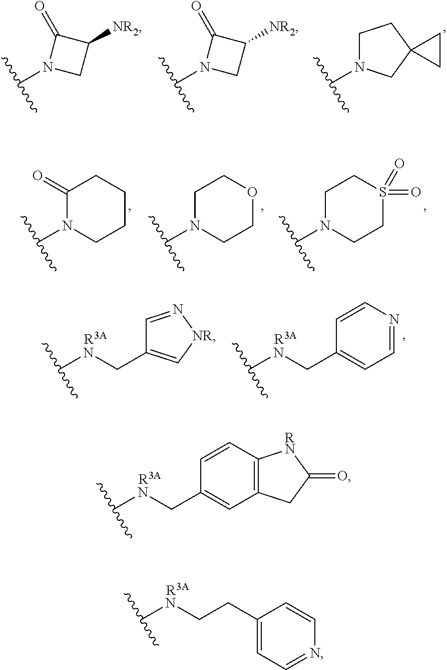

an optionally substituted phenyl, and an optionally substituted 5-6 membered heteroaryl having 1-3 ring heteroatoms selected from nitrogen, oxygen, and sulfur, wherein R^{3A}, R^{3C}, R^{3D}, and R are as defined above and described in embodiments herein, both singly and in combination.

In certain embodiments, R³ is —N(R^{3A})iBu,

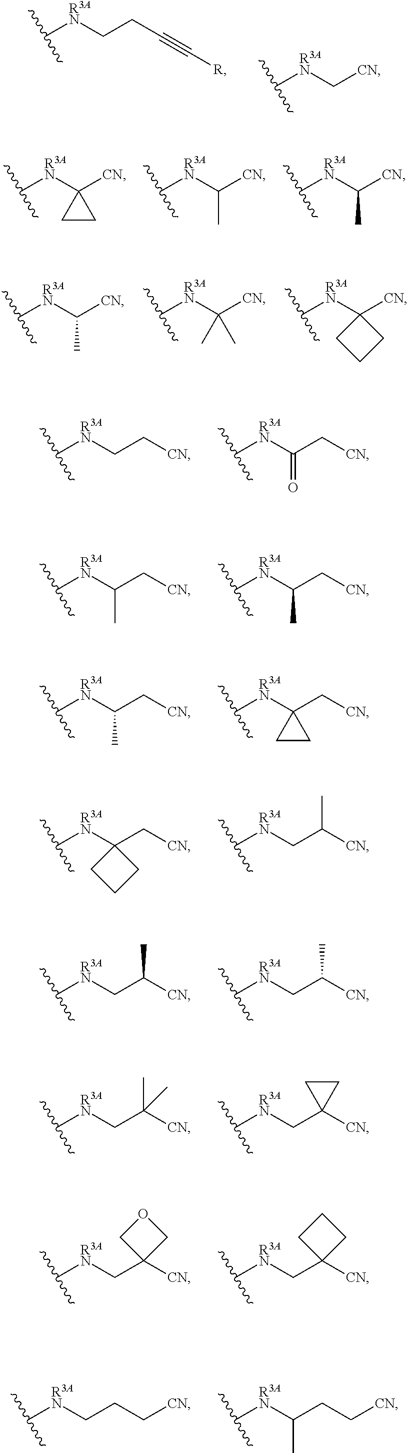

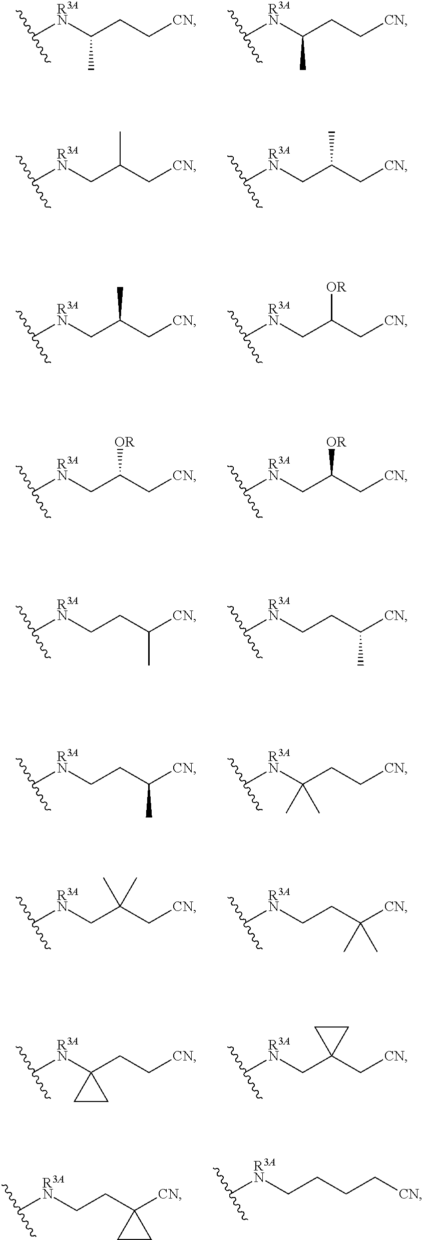

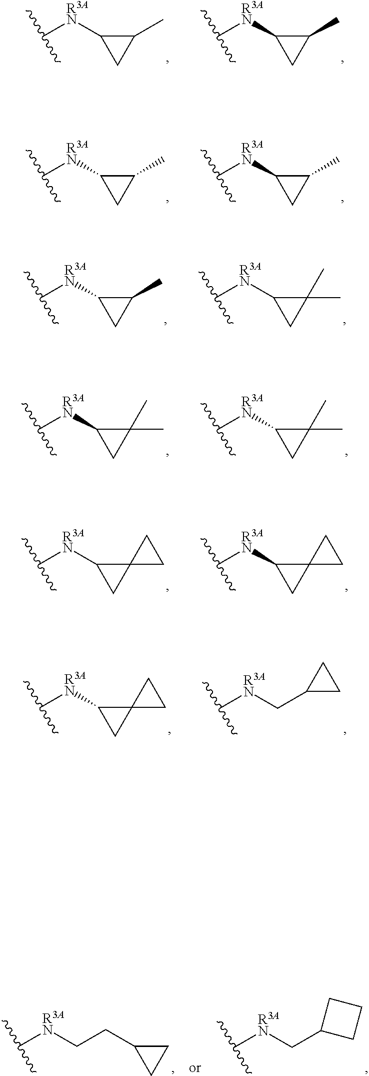

wherein R^{3A} and R are as defined above and described in embodiments herein, both singly and in combination.

In certain embodiments. R³ is —CN, isopentyl,

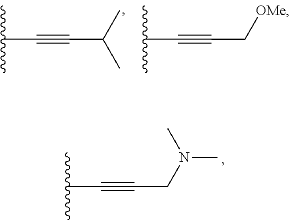

—CH₂OMc, —CH₂NHCOMc, —CH₂NHSO₂Mc,

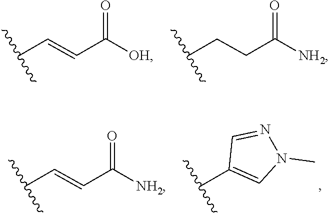

—CO₂H, —CO₂Me, —CON(Me)OMe, —OMe,

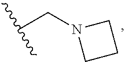 [SMILES: *CN1CCC1]

### —OMe, —OiBu,

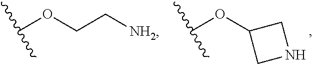 [SMILES: *OCCN ; *OC1CNC1]

—NHnPr, —NHiBu, —NHCOiPr, —NHC(NH)iPr, —NHCO₂Me, —NHCONHMe, —NHCONMe₂, —NHSO₂Et,

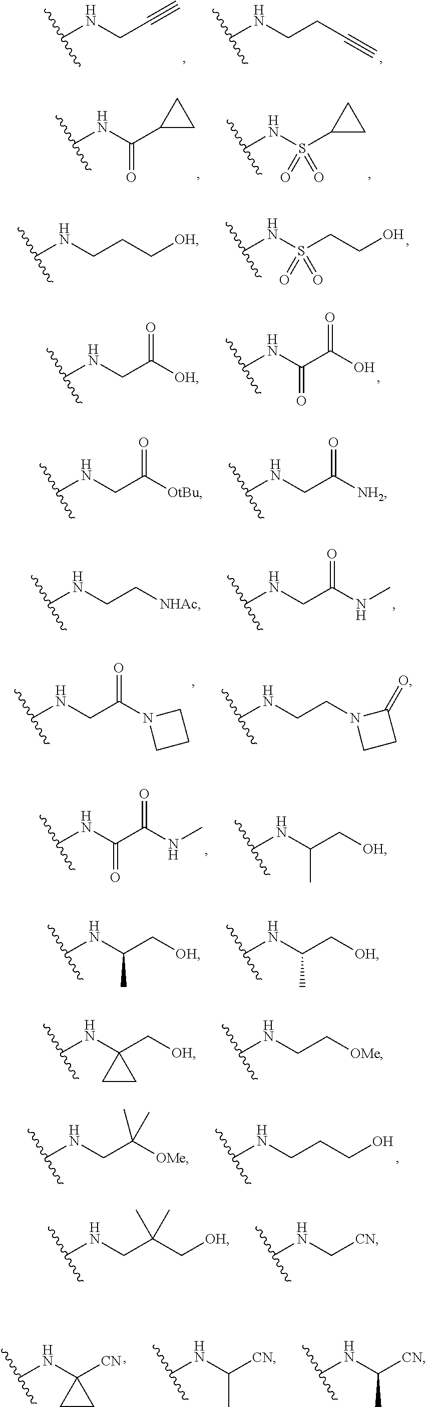

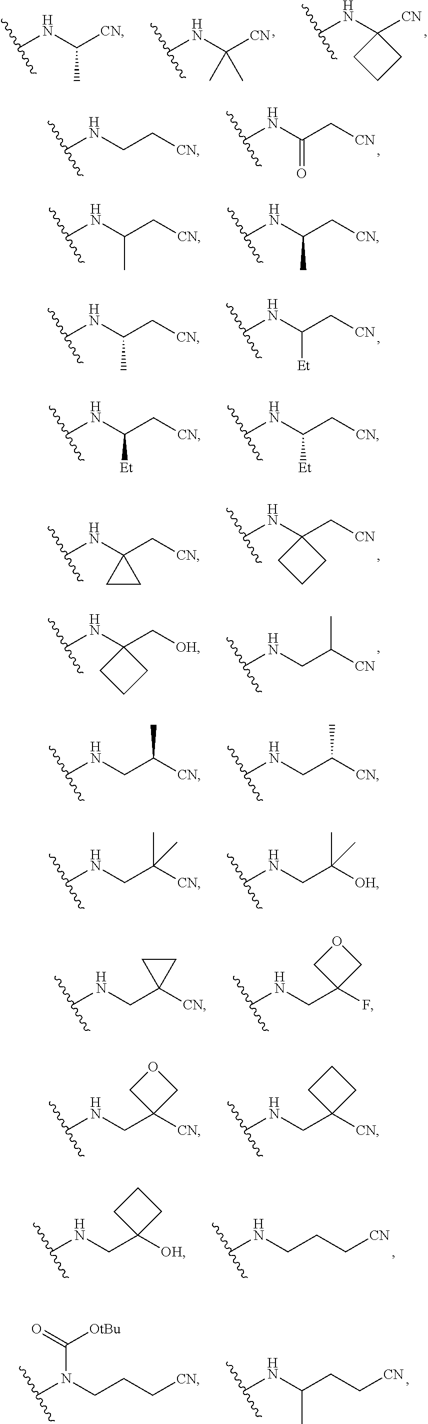

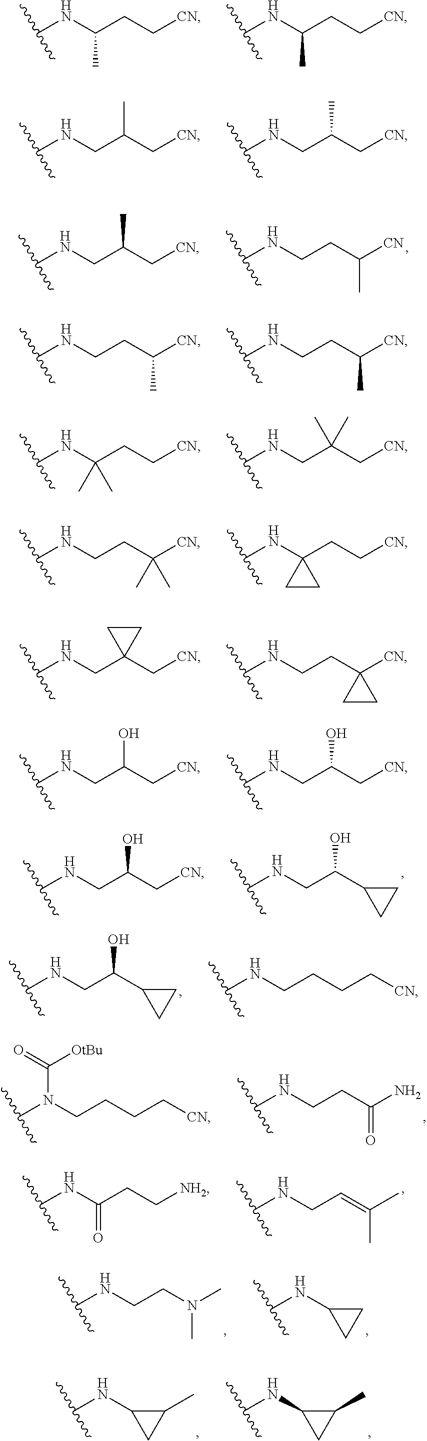

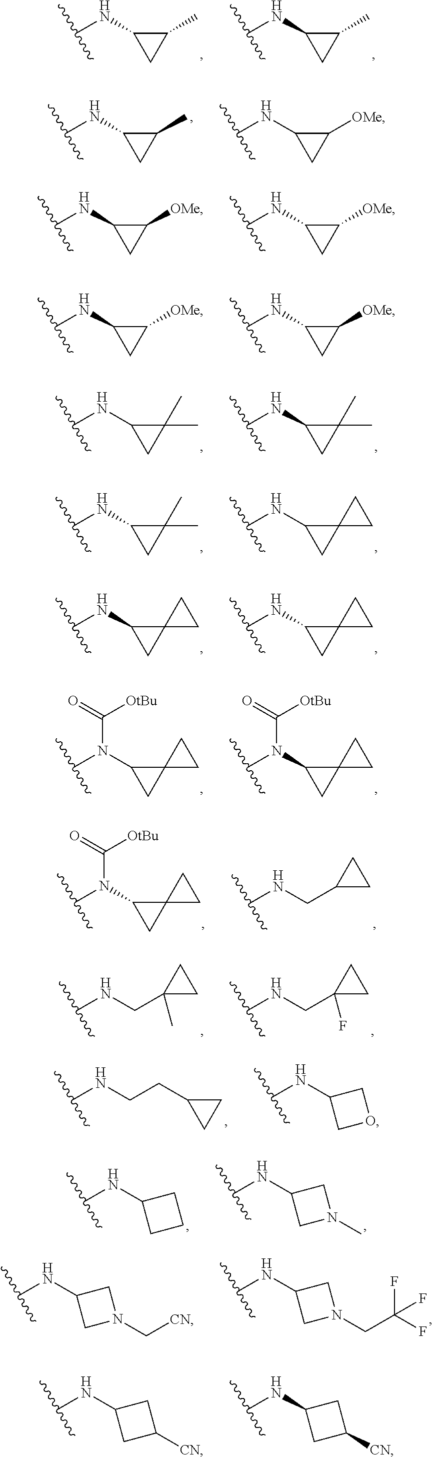

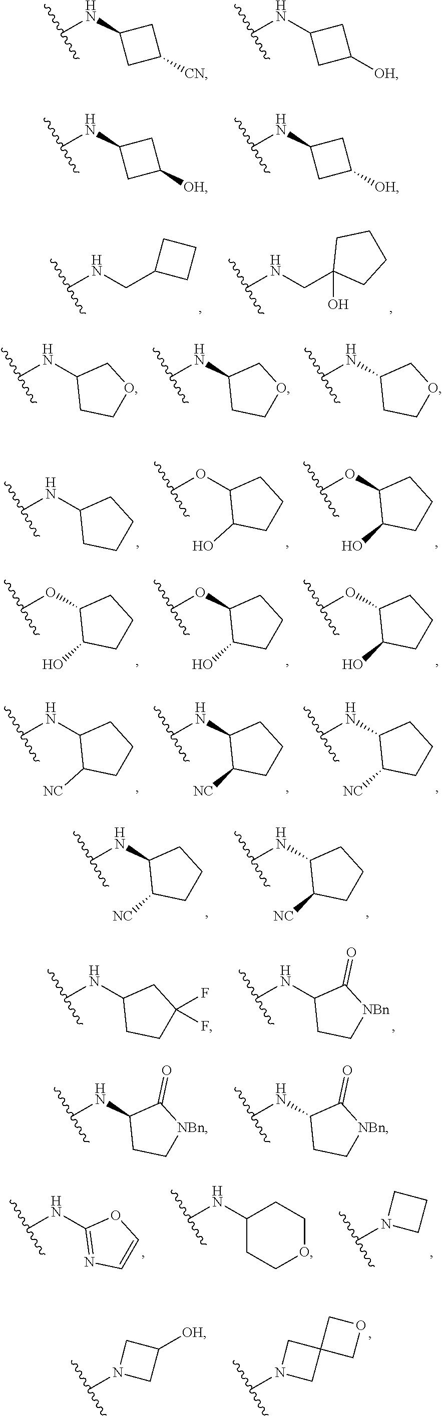

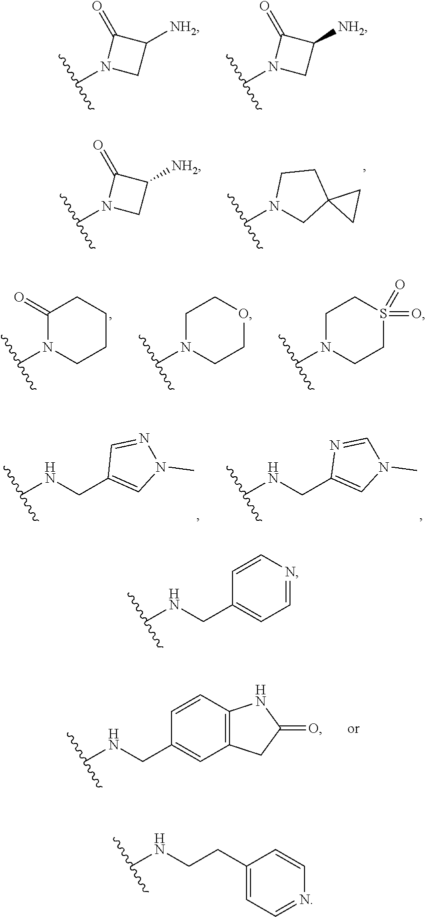

In certain embodiments, R³ is —NHiBu,

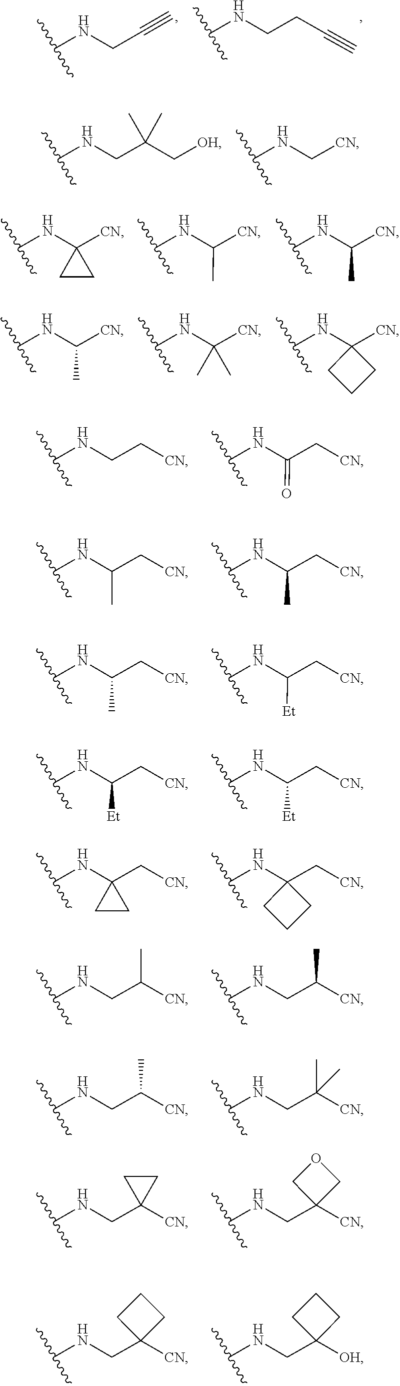

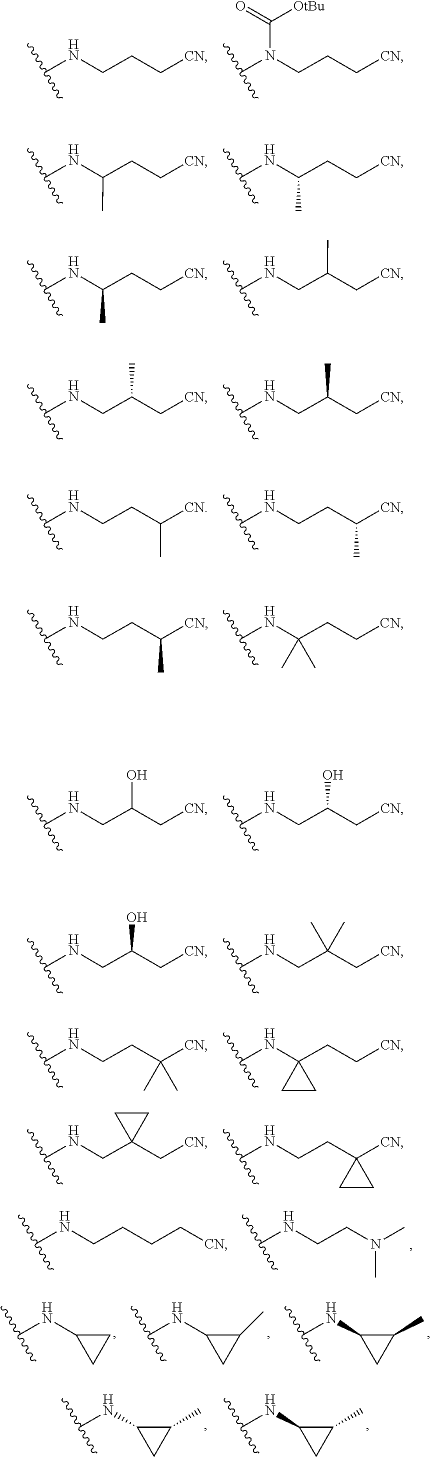

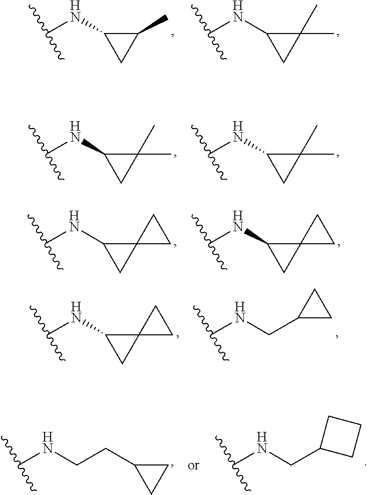

In some embodiments, R³ is selected from those depicted in Table 1, below.

As defined generally above, each R⁸ is independently hydrogen, halogen, —CN, —NO₂, —CRF₂, —CF₃, —OR, —(OCH₂CH₂)₁₋₁₀NR₂, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —NRC(O)OR, —NRC(O)R, —NRC(O)NR₂, —NRC(NR)NR₂, —NRNR₂, —NRS(O)₂NR₂, —NRS(O)₂R, —N═S(O)R₂, —S(NR)(O)R, —NRS(O)R, —NRCN, —P(O)(R)NR₂, —P(O)(R)OR, —P(O)R₂, —CH₂NR(CH₂CH₂O)₁₋₁₀CH₂CH₂NR₂; or an optionally substituted C₁₋₆ aliphatic.

In some embodiments, R⁸ is hydrogen. In some embodiments, R⁸ is halogen. In some embodiments. R⁸ is —CN. In some embodiments, R⁸ is —NO₂. In some embodiments, R⁸ is —CRF₂. In some embodiments, R⁸ is —CF₃. In some embodiments, R⁸ is —OR. In some embodiments, R⁸ is —(OCH₂CH₂)₁₋₁₀NR₂. In some embodiments, R⁸ is —SR. In some embodiments, R⁸ is —NR₂. In some embodiments, R⁸ is —S(O)₂R. In some embodiments, R⁸ is —S(O)₂NR₂. In some embodiments, R⁸ is —S(O)R. In some embodiments, R⁸ is —S(O)NR₂. In some embodiments, R⁸ is —C(O)R. In some embodiments, R⁸ is —C(O)OR. In some embodiments, R⁸ is —C(O)NR₂. In some embodiments, R⁸ is —C(O)N(R)OR. In some embodiments, R⁸ is —OC(O)R. In some embodiments, R⁸ is —OC(O)NR₂. In some embodiments, R⁸ is —NRC(O)OR. In some embodiments, R⁸ is —NRC(O)R. In some embodiments, R⁸ is —NRC(O)NR₂. In some embodiments, R⁸ is —NRC(NR)NR₂. In some embodiments, R⁸ is —NRNR₂. In some embodiments, R⁸ is —NRS(O)₂NR₂. In some embodiments, R⁸ is —NRS(O)₂P. In some embodiments, R⁸ is —N═S(O)R₂. In some embodiments, R⁸ is —S(NR)(O)R. In some embodiments, R⁸ is —NRS(O)R. In some embodiments, R⁸ is —NRCN. In some embodiments, R⁸ is —P(O)R₂. In some embodiments. R⁸ is —P(O)(OR)₂. In some embodiments, R⁸ is —CH₂NR(CH₂CH₂O)₁₋₁₀CH₂CH₂NR₂. In some embodiments, R⁸ is an optionally substituted C₁₋₆ aliphatic.

In some embodiments, R⁸ is selected from halogen, —CF₃, —OR, —(OCH₂CH₂)₁₋₁₀NR₂, —CH₂NR(CH₂CH₂O)₁₋₁₀CH₂CH₂NR₂, and an optionally substituted C₁₋₆ aliphatic.

In some embodiments, R⁸ is selected from fluoro, chloro, methyl, —CF₃, —OR, —(OCH₂CH₂)₁₋₁₀NR₂, —CH₂OR, —CH₂NR(CH₂CH₂O)₁₋₁₀CH₂CH₂NR₂, and —CH₂NR₂.

In some embodiments, a first R⁸ is hydrogen or —CF₃ and a second R⁸ is —CH₂OR or —CH₂NR₂.

In some embodiments, a first R⁸ is hydrogen or —CF₃ and a second R⁸ is —CH₂OR or —CH₂NR₂, wherein —CH₂OR or —CH₂NR₂ is selected from —CH₂OH,

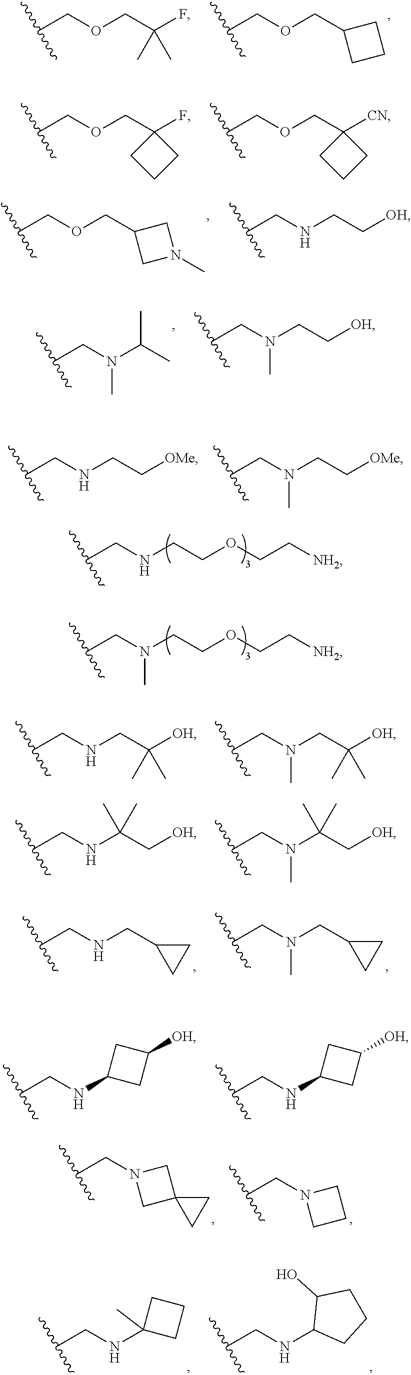

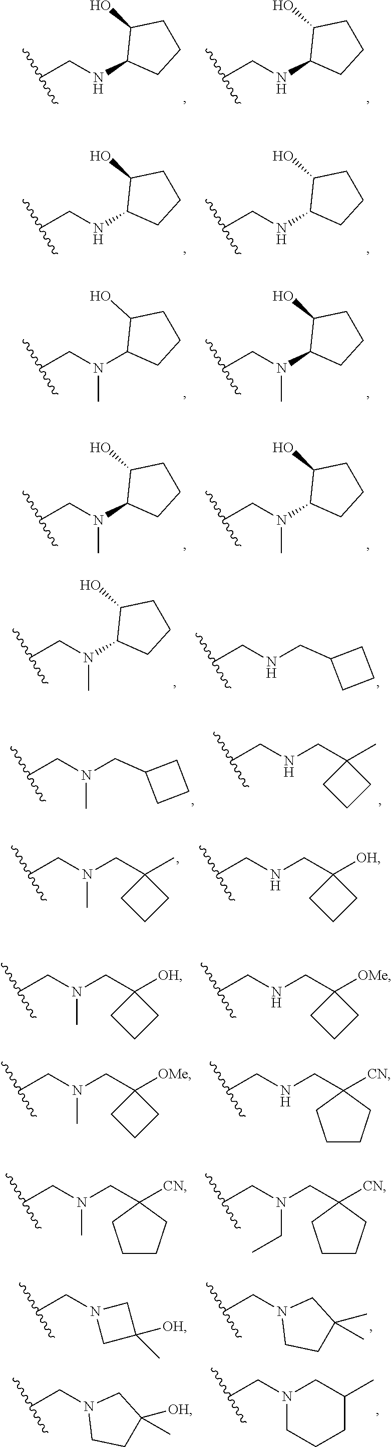

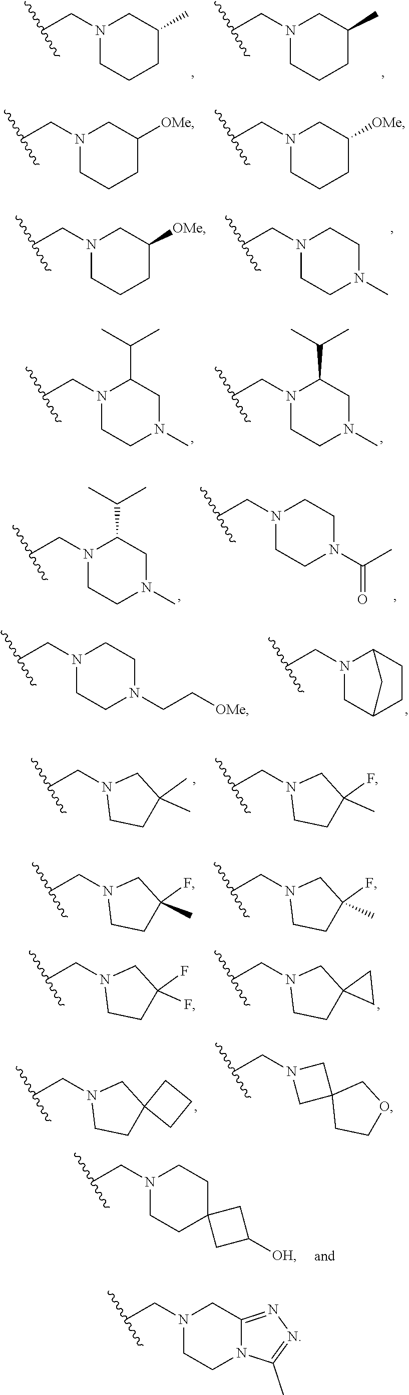

In some embodiments, W is selected from fluoro, chloro, methyl, —CF₃, —OR,

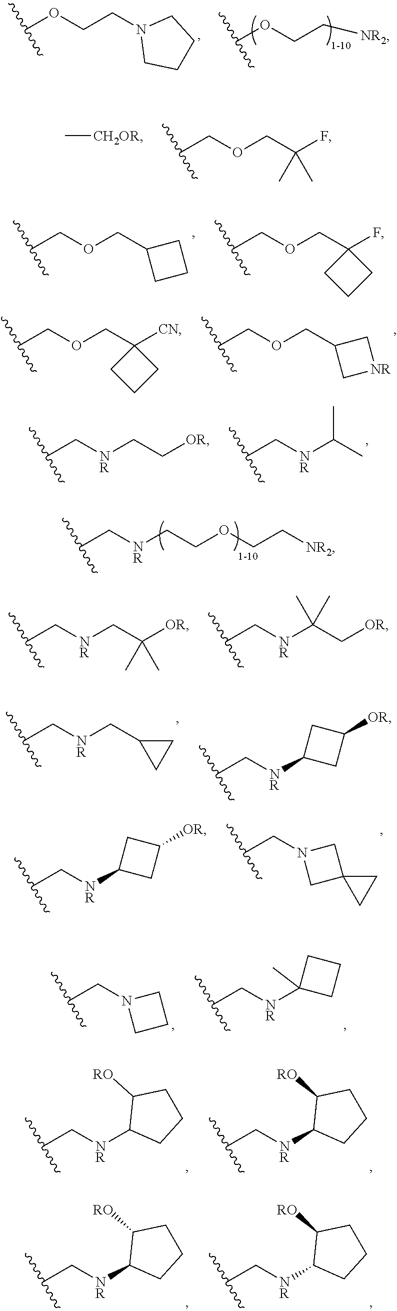

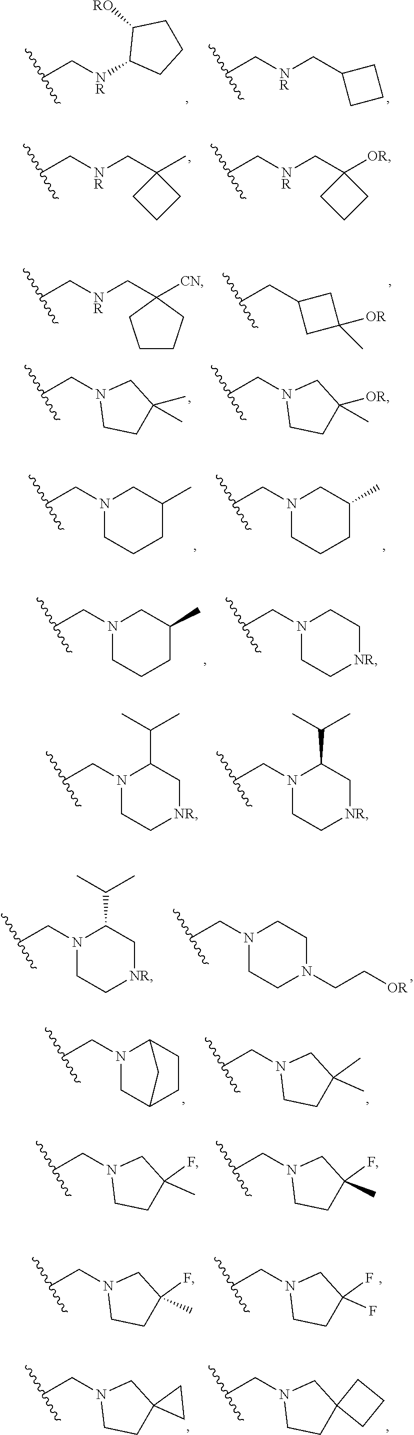

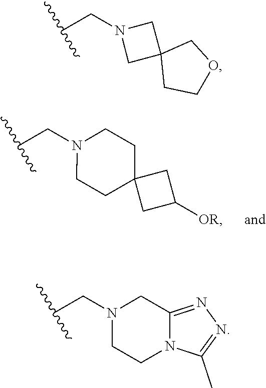

wherein R is as defined above and described in embodiments herein, both singly and in combination.

In certain embodiments, R is fluoro, chloro, methyl, —CF₃, —OH,

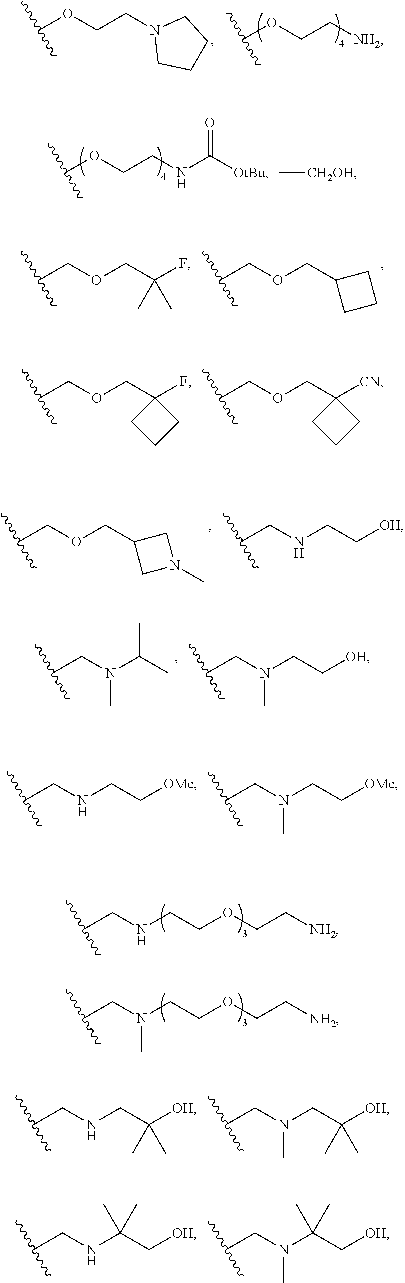

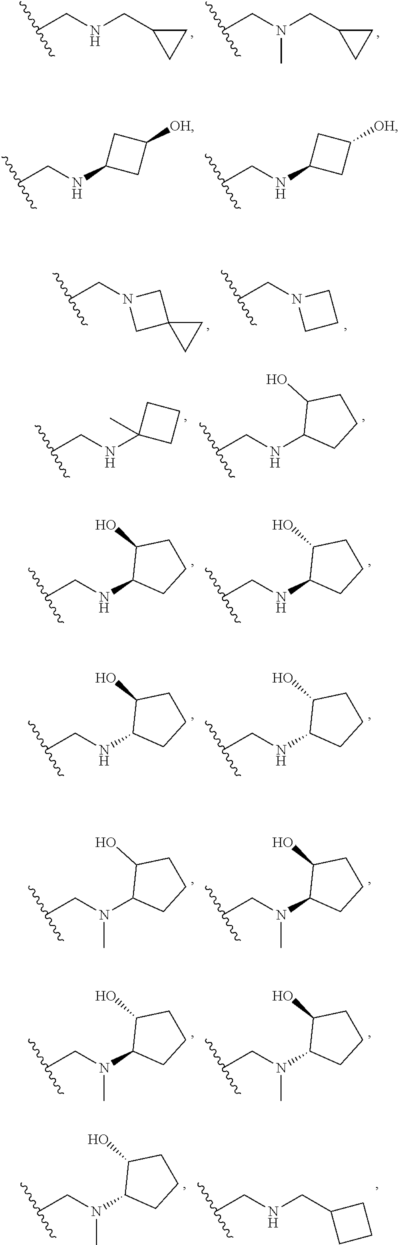

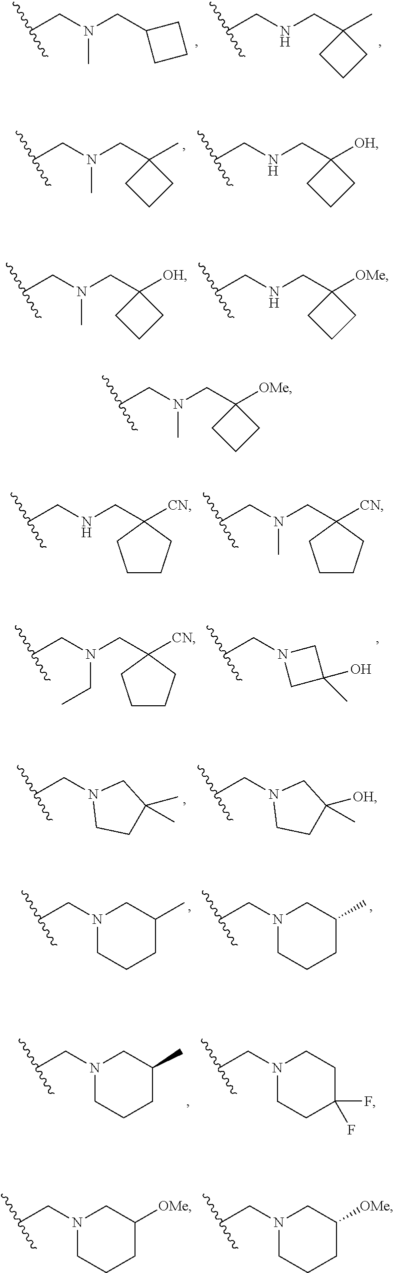

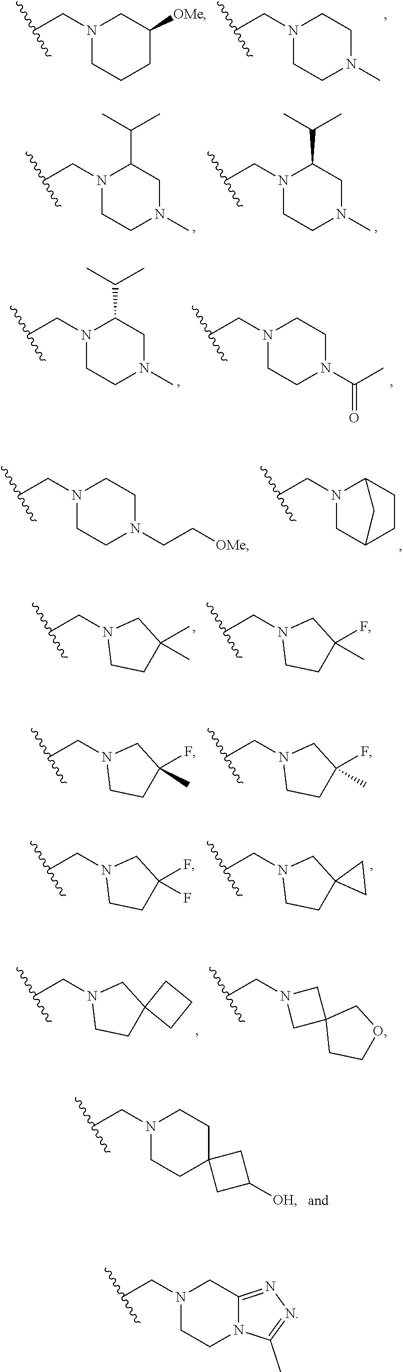

In some embodiments, R⁸ is selected from those depicted in Table 1, below.

As defined generally above, R¹³ is hydrogen, halogen, —CN, —OR^{3A}, —SR^{3A}, N(R^{3A})₂, —S(O)₂R^{3A}, —S(O)₂N(R^{3A})₂, —S(O)R^{3A}, —S(O)N(R^{3A})₂, —C(O)R^{3A}, —C(O)OR, —C(O)N(R^{3A})₂, —C(O)N(R^{3A})OR^{3A}, —OC(O)R^{3A}, —OC(O)N(R^{3A})₂, —N(R^{3A})C(O)OR^{3A}, —N(R^{3A})C(O)R^{3A}, —N(R^{3A})C(O)N(R^{3A})₂, —N(R^{3A})C(NR^{3A})R^{3A}, —N(R^{3A})C(NR^{3A})N(R^{3A})₂, —N(R^{3A})N(R^{3A}), —N(R^{3A})S(O)₂N(R^{3A})₂—N(R^{3A})S(O)₂R^{3A}, —N═S(O)(R^{3A})₂, —S(NR^{3A})(O)R^{3A}, —N(R^{3A})S(O)R^{3A}, —N(R^{3A})CN, —P(O)(R^{3A})OR^{3A}, —P(O)(R^{3A})₂, or an optionally substituted group selected from C₁₋₆ aliphatic; a phenyl ring; a 4-7 membered saturated or partially unsaturated monocyclic carbocyclic ring; a 5-6 membered monocyclic heteroaryl ring having 1-4 heteroatoms independently selected from nitrogen, oxygen, and sulfur; a 4-8 membered saturated or partially unsaturated monocyclic heterocyclic ring having 1-2 heteroatoms independently selected from nitrogen, oxygen, and sulfur; and a 6-10 membered saturated or partially unsaturated spirocyclic ring having 0-3 heteroatoms independently selected from nitrogen, oxygen, and sulfur.

In some embodiments, R¹³ is hydrogen. In some embodiments, R¹³ is halogen. In some embodiments, R¹³ is —CN. In some embodiments. R¹³ is —OR^{3A}. In some embodiments, R¹³ is —SR^{3A}. In some embodiments, R⁸ is —N(R^{3A})₂. In some embodiments, R¹³ is —S(O)₂R^{3A}. In some embodiments, R¹³ is —S(O)₂N(R^{3A})₂. In some embodiments. R¹³ is —S(O)R^{3A}. In some embodiments, R¹³ is —S(O)N(R^{3A})₂. In some embodiments, R¹³ is —C(O)R^{3A}. In some embodiments, R¹³ is —C(O)OR^{3A}. In some embodiments, R¹³ is —C(O)N(R^{3A})₂. In some embodiments, R¹³ is —C(O)N(R^{3A})OR^{3A}. In some embodiments, R¹³ is —OC(O)R^{3A}. In some embodiments, R¹³ is —OC(O)N(R^{3A})₂. In some embodiments, R¹³ is —N(R^{3A})C(O)OR^{3A}. In some embodiments, R³ is —N(R^{3A})C(O)R^{3A}. In some embodiments, R¹³ is —N(R^{3A})C(O)N(R^{3A})₂. In some embodiments, R¹³ is —N(R^{3A})C(NR^{3A})R^{3A}. In some embodiments. R¹³ is —N(R^{3A})C(NR^{3A})N(R^{3A})₂. In some embodiments, R¹³ is —N(R^{3A})N(R^{3A})₂. In some embodiments, R³ is —N(R^{3A})S(O)₂N(R^{3A})₂. In some embodiments, R¹³ is —N(R^{3A})S(O)₂R^{3A}. In some embodiments, R¹³ is —N═S(O)(R^{3A})₂. In some embodiments, R¹³ is —S(NR^{3A})(O)R^{3A}. In some embodiments, R¹³ is —N(R^{3A})S(O)R^{3A}. In some embodiments, R¹³ is —N(R^{3A})CN. In some embodiments, R¹³ is —P(O)(R^{3A})OR^{3A}. In some embodiments, R¹³ is —P(O)(R^{3A})₂. In some embodiments, R¹³ is an optionally substituted C₁₋₆ aliphatic. In some embodiments, R¹³ is an optionally substituted phenyl. In some embodiments, R³ is an optionally substituted 4-7 membered saturated or partially unsaturated monocyclic carbocyclic ring. In some embodiments, R¹³ is an optionally substituted 5-6 membered monocyclic heteroaryl ring having 1-4 heteroatoms independently selected from nitrogen, oxygen, and sulfur. In some embodiments, R¹³ is an optionally substituted 4-8 membered saturated or partially unsaturated monocyclic heterocyclic ring having 1-2 heteroatoms independently selected from nitrogen, oxygen, and sulfur. In some embodiments. R³ is an optionally substituted 6-10 membered saturated or partially unsaturated spirocyclic ring having 0-3 heteroatoms independently selected from nitrogen, oxygen, and sulfur.

In some embodiments, R¹³ is —CN, —OR^{3A}, —N(R^{3A}), —C(O)OR^{3A}, —C(O)N(R^{3A})OR^{3A}, —N(R^{3A})C(O)OR^{3A}, —N(R^{3A})C(O)R^{3A}, —N(R^{3A})C(O)N(R^{3A})₂, —N(R^{3A})C(NR^{3A})R^{3A}, —N(R^{3A})S(O)₂R^{3A}, or an optionally substituted group selected from C₁₋₆ aliphatic: a 4-8 membered saturated or partially unsaturated monocyclic heterocyclic ring having 1-2 heteroatoms independently selected from nitrogen, oxygen, and sulfur; and a 6-10 membered saturated or partially unsaturated spirocyclic ring having 0-3 heteroatoms independently selected from nitrogen, oxygen, and sulfur.

In some embodiments, R¹³ is selected from Cl, —CN, cyclopropyl, isopentyl,

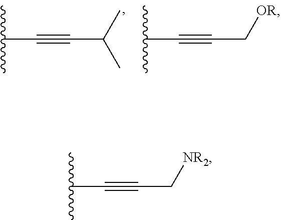

—CF₃, —CH₂OR^{3A}, —CH₂N(R^{3A})COR, —CH₂N(R^{3A})SO₂R,

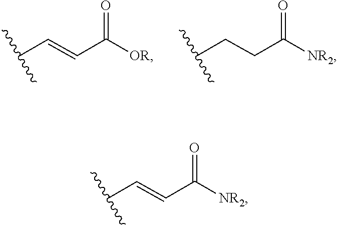

—C(O)OR^{3A}, —C(O)N(R^{3A})OMe, —C(O)N(Me)OR^{3A},

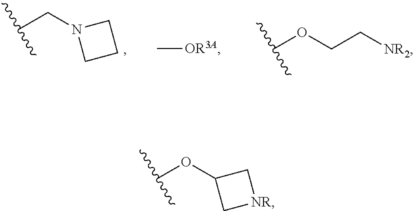

—N(R^{3A})₂, —N(R^{3A})COiPr, —N(R^{3A})C(NR^{3A})iPr, —NHC(NR^{3A})R^{3A}, —N(R^{3A})C(NH)R^{3A}—N(R^{3A})C(O)OMe, —NHC(O)OR^{3A}, —N(R^{3A})CON(R^{3A})Me, —NHCON(R^{3A})₂, —N(R^{3A})CONMe₂, —NHSO₂R^{3A},

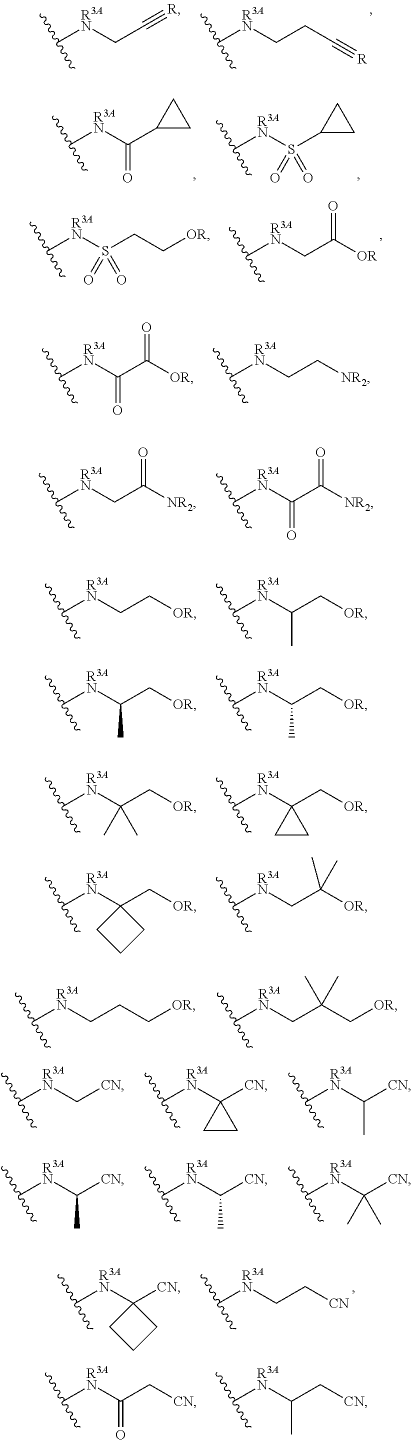

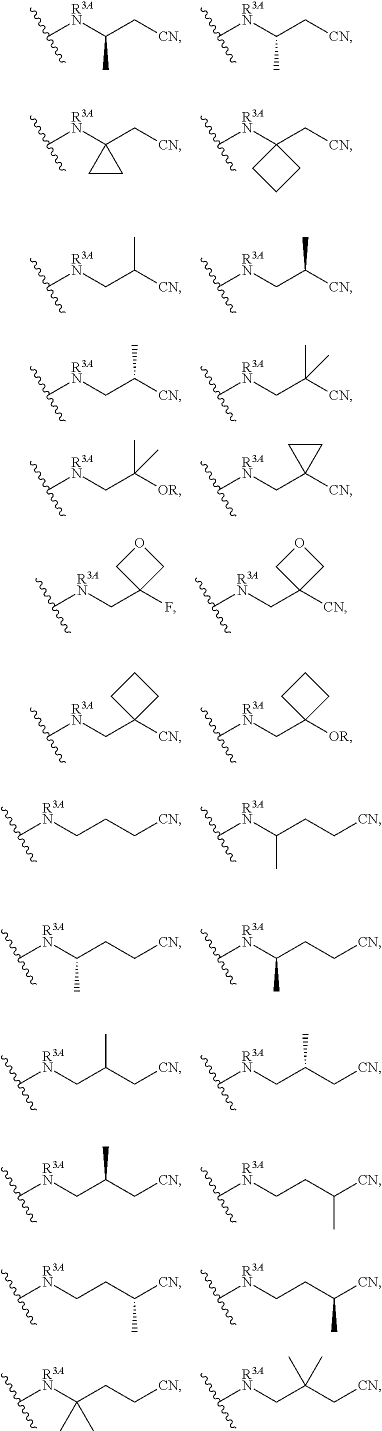

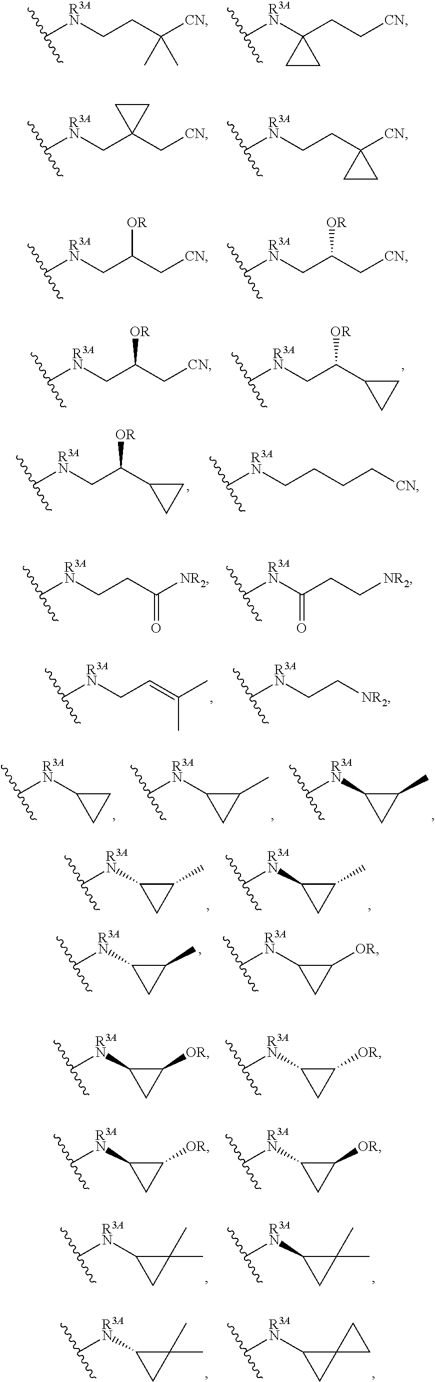

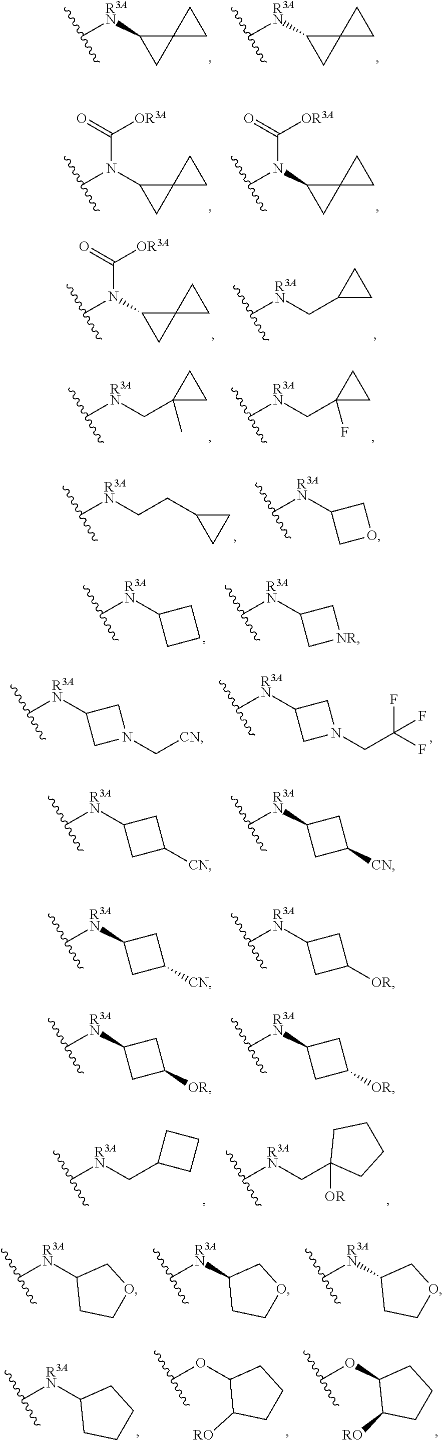

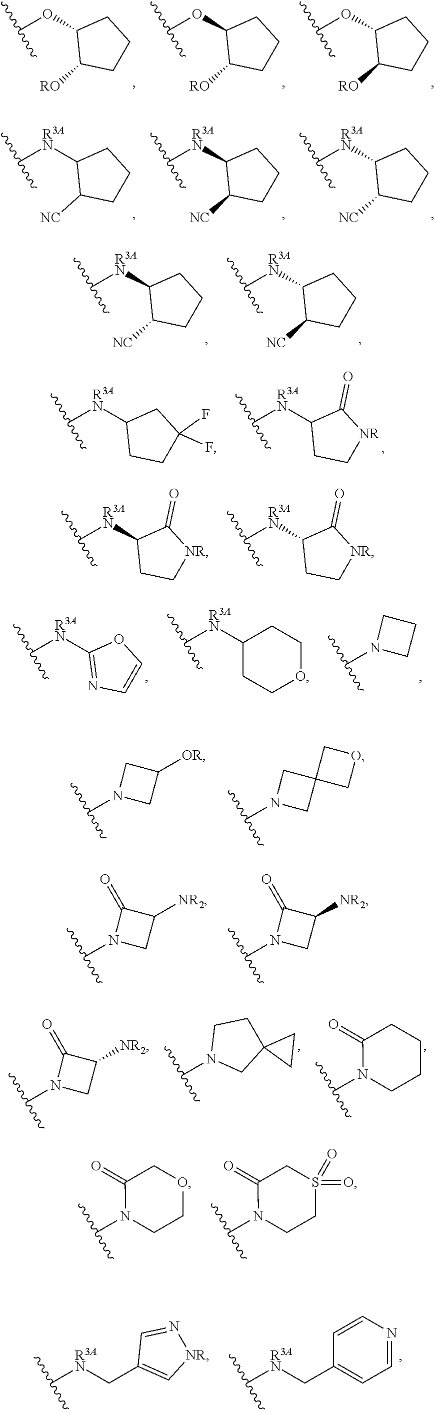

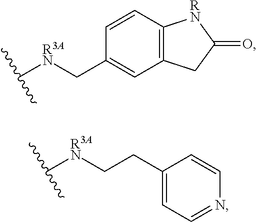

an optionally substituted phenyl, or an optionally substituted 5-6 membered heteroaryl having 1-3 ring heteroatoms selected from nitrogen, oxygen, and sulfur, wherein R^{3A} and R are as defined above and described in embodiments herein, both singly and in combination.

In certain embodiments, R¹³ is —N(R^{3A})₂,

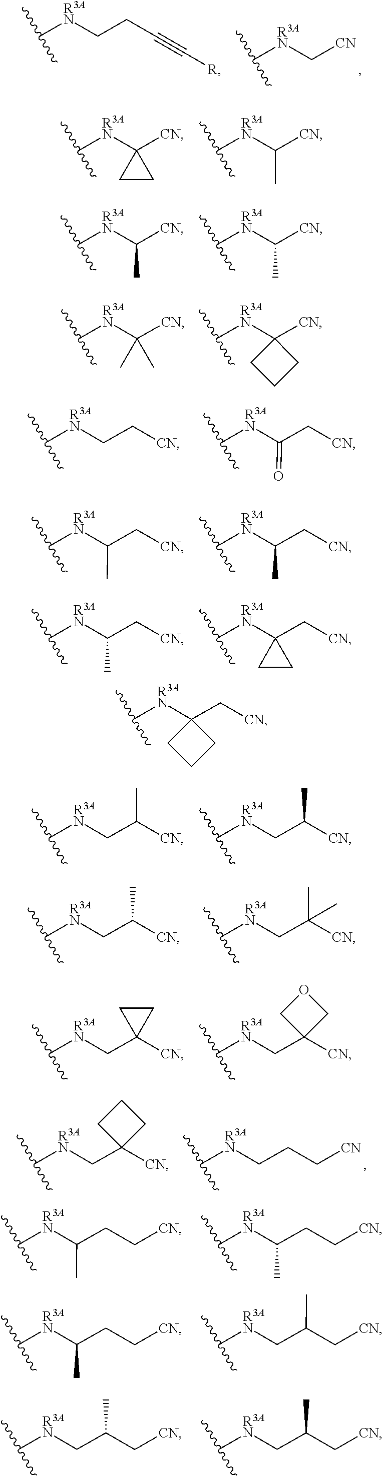

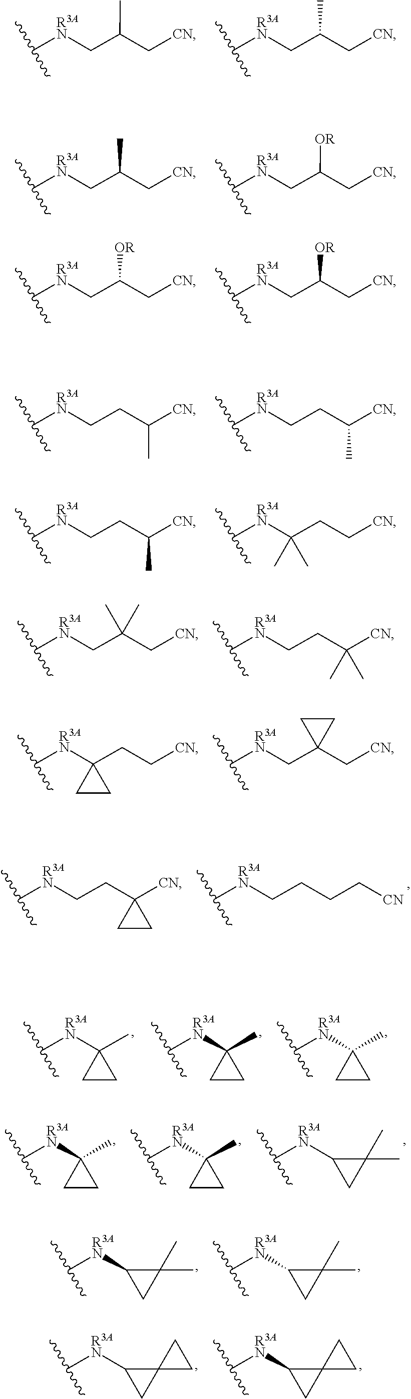

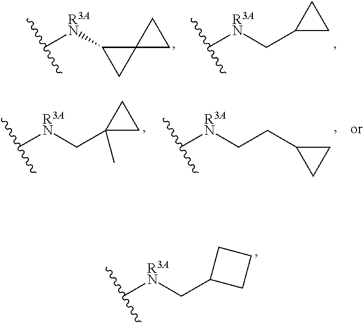

wherein R^{3A} is as defined above and described in embodiments herein, both singly and in combination.

In certain embodiments R¹³ is selected from Cl, —CN, cyclopropyl, isopentyl

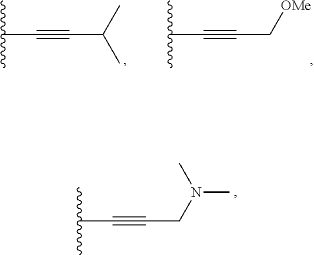

—CF₃, —CH₂OH, —CH₂OMe, —CH₂NHCOMe, —CH₂NHSO₂Me,

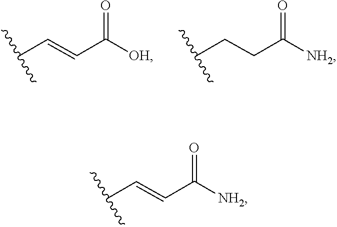 [SMILES: */C=C/C(=O)O ; *CCC(N)=O ; */C=C/C(N)=O]

—CO₂H, —CO₂Me, —CON(Me)OMe,

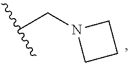 [SMILES: *CN1CCC1]

### —OH, —OMe, —OEt, —OiBu,

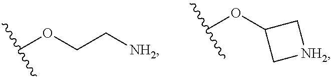

—NHEt, —NHnPr, —NHiPr, —NHiBu, —NHCOiPr, —NHC(NH)iPr, —NHCO₂Me, —NHCONHMe, —NHCONMe₂, —NHSO₂Me, —NHSO₂Et,

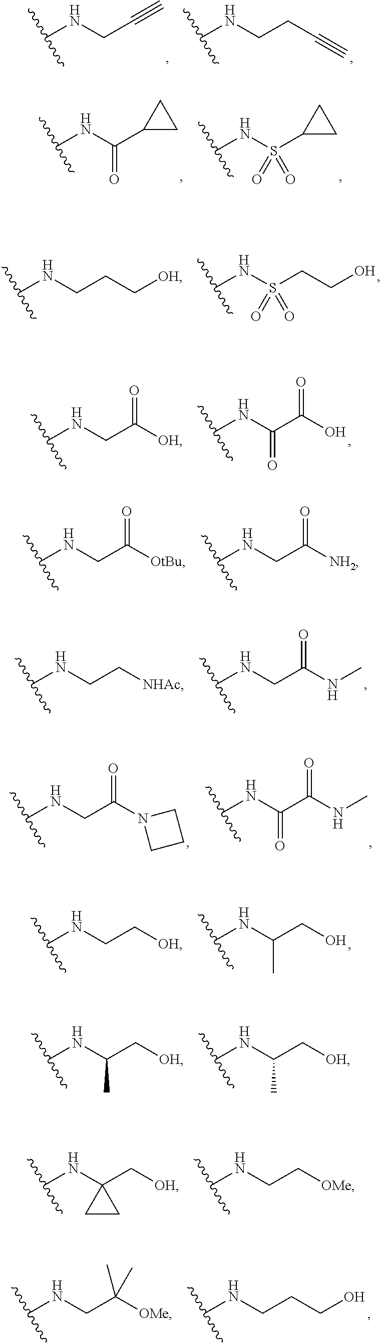

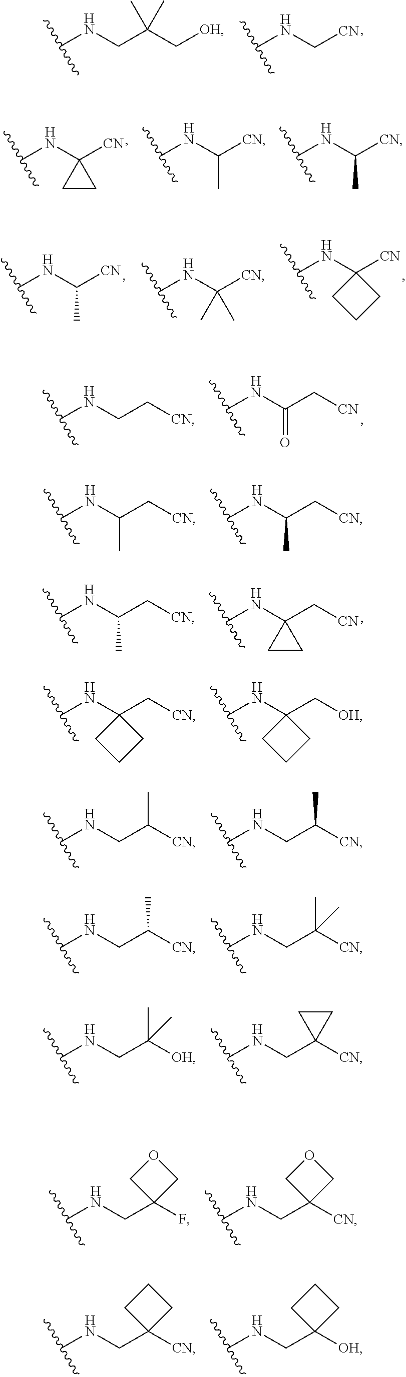

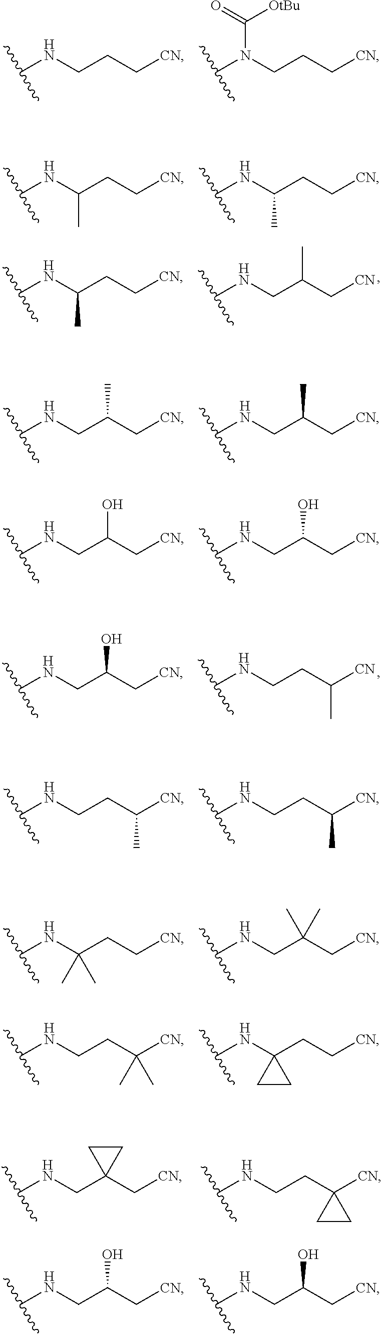

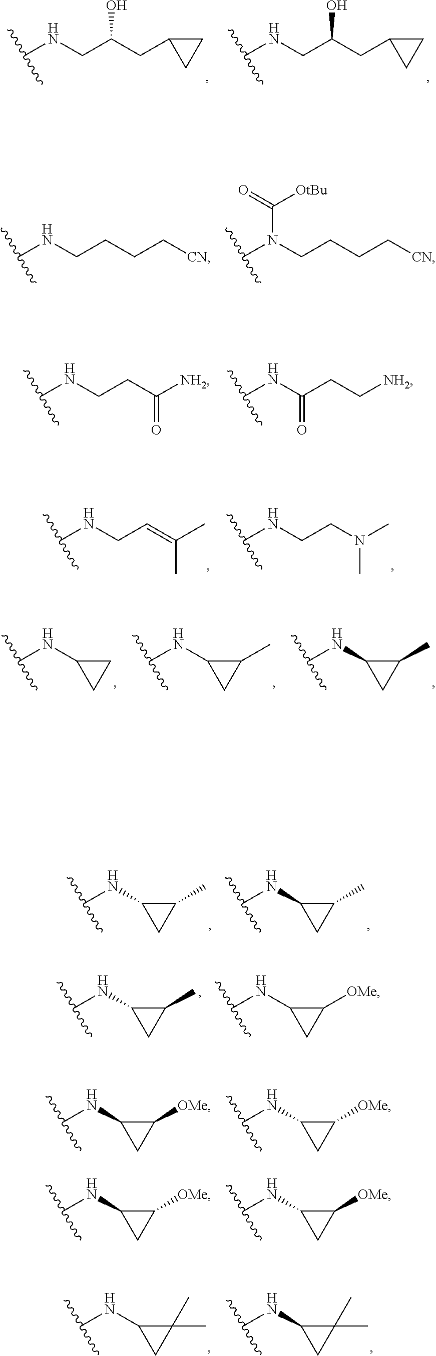

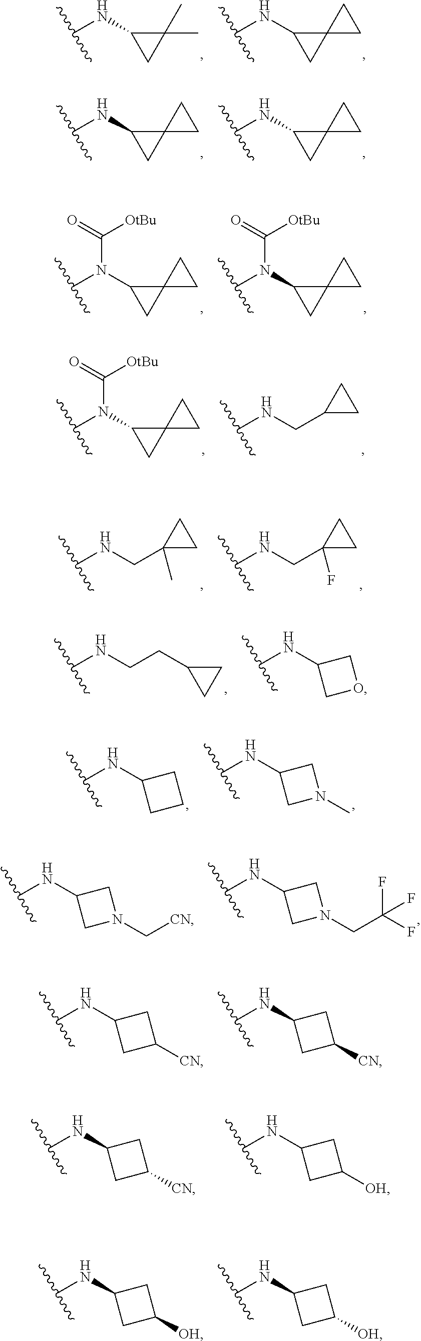

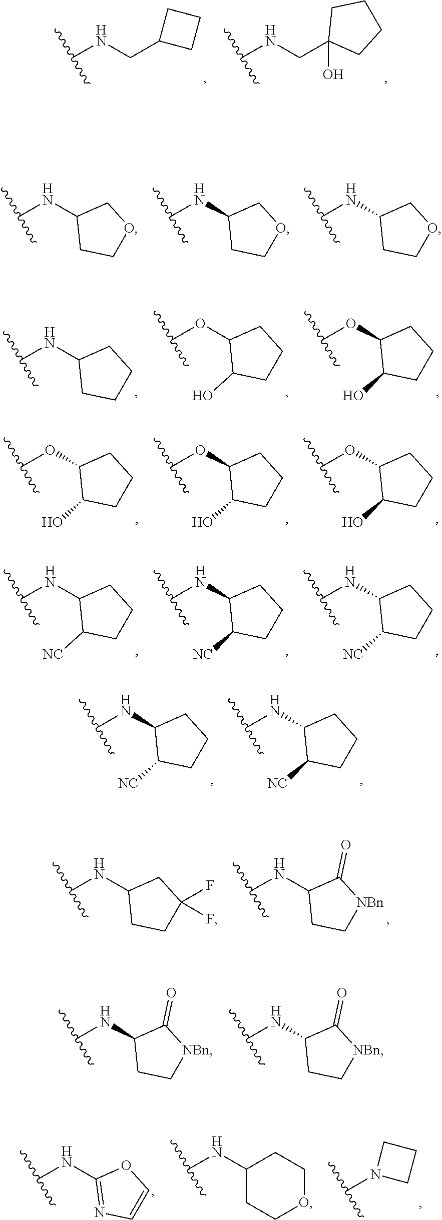

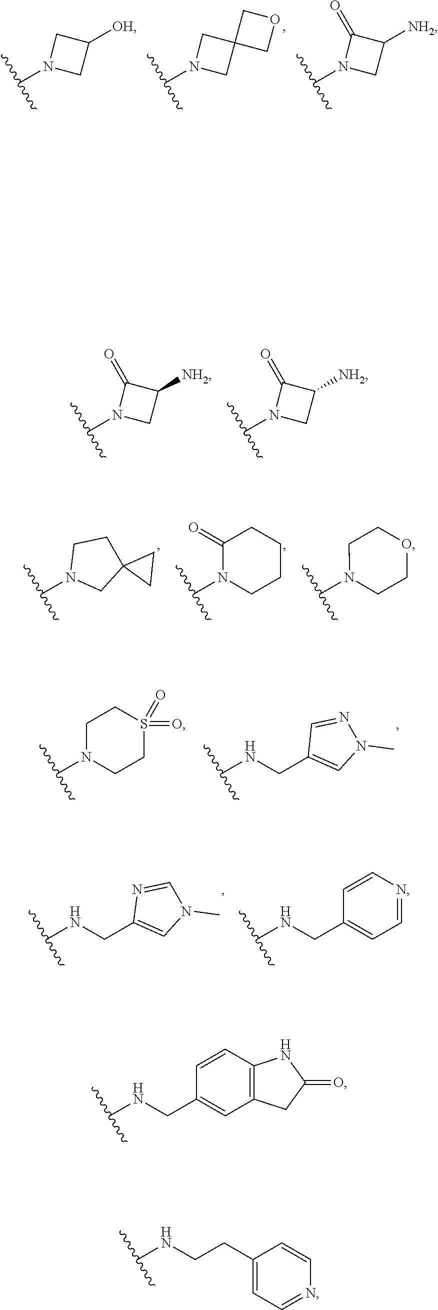

an optionally substituted phenyl, and an optionally substituted 5-6 membered heteroaryl having 1-3 ring heteroatoms selected from nitrogen, oxygen, and sulfur.

In certain embodiments, R¹³ is —NHiBu,

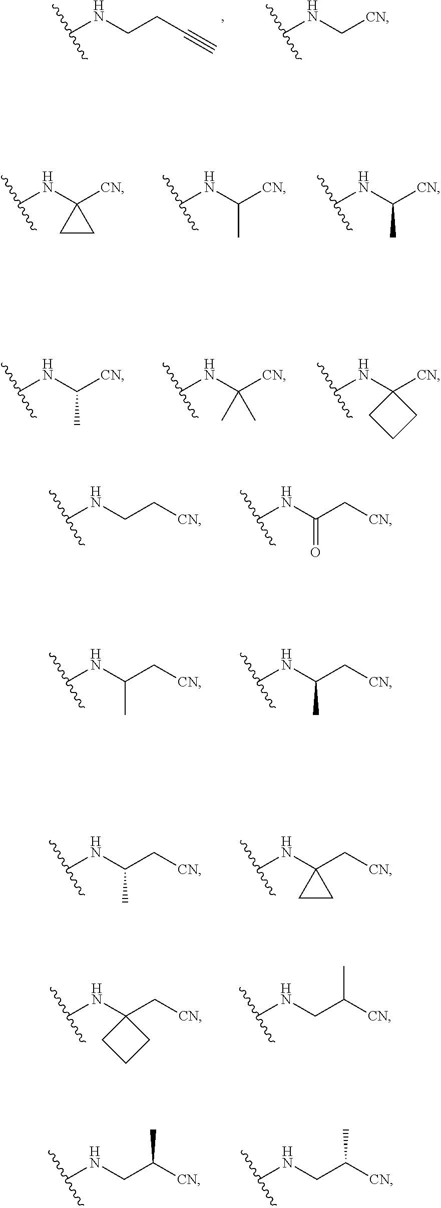

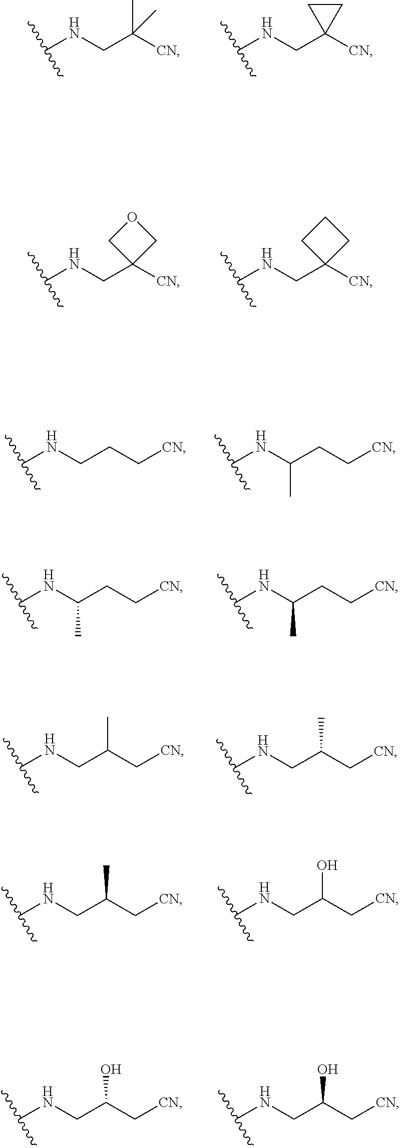

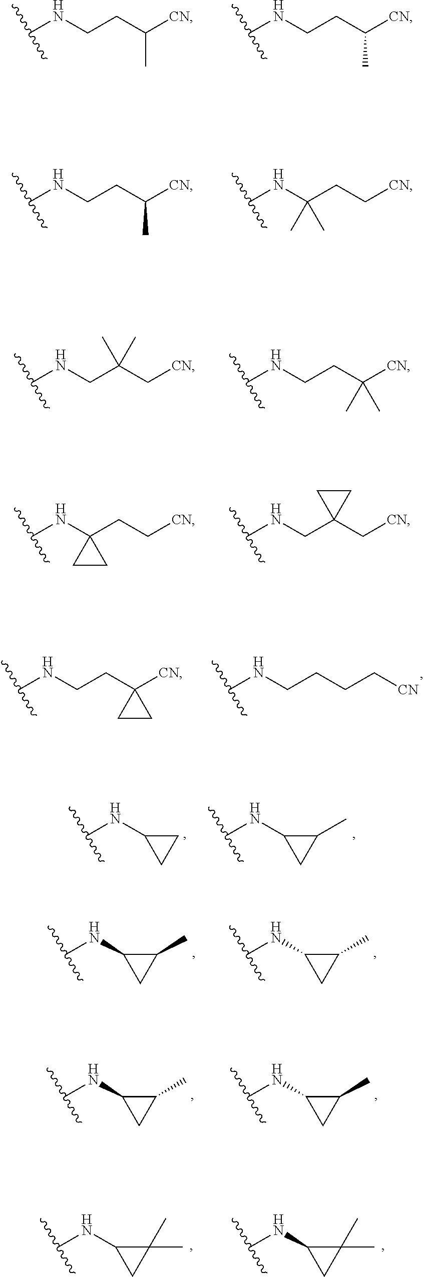

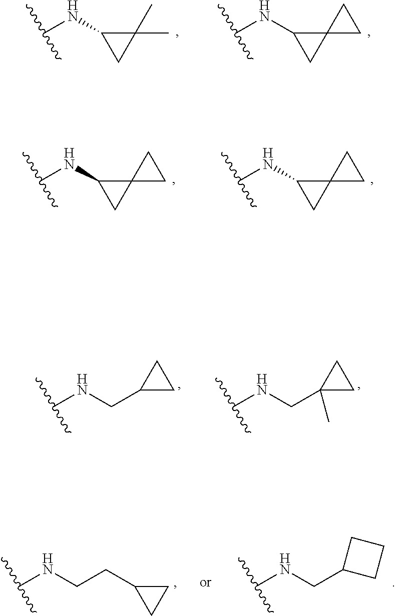

In some embodiments, R¹³ is selected from those depicted in Table 1, below.

As defined generally above, q is 0, 1, 2, or 3.

In some embodiments, q is 0, 1, 2, or 3.

In some embodiments, q is 0. In some embodiments, q is 1. In some embodiments, q is 2. In some embodiments, q is 3.

In some embodiments, q is 0, 1, or 2. In some embodiments, q is 1, 2, or 3. In some embodiments, q is 1 or 2. In some embodiments, q is 2 or 3.

In some embodiments, q is selected from the values represented in the compounds depicted in Table 1, below.

As defined generally above, each R is independently hydrogen, or an optionally substituted group selected from C₁₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; a 6-11 membered saturated or partially unsaturated bicyclic carbocyclic ring having 1-2 heteroatoms independently selected from nitrogen, oxygen, and sulfur, or two R groups on the same atom are optionally taken together with the atom to form an optionally substituted 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorus, and sulfur.

In some embodiments, R is hydrogen. In some embodiments, R is an optionally substituted C₁₋₆ aliphatic. In some embodiments. R is an optionally substituted phenyl. In some embodiments, R is an optionally substituted naphthyl. In some embodiments. R is an optionally substituted 3-7 membered saturated or partially unsaturated monocyclic carbocyclic ring. In some embodiments, R is an optionally substituted 3-7 membered saturated or partially unsaturated monocyclic heterocyclic ring having 1-2 heteroatoms independently selected from nitrogen, oxygen, and sulfur. In some embodiments, R is an optionally substituted 5-6 membered monocyclic heteroaryl ring having 1-4 heteroatoms independently selected from nitrogen, oxygen, and sulfur. In some embodiments, R is an optionally substituted 8-10 membered bicyclic heteroaryl ring having 1-4 heteroatoms independently selected from nitrogen, oxygen, and sulfur. In some embodiments, R is an optionally substituted 7-12 membered saturated or partially unsaturated bicyclic heterocyclic ring having 1-4 heteroatoms independently selected from nitrogen, oxygen, and sulfur. In some embodiments, R is an optionally substituted 5-8 membered saturated or partially unsaturated bridged bicyclic ring having 0-3 heteroatoms independently selected from nitrogen, oxygen, and sulfur. In some embodiments, R is an optionally substituted 6-10 membered saturated or partially unsaturated spirocyclic ring having 0-3 heteroatoms independently selected from nitrogen, oxygen, and sulfur. In some embodiments, R is an optionally substituted 6-11 membered saturated or partially unsaturated bicyclic carbocyclic ring having 1-2 heteroatoms independently selected from nitrogen, oxygen, and sulfur. In some embodiments, two R groups on the same atom are taken together with the atom to form an optionally substituted 3-7 membered saturated or partially unsaturated monocyclic carbocyclic ring. In some embodiments, two R groups on the same atom are taken together with the atom to form an optionally substituted 3-7 membered saturated or partially unsaturated monocyclic heterocyclic ring having 1-2 heteroatoms independently selected from nitrogen, oxygen, phosphorus, and sulfur.

In some embodiments, R is selected from those depicted in Table 1, below.

In some embodiments, the present invention provides a compound represented by any one of the following formulae:

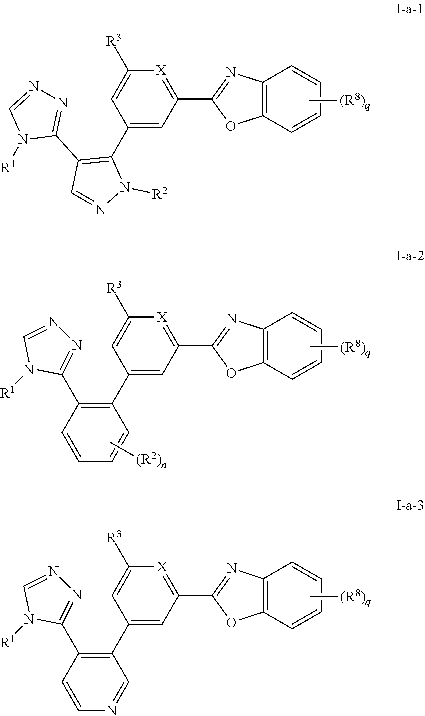

or a pharmaceutically acceptable salt thereof, wherein each of X, R¹, R², R³, R⁸, q and n are as defined above and described in embodiments herein, both singly and in combination.

In some embodiments, the present invention provides a compound represented by any one of the following formulae:

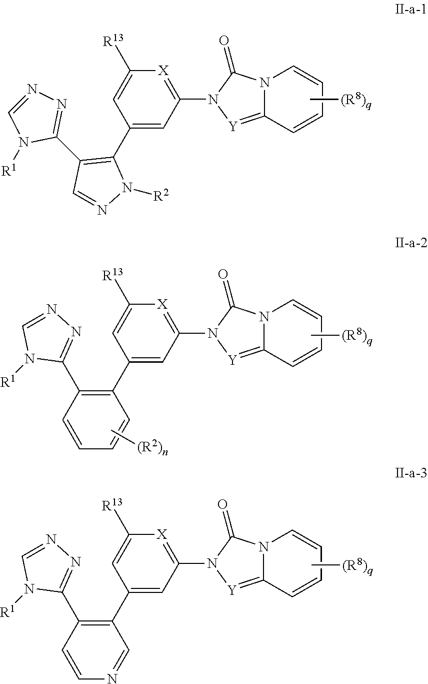

or a pharmaceutically acceptable salt thereof, wherein each of X, Y, R¹, R², R¹³, R⁸, q and n are as defined above and described in embodiments herein, both singly and in combination.

In some embodiments, the present invention provides a compound represented by the following formulae:

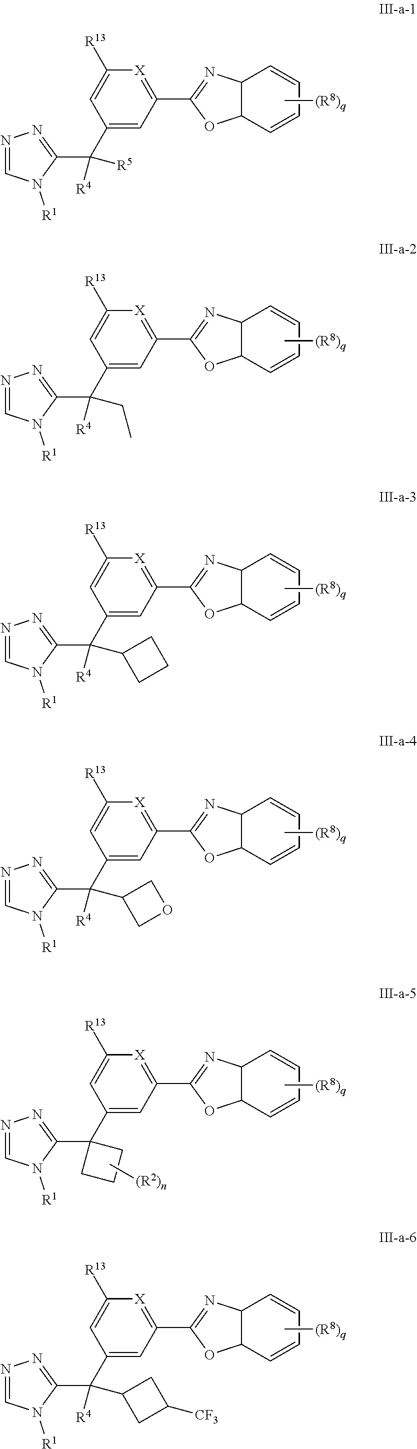

or a pharmaceutically acceptable salt thereof, wherein each of R¹, R², R⁴, R⁵, R¹³, R⁸, X, q and n are as defined above and described in embodiments herein, both singly and in combination.

In some embodiments, the present invention provides a compound represented by any one of the following formulae:

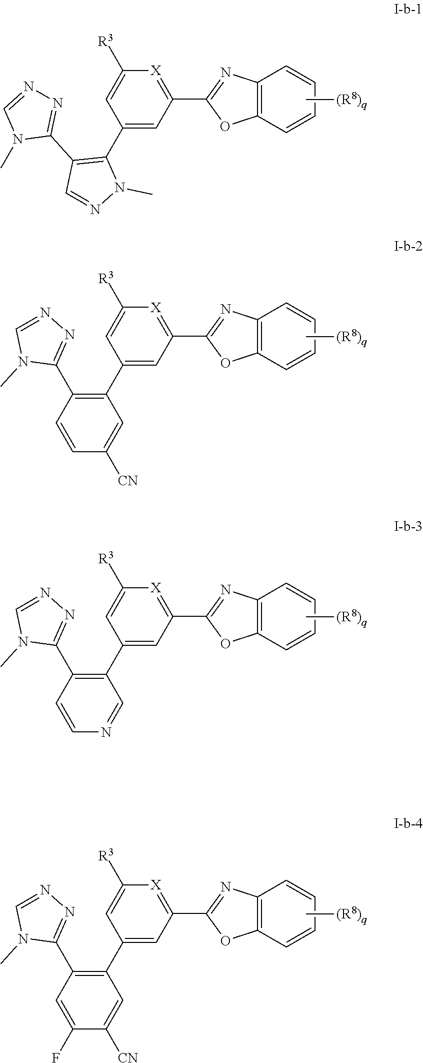

or a pharmaceutically acceptable salt thereof, wherein each of X, R³, R⁸, and q are as defined above and described in embodiments herein, both singly and in combination.

In some embodiments, the present invention provides a compound represented by any one of the following formulae:

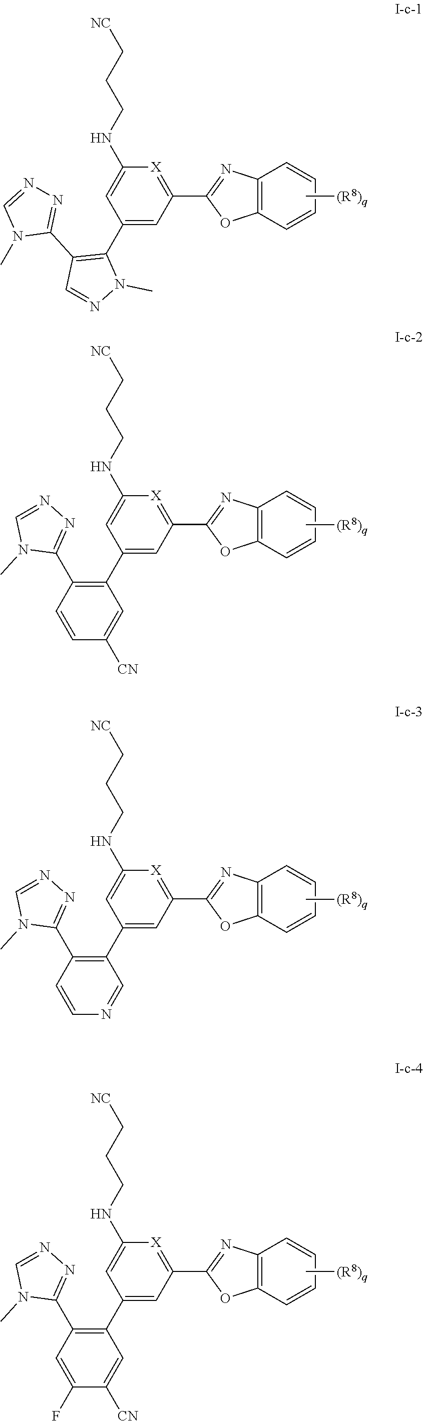

or a pharmaceutically acceptable salt thereof, wherein each of R⁸ and q are as defined above and described in embodiments herein, both singly and in combination.

In some embodiments, the present invention provides a compound represented by any one of the following formulae:

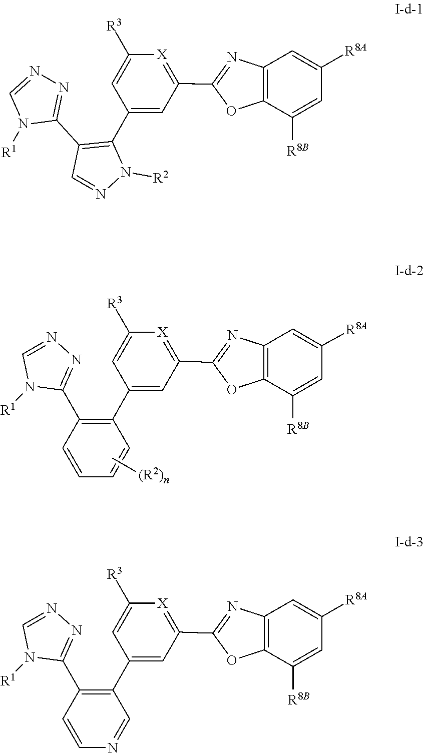

or a pharmaceutically acceptable salt thereof, wherein R^{8A} and R^{8B} are each independently selected from hydrogen, halogen, —CF₃, —OR, —NR₂, and an optionally substituted C₁₋₆ aliphatic, and wherein each of X, R¹, R², R³, and n are as defined above and described in embodiments herein, both singly and in combination.

In some embodiments, the present invention provides a compound represented by any one of the following formulae:

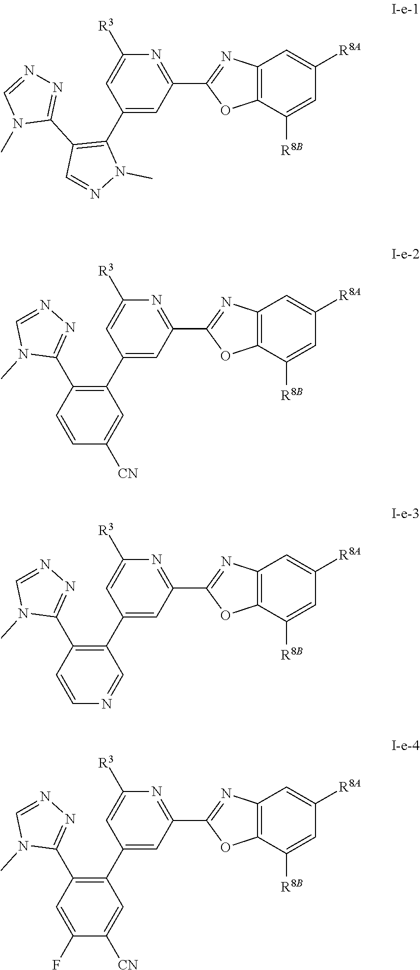

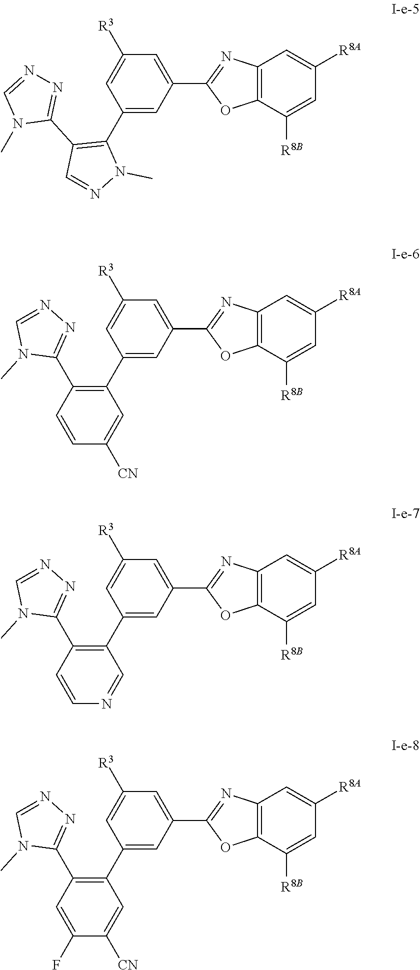

or a pharmaceutically acceptable salt thereof, wherein R^{8A} and R^{8B} are each independently selected from hydrogen, halogen, —CF₃, —OR, —NR₂, and an optionally substituted C₁₋₆ aliphatic, and wherein each of R³ and R are as defined above and described in embodiments herein, both singly and in combination.

In some embodiments, R^{8A} is selected from hydrogen, halogen, —CF₃, —OR, —NR₂, and an optionally substituted C₁₋₆ aliphatic.

In some embodiments. R^{8A} is hydrogen. In some embodiments, R^{8A} is halogen. In some embodiments, R^{8A} is —CF₃. In some embodiments, R^{8A} is —OR. In some embodiments, R^{8A} is —NR₂. In some embodiments, R^{8A} is an optionally substituted C₁₋₆ aliphatic.

In some embodiments, R^{8A} is selected from halogen, C₁₋₆alkyl, —CF₃, —OR, —CH₂OR and —CH₂NR₂.

In some embodiments, R^{8A} is selected from fluoro, chloro, methyl, —CF₃, —OR,

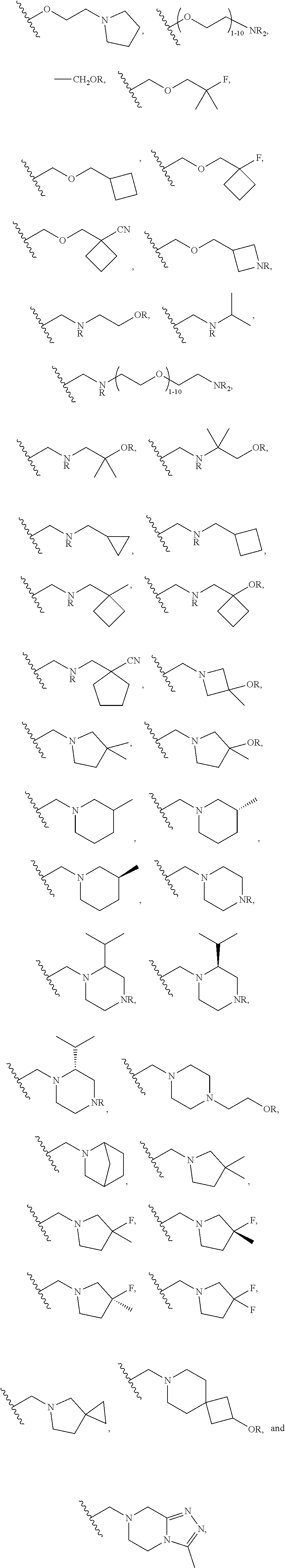

wherein R is as defined above and described in embodiments herein, both singly and in combination.

In some embodiments, R^{8B} is selected from hydrogen, halogen, —CF₃, —OR, —NR₂, and an optionally substituted C₁₋₆ aliphatic.

In some embodiments, R^{8B} is hydrogen. In some embodiments, R^{8B} is halogen. In some embodiments, R^{8B} is —CF₃. In some embodiments, R^{8B} is —OR. In some embodiments, R^{8B} is —NR₂. In some embodiments, R^{8B} is an optionally substituted C₁₋₆aliphatic.

In some embodiments, R^{8B} is hydrogen or —CF₃.

In some embodiments, R^{8B} is hydrogen, halogen, or —CF₃ and R^{3A} is —CH₂OR or —CH₂NR₂.

In some embodiments, R^{8B} is hydrogen or —CF₃ and R^{3A} is —CH₂OR or —CH₂NR₂.

In some embodiments, R^{8B} is hydrogen or —CF₃ and R^{3A} is —CH₂OR or —CH₂NR₂, wherein —C₂OR or —CH₂NR₂ is selected from —CH₂OH,

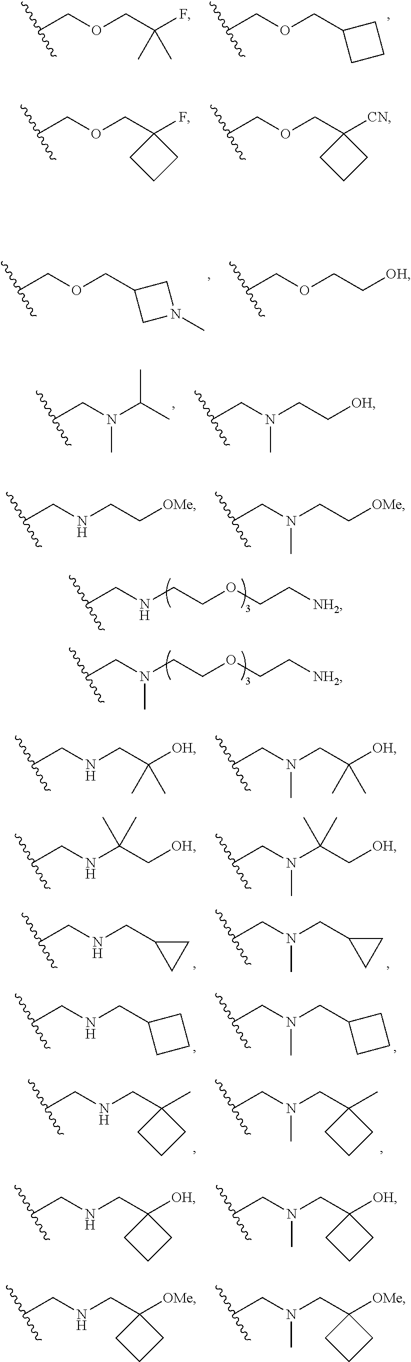

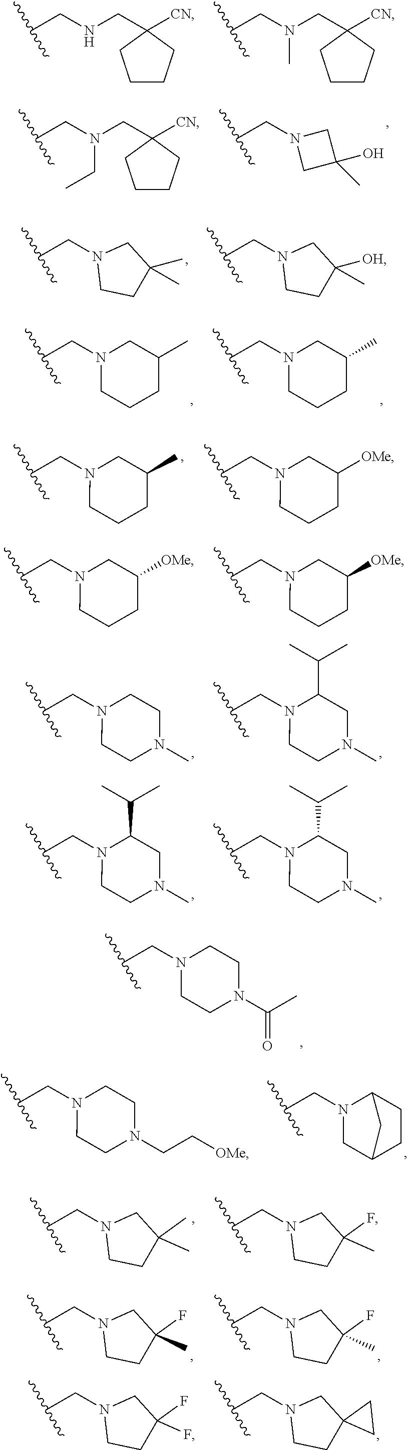

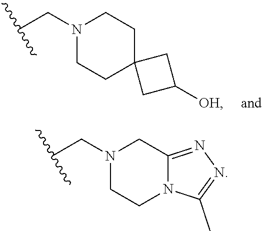

In some embodiments, R^{8A} and R^{8B} are selected from those depicted in Table 1, below.

In some embodiments, the present invention provides a compound represented by any one of the following formulae:

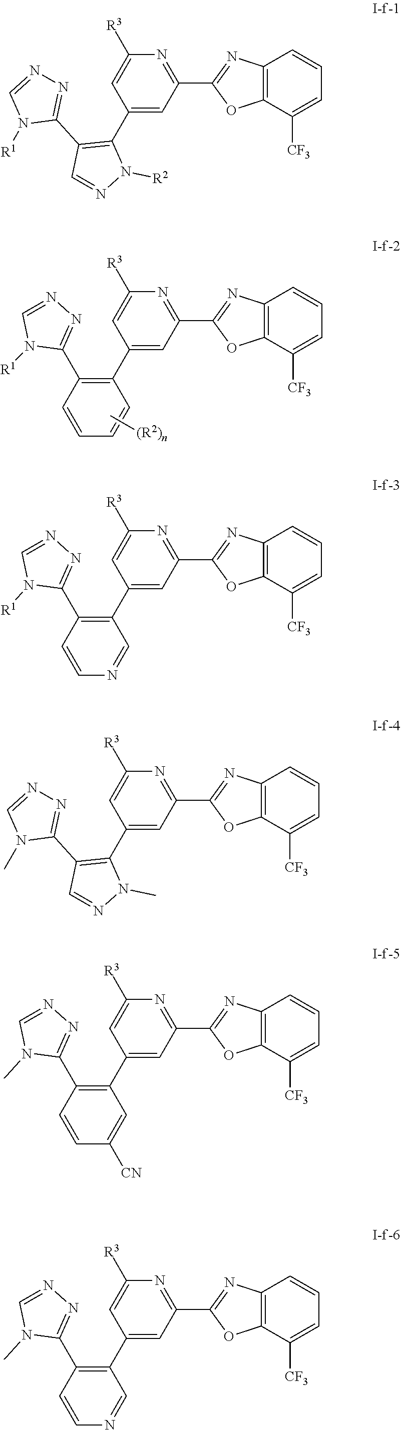

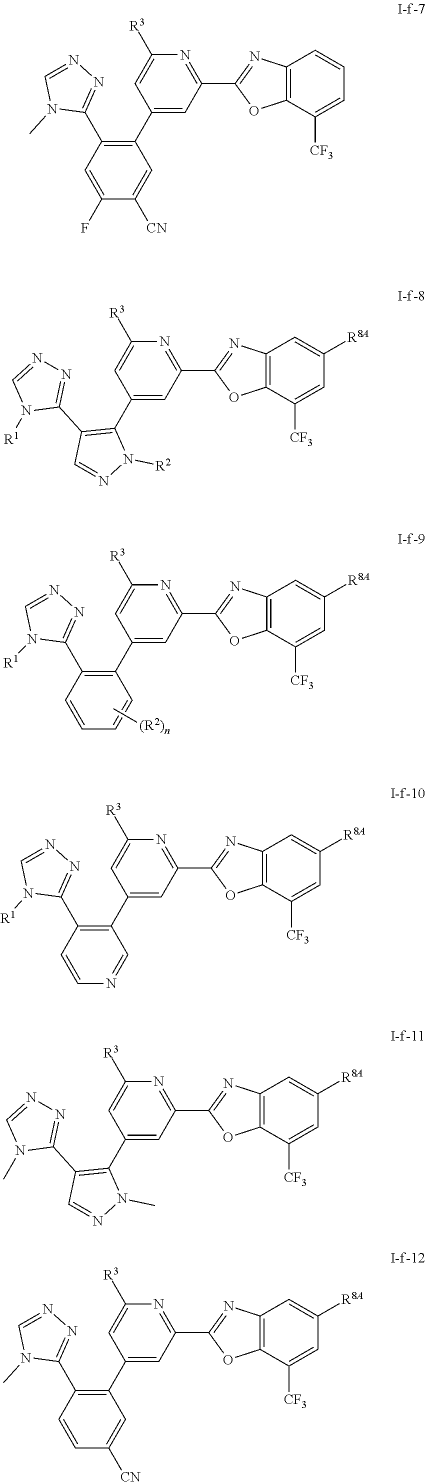

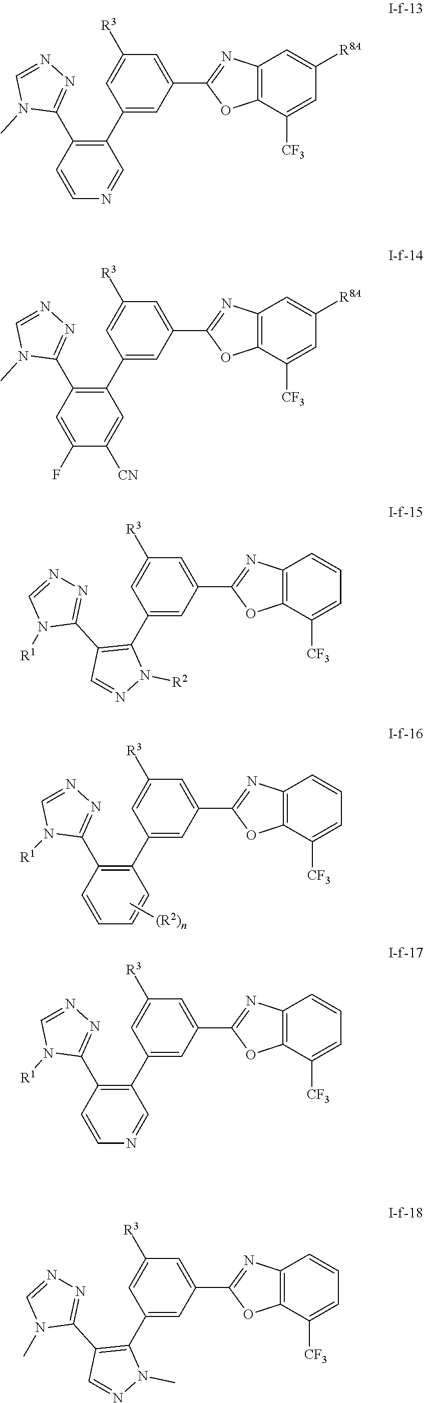

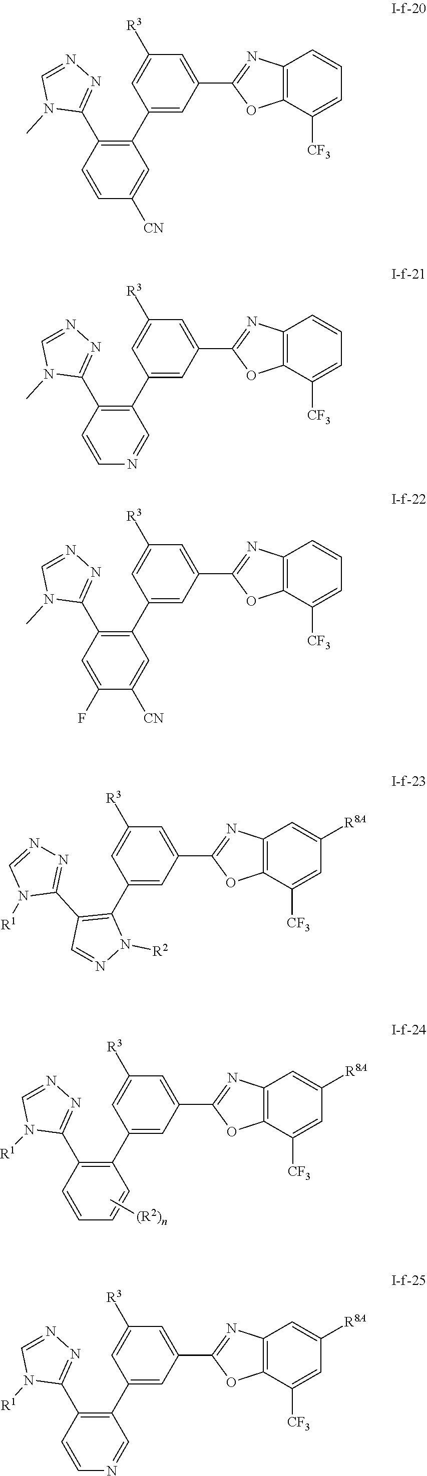

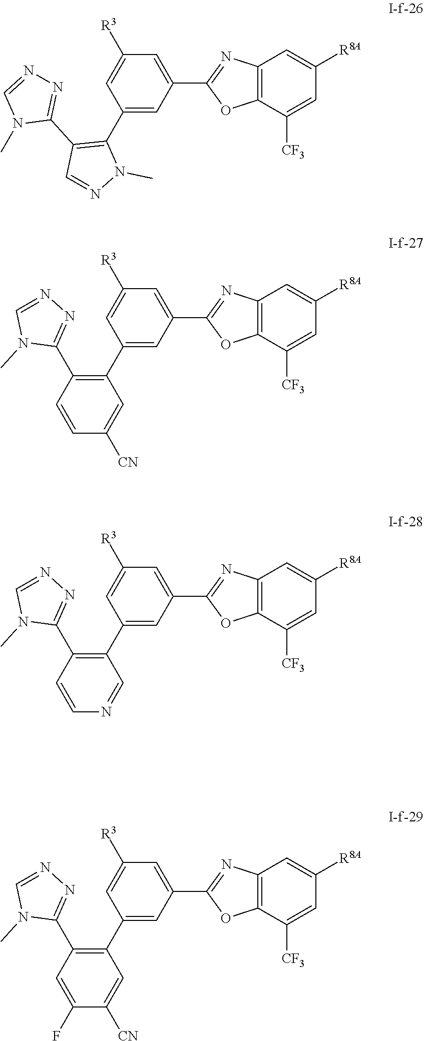

or a pharmaceutically acceptable salt thereof, wherein R^{3A} is selected from hydrogen, halogen, —CF₃, —OR, —NR₂, and an optionally substituted C₁₋₆ aliphatic, and wherein each of R¹, R², R³, and n are as defined above and described in embodiments herein, both singly and in combination.

In some embodiments, the present invention provides a compound represented by any one of the following formulae

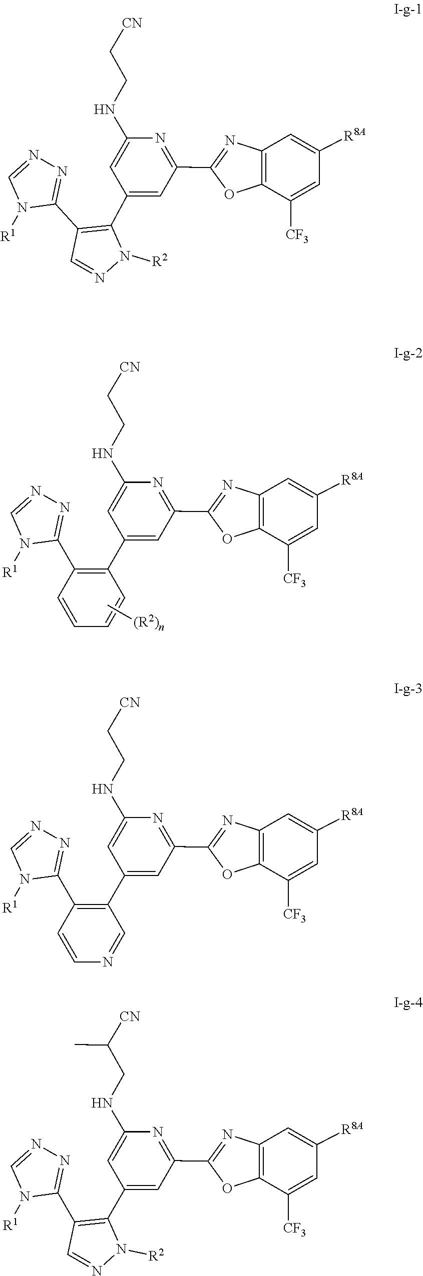

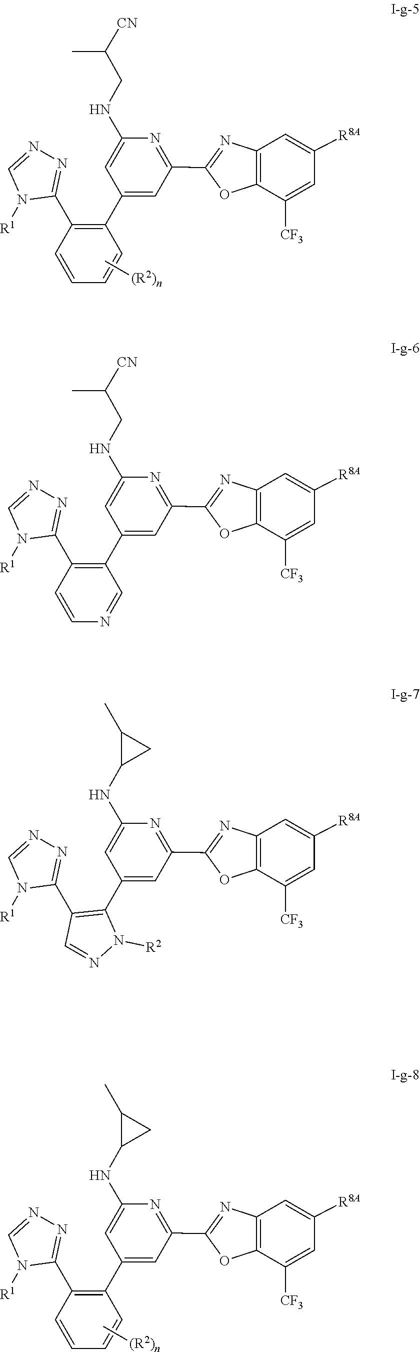

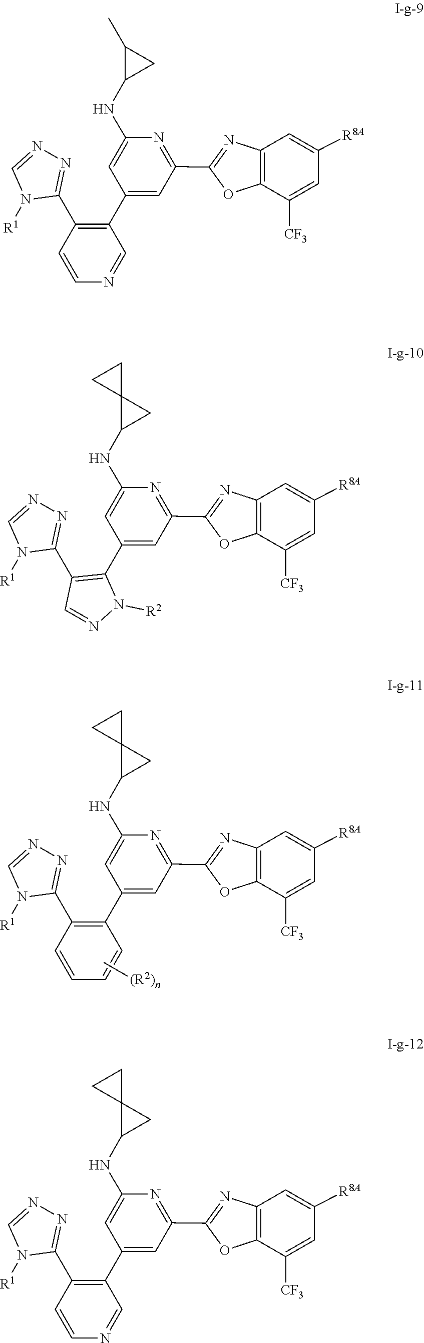

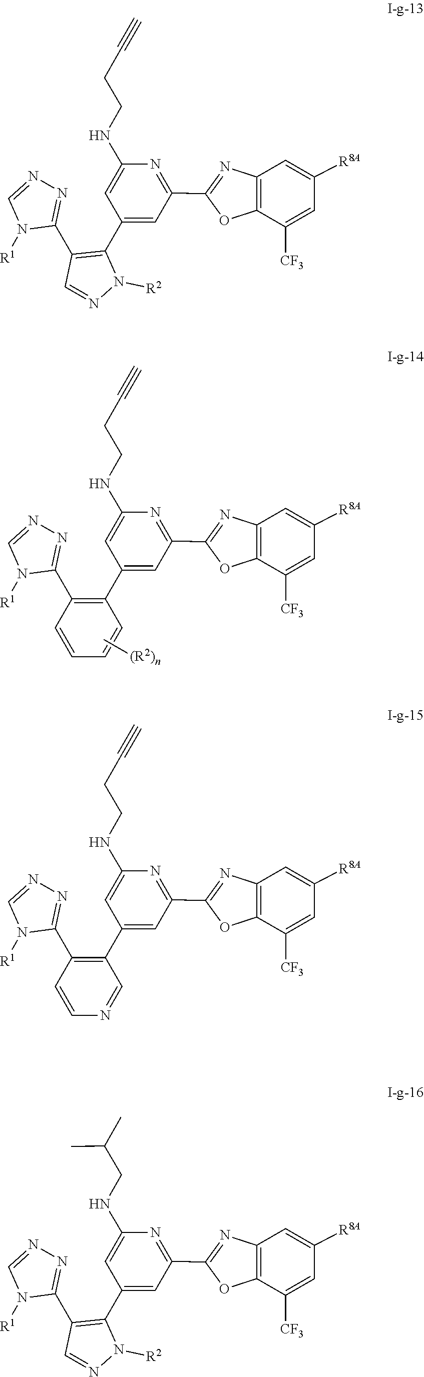

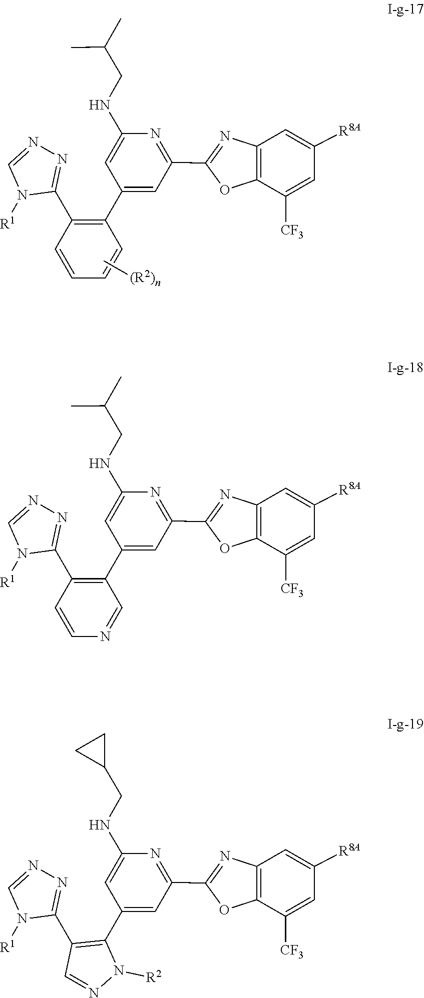

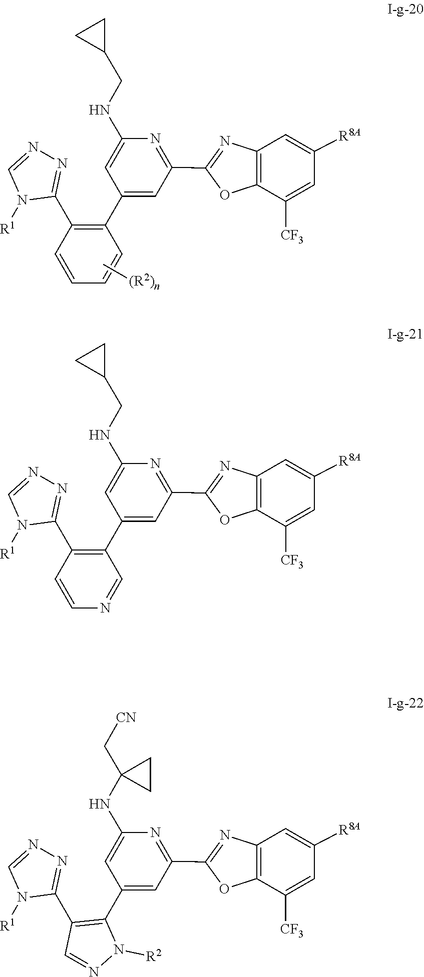

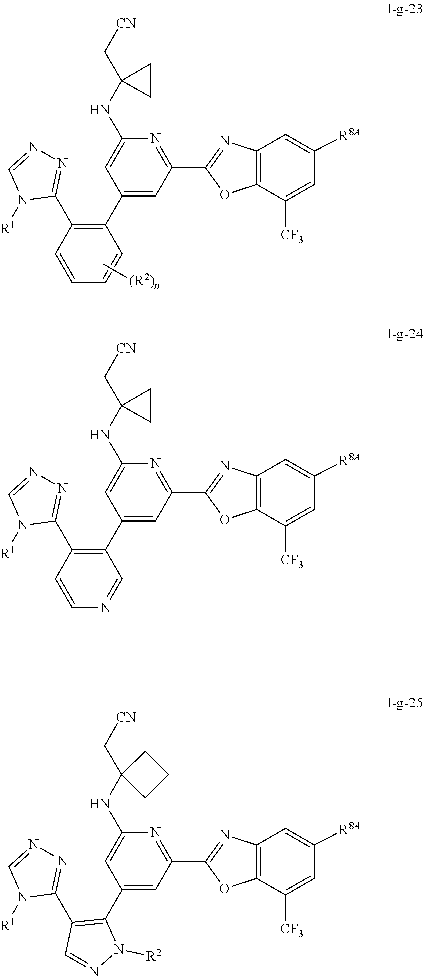

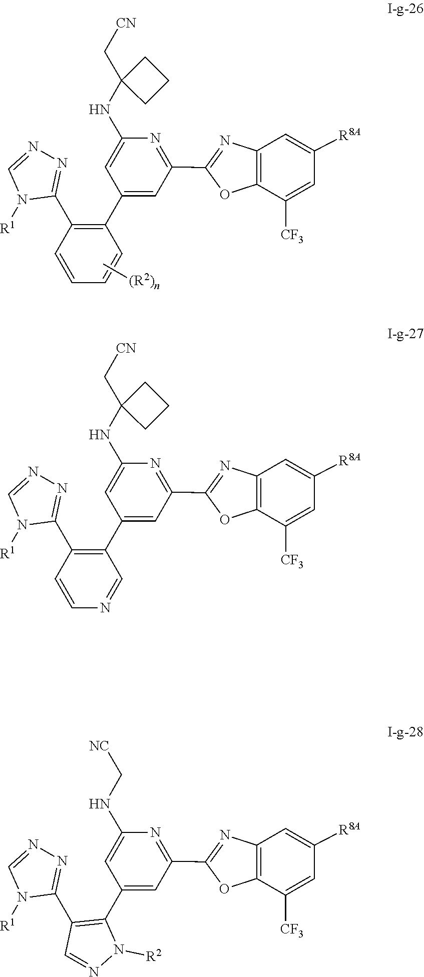

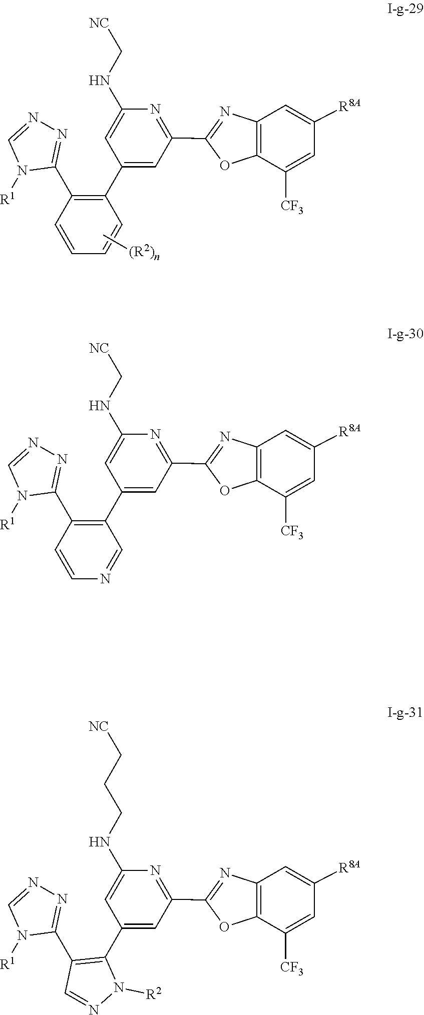

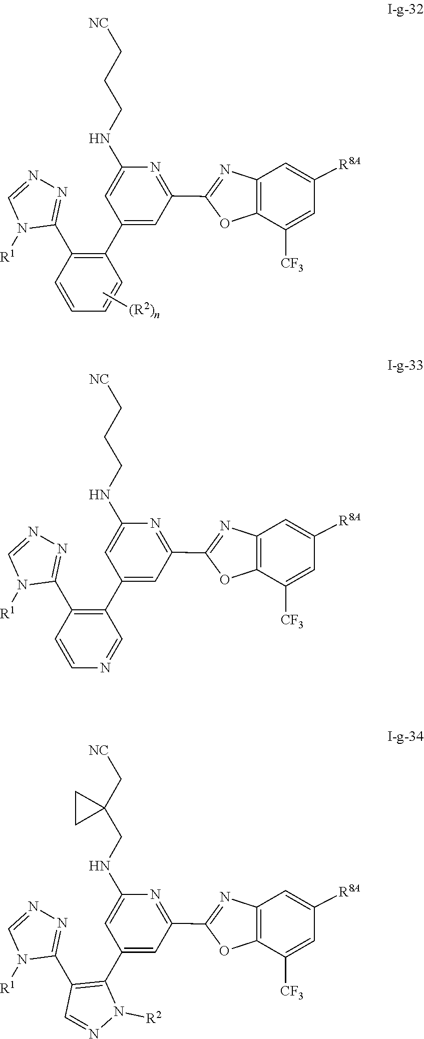

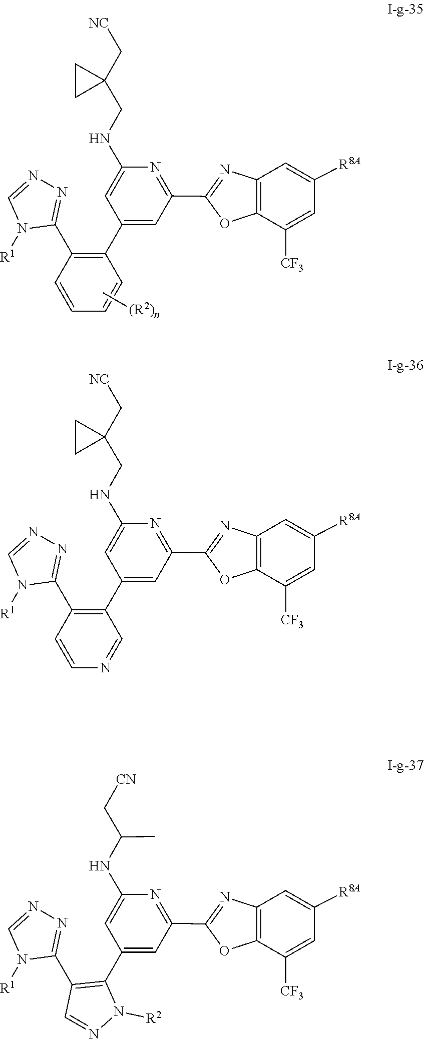

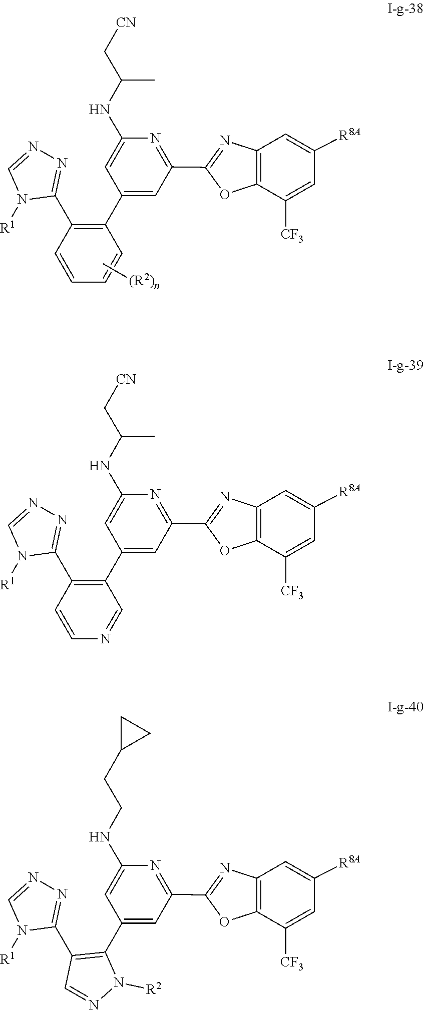

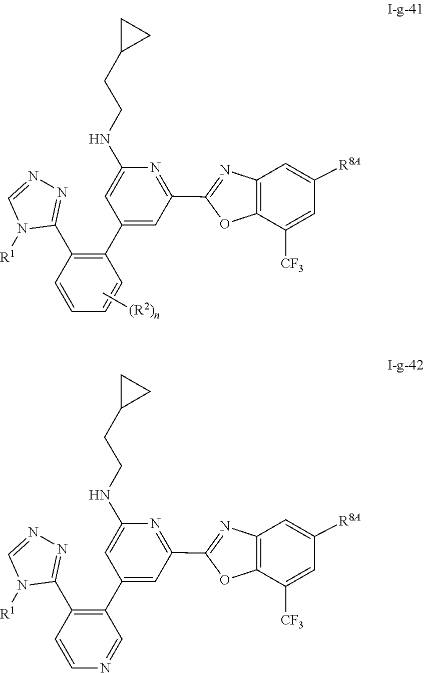

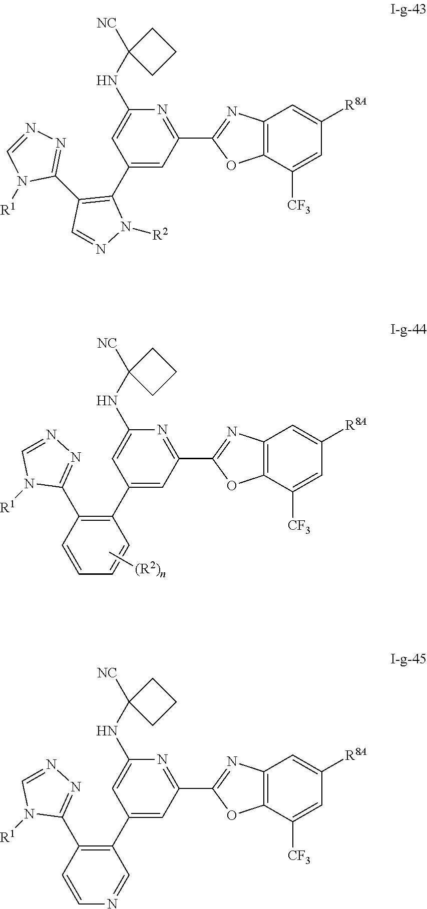

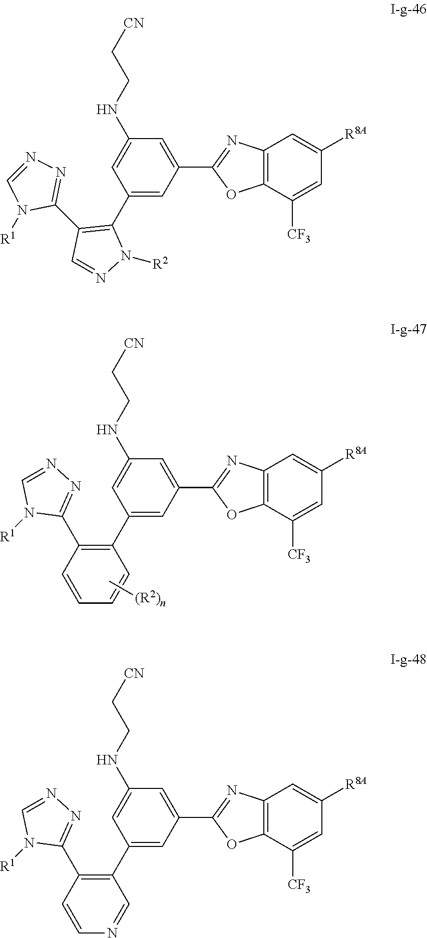

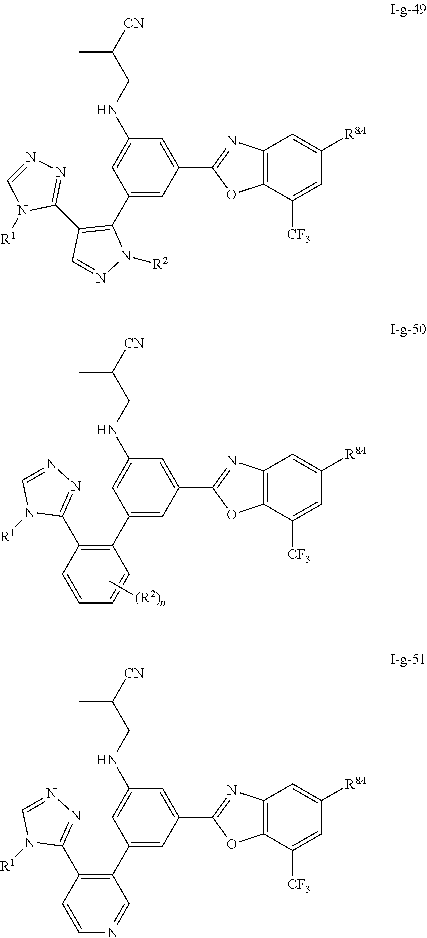

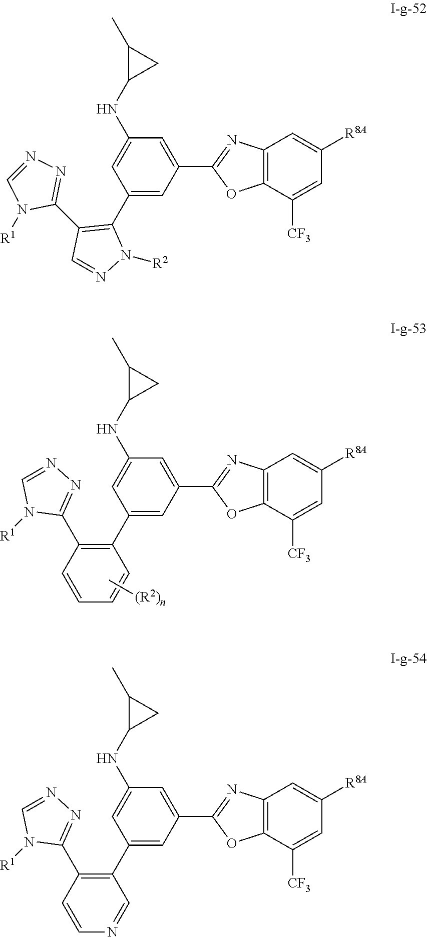

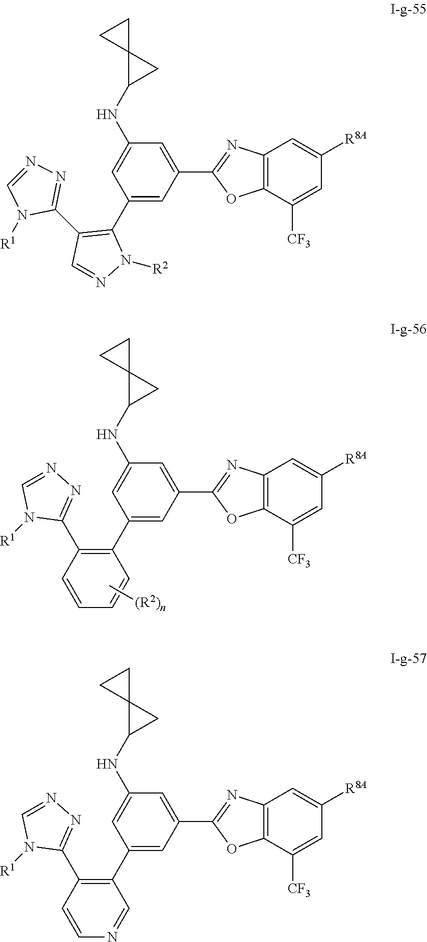

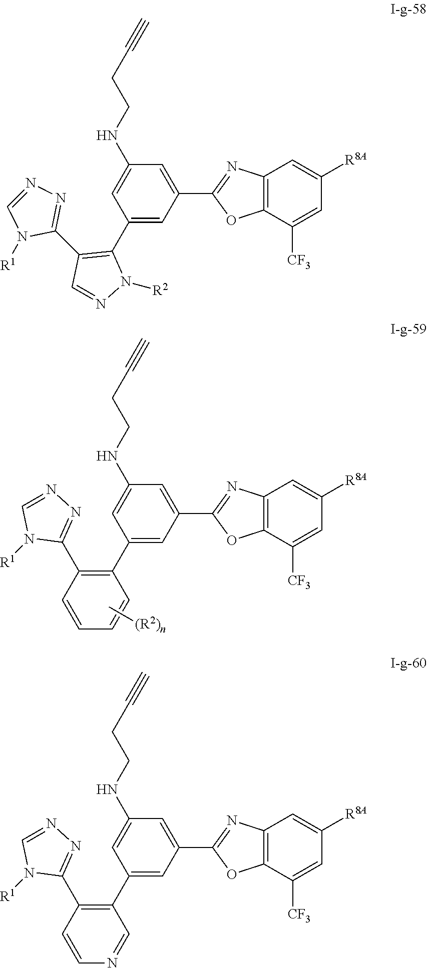

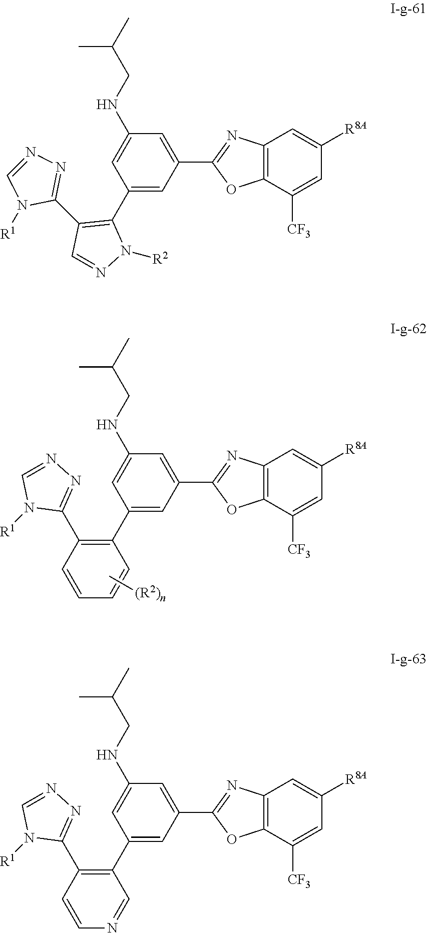

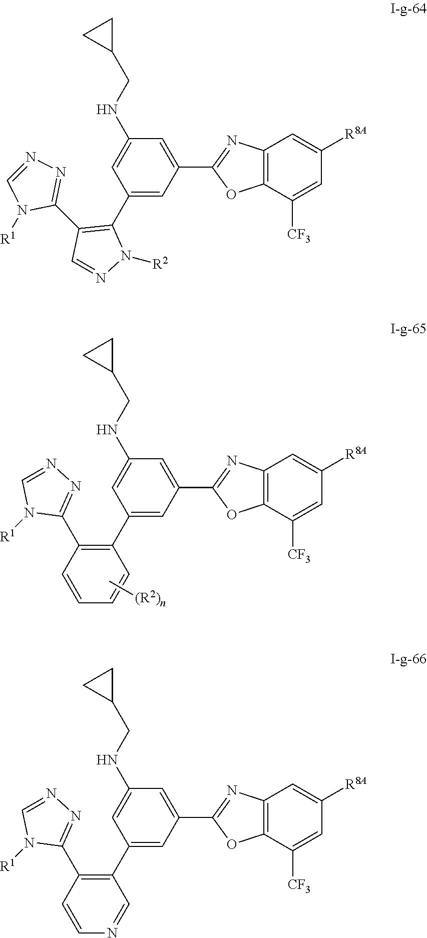

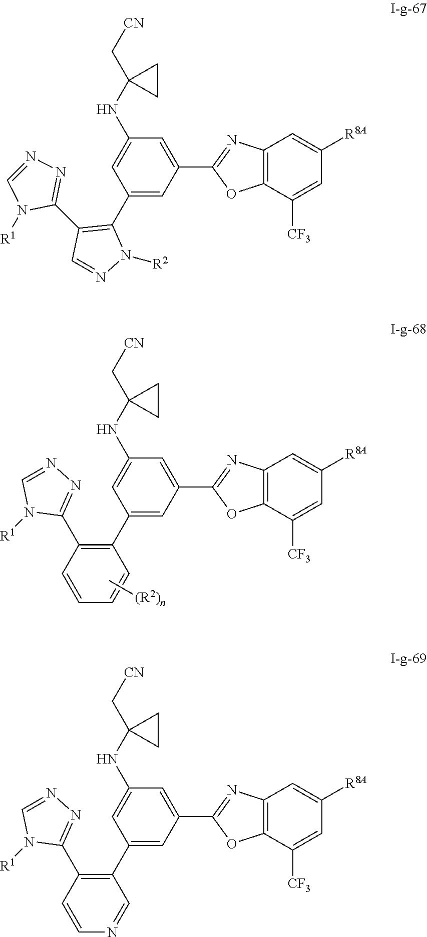

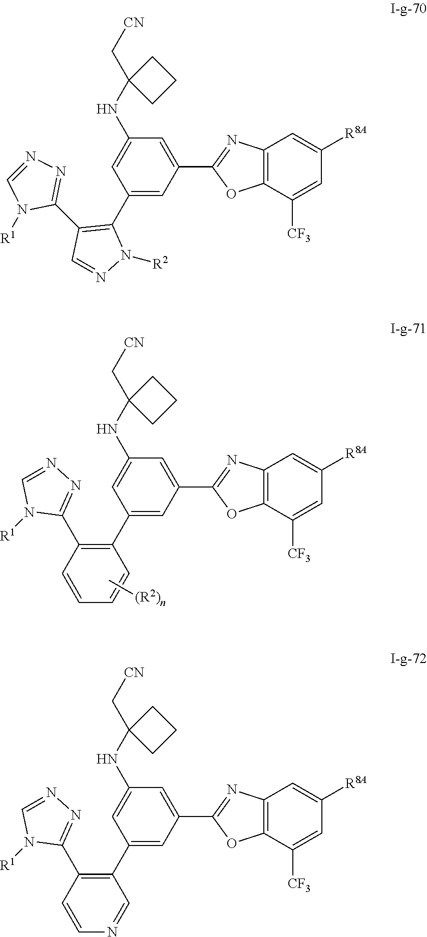

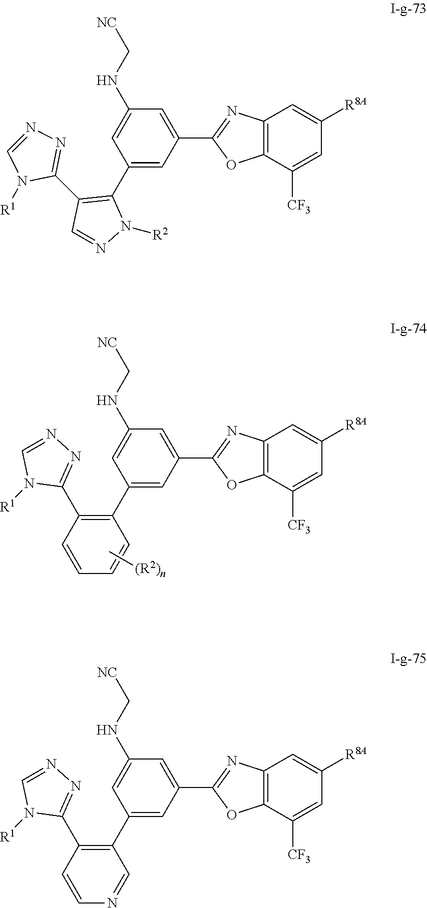

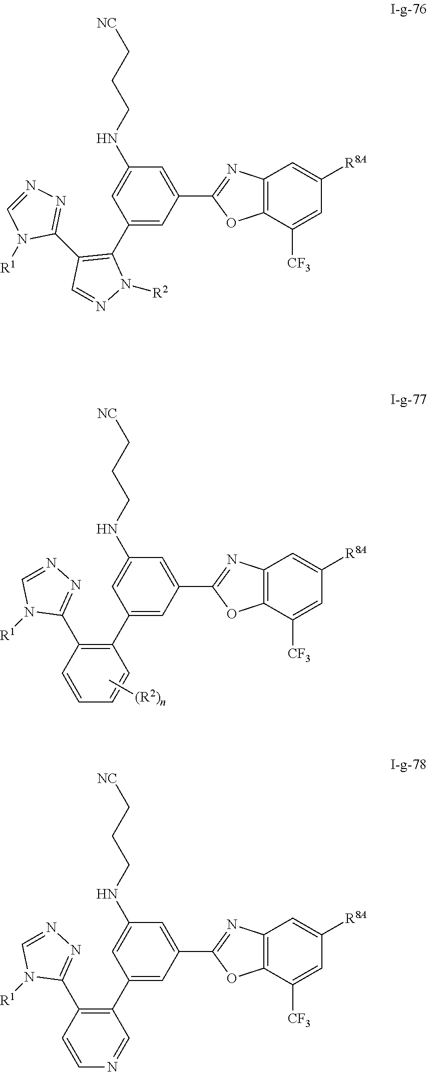

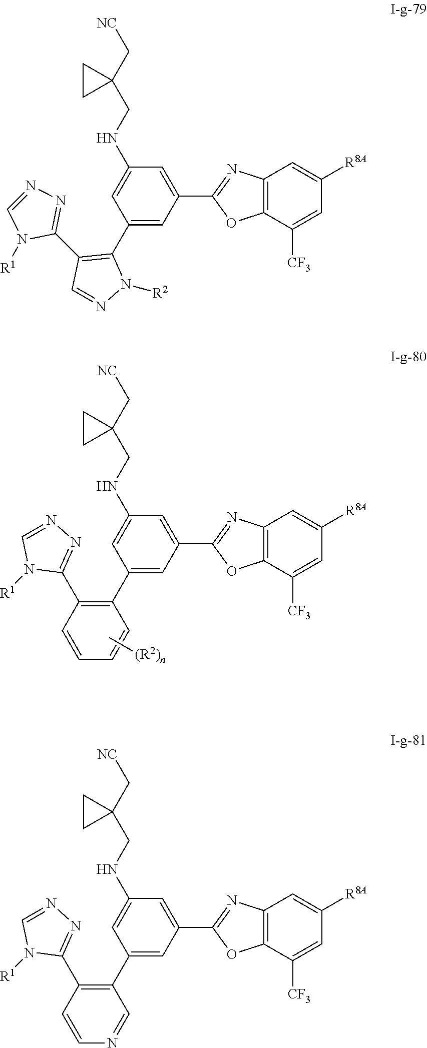

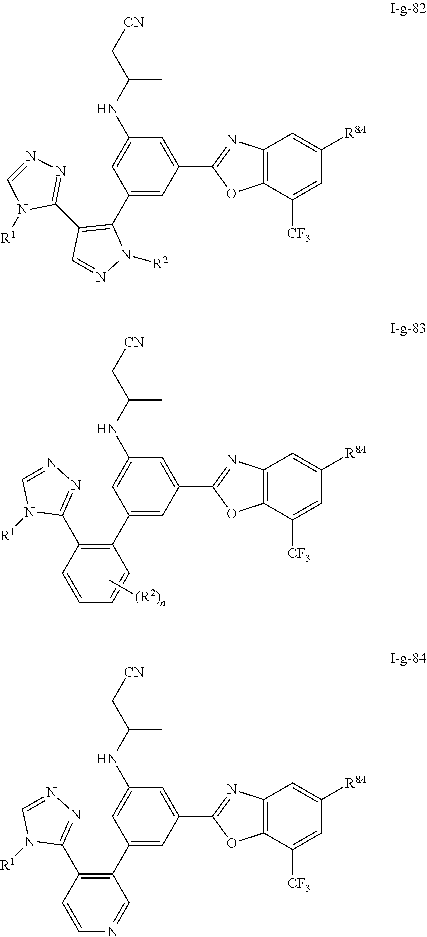

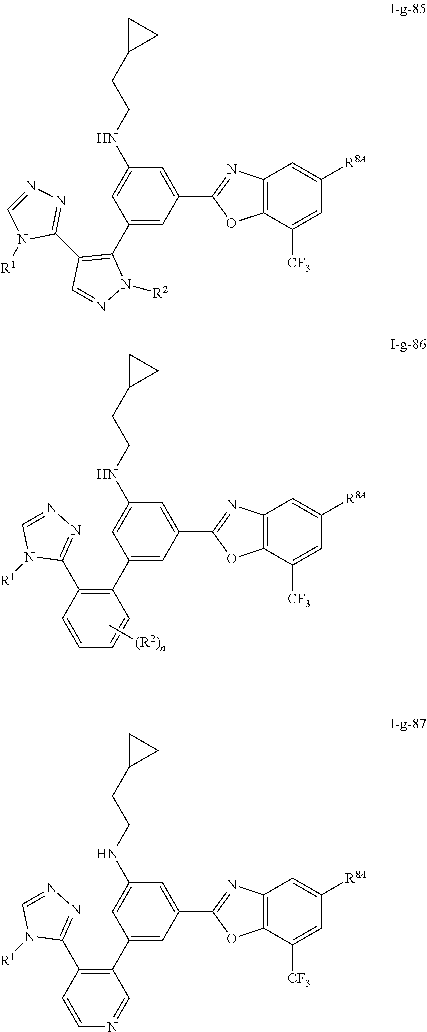

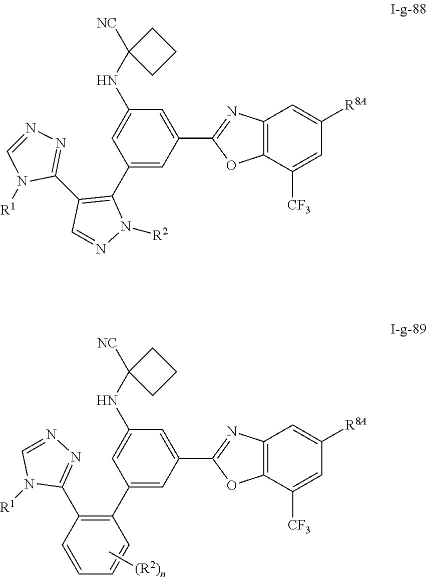

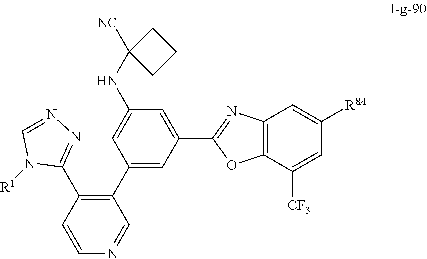

or a pharmaceutically acceptable salt thereof, wherein each of R¹, R², R³, R⁸, q and n is as defined above and described in embodiments herein, both singly and in combination.

Exemplary compounds of the invention are set forth in Table 1, below.

TABLE 1
Selected Compounds

| I-# | Compound |
|---|---|
| I-1 | 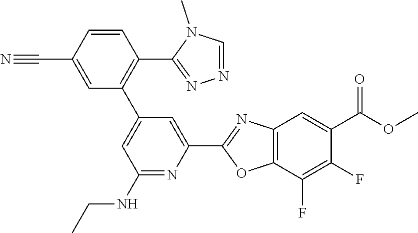 [SMILES: CCNc1cc(-c2cc(C#N)ccc2-c2nncn2C)cc(-c2nc3cc(C(=O)OC)c(F)c(F)c3o2)n1] |
| I-2 | 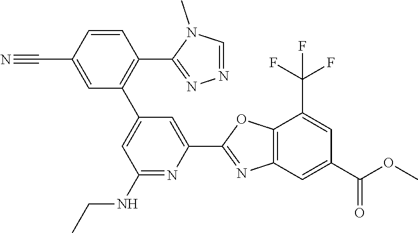 [SMILES: CCNc1cc(-c2cc(C#N)ccc2-c2nncn2C)cc(-c2nc3cc(C(=O)OC)cc(C(F)(F)F)c3o2)n1] |
| I-3 | 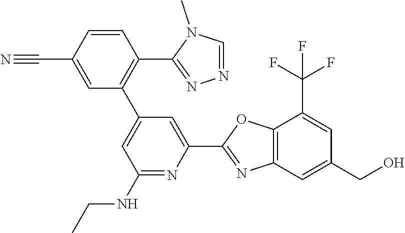 [SMILES: CCNc1cc(-c2cc(C#N)ccc2-c2nncn2C)cc(-c2nc3cc(CO)cc(C(F)(F)F)c3o2)n1] |
| I-4 | 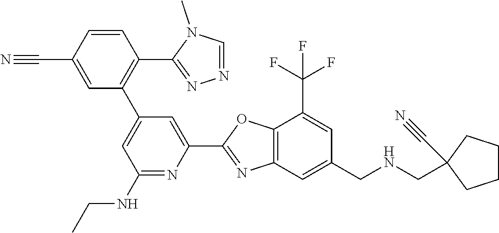 [SMILES: CCNc1cc(-c2cc(C#N)ccc2-c2nncn2C)cc(-c2nc3cc(CNCC4(C#N)CCCC4)cc(C(F)(F)F)c3o2)n1] |
| I-5 | 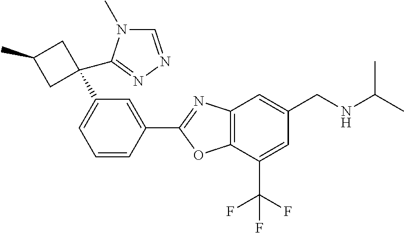 [SMILES: CC(C)NCc1cc(C(F)(F)F)c2oc(-c3cccc([C@]4(c5nncn5C)C[C@@H](C)C4)c3)nc2c1] |
| I-6 | 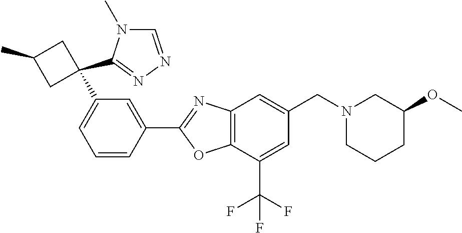 |
| I-7 | 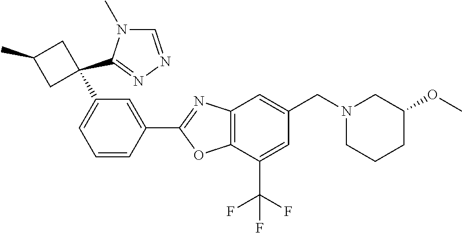 |
| I-8 | 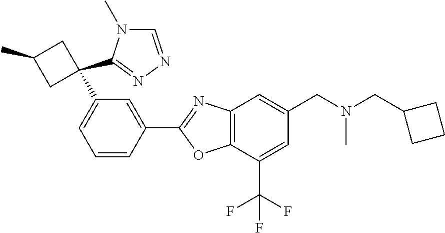 [SMILES: CN(Cc1cc(C(F)(F)F)c2oc(-c3cccc([C@]4(c5nncn5C)C[C@@H](C)C4)c3)nc2c1)CC1CCC1] |
| I-9 | 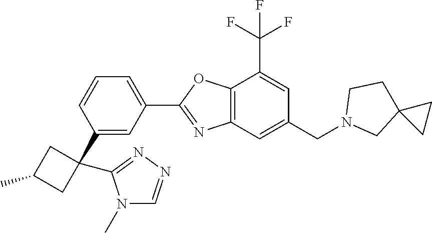 [SMILES: Cn1cnnc1[C@]1(c2cccc(-c3nc4cc(CN5CCC6(CC6)C5)cc(C(F)(F)F)c4o3)c2)C[C@H](C)C1] |
| I-10 | 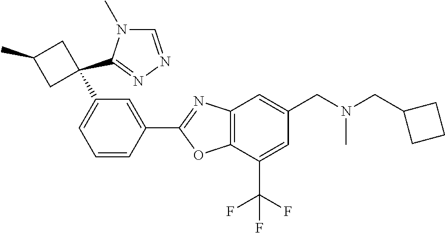 [SMILES: CN(Cc1cc(C(F)(F)F)c2oc(-c3cccc([C@]4(c5nncn5C)C[C@@H](C)C4)c3)nc2c1)CC1CCC1] |
| I-11 | 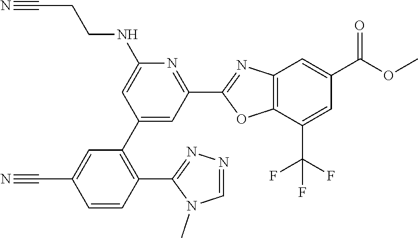 [SMILES: COC(=O)c1cc(C(F)(F)F)c2oc(-c3cc(-c4cc(C#N)ccc4-c4nncn4C)cc(NCCC#N)n3)nc2c1] |
| I-12 | 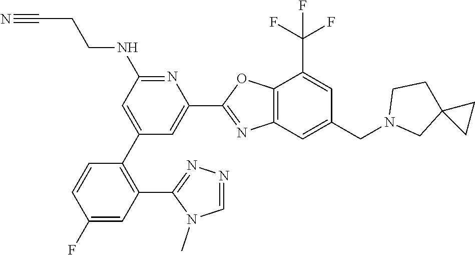 [SMILES: Cn1cnnc1-c1cc(F)ccc1-c1cc(NCCC#N)nc(-c2nc3cc(CN4CCC5(CC5)C4)cc(C(F)(F)F)c3o2)c1] |
| I-13 | 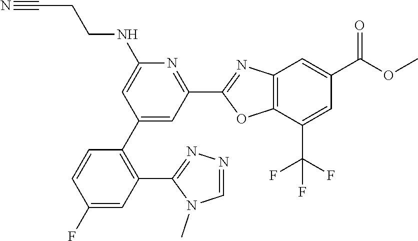 [SMILES: COC(=O)c1cc(C(F)(F)F)c2oc(-c3cc(-c4ccc(F)cc4-c4nncn4C)cc(NCCC#N)n3)nc2c1] |
| I-14 | 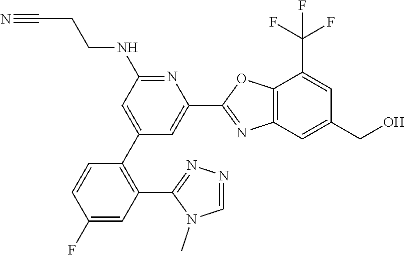 [SMILES: Cn1cnnc1-c1cc(F)ccc1-c1cc(NCCC#N)nc(-c2nc3cc(CO)cc(C(F)(F)F)c3o2)c1] |
| I-15 | 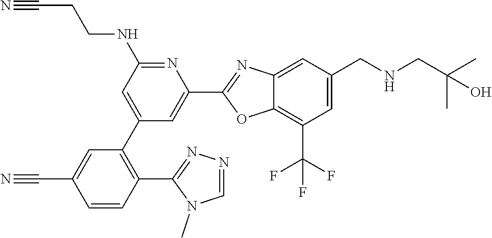 [SMILES: Cn1cnnc1-c1ccc(C#N)cc1-c1cc(NCCC#N)nc(-c2nc3cc(CNCC(C)(C)O)cc(C(F)(F)F)c3o2)c1] |
| I-16 | 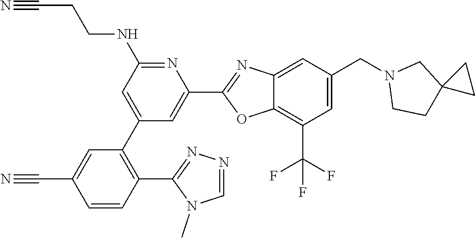 [SMILES: Cn1cnnc1-c1ccc(C#N)cc1-c1cc(NCCC#N)nc(-c2nc3cc(CN4CCC5(CC5)C4)cc(C(F)(F)F)c3o2)c1] |
| I-17 | 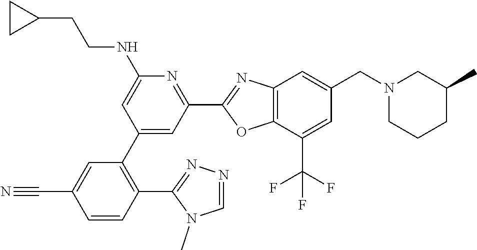 [SMILES: C[C@H]1CCCN(Cc2cc(C(F)(F)F)c3oc(-c4cc(-c5cc(C#N)ccc5-c5nncn5C)cc(NCCC5CC5)n4)nc3c2)C1] |
| I-18 | 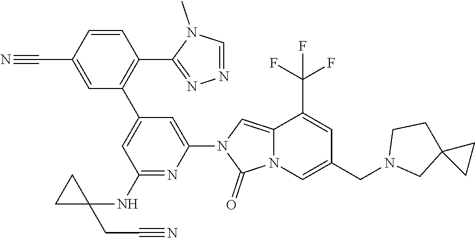 [SMILES: Cn1cnnc1-c1ccc(C#N)cc1-c1cc(NC2(CC#N)CC2)nc(-n2cc3c(C(F)(F)F)cc(CN4CCC5(CC5)C4)cn3c2=O)c1] |
| I-19 | 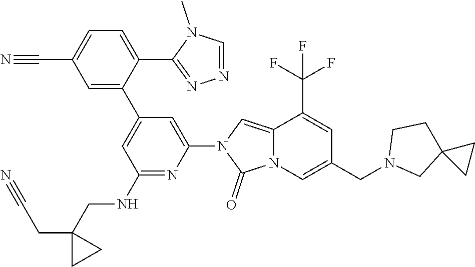 [SMILES: Cn1cnnc1-c1ccc(C#N)cc1-c1cc(NCC2(CC#N)CC2)nc(-n2cc3c(C(F)(F)F)cc(CN4CCC5(CC5)C4)cn3c2=O)c1] |
| I-20 | 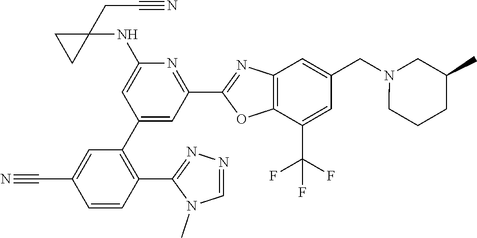 [SMILES: C[C@H]1CCCN(Cc2cc(C(F)(F)F)c3oc(-c4cc(-c5cc(C#N)ccc5-c5nncn5C)cc(NC5(CC#N)CC5)n4)nc3c2)C1] |
| I-21 | 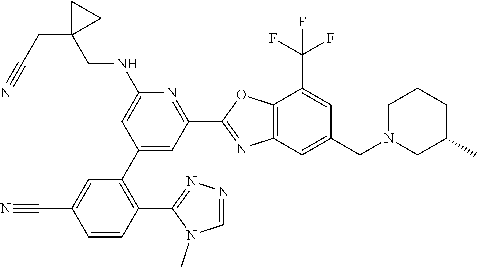 [SMILES: C[C@H]1CCCN(Cc2cc(C(F)(F)F)c3oc(-c4cc(-c5cc(C#N)ccc5-c5nncn5C)cc(NCC5(CC#N)CC5)n4)nc3c2)C1] |
| I-22 | 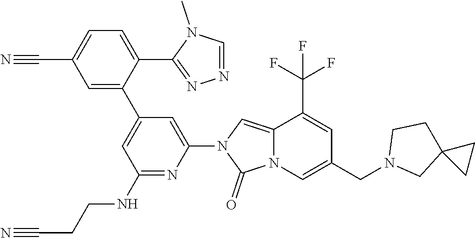 [SMILES: Cn1cnnc1-c1ccc(C#N)cc1-c1cc(NCCC#N)nc(-n2cc3c(C(F)(F)F)cc(CN4CCC5(CC5)C4)cn3c2=O)c1] |
| I-24 | 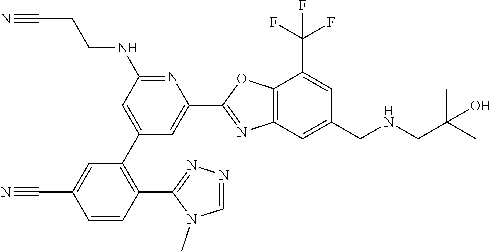 [SMILES: Cn1cnnc1-c1ccc(C#N)cc1-c1cc(NCCC#N)nc(-c2nc3cc(CNCC(C)(C)O)cc(C(F)(F)F)c3o2)c1] |
| I-27 | 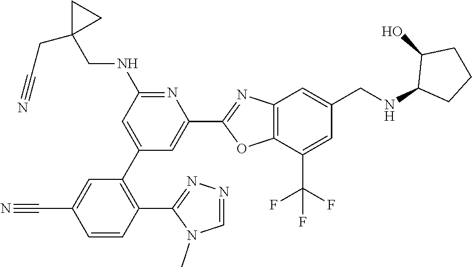 [SMILES: Cn1cnnc1-c1ccc(C#N)cc1-c1cc(NCC2(CC#N)CC2)nc(-c2nc3cc(CN[C@@H]4CCC[C@@H]4O)cc(C(F)(F)F)c3o2)c1] |
| I-29 | 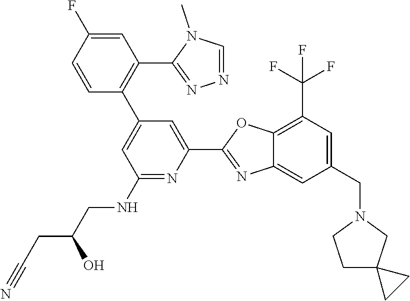 [SMILES: Cn1cnnc1-c1cc(F)ccc1-c1cc(NC[C@@H](O)CC#N)nc(-c2nc3cc(CN4CCC5(CC5)C4)cc(C(F)(F)F)c3o2)c1] |
| I-33 | 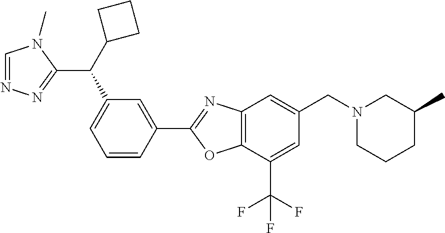 [SMILES: C[C@H]1CCCN(Cc2cc(C(F)(F)F)c3oc(-c4cccc([C@H](c5nncn5C)C5CCC5)c4)nc3c2)C1] |
| I-34 | 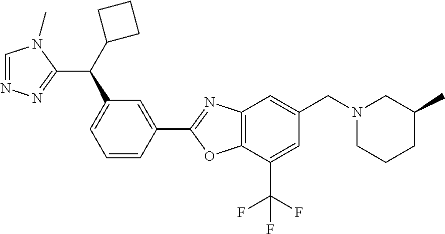 [SMILES: C[C@H]1CCCN(Cc2cc(C(F)(F)F)c3oc(-c4cccc([C@@H](c5nncn5C)C5CCC5)c4)nc3c2)C1] |
| I-48 | 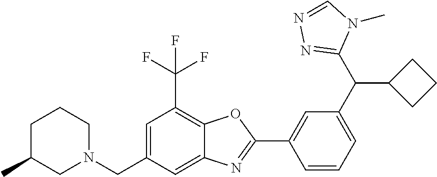 [SMILES: C[C@H]1CCCN(Cc2cc(C(F)(F)F)c3oc(-c4cccc(C(c5nncn5C)C5CCC5)c4)nc3c2)C1] |
| I-49 | 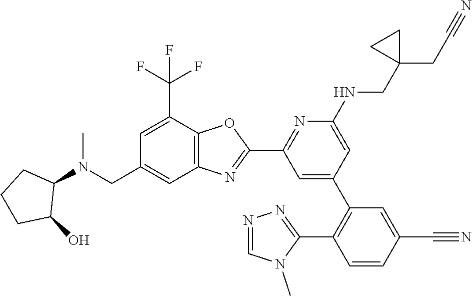 [SMILES: CN(Cc1cc(C(F)(F)F)c2oc(-c3cc(-c4cc(C#N)ccc4-c4nncn4C)cc(NCC4(CC#N)CC4)n3)nc2c1)[C@@H]1CCC[C@@H]1O] |
| I-50 | 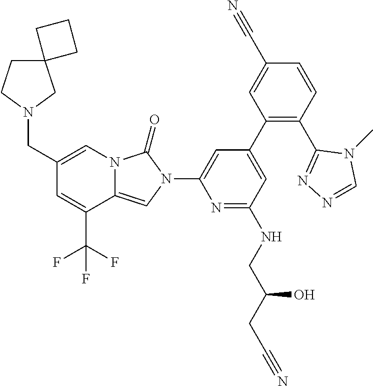 [SMILES: Cn1cnnc1-c1ccc(C#N)cc1-c1cc(NC[C@@H](O)CC#N)nc(-n2cc3c(C(F)(F)F)cc(CN4CCC5(CCC5)C4)cn3c2=O)c1] |
| I-51 | 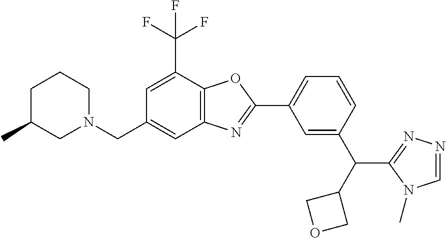 [SMILES: C[C@H]1CCCN(Cc2cc(C(F)(F)F)c3oc(-c4cccc(C(c5nncn5C)C5COC5)c4)nc3c2)C1] |
| I-52 | 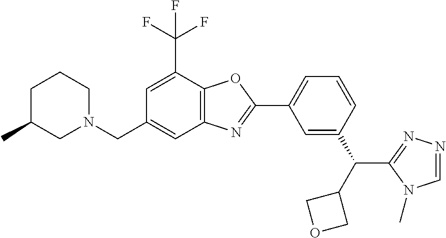 [SMILES: C[C@H]1CCCN(Cc2cc(C(F)(F)F)c3oc(-c4cccc([C@H](c5nncn5C)C5COC5)c4)nc3c2)C1] |
| I-53 | 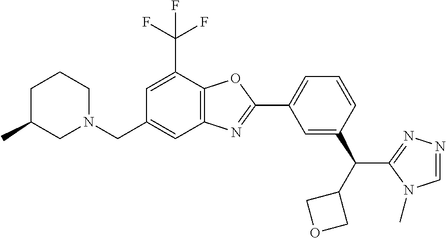 [SMILES: C[C@H]1CCCN(Cc2cc(C(F)(F)F)c3oc(-c4cccc([C@@H](c5nncn5C)C5COC5)c4)nc3c2)C1] |
| I-54 | 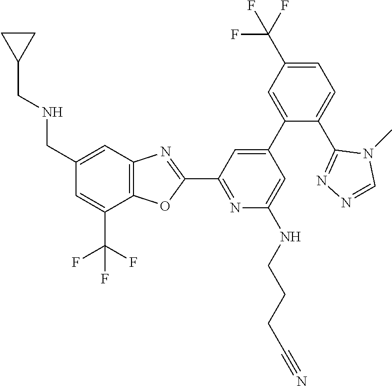 [SMILES: Cn1cnnc1-c1ccc(C(F)(F)F)cc1-c1cc(NCCCC#N)nc(-c2nc3cc(CNCC4CC4)cc(C(F)(F)F)c3o2)c1] |
| I-55 | 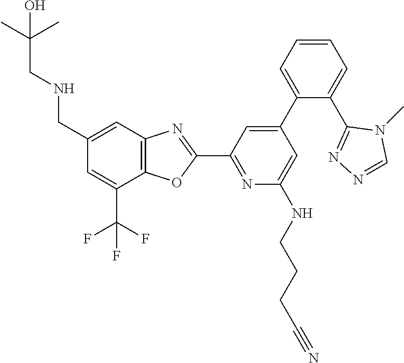 [SMILES: Cn1cnnc1-c1ccccc1-c1cc(NCCCC#N)nc(-c2nc3cc(CNCC(C)(C)O)cc(C(F)(F)F)c3o2)c1] |
| I-56 | 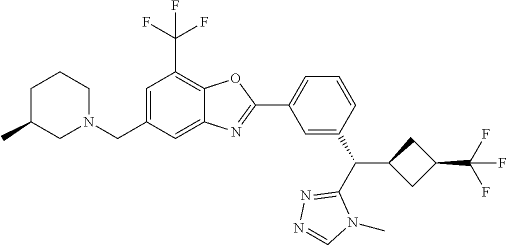 [SMILES: C[C@H]1CCCN(Cc2cc(C(F)(F)F)c3oc(-c4cccc([C@@H](c5nncn5C)[C@H]5C[C@@H](C(F)(F)F)C5)c4)nc3c2)C1] |
| I-57 | 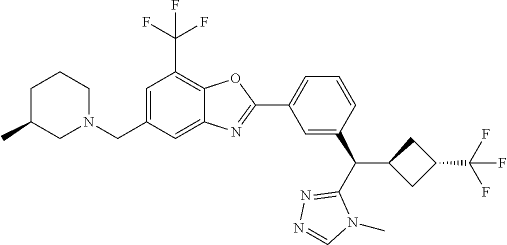 [SMILES: C[C@H]1CCCN(Cc2cc(C(F)(F)F)c3oc(-c4cccc([C@H](c5nncn5C)[C@H]5C[C@H](C(F)(F)F)C5)c4)nc3c2)C1] |
| I-58 | 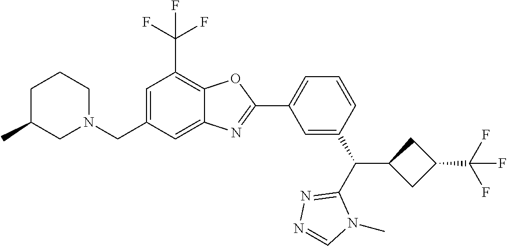 [SMILES: C[C@H]1CCCN(Cc2cc(C(F)(F)F)c3oc(-c4cccc([C@@H](c5nncn5C)[C@H]5C[C@H](C(F)(F)F)C5)c4)nc3c2)C1] |
| I-59 | 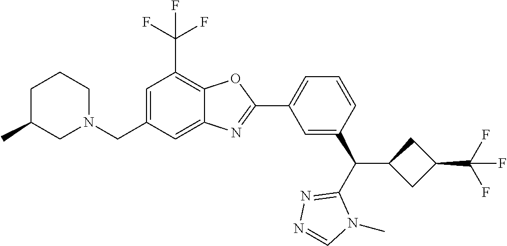 [SMILES: C[C@H]1CCCN(Cc2cc(C(F)(F)F)c3oc(-c4cccc([C@H](c5nncn5C)[C@H]5C[C@@H](C(F)(F)F)C5)c4)nc3c2)C1] |
| I-60 | 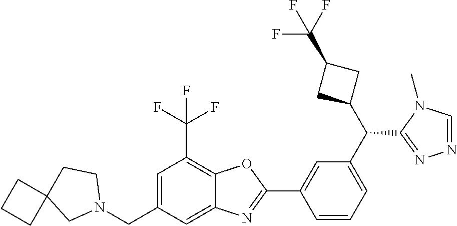 [SMILES: Cn1cnnc1[C@H](c1cccc(-c2nc3cc(CN4CCC5(CCC5)C4)cc(C(F)(F)F)c3o2)c1)[C@H]1C[C@@H](C(F)(F)F)C1] |
| I-61 | 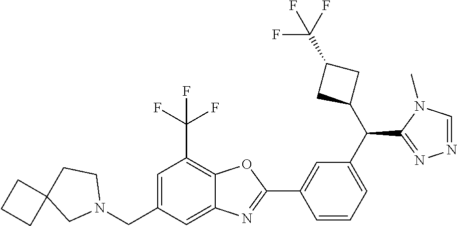 [SMILES: Cn1cnnc1[C@@H](c1cccc(-c2nc3cc(CN4CCC5(CCC5)C4)cc(C(F)(F)F)c3o2)c1)[C@H]1C[C@H](C(F)(F)F)C1] |
| I-62 | 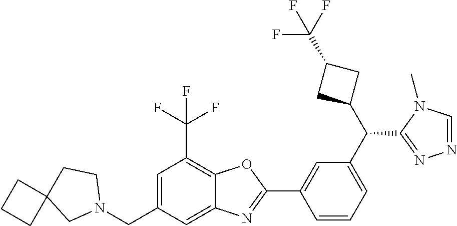 [SMILES: Cn1cnnc1[C@H](c1cccc(-c2nc3cc(CN4CCC5(CCC5)C4)cc(C(F)(F)F)c3o2)c1)[C@H]1C[C@H](C(F)(F)F)C1] |
| I-63 | 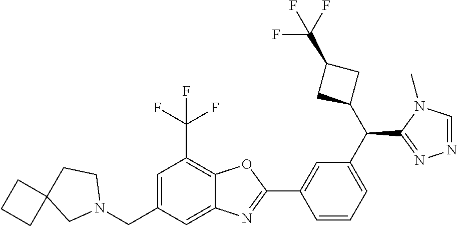 [SMILES: Cn1cnnc1[C@@H](c1cccc(-c2nc3cc(CN4CCC5(CCC5)C4)cc(C(F)(F)F)c3o2)c1)[C@H]1C[C@@H](C(F)(F)F)C1] |

In some embodiments, the present invention provides a compound set forth in Table 1, above, or a pharmaceutically acceptable salt thereof. In some embodiments, the present invention provides a compound set forth in Table 1, above.

In some embodiments, the present invention provides a pharmaceutical composition comprising a compound disclosed herein (described in embodiments herein, both singly and in combination), or a pharmaceutically acceptable salt thereof, together with a pharmaceutically acceptable carrier, excipient, or diluent. For example, in some embodiments, the present invention provides a pharmaceutical composition comprising a compound of formula I as defined above, or a pharmaceutically acceptable salt thereof, together with a pharmaceutically acceptable carrier, excipient, or diluent. In some embodiments, the present invention provides a pharmaceutical composition comprising a compound of formula I as defined above, together with a pharmaceutically acceptable carrier, excipient, or diluent. In some embodiments, the present invention provides a pharmaceutical composition comprising a compound set forth in Table 1 above, or a pharmaceutically acceptable salt thereof, together with a pharmaceutically acceptable carrier, excipient, or diluent. In some embodiments, the present invention provides a pharmaceutical composition comprising a compound set forth in Table 1 above, together with a pharmaceutically acceptable carrier, excipient, or diluent.

Exemplary compounds of the invention are set forth in Table 2, below.

TABLE 2
Selected Compounds

| I-# | Compound |
|---|---|
| I-23 | 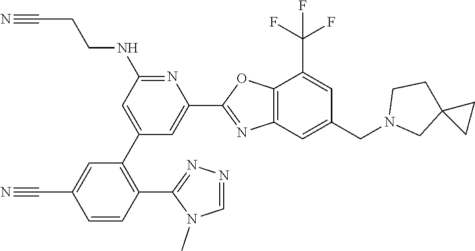 [SMILES: Cn1cnnc1-c1ccc(C#N)cc1-c1cc(NCCC#N)nc(-c2nc3cc(CN4CCC5(CC5)C4)cc(C(F)(F)F)c3o2)c1] |
| I-25 | 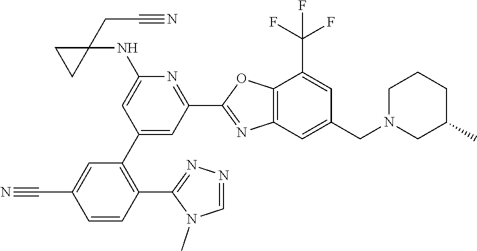 [SMILES: C[C@H]1CCCN(Cc2cc(C(F)(F)F)c3oc(-c4cc(-c5cc(C#N)ccc5-c5nncn5C)cc(NC5(CC#N)CC5)n4)nc3c2)C1] |
| I-26 | 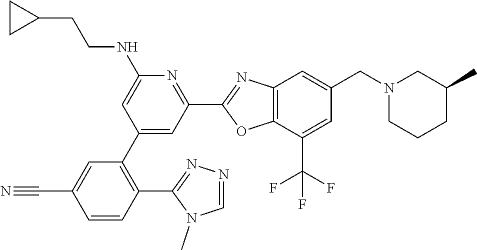 [SMILES: C[C@H]1CCCN(Cc2cc(C(F)(F)F)c3oc(-c4cc(-c5cc(C#N)ccc5-c5nncn5C)cc(NCCC5CC5)n4)nc3c2)C1] |
| I-28 | 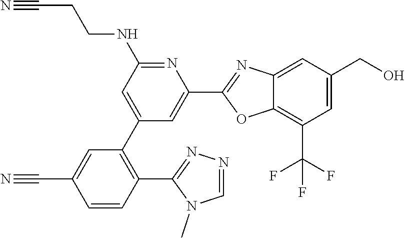 [SMILES: Cn1cnnc1-c1ccc(C#N)cc1-c1cc(NCCC#N)nc(-c2nc3cc(CO)cc(C(F)(F)F)c3o2)c1] |
| I-30 | 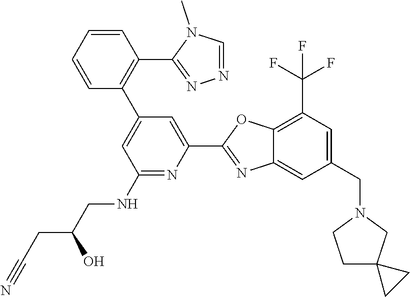 [SMILES: Cn1cnnc1-c1ccccc1-c1cc(NC[C@@H](O)CC#N)nc(-c2nc3cc(CN4CCC5(CC5)C4)cc(C(F)(F)F)c3o2)c1] |
| I-31 | 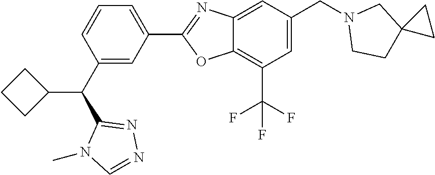 [SMILES: Cn1cnnc1[C@H](c1cccc(-c2nc3cc(CN4CCC5(CC5)C4)cc(C(F)(F)F)c3o2)c1)C1CCC1] |
| I-32 | 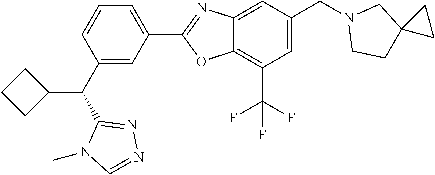 [SMILES: Cn1cnnc1[C@@H](c1cccc(-c2nc3cc(CN4CCC5(CC5)C4)cc(C(F)(F)F)c3o2)c1)C1CCC1] |
| I-35 | 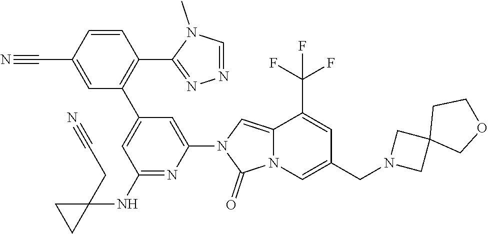 [SMILES: Cn1cnnc1-c1ccc(C#N)cc1-c1cc(NC2(CC#N)CC2)nc(-n2cc3c(C(F)(F)F)cc(CN4CC5(CCOC5)C4)cn3c2=O)c1] |
| I-36 | 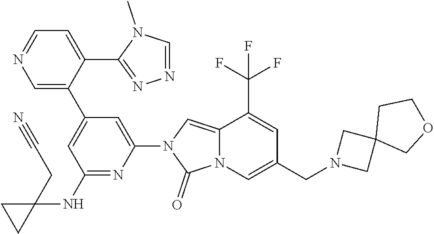 [SMILES: Cn1cnnc1-c1ccncc1-c1cc(NC2(CC#N)CC2)nc(-n2cc3c(C(F)(F)F)cc(CN4CC5(CCOC5)C4)cn3c2=O)c1] |
| I-37 | 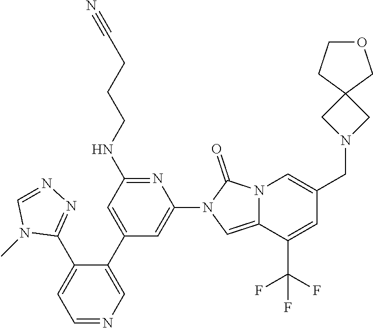 [SMILES: Cn1cnnc1-c1ccncc1-c1cc(NCCCC#N)nc(-n2cc3c(C(F)(F)F)cc(CN4CC5(CCOC5)C4)cn3c2=O)c1] |
| I-38 | 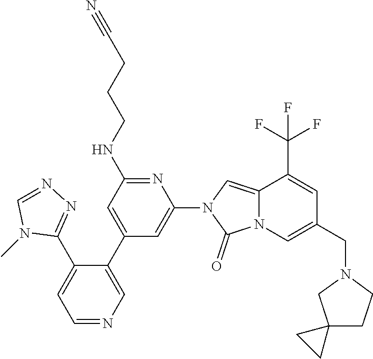 [SMILES: Cn1cnnc1-c1ccncc1-c1cc(NCCCC#N)nc(-n2cc3c(C(F)(F)F)cc(CN4CCC5(CC5)C4)cn3c2=O)c1] |
| I-39 | 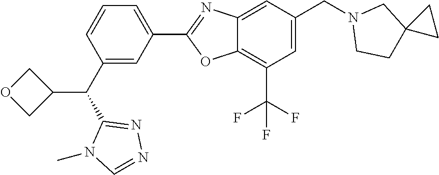 [SMILES: Cn1cnnc1[C@@H](c1cccc(-c2nc3cc(CN4CCC5(CC5)C4)cc(C(F)(F)F)c3o2)c1)C1COC1] |
| I-40 | 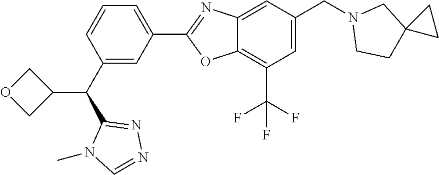 [SMILES: Cn1cnnc1[C@H](c1cccc(-c2nc3cc(CN4CCC5(CC5)C4)cc(C(F)(F)F)c3o2)c1)C1COC1] |
| I-41 | 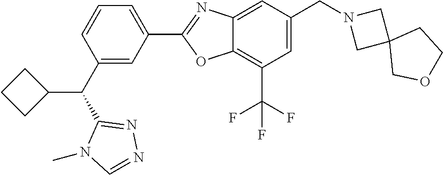 [SMILES: Cn1cnnc1[C@@H](c1cccc(-c2nc3cc(CN4CC5(CCOC5)C4)cc(C(F)(F)F)c3o2)c1)C1CCC1] |
| I-42 | 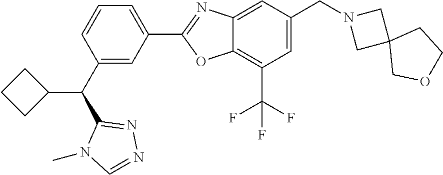 [SMILES: Cn1cnnc1[C@H](c1cccc(-c2nc3cc(CN4CC5(CCOC5)C4)cc(C(F)(F)F)c3o2)c1)C1CCC1] |
| I-43 | 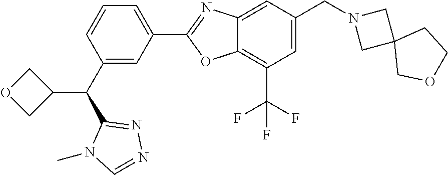 [SMILES: Cn1cnnc1[C@H](c1cccc(-c2nc3cc(CN4CC5(CCOC5)C4)cc(C(F)(F)F)c3o2)c1)C1COC1] |
| I-44 | 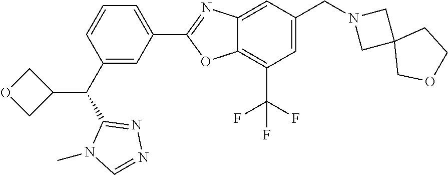 [SMILES: Cn1cnnc1[C@@H](c1cccc(-c2nc3cc(CN4CC5(CCOC5)C4)cc(C(F)(F)F)c3o2)c1)C1COC1] |
| I-45 | 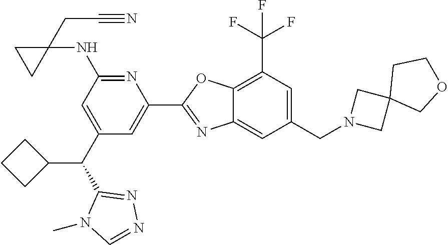 [SMILES: Cn1cnnc1[C@@H](c1cc(NC2(CC#N)CC2)nc(-c2nc3cc(CN4CC5(CCOC5)C4)cc(C(F)(F)F)c3o2)c1)C1CCC1] |
| I-46 | 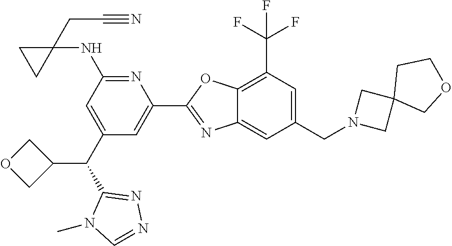 [SMILES: Cn1cnnc1[C@@H](c1cc(NC2(CC#N)CC2)nc(-c2nc3cc(CN4CC5(CCOC5)C4)cc(C(F)(F)F)c3o2)c1)C1COC1] |
| I-47 | 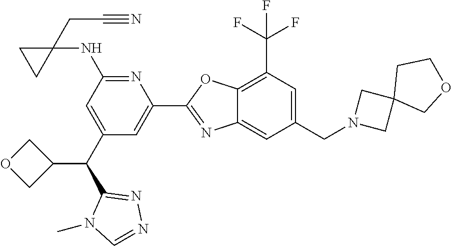 [SMILES: Cn1cnnc1[C@H](c1cc(NC2(CC#N)CC2)nc(-c2nc3cc(CN4CC5(CCOC5)C4)cc(C(F)(F)F)c3o2)c1)C1COC1] |

### 4. General Methods of Providing the Present Compounds

The compounds of this invention may be prepared or isolated in general by synthetic and/or semi-synthetic methods known to those skilled in the art for analogous compounds and by methods described in detail in the Examples, herein. In some embodiments, the compounds of the invention are prepared by the methods described in PCT/US2022/071633.

In the Schemes below, where a particular protecting group (“PG”), leaving group (“LG”), or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March, 5^{th} Edition, John Wiley & Sons, 2001, Comprehensive Organic Transformations, R. C. Larock, 2^{nd} Edition. John Wiley & Sons, 1999, and Protecting Groups in Organic Synthesis, T. W. Greene and P. G. M. Wuts, 3^{rd} edition, John Wiley & Sons, 1999.

As used herein, the phrase “leaving group” (LG) includes, but is not limited to, halogens (e.g., fluoride, chloride, bromide, iodide), sulfonates (e.g., mesylate, tosylate, benzenesulfonate, brosylate, nosylate, triflate), diazonium, and the like.

As used herein, the phrase “oxygen protecting group” includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3^{rd} edition, John Wiley & Sons, 1999. Examples of suitable hydroxyl protecting groups include, but are not limited to, esters and ethers. Examples of such ethers include allyl ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benzylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protecting Groups in Organic Synthesis, T. W. Greene and P. G. M. Wuts, 3^{rd} edition, John Wiley & Sons, 1999. Suitable amino protecting groups include, but are not limited to, aralkylamines, carbamates, cyclic imides, allyl amines, amides, and the like. Examples of such groups include t-butyloxycarbonyl (Boc), ethyloxycarbonyl, methyloxycarbonyl, trichloroethyloxycarbonyl, allyloxycarbonyl (Alloc), benzyloxocarbonyl (Cbz), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

In certain embodiments, compounds of the present invention are generally prepared according to any one of the schemes set forth below:

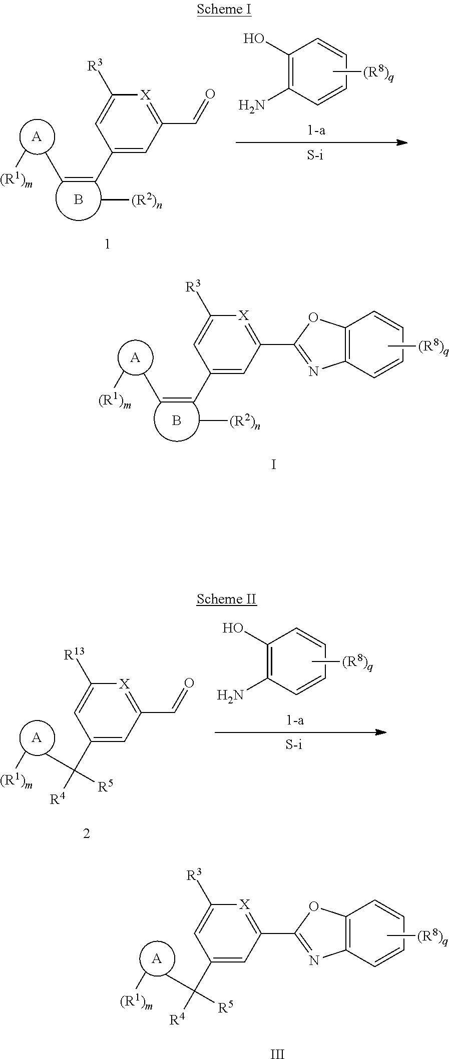

In the schemes above, each of R¹, R², R³, R⁴, R⁵, R⁸, R¹³, X, Ring A, Ring B, in, n and q, is as defined above and below and in classes and subclasses as described herein.

In one aspect, the present invention provides methods for preparing compounds of formula I or III according to the steps depicted in Scheme I or Scheme II above. In some embodiments, step S-i comprises contacting the compound of formula 1 or 2 with the compound of formula 1-a in the presence of a solvent at a temperature. In some embodiments, the solvent is a polar aprotic solvent (e.g., DCM). In some embodiments, the temperature is above room temperature (e.g., 60° C.). In some embodiments, step S-i above further comprises contacting the mixture of the compound of formula 1 or 2 with the compound of formula 1-a with an oxidant at a temperature. In some embodiments, the oxidant is DDQ. In some embodiments, the temperature is room temperature. In some embodiments, step S-i comprises the compounds, reagents, and conditions described in the below Examples section.

In certain embodiments, compounds of the present invention are generally prepared according to any one of the schemes set forth below:

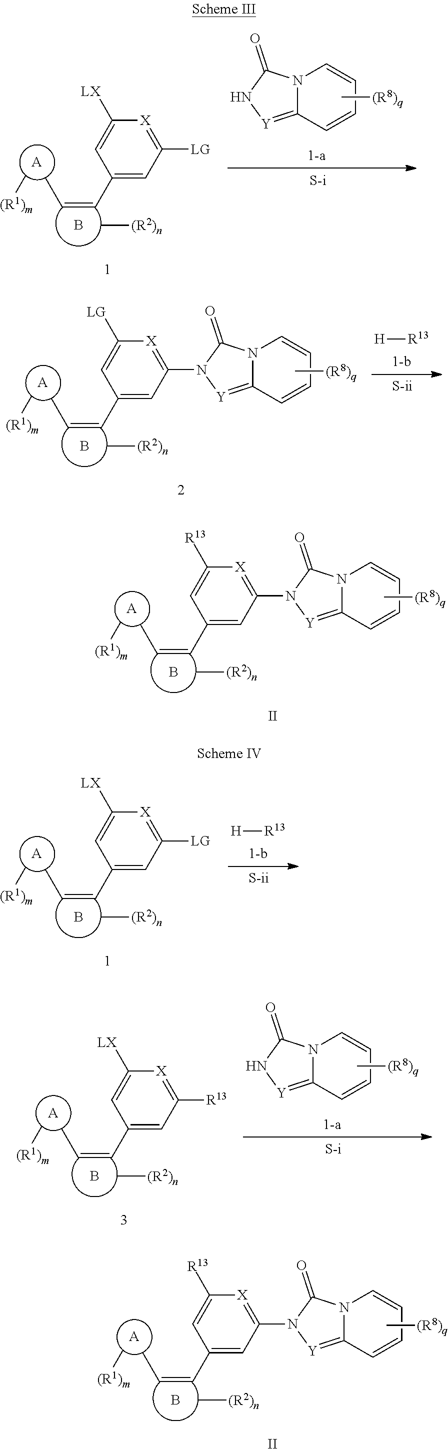

In the schemes above, each of R¹, R², R⁸, R¹³, X, Y, Ring A, Ring B, m, n and q, is as defined above and below and in classes and subclasses as described herein, each LG is independently a leaving group. In some embodiments, each LG is an appropriate halide.

In one aspect, the present invention provides methods for preparing compounds of formula II according to the steps depicted in Scheme III or Scheme IV above. In some embodiments, step S-i comprises contacting the compound of formula 1 or 3 with the compound of formula 1-a in the presence of a base in a solvent. In some embodiments, the base is a carbonate, such as K₂CO₂ or Cs₂CO₃. In some embodiments, the solvent is a polar aprotic solvent, such as NMP. In some embodiments, step S-i comprises contacting the compound of formula 1 or 3 with the compound of formula 1-a in the presence of a palladium catalyst and a base in a solvent. In some embodiments, the palladium catalyst includes a phosphine ligand, such as RuPhos. In some embodiments, the base is a carbonate, such as K₂CO₂ or Cs₂CO₃, or a phosphate, such as K₃PO₄. In some embodiments, the solvent is a polar aprotic solvent, such as dioxane. In some embodiments, step S-i comprises the compounds, reagents, and conditions described in the below Examples section.

In some embodiments, step S-ii comprises contacting the compound of formula 1 or 2 with the compound of formula 1-b in the presence of a base in a solvent. In some embodiments, the compound of formula 1-b is a primary amine and optionally comprises an amino protecting group (PG), such as t-butyloxycarbonyl (Boc). In some embodiments, the base is a carbonate, such as K₂CO₂ or Cs₂CO₃. In some embodiments, the solvent is a polar aprotic solvent, such as NMP. In some embodiments, step S-ii comprises contacting the compound of formula 1 or 2 with the compound of formula 1-b in the presence of a palladium catalyst and a base in a solvent. In some embodiments, the palladium catalyst includes a phosphine ligand, such as RuPhos. In some embodiments, the base is a carbonate, such as K₂CO₂ or Cs₂CO₃, or a phosphate, such as K₃PO₄. In some embodiments, the solvent is a polar aprotic solvent, such as dioxane. In some embodiments, step S-ii comprises the compounds, reagents, and conditions described in the below Examples section.

### 5. Uses, Formulation and Administration

### Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. In certain embodiments, the amount of compound in compositions of this invention is such that is effective to measurably inhibit Cbl-b, or a mutant thereof, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitory active metabolite or residue thereof.

As used herein, the term “active metabolite or residue thereof” means that a metabolite or residue thereof is also an inhibitor of Cbl-b, or a mutant thereof.

The subject matter disclosed herein includes prodrugs, metabolites, derivatives, and pharmaceutically acceptable salts of compounds of the invention. Metabolites include compounds produced by a process comprising contacting a compound of the invention with a mammal for a period of time sufficient to yield a metabolic product thereof. If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. If the compound of the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

A compound of the invention can be in the form of a “prodrug,” which includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Prodrugs which are converted to active forms through other mechanisms in vivo are also included. In aspects, the compounds of the invention are prodrugs of any of the formulae herein.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Alternatively, pharmaceutically acceptable compositions of this invention may be administered in the form of suppositories for rectal or vaginal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

The amount of compounds of the present invention that may be combined with the carrier materials to produce a composition in a single dosage form will vary depending upon the host treated, the particular mode of administration. Preferably, provided compositions should be formulated so that a dosage of between 0.01 and 100 mg/kg, 0.01 and 50 mg/kg, or 1 and 25 mg/kg, body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment.

### Uses of Compounds and Pharmaceutically Acceptable Compositions

The compounds and compositions described herein are generally useful for the inhibition of the activity of Cbl E3 ligases. In some embodiments the E3 ligase inhibited by the compounds and methods of the invention is Cbl-b.

The presently disclosed compounds find use in inhibiting the enzyme Cbl-b. In one embodiment, the subject matter disclosed herein is directed to a method of inhibiting Cbl-b, the method comprising contacting Cbl-b with an effective amount of a compound of the invention or a pharmaceutical composition described herein.

The presently disclosed compounds can be used in a method for inhibiting Cbl-b. Such methods comprise contacting Cbl-b with an effective amount of a presently disclosed compound. By “contact” is intended bringing the compound within close enough proximity to an isolated Cbl-b enzyme or a cell expressing Cbl-b such that the compound is able to bind to and inhibit the Cbl-b. The compound can be contacted with Cbl-b in vitro or in vivo via administration of the compound to a subject.

In one aspect, provided herein is a method of inhibiting Cbl-b in a biological sample. The method comprises contacting the sample with a compound disclosed herein (such as a compound of formula I), a pharmaceutically acceptable salt thereof, or a pharmaceutical composition disclosed herein (such as a composition comprising a compound disclosed herein [such as a compound of formula I] and a pharmaceutically acceptable carrier, adjuvant, or vehicle). The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

The present disclosure provides methods of inhibiting Cbl-b in a patient. The method comprises administering to a patient a compound disclosed herein (such as a compound of formula I), a pharmaceutically acceptable salt thereof, or a pharmaceutical composition disclosed herein (such as a composition comprising a compound disclosed herein [such as a compound of formula 1] and a pharmaceutically acceptable carrier, adjuvant, or vehicle).

The presently disclosed compounds may or may not be selective Cbl-b inhibitors. A selective Cbl-b inhibitor inhibits the biological activity of Cbl-b by an amount that is statistically greater than the inhibiting effect of the inhibitor on any other protein (e.g., other E3 ligases). In some embodiments, the activity of a selective inhibitor (measured as any one of EC₅₀, IC₅₀, K_{D} or Kᵢ) for Cbl-b is about 10 fold greater than the corresponding inhibitory activity for another target (e.g., other E3 ligase such as c-Cbl). In other embodiments the activity of the selective inhibitor for Cbl-b is at least about 15 fold greater, 20 fold greater, 25 fold greater, 30 fold greater, 40 fold greater or 50 fold greater than the corresponding inhibitory activity for another target (e.g., other E3 ligases such as c-Cbl).

Any method known in the art to measure the ligase activity of Cbl-b may be used to determine if Cbl-b has been inhibited, including in vitro kinase assays, immunoblots with antibodies specific for ubiquitinated targets of Cbl-b, or the measurement of a downstream biological effect of Cbl-b ligase activity.

The presently disclosed compounds can be used to treat an Cbl-b-dependent disorder. As used herein, a “Cbl-b-dependent disorder” is a pathological condition in which Cbl-b activity is necessary for the genesis or maintenance of the pathological condition.

Accordingly, in one aspect, provided herein is a method of treating a Cbl-b-mediated disorder, disease, or condition in a patient. The method comprises administering to said patient a compound disclosed herein (such as a compound of formula I), a pharmaceutically acceptable salt thereof, or a pharmaceutical composition disclosed herein (such as a composition comprising a compound disclosed herein [such as a compound of formula I] and a pharmaceutically acceptable carrier, adjuvant, or vehicle).

Provided herein are compounds and pharmaceutical compositions that inhibit the Cbl-b enzyme, as well as methods of treatment using such compounds and pharmaceutical compositions. The compounds and compositions can be used in methods of modulating the immune system, for treatment of diseases, and for treatment of cells in vivo, in vitro, or ex vivo.

T-cell activation and T-cell tolerance are tightly controlled processes regulating the immune response to tumors while preventing autoimmunity. Tolerance prevents the immune system from attacking cells expressing “self” antigens. During peripheral tolerance, T-cells that recognize “self” antigens (i.e., self-reactive T-cells) become functionally unresponsive or are deleted after encountering “self” antigens outside of the thymus. Peripheral tolerance processes therefore are important for preventing autoimmune diseases. Normally, cancer cells are removed by activated T-cells that recognize tumor antigens expressed on the surface of the cancer cells. However, in cancer, the tumor microenvironment can support T-cell tolerance to cancer cells, which allows cancer cells to avoid recognition and removal by the immune system. The ability of cancer cells to avoid tumor immunosurveillance can contribute to uncontrolled tumor growth. Therefore, T-cell tolerance can be a form of T-cell dysfunction. General principles of T-cell dysfunction are well known in the art (see Schietinger et al, Trends Immunol., 35: 51-60, 2014). Additional types of T-cell dysfunction that can contribute to uncontrolled tumor growth include T-cell exhaustion, T-cell senescence, and/or T-cell anergy. Therefore, treating T-cell dysfunction, for example, by increasing T-cell activation, increasing T-cell proliferation, decreasing T-cell tolerance, and/or decreasing T-cell exhaustion, is beneficial for preventing or treating cancer. Additional cells of the immune system are important for recognition and removal of cancer cells during immune surveillance. For example, Natural Killer (NK)-cells are lymphocytes of the innate immune system that are able to identify and kill cancer cells (see Martinez-Losato et al, Clin Cancer Res., 21: 5048-5056, 2015). Recent studies have also shown that B-cell subsets with distinct phenotypes and functions exhibit diverse roles in the anti-tumor response (see Saravaria et al, Cell Mol Immunol., 14: 662-674, 2017). Due to their role in tumor surveillance, NK-cells and B-cells may also be amenable as therapeutic targets for the prevention or treatment of cancer.

Cbl-b is a RING-type E3 ligase that plays an important role in the immune system due to its function as a negative regulator of immune activation. Cbl-b has an essential role in decreasing the activation of T-cells, thereby enhancing T-cell tolerance. Studies have found that Cbl-b-deficient T-cells display lower thresholds for activation by antigen recognition receptors and co-stimulatory molecules (e.g., CD28). For example, loss of Cbl-b in T-cells uncouples the requirement for CD28 costimulation during T-cell activation and proliferation (see Bachmaier el al, Nature, 403: 211-216, 2000). Such cbl-b−/− T-cells are largely resistant to T-cell anergy, a tolerance mechanism in which T-cells are functionally inactivated and T-cell proliferation is greatly impaired (see Jeon el al. Immunity, 21: 167-177, 2004; and Schwartz et al, Annu Rev Immunol., 21: 305-34, 2003). In support of this, loss of Cbl-b in cbl-b knockout mice resulted in impaired induction of T-cell tolerance and exacerbated autoimmunity (see Jeon el al, Immunity, 21: 167-177, 2004). Importantly, loss of Cbl-b in mice also resulted in a robust anti-tumor response that depends primarily on cytotoxic T-cells. One study showed that cbl-b−/− CD8+ T-cells are resistant to T regulatory cell-mediated suppression and exhibit enhanced activation and tumor infiltration. Therapeutic transfer of naive cbl-b−/− CD8+ T-cells was sufficient to mediate rejection of established tumors (see Loeser et al, J Exp Med., 204: 879-891, 2007). Recent studies have shown that Cbl-b also plays a role in NK-cell activation. Genetic deletion of Cbl-b or targeted inactivation of its E3 ligase activity allowed NK-cells to spontaneously reject metastatic tumors in a mouse model (see Paolino et al, Nature, 507: 508-512, 2014).

Provided herein are compounds and compositions that are potent inhibitors of Cbl-b and can be used in novel approaches to treat diseases such as cancer. In some embodiments, the compounds and compositions provided herein can be used in methods of modulating the immune system, such as increasing activation of T-cells, NK-cells and B-cells, as well as in the treatment of such cells in vivo, in vitro, or ex vivo.

Provided herein are methods for modulating activity of an immune cell (e.g., a T-cell, a B-cell, or a NK-cell) such as by contacting the immune cell with an effective amount of a Cbl-b inhibitor described herein or a composition thereof. Further provided are in vivo methods of modulating a response in an individual in need thereof (e.g., an individual with cancer), wherein the method comprises administration of an effective amount of a Cbl-b inhibitor described herein or a composition thereof.

Additionally, provided are Cbl-b inhibitors for use as therapeutic active substances. A Cbl-b inhibitor for use in treating or preventing a disease or condition associated with Cbl-b activity is provided. Also, a Cbl-b inhibitor for use in treating cancer is provided. Further provided is the use of a Cbl-b inhibitor in the manufacture of a medicament for treating or preventing a disease or condition associated with Cbl-b activity. Also provided is the use of a Cbl-b inhibitor in the manufacture of a medicament for treating cancer.

Moreover, this disclosure provides treatment methods, medicaments, and uses comprising a Cbl-b inhibitor as part of a combination therapy for treating cancer involving one or more of an immune checkpoint inhibitor, an antineoplastic agent, and radiation therapy.

In some embodiments of the treatment methods, medicaments, and uses of this disclosure, the cancer is a hematologic cancer such as lymphoma, a leukemia, or a myeloma. In other embodiments of the treatment methods, medicaments, and uses of this disclosure, the cancer is a non-hematologic cancer such as a sarcoma, a carcinoma, or a melanoma.

Hematologic cancers include, but are not limited to, one or more leukemias such as B-cell acute lymphoid leukemia (“BALL”), T-cell acute lymphoid leukemia (“TALL”), acute lymphoid leukemia (ALL); one or more chronic leukemias including, but not limited to, chronic myelogenous leukemia (CML) and chronic lymphocytic leukemia (CLL); additional hematologic cancers or hematologic conditions including, but not limited to, B-cell prolymphocytic leukemia, blastic plasmacytoid dendritic cell neoplasm, Burkitt's lymphoma, diffuse large B-cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm. Waldenstrom macroglobulinemia, and “preleukemia.” which are a diverse collection of hematological conditions united by ineffective production (or dysplasia) of myeloid blood cells.

Non-hematologic cancers include, but are not limited to, a neuroblastoma, renal cell carcinoma, colon cancer, colorectal cancer, breast cancer, epithelial squamous cell cancer, melanoma, stomach cancer, brain cancer, lung cancer (e.g. NSCLC), pancreatic cancer, cervical cancer, ovarian cancer, liver cancer, bladder cancer, prostate cancer, testicular cancer, thyroid cancer, uterine cancer, adrenal cancer, and head and neck cancer.

In some aspects, the effectiveness of administration of a Cbl-b inhibitor in the treatment of a disease or disorder such as cancer is measured by assessing clinical outcome, such as reduction in tumor size or number of tumors, and/or survival. In certain embodiments, “treating cancer” comprises assessing a patient's response to the treatment regimen according to the Response Evaluation Criteria in Solid Tumors (RECIST version 1.1) as described (see, e.g., Eisenhauer et al, Eur J Cancer, 45:228-247, 2009; and Nishino et al., Am J Roentgenol, 195: 281-289, 2010). Response criteria to determine objective anti-tumor responses per RECIST 1.1 include complete response (CR); partial response (PR); progressive disease (PD); and stable disease (SD).

Accordingly, in some embodiments, the Cbl-b-mediated disorder is a hematologic cancer. In one aspect, provided herein is a method of treating a hematologic cancer in a patient. The method comprises administering to said patient a compound disclosed herein (such as a compound of formula I), a pharmaceutically acceptable salt thereof, or a pharmaceutical composition disclosed herein (such as a composition comprising a compound disclosed herein [such as a compound of formula I] and a pharmaceutically acceptable carrier, adjuvant, or vehicle).

More generally, in some embodiments, the Cbl-b-mediated disorder is a non-hematologic cancer. In one aspect, provided herein is a method of treating a non-hematologic cancer in a patient. The method comprises administering to said patient a compound disclosed herein (such as a compound of formula I), a pharmaceutically acceptable salt thereof, or a pharmaceutical composition disclosed herein (such as a composition comprising a compound disclosed herein [such as a compound of formula I] and a pharmaceutically acceptable carrier, adjuvant, or vehicle). In some embodiments, the non-hematologic cancer is a neuroblastoma, renal cell carcinoma, colon cancer, colorectal cancer, breast cancer, epithelial squamous cell cancer, melanoma, stomach cancer, brain cancer, lung cancer (e.g., NSCLC), pancreatic cancer, cervical cancer, ovarian cancer, liver cancer, bladder cancer, prostate cancer, testicular cancer, thyroid cancer, uterine cancer, adrenal cancer, and head and neck cancer. In some embodiments, the non-hematologic cancer is colon cancer. In some embodiments, the non-hematologic cancer is liver cancer. In some embodiments, the non-hematologic cancer is lung cancer. In some embodiments, the non-hematologic cancer is breast cancer. In some embodiments, the non-hematologic cancer is brain cancer.

It has also been reported that Cbl-b inhibitors may provide benefit to patients suffering from cancer. Accordingly, in some embodiments, provided herein is a method of treating a patient suffering from cancer. The method comprises administering to said patient a compound disclosed herein (such as a compound of formula I), a pharmaceutically acceptable salt thereof, or a pharmaceutical composition disclosed herein (such as a composition comprising a compound disclosed herein [such as a compound of formula I] and a pharmaceutically acceptable carrier, adjuvant, or vehicle).

Examples of cancers that are treatable using the compounds of the present disclosure include, but are not limited to, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers.

In some embodiments, cancers that are treatable using the compounds of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including relapsed or refractory NHL and recurrent follicular), Hodgkin lymphoma or multiple myeloma) and combinations of said cancers.

In certain embodiments, the cancer is brain cancer, leukemia, skin cancer, prostate cancer, thyroid cancer, colon cancer, lung cancer or sarcoma. In another embodiment the cancer is selected from the group consisting of glioma, glioblastoma multiforme, paraganglioma, supratentorial primordial neuroectodermal tumors, acute myeloid leukemia, myelodysplastic syndrome, chronic myelogenous leukemia, melanoma, breast, prostate, thyroid, colon, lung, central chondrosarcoma, central and periosteal chondroma tumors, fibrosarcoma, and cholangiocarcinoma.

In certain embodiments, the cancer is selected from brain and spinal cancers, cancers of the head and neck, leukemia and cancers of the blood, skin cancers, cancers of the reproductive system, cancers of the gastrointestinal system, liver and bile duct cancers, kidney and bladder cancers, bone cancers, lung cancers, malignant mesothelioma, sarcomas, lymphomas, glandular cancers, thyroid cancers, heart tumors, germ cell tumors, malignant neuroendocrine (carcinoid) tumors, midline tract cancers, and cancers of unknown primary (cancers in which a metastasized cancer is found but the original cancer site is not known). In particular embodiments, the cancer is present in an adult patient; in additional embodiments, the cancer is present in a pediatric patient. In particular embodiments, the cancer is AIDS-related.

In a further embodiment, the cancer is selected from brain and spinal cancers. In particular embodiments, the cancer is selected from the group consisting of anaplastic astrocytomas, glioblastomas, astrocytomas, and estheosioneuroblastomas (olfactory blastomas). In particular embodiments, the brain cancer is selected from the group consisting of astrocytic tumor (e.g., pilocytic astrocytoma, subependymal giant-cell astrocytoma, diffuse astrocytoma, pleomorphic xanthoastrocytoma, anaplastic astrocytoma, astrocytoma, giant cell glioblastoma, glioblastoma, secondary glioblastoma, primary adult glioblastoma, and primary pediatric glioblastoma), oligodendroglial tumor (e.g., oligodendroglioma, and anaplastic oligodendroglioma), oligoastrocytic tumor (e.g., oligoastrocytoma, and anaplastic oligoastrocytoma), ependymoma (e.g., myxopapillary ependymoma, and anaplastic ependymoma); medulloblastoma, primitive neuroectodermal tumor, schwannoma, meningioma, atypical meningioma, anaplastic meningioma, pituitary adenoma, brain stem glioma, cerebellar astrocytoma, cerebral astrocytoma/malignant glioma, visual pathway and hypothalmic glioma, and primary central nervous system lymphoma. In specific instances of these embodiments, the brain cancer is selected from the group consisting of glioma, glioblastoma multiforme, paraganglioma, and supratentorial primordial neuroectodermal tumors (sPNET).

In specific embodiments, the cancer is selected from cancers of the head and neck, including nasopharyngeal cancers, nasal cavity and paranasal sinus cancers, hypopharyngeal cancers, oral cavity cancers (e.g., squamous cell carcinomas, lymphomas, and sarcomas), lip cancers, oropharyngeal cancers, salivary gland tumors, cancers of the larynx (e.g., laryngeal squamous cell carcinomas, rhabdomyosarcomas), and cancers of the eye or ocular cancers. In particular embodiments, the ocular cancer is selected from the group consisting of intraocular melanoma and retinoblastoma.

In specific embodiments, the cancer is selected from leukemia and cancers of the blood. In particular embodiments, the cancer is selected from the group consisting of myeloproliferative neoplasms, myelodysplastic syndromes, myelodysplastic/myeloproliferative neoplasms, acute myeloid leukemia (AML), myelodysplastic syndrome (MDS), chronic myelogenous leukemia (CML), myeloproliferative neoplasm (MPN), post-MPN AML, post-MDS AML, del(5q)-associated high risk MDS or AML, blast-phase chronic myelogenous leukemia, angioimmunoblastic lymphoma, acute lymphoblastic leukemia, Langerans cell histiocytosis, hairy cell leukemia, and plasma cell neoplasms including plasmacytomas and multiple myelomas. Leukemias referenced herein may be acute or chronic.

In specific embodiments, the cancer is selected from skin cancers. In particular embodiments, the skin cancer is selected from the group consisting of melanoma, squamous cell cancers, and basal cell cancers.

In specific embodiments, the cancer is selected from cancers of the reproductive system. In particular embodiments, the cancer is selected from the group consisting of breast cancers, cervical cancers, vaginal cancers, ovarian cancers, prostate cancers, penile cancers, and testicular cancers. In specific instances of these embodiments, the cancer is a breast cancer selected from the group consisting of ductal carcinomas and phyllodes tumors. In specific instances of these embodiments, the breast cancer may be male breast cancer or female breast cancer. In specific instances of these embodiments, the cancer is a cervical cancer selected from the group consisting of squamous cell carcinomas and adenocarcinomas. In specific instances of these embodiments, the cancer is an ovarian cancer selected from the group consisting of epithelial cancers.

In specific embodiments, the cancer is selected from cancers of the gastrointestinal system. In particular embodiments, the cancer is selected from the group consisting of esophageal cancers, gastric cancers (also known as stomach cancers), gastrointestinal carcinoid tumors, pancreatic cancers, gallbladder cancers, colorectal cancers, and anal cancer. In instances of these embodiments, the cancer is selected from the group consisting of esophageal squamous cell carcinomas, esophageal adenocarcinomas, gastric adenocarcinomas, gastrointestinal carcinoid tumors, gastrointestinal stromal tumors, gastric lymphomas, gastrointestinal lymphomas, solid pseudopapillary tumors of the pancreas, pancreatoblastoma, islet cell tumors, pancreatic carcinomas including acinar cell carcinomas and ductal adenocarcinomas, gallbladder adenocarcinomas, colorectal adenocarcinomas, and anal squamous cell carcinomas.

In specific embodiments, the cancer is selected from liver and bile duct cancers. In particular embodiments, the cancer is liver cancer (hepatocellular carcinoma). In particular embodiments, the cancer is bile duct cancer (cholangiocarcinoma); in instances of these embodiments, the bile duct cancer is selected from the group consisting of intrahepatic cholangiocarcinoma and extrahepatic cholangiocarcinoma.

In specific embodiments, the cancer is selected from kidney and bladder cancers. In particular embodiments, the cancer is a kidney cancer selected from the group consisting of renal cell cancer, Wilms tumors, and transitional cell cancers. In particular embodiments, the cancer is a bladder cancer selected from the group consisting of urothelial carcinoma (a transitional cell carcinoma), squamous cell carcinomas, and adenocarcinomas.

In specific embodiments, the cancer is selected from bone cancers. In particular embodiments, the bone cancer is selected from the group consisting of osteosarcoma, malignant fibrous histiocytoma of bone. Ewing sarcoma, and chordoma.

In specific embodiments, the cancer is selected from lung cancers. In particular embodiments, the lung cancer is selected from the group consisting of non-small cell lung cancer, small cell lung cancers, bronchial tumors, and pleuropulmonary blastomas.

In specific embodiments, the cancer is selected from malignant mesothelioma. In particular embodiments, the cancer is selected from the group consisting of epithelial mesothelioma and sarcomatoids.

In specific embodiments, the cancer is selected from sarcomas. In particular embodiments, the sarcoma is selected from the group consisting of central chondrosarcoma, central and periosteal chondroma, fibrosarcoma, clear cell sarcoma of tendon sheaths, and Kaposi's sarcoma.

In specific embodiments, the cancer is selected from lymphomas. In particular embodiments, the cancer is selected from the group consisting of Hodgkin lymphoma (e.g., Reed-Sternberg cells), non-Hodgkin lymphoma (e.g., diffuse large B-cell lymphoma, follicular lymphoma, mycosis fungoides, Sezary syndrome, primary central nervous system lymphoma), cutaneous T-cell lymphomas, and primary central nervous system lymphomas.

In specific embodiments, the cancer is selected from glandular cancers. In particular embodiments, the cancer is selected from the group consisting of adrenocortical cancer, pheochromocytomas, paragangliomas, pituitary tumors, thymoma, and thymic carcinomas.

In specific embodiments, the cancer is selected from thyroid cancers. In particular embodiments, the thyroid cancer is selected from the group consisting of medullary thyroid carcinomas, papillary thyroid carcinomas, and follicular thyroid carcinomas.

In specific embodiments, the cancer is selected from germ cell tumors. In particular embodiments, the cancer is selected from the group consisting of malignant extracranial germ cell tumors and malignant extragonadal germ cell tumors. In specific instances of these embodiments, the malignant extragonadal germ cell tumors are selected from the group consisting of nonseminomas and seminomas.

In specific embodiments, the cancer is selected from heart tumors. In particular embodiments, the heart tumor is selected from the group consisting of malignant teratoma, lymphoma, rhabdomyosarcoma, angiosarcoma, chondrosarcoma, infantile fibrosarcoma, and synovial sarcoma.

In some embodiments, cancers treatable with compounds of the present disclosure include melanoma (e.g., metastatic malignant melanoma), renal cancer (e.g., clear cell carcinoma), prostate cancer (e.g. hormone refractory prostate adenocarcinoma), breast cancer, triple-negative breast cancer, colon cancer and lung cancer (e.g., non-small cell lung cancer and small cell lung cancer). Additionally, the disclosure includes refractory or recurrent malignancies whose growth may be inhibited using the compounds of the disclosure.

In some embodiments, diseases and indications that are treatable using the compounds of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular). Hodgkin lymphoma, myeloproliferative diseases (e.g., primary myelofibrosis (PMF), polycythemia vera (PV), essential thrombocytosis (ET)), myelodysplasia syndrome (MDS), T-cell acute lymphoblastic lymphoma (T-ALL), multiple myeloma, cutaneous T-cell lymphoma, Waldenstrom's Macroglobulinemia, hairy cell lymphoma, chronic myelogenic lymphoma and Burkitt's lymphoma.

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, hematoma, and teratoma.

Exemplary lung cancers include non-small cell lung cancer (NSCLC), small cell lung cancer, bronchogenic carcinoma (squamous cell, undifferentiated small cell, undifferentiated large cell, adenocarcinoma), alveolar (bronchiolar) carcinoma, bronchial adenoma, chondromatous hamartoma, and mesothelioma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer.

Exemplary genitourinary tract cancers include cancers of the kidney (adenocarcinoma, Wilm's tumor [nephroblastoma]), bladder and urethra (squamous cell carcinoma, transitional cell carcinoma, adenocarcinoma), prostate (adenocarcinoma, sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma).

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, ostcoid osteoma, and giant cell tumors

Exemplary nervous system cancers include cancers of the skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma, glioblastoma multiform, oligodendroglioma, schwannoma, retinoblastoma, congenital tumors), and spinal cord (neurofibroma, meningioma, glioma, sarcoma), as well as neuroblastoma and Lhermitte-Duclos disease.

Exemplary gynecological cancers include cancers of the uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, squamous cell carcinoma, Kaposi's sarcoma, Merkel cell skin cancer, moles dysplastic nevi, lipoma, angioma, dermatofibroma, and keloids. In some embodiments, diseases and indications that are treatable using the compounds of the present disclosure include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

Exemplary head and neck cancers include glioblastoma, melanoma, rhabdosarcoma, lymphosarcoma, osteosarcoma, squamous cell carcinomas, adenocarcinomas, oral cancer, laryngeal cancer, nasopharyngeal cancer, nasal and paranasal cancers, thyroid and parathyroid cancers.

In some embodiments, provided herein is a method of treating aberrant Cbl-b activity in a patient comprising administering to said patient a compound disclosed herein (such as a compound of formula I), a pharmaceutically acceptable salt thereof, or a pharmaceutical composition disclosed herein (such as a composition comprising a compound disclosed herein [such as a compound of formula I] and a pharmaceutically acceptable carrier, adjuvant, or vehicle), wherein the aberrant Cbl-b activity contributes to a disease pathology, such as any of the cancers described herein.

The presently disclosed compounds may be administered in any suitable manner known in the art. In some embodiments, the compound of the invention or a pharmaceutically acceptable salt, prodrug, metabolite, or derivative thereof is administered intravenously, intramuscularly, subcutaneously, topically, orally, transdermally, intraperitoneally, intraorbitally, by implantation, by inhalation, intrathecally, intraventricularly, intratumorally, or intranasally.

In some embodiments, the Cbl-b inhibitor is administered continuously. In other embodiments, the Cbl-b inhibitor is administered intermittently. Moreover, treatment of a subject with an effective amount of a Cbl-b inhibitor can include a single treatment or can include a series of treatments.

It is understood that appropriate doses of the active compound depends upon a number of factors within the knowledge of the ordinarily skilled physician or veterinarian. The dose(s) of the active compound will vary, for example, depending upon the age, body weight, general health, gender, and diet of the subject, the time of administration, the route of administration, the rate of excretion, and any drug combination.

It will also be appreciated that the effective dosage of a compound of the invention or a pharmaceutically acceptable salt, prodrug, metabolite, or derivative thereof used for treatment may increase or decrease over the course of a particular treatment. Changes in dosage may result and become apparent from the results of diagnostic assays.

In some embodiments, the Cbl-b inhibitor is administered to the subject at a dose of between about 0.001 μg/kg and about 1000 mg/kg, including but not limited to about 0.001 μg/kg, 0.01 μg/kg, 0.05 μg/kg, 0.1 μg/kg, 0.5 μg/kg, 1 μg/kg, 10 μg/kg, 25 μg/kg, 50 μg/kg, 100 μg/kg, 250 μg/kg, 500 μg/kg, 1 mg/kg, 5 mg/kg, 10 mg/kg, 25 mg/kg, 50 mg/kg, 100 mg/kg, and 200 mg/kg.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

In some embodiments, the compounds of the invention are useful in preventing or reducing the risk of developing any of the diseases referred to herein: e.g., preventing or reducing the risk of developing a disease, condition or disorder in an individual who may be predisposed to the disease, condition or disorder but does not yet experience or display the pathology or symptomatology of the disease.

The term “administration” or “administering” includes routes of introducing the compound(s) to a subject to perform their intended function. Examples of routes of administration which can be used include injection (subcutaneous, intravenous, parenterally, intraperitoneally, intrathecal), topical, oral, inhalation, rectal and transdermal.

The term “effective amount” includes an amount effective, at dosages and for periods of time necessary, to achieve the desired result. An effective amount of compound may vary according to factors such as the disease state, age, and weight of the subject, and the ability of the compound to elicit a desired response in the subject. Dosage regimens may be adjusted to provide the optimum therapeutic response.

The phrases “systemic administration,” “administered systemically”, “peripheral administration” and “administered peripherally” as used herein mean the administration of a compound(s), drug or other material, such that it enters the patient's system and, thus, is subject to metabolism and other like processes.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “subject” refers to animals such as mammals, including, but not limited to, primates (e.g., humans), cows, sheep, goats, horses, dogs, cats, rabbits, rats, mice and the like. In certain embodiments, the subject is a human.

### Combination Therapies

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated.” In certain embodiments, a provided combination, or composition thereof, is administered in combination with another therapeutic agent.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination.” “combined.” and related terms refers to the simultaneous or sequential administration of therapeutic agents in accordance with this invention. For example, a combination of the present invention may be administered with another therapeutic agent simultaneously or sequentially in separate unit dosage forms or together in a single unit dosage form.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In one embodiment, the present invention provides a composition comprising a compound of formula I and one or more additional therapeutic agents. The therapeutic agent may be administered together with a compound of formula I, or may be administered prior to or following administration of a compound of formula I. Suitable therapeutic agents are described in further detail below. In certain embodiments, a compound of formula I may be administered up to 5 minutes, 10 minutes, 15 minutes, 30 minutes. 1 hour, 2 hours, 3 hours, 4 hours. 5, hours, 6 hours, 7 hours, 8 hours, 9 hours, 10 hours, 11 hours, 12 hours, 13 hours, 14 hours. 15 hours. 16 hours. 17 hours, or 18 hours before the therapeutic agent. In other embodiments, a compound of formula I may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In another embodiment, the present invention provides a method of treating a hematological malignancy comprising administering to a patient in need thereof a compound of formula I and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a PI3K inhibitor, a SYK inhibitor, and combinations thereof.

In another embodiment, the present invention provides a method of treating a solid tumor comprising administering to a patient in need thereof a compound of formula I and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a PI3K inhibitor, a SYK inhibitor, and combinations thereof.

In another embodiment, the present invention provides a method of treating a hematological malignancy comprising administering to a patient in need thereof a compound of formula I and a Hedgehog (Hh) signaling pathway inhibitor. In some embodiments, the hematological malignancy is DLBCL (Ramirez et al “Defining causative factors contributing in the activation of hedgehog signaling in diffuse large B-cell lymphoma” Leuk. Res. (2012), published online July 17).

In another embodiment, the present invention provides a method of treating diffuse large B-cell lymphoma (DLBCL) comprising administering to a patient in need thereof a compound of formula I and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, and combinations thereof.

In another embodiment, the present invention provides a method of treating multiple myeloma comprising administering to a patient in need thereof a compound of formula I and one or more additional therapeutic agents selected from bortezomib (Velcade®), and dexamethasone (Decadron®), a hedgehog signaling inhibitor, a BTK inhibitor, a JAK/pan-JAK inhibitor, a TYK2 inhibitor, a PI3K inhibitor, a SYK inhibitor in combination with lenalidomide (Revlimid®).

In another embodiment, the present invention provides a method of treating or lessening the severity of a disease comprising administering to a patient in need thereof a compound of formula I and a PI3K inhibitor, wherein the disease is selected from a cancer, a neurodegenerative disorder, an angiogenic disorder, a viral disease, a hormone-related disease, conditions associated with organ transplantation, immunodeficiency disorders, a destructive bone disorder, a proliferative disorder, an infectious disease, a condition associated with cell death, thrombin-induced platelet aggregation, chronic myelogenous leukemia (CML), chronic lymphocytic leukemia (CLL), liver disease, pathologic immune conditions involving T cell activation, a cardiovascular disorder, and a CNS disorder.

In another embodiment, the present invention provides a method of treating or lessening the severity of a disease comprising administering to a patient in need thereof a compound of formula I and a PI3K inhibitor, wherein the disease is selected from benign or malignant tumor, carcinoma or solid tumor of the brain, kidney (e.g., renal cell carcinoma (RCC)), liver, adrenal gland, bladder, breast, stomach, gastric tumors, ovaries, colon, rectum, prostate, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukemia, diseases include Cowden syndrome, Lhermitte-Dudos disease and Bannayan-Zonana syndrome.

In some embodiments, the proliferative disorder is chronic lymphocytic leukemia, diffuse large B-cell lymphoma, Hodgkin's disease, small-cell lung cancer, non-small-cell lung cancer, myelodysplastic syndrome, lymphoma, a hematological neoplasm, or solid tumor.

In some embodiments, the other therapeutic compounds are antiproliferative compounds. Such antiproliferative compounds include, but are not limited to aromatase inhibitors; antiestrogens; topoisomerase I inhibitors; topoisomerase II inhibitors: microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists: anti-androgens; methionine aminopeptidase inhibitors: matrix metalloproteinase inhibitors; bisphosphonates: biological response modifiers; antiproliferative antibodies; heparinase inhibitors; inhibitors of Ras oncogenic isoforms: telomerase inhibitors; proteasome inhibitors: compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylanino-17-demethoxv-geldananwcin, NSC707545), IPI-504, CNF1010, CNF2024, CNF1010 from Conforma Therapeutics; temozolomide (Temodal®): kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin. The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™. Letrozole is marketed under the trade names Femara™ or Femar™. Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™ Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™

The term “topoisomerase II inhibitor” as used herein includes, but is not limited to the anthracyclines such as doxorubicin (including liposomal formulation, such as Caelyx™), daunorubicin, epirubicin, idarubicin and nemorubicin, the anthraquinones mitoxantrone and losoxantrone, and the podophillotoxines etoposide and teniposide. Etoposide is marketed under the trade name Etopophos™. Teniposide is marketed under the trade name VM 26-Bristol Doxorubicin is marketed under the trade name Acriblastin™ or Adriamycin™. Epirubicin is marketed under the trade name Farmorubicin™ Idarubicin is marketed, under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™. Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to. 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xcloda™. Gemcitabine is marketed under the trade name Gemzar™

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family: f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107): PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt. Ras/MAPK, PI3K, SYK, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosinc; RO 318220 and RO 320432: GO 6976; lsis 3521; LY333531/LY379196; isochinoline compounds; FTIs; PD184352 or QAN697 (a PI3K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester: NSC 680410, adaphostin): 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor. ErbB2. ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, Cl-1033, EKB-569, GW-2016, E1.1, E2.4. E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; see WO2008118802), navitoclax (and analogs thereof, see U.S. Pat. No. 7,390,799), NH-1 (Shenayng Pharmaceutical University), obatoclax (and analogs thereof, see WO2004106328), S-001 (Gloria Pharmaceuticals), TW series compounds (Univ, of Michigan), and venetoclax. In some embodiments the Bcl-2 inhibitor is a small molecule therapeutic. In some embodiments the Bcl-2 inhibitor is a peptidomimetic.

Further examples of BTK inhibitory compounds, and conditions treatable by such compounds in combination with compounds of this invention can be found in WO2008039218 and WO2011090760.

Further examples of SYK inhibitory compounds, and conditions treatable by such compounds in combination with compounds of this invention can be found in WO2003063794, WO2005007623, and WO2006078846.

Further examples of PI3K inhibitory compounds, and conditions treatable by such compounds in combination with compounds of this invention can be found in WO2004019973, WO2004089925, WO2007016176, U.S. Pat. No. 8,138,347, WO2002088112, WO2007084786, WO2007129161, WO2006122806, WO2005113554, and WO2007044729.

Further examples of JAK inhibitory compounds, and conditions treatable by such compounds in combination with compounds of this invention can be found in WO2009114512. WO2008109943, WO2007053452, WO2000142246, and WO2007070514.

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib. ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™ Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to. Bortezomib (Velcade™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamatc peptidomimctic inhibitor batimastat and its orally bioavailablc analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; ALK inhibitors, which are compounds which target, decrease or inhibit anaplastic lymphoma kinase, and Bcl-2 inhibitors.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino, 17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DMI, erbitux, bevacizumab (Avastin™), rituximab (Rituxan®), PR064553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412. In some embodiments, the present invention provides a method of treating AML associated with an ITD and/or D835Y mutation, comprising administering a compound of the present invention together with a one or more FLT3 inhibitors. In some embodiments, the FLT3 inhibitors are selected from quizartinib (AC220), a staurosporine derivative (e.g. midostaurin or lestaurtinib), sorafenib, tandutinib, LY-2401401, LS-104, EB-10, famitinib, NOV-110302, NMS-P948, AST-487, G-749, SB-1317, S-209, SC-110219, AKN-028, fedratinib, tozasertib, and sunitinib. In some embodiments, the FLT3 inhibitors are selected from quizartinib, midostaurin, lestaurtinib, sorafenib, and sunitinib.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy. Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine. 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides: ZD4190: ZD6474: SU5416: SU6668; bevacizumab: or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgG1 antibody, Angiozyme (RPI 4610) and Bevacizumab (Avastin™)

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The structure of the active compounds identified by code numbers, generic or trade names may be taken from the actual edition of the standard compendium “The Merck Index” or from databases, e.g., Patents International (e.g. IMS World Publications).

### Exemplary Immuno-Oncology Agents

In some embodiments, one or more other therapeutic agent is an immuno-oncology agent. As used herein, the term “an immuno-oncology agent” refers to an agent which is effective to enhance, stimulate, and/or up-regulate immune responses in a subject. In some embodiments, the administration of an immuno-oncology agent with a compound of the invention has a synergic effect in treating a cancer.

An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTβR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin α1β2, FAS, FASL, RELT, DR6, TROY, NGFR.

In some embodiments, an immuno-oncology agent is a cytokine that inhibits T cell activation (e.g., IL-6, IL-10, TGF-β, VEGF, and other immunosuppressive cytokines) or a cytokine that stimulates T cell activation, for stimulating an immune response.

In some embodiments, a combination of a compound of the invention and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1. PD-L1, PD-L2, LAG-3. TIM-3, Galectin 9, CEACAM-1. BTLA, CD69. Galectin-1, TIGIT, CD113, GPR56, VISTA, 2B4, CD48, GARP, PD1H, LAIR1, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, an immuno-oncology agent is an antagonist of inhibitory receptors on NK cells or an agonists of activating receptors on NK cells. In some embodiments, an immuno-oncology agent is an antagonist of KIR, such as lirilumab.

In some embodiments, an immuno-oncology agent is an agent that inhibits or depletes macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fc portion of IgG1, called AMP-224.

In some embodiments, an immuno-oncology agent is a PD-L1 antagonist. In some embodiments, a PD-L1 antagonist is an antagonistic PD-L1 antibody. In some embodiments, a PD-L1 antibody is MPDL3280A (RG7446; WO2010/077634), durvalumab (MED14736), BMS-936559 (WO2007/005874), and MSB0010718C (WO2013/79174).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO10/19570, WO14/08218), or IMP-731 or IMP-321 (WO08/132601, WO009/44273).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO006/105021, WO009/009116), or MK-4166 (WO11/028683).

In some embodiments, an immuno-oncology agent is an indoleamine (2,3)-dioxygenase (IDO) antagonist. In some embodiments, an IDO antagonist is selected from epacadostat (INCB024360, Incyte); indoximod (NLG-8189, NewLink Genetics Corporation): capmanitib (INC280, Novartis); GDC-0919 (Genentech/Roche); PF-06840003 (Pfizer); BMS:F001287 (Bristol-Myers Squibb); Phy906/KD108 (Phytoceutica); an enzyme that breaks down kynurenine (Kynase, Ikena Oncology, formerly known as Kyn Therapeutics); and NLG-919 (WO09/73620, WO009/1156652, WO11/56652, WO12/142237).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO06/029879).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3) (WO11/109400).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadostat, epratuzumab, indoximod, inotuzumab ozogamicin, intetumumab, ipilimumab, isatuximab, lambrolizumab, MED14736, MPDL3280A, nivolumab, obinutuzumab, ocaratuzumab, ofatumumab, olaratumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. See, e.g., Okazaki, T. et al. (2013) Nat. Immunol. 14, 1212-1218; Zou et al. (2016) Sci. Transl. Med. 8. The anti-PD-1 antibody nivolumab (OPDIVO®, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (POMALYST®, Celgene); lenalidomide (REVLIMID®, Celgene); ingenol mebutate (PICATO®, LEO Pharma).

In some embodiments, an immuno-oncology agent is a cancer vaccine. In some embodiments, the cancer vaccine is selected from sipuleucel-T (PROVENGE®, Dendreon/Valeant Pharmaceuticals), which has been approved for treatment of asymptomatic, or minimally symptomatic metastatic castrate-resistant (hormone-refractory) prostate cancer; and talimogene laherparepvec (IMLYGIC®, BioVex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (REOLYSIN®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAd1), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In some embodiments, an immuno-oncology agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil: TG01 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8⁺ T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682, which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors&pg=1].

In some embodiments, an immunostimulatory agent is an activator of retinoic acid receptor-related orphan receptor γ (RORγt). RORγt is a transcription factor with key roles in the differentiation and maintenance of Type 17 effector subsets of CD4+ (Th17) and CD8+ (Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337, VentiRx Pharmaceuticals) which is being studied for squamous cell cancer of the head and neck (NCT02124850) and ovarian cancer (NCT02431559).

Other immuno-oncology agents that can be used in the present invention include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201. Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORγt.

In some embodiments, an immunostimulatory therapeutic is recombinant human interleukin 15 (rhIL-15), rhIL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhIL-12). In some embodiments, an IL-15 based immunotherapeutic is heterodimeric IL-15 (hetIL-15, Novartis/Admune), a fusion complex composed of a synthetic form of endogenous IL-15 complexed to the soluble IL-15 binding protein IL-15 receptor alpha chain (IL15:sIL-15RA), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhIL-12) is NM-IL-12 (Neumedicines, Inc.), NCT02544724, or NCT02542124.

In some embodiments, an immuno-oncology agent is selected from those descripted in Jerry L. Adams et al., “Big opportunities for small molecules in immuno-oncology,” Cancer Therapy 2015, Vol. 14, pages 603-622y. In some embodiment, an immuno-oncology agent is selected from the examples described in Table 1 of Jerry L. Adams et al. In some embodiments, an immuno-oncology agent is a small molecule targeting an immuno-oncology target selected from those listed in Table 2 of Jerry L. Adams et al. In some embodiments, an immuno-oncology agent is a small molecule agent selected from those listed in Table 2 of Jerry L. Adams et al.

In some embodiments, an immuno-oncology agent is selected from the small molecule immuno-oncology agents described in Peter L. Toogood, “Small molecule immuno-oncology therapeutic agents,” Bioorganic & Medicinal Chemistry Letters 2018. Vol. 28, pages 319-329.

In some embodiments, an immuno-oncology agent is selected from those described in Sandra L. Ross et al., “Bispecific T cell engager (BITE®) antibody constructs can mediate bystander tumor cell killing”, PLoS ONE 12(8): e0183390. In some embodiments, an immuno-oncology agent is a bispecific T cell engager (BITE®) antibody construct. In some embodiments, a bispecific T cell engager (BITE®) antibody construct is a CD19/CD3 bispecific antibody construct. In some embodiments, a bispecific T cell engager (BITE®) antibody construct is an EGFR/CD3 bispecific antibody construct. In some embodiments, a bispecific T cell engager (BITE(®) antibody construct activates T cells. In some embodiments, a bispecific T cell engager (BITER) antibody construct activates T cells, which release cytokines inducing upregulation of intercellular adhesion molecule 1 (ICAM-1) and FAS on bystander cells. In some embodiments, a bispecific T cell engager (BITE®) antibody construct activates T cells which result in induced bystander cell lysis. In some embodiments, the bystander cells are in solid tumors. In some embodiments, the bystander cells being lysed are in proximity to the BITE®-activated T cells. In some embodiment, the bystander cells comprises tumor-associated antigen (TAA) negative cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

### Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA: CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In some embodiments, the checkpoint inhibitor is a biologic therapeutic or a small molecule. In some embodiments, the checkpoint inhibitor is a monoclonal antibody, a humanized antibody, a fully human antibody, a fusion protein or a combination thereof. In some embodiments, the checkpoint inhibitor inhibits a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In some embodiments, the checkpoint inhibitor interacts with a ligand of a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In some embodiments, the checkpoint inhibitor is an immunostimulatory agent, a T cell growth factor, an interleukin, an antibody, a vaccine or a combination thereof. In some embodiments, the interleukin is IL-7 or IL-15. In some embodiments, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors can include small molecule inhibitors or can include antibodies, or antigen binding fragments thereof, that bind to and block or inhibit immune checkpoint receptors or antibodies that bind to and block or inhibit immune checkpoint receptor ligands. Illustrative checkpoint molecules that can be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8⁺ (αβ) T cells), CD160 (also referred to as BY55), CGEN-15049, CHK 1 and CHK2 kinases, A2aR, and various B-7 family ligands. B7 family ligands include, but are not limited to. B7-1, B7-2. B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include, but are not limited to, Tremelimumab (CTLA-4 blocking antibody), anti-OX40, PD-L1 monoclonal Antibody (Anti-B7-H1; MED14736). MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (OPDIVO®), ipilimumab (YERVOY®), and pembrolizumab (KEYTRUDA®). In some embodiments, the checkpoint inhibitor is selected from nivolumab (anti-PD-1 antibody, OPDIVO®, Bristol-Myers Squibb): pembrolizumab (anti-PD-1 antibody, KEYTRUDA®, Merck); ipilimumab (anti-CTLA-4 antibody, YERVOY®, Bristol-Myers Squibb); durvalumab (anti-PD-L1 antibody, IMFINZI®, AstraZeneca); and atezolizumab (anti-PD-L1 antibody, TECENTRIQ®, Genentech).

In some embodiments, the checkpoint inhibitor is selected from the group consisting of lambrolizumab (MK-3475), nivolumab (BMS-936558), pidilizumab (CT-011), AMP-224, MDX-1105, MED14736, MPDL3280A, BMS-936559, ipilimumab, lirlumab, IPH2101, pembrolizumab (KEYTRUDA®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple mycloma; avchmuab (BAVENCIO®, Pfizer/Merck KGaA), also known as MSB0010718C), a fully human IgG1 anti-PD-L1 antibody, in clinical trials for non-small cell lung cancer. Merkel cell carcinoma, mesothelioma, solid tumors, renal cancer, ovarian cancer, bladder cancer, head and neck cancer, and gastric cancer: or PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IMP321 (Immutep S.A.) is an LAG-3-Ig fusion protein, being studied in melanoma (NCT02676869): adenocarcinoma (NCT02614833); and metastatic breast cancer (NCT00349934).

Checkpoint inhibitors that can be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812: NCT05082566); GSK3174998 (Merck), an agonistic anti-OX40 antibody, in Phase 1 cancer trials (NCT02528357); MEDI0562 (Medimmune/AstraZeneca), an agonistic anti-OX40 antibody, in advanced solid tumors (NCT02318394 and NCT02705482); MEDI6469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCT01303705); and BMS-986178 (Bristol-Myers Squibb) an agonistic anti-OX40 antibody, in advanced cancers (NCT02737475).

Checkpoint inhibitors that can be used in the present invention include CD137 (also called 4-1BB) agonists. CD137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981); and CTX-471 (Compass Therapeutics), an agonistic anti-CD137 antibody in metastatic or locally advanced malignancies (NCT03881488).

Checkpoint inhibitors that can be used in the present invention include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that can be used in the present invention include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MED11873 (Medimmune/AstraZeneca), an agonistic hexameric GITR-ligand molecule with a human IgG1 Fc domain, in advanced solid tumors (NCT02583165).

Checkpoint inhibitors that can be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that can be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that can be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgG1, acts by binding human CD47, and preventing it from delivering its “do not eat” signal to macrophages, is in clinical trials in Phase 1 (NCT02890368 and NCT02663518): CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Checkpoint inhibitors that can be used in the present invention include CD73 inhibitors. CD73 inhibitors that are being studied in clinical trials include MEDI9447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that can be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

Checkpoint inhibitors that can be used in the present invention include CSF1R inhibitors. CSF1R inhibitors that are being studied in clinical trials include pexidartinib (PLX3397, Plexxikon), a CSF1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF—IR antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Checkpoint inhibitors that can be used in the present invention include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the current invention, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein. Additional compounds of the invention were prepared by methods substantially similar to those described herein in the Examples and methods known to one skilled in the art.

Abbreviations

| | |
|---|---|
| Ac: acetyl | ACN: acetonitrile |
| AcOH: acetic acid | Ad: adamantly |
| AIBN: 2,2′-azo bisisobutyronitrile | DMSO-dimethyl sulfoxide |
| Anhyd: anhydrous | DPPA: diphenylphosphoryl azide |
| Aq: aqueous | dppf: 1,1′-bis(diphenylphosphino)ferrocene |
| B₂Pin₂: bis (pinacolato)diboron- | EDC or EDCI: 1-(3-dimethylaminopropyl)-3- |
| 4,4,4′,4′,5,5,5′,5′-octamethyl-2,2′-bi(1,3,2- | ethylcarbodiimide hydrochloride |
| dioxaborolane) | ee: enantiomeric excess |
| BINAP: 2,2′-bis(diphenylphosphino)-1,1′- | ESI: electrospray ionization |
| binaphthyl | EA: ethyl acetate |
| BH₃: borane | EtOAc: ethyl acetate |
| Bn: benzyl | EtOH: ethanol |
| Boc: tert-butoxycarbonyl | FA: formic acid |
| Boc₂O: di-tert-butyl dicarbonate | h or hrs: hours |
| BPO: benzoyl peroxide | HATU: N,N,N′,N′-tetramethyl-O-(7- |
| ⁿBuOH: n-butanol | azabenzotriazol-1-yl)uronium |
| cataCXium: di-adamantylalkylphosphine | hexafluorophosphate |
| CDI: carbonyldiimidazole | HCl: hydrochloric acid |
| COD: cyclooctadiene | HPLC: high performance liquid |
| d: days | chromatography |
| DABCO: 1,4-diazobicyclo[2.2.2]octane | HOAc: acetic acid |
| DAST: diethylaminosulfur trifluoride | IBX: 2-iodoxybenzoic acid |
| dba: dibenzylideneacetone | IPA: isopropyl alcohol |
| DBU: 1,8-diazobicyclo[5.4.0]undec-7-ene | KHMDS: potassium hexamethyldisilazide |
| DCE: 1,2-dichloroethane | K₂CO₃: potassium carbonate |
| DCM: dichloromethane | LAH: lithium aluminum hydride |
| DEA: diethylamine | LDA: lithium diisopropylamide |
| DHP: dihydropyran | m-CPBA: meta-chloroperbenzoic acid |
| DIBAL-H: diisobutylaluminum hydride | M: molar |
| DIPA: diisopropylamine | MeCN: acetonitrile |
| DIPEA or DIEA: N,N-diisopropylethylamine | MeOH: methanol |
| DMA: N,N-dimethylacetamide | Me₂S: dimethyl sulfide |
| DME: 1,2-dimethoxyethane | MeONa: sodium methylate |
| DMAP: 4-dimethylaminopyridine | MeI: iodomethane |
| DMF: N,N-dimethylformamide | min: minutes |
| DMP: Dess-Martin periodinane | mL: milliliters |
| mM: millimolar | Pd(PPh₃)₄: palladium |
| mmol: millimoles | tetrakis(triphenylphosphine) |
| MPa: mega pascal | PBS: phosphate buffered saline |
| MOMCl: methyl chloromethyl ether | PE: petroleum ether |
| MsCl: methanesulfonyl chloride | POCl₃: phosphorus oxychloride |
| MTBE: methyl tert-butyl ether | PPh₃: triphenylphosphine |
| nBuLi: n-butyllithium | PyBOP: (benzotriazol-1- |
| NaNO₂: sodium nitrite | yloxy)tripyrrolidinophosphonium |
| NaOH: sodium hydroxide | hexafluorophosphate |
| Na₂SO₄: sodium sulfate | Rel: relative |
| NBS: N-bromosuccinimide | RuPhos: 2-Dicyclohexylphosphino-2′,6′- |
| NCS: N-chlorosuccinimide | diisopropoxybiphenyl |
| NFSI: N-fluorobenzenesulfonimide | R.T. or rt: room temperature |
| NMO: N-methylmorpholine N-oxide | sat: saturated |
| NMP: N-methylpyrrolidine | SEMCl: chloromethyl-2-trimethylsilylethyl |
| NMR: Nuclear Magnetic Resonance | ether |
| ° C: degrees Celsius | SFC: supercritical fluid chromatography |
| o/n: overnight | SOCl₂: sulfur dichloride |
| Palladacycle G2 (Pd G2): [2-(2′-amino-1,1′- | tBuOK: potassium tert-butoxide |
| biphenyl)]palladium chloride | TBAB: tetrabutylammonium bromide |
| Palladacycle G3 (Pd G3): [2-(2′-amino-1,1′- | TBAI: tetrabutylammonium iodide |
| biphenyl)]palladium methanesulfonate | TEA: triethylamine |
| Pd/C: palladium on Carbon | Tf: trifluoromethanesulfonate |
| Pd(dba)₂: palladium | TfAA, TFMSA or Tf₂O: |
| bis(dibenzylideneacetone) | trifluoromethanesulfonic anhydride |
| Pd(dtdpf)Cl₂: [1,1′-Bis(di-tert- | TFA: trifluoracetic acid |
| butylphosphino)ferrocene]dichloro palladium | TIPS: triisopropylsilyl |
| Pd(OAc)₂: palladium acetate | THF: tetrahydrofuran |
| Pd-PEPPSI-IPentCl: Dichloro[1,3-bis(2,6-Di- | THP: tetrahydropyran |
| 3-pentylphenyl)imidazol-2-ylidene](3- | TLC: thin layer chromatography |
| chloropyridyl)palladium | TMEDA: tetramethylethylenediamine |
| Pd(PPh₃)₂Cl₂: palladium | T₃P: propylphosphonic anhydride |
| (bis(triphenylphosphine) dichloride | pTSA: para-toluenesulfonic acid |
| Xantphos: 4,5-bis(diphenylphosphino)-9,9- | wt: weight |
| dimethylxanthene | XPhos: 2-dicyclohexylphosphino-2′,4′,6′- |
| | triisopropylbiphenyl |

### General Synthetic Methods

All starting materials, building blocks, reagents, acids, bases, dehydrating agents, solvents, and catalysts utilized to synthesis the compounds of the present invention were either commercially available or can be produced by organic synthesis methods known to one of ordinary skill in the art (Houben-Weyl 4th Ed. 1952. Methods of Organic Synthesis, Thieme, Volume 21). Further, the compounds of the present invention can be produced by organic synthesis methods known to one of ordinary skill in the art as shown in the following examples.

Proton NMR (¹H NMR) was conducted in deuterated solvent. In certain compounds disclosed herein, one or more ¹H shifts overlap with residual proteo solvent signals; these signals have not been reported in the experimental provided hereinafter.

### Example 1. Methyl 2-{4-[5-cyano-2-(4-methyl-1,2,4-triazol-3-yl)phenyl]-6-[(2-cyanoethyl) amino]pyridin-2-yl}-7-(trifluoromethyl)-1,3-benzoxazole-5-carboxylate (I-11)

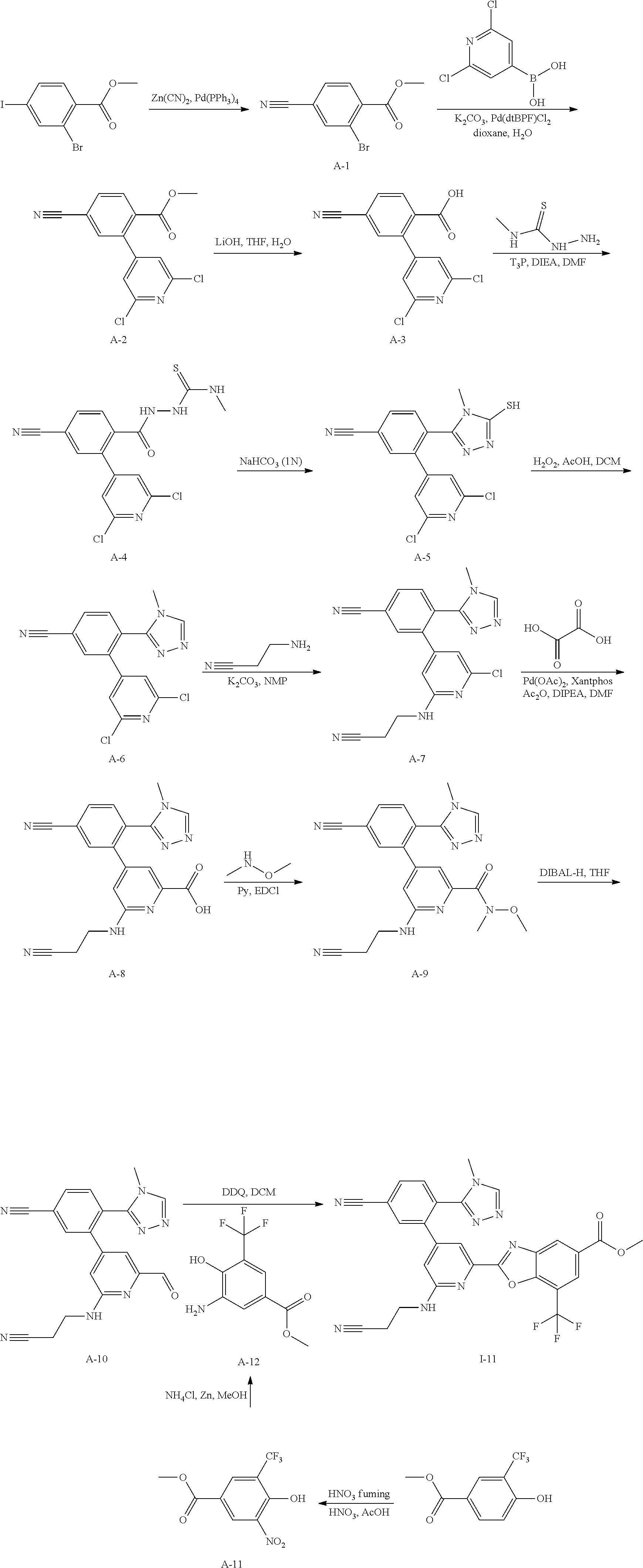

### Synthesis of methyl 2-bromo-4-cyanobenzoate (A-1)

To a stirred solution of methyl 2-bromo-4-iodobenzoate (3.00 g, 1 eq, 8.80 mmol) and Zn(CN)₂ (1.34 g, 1.3 eq, 11.4 mmol) in DMF (20 mL) was added Pd(PPh₃)₄ (1.02 g, 0.1 eq, 0.88 mmol) at room temperature under nitrogen atmosphere. The resulting mixture was stirred for 4 h at 60° C. under nitrogen atmosphere. The mixture was cooled to room temperature, diluted with water and extracted with ethyl acetate (3×200 mL). The combined organic layers were washed with brine (2×200 mL), dried over anhdrous sodium sulfate. After filtration, the filtrate was concentrated under reduced pressure. The residue was applied on a silica gel column chromatography with petroleum ether/ethyl acetate (20/1) to afford A-1 (1.5 g, 6.27 mmol, 71%, LCMS purity 90%) as a yellow solid. MS (ES): m/z 420.1/422.1 [M+H]⁺.

### Synthesis of methyl 4-cyano-2-(2,6-dichloropyridin-4-yl)benzoate (A-2)

To a stirred solution of methyl 2-bromo-4-cyanobenzoate (2.00 g, 1 eq, 8.33 mmol), 2,6-dichloropyridin-4-ylboronic acid (1.92 g. 1.2 eq. 10.0 mmol) and K₂CO₃ (3.45 g, 25.0 mmol, 3 eq) in dioxane (20 mL) and H₂O (2 mL) was added Pd(DtBPF)Cl₂ (540 g, 0.1 eq, 0.83 mmol) at room temperature under nitrogen atmosphere. The resulting mixture was stirred for 4 h at 60° C. under nitrogen atmosphere. The mixture was allowed to cool down to room temperature. The resulting mixture was concentrated under reduced pressure. The residue was applied on a silica gel column chromatography with petroleum ether/ethyl acetate (5/1) to afford methyl A-2 (4.92 g. 16.0 mmol, 192%. LCMS purity 52%) as a brown solid. MS (ES): m/z 307.1/309.1 [M+H]⁺.

### Synthesis of 4-cyano-2-(2,6-dichloropyridin-4-yl)benzoic acid (A-3)

To a stirred solution of methyl 4-cyano-2-(2,6-dichloropyridin-4-yl)benzoate (3.00 g, 1 eq, 9.77 mmol) and LiOH (1.17 g, 48.8 mmol, 5 eq) in THE (60 ml) and H₂O (20 mL) at room temperature. The resulting mixture was stirred for 3 h at room temperature. The mixture was acidified to pH 2 with cone. HCl at 0° C. The resulting mixture was diluted with water and extracted with ethyl acetate (3×200 mL). The combined organic layers were washed with brine (2×200 mL), dried over anhydrous sodium sulfate. After filtration, the filtrate was concentrated under reduced pressure to afford A-3. (7.1 g, crude) as a brown solid. MS (ES): m, z 293.1/295.1 [M+H]⁺.

### Synthesis of 4-cyano-2-(2,6-dichloropyridin-4-yl)-N-[(methylcarbamothioyl)amino]benzamide (A-4)

To a stirred solution of 4-cyano-2-(2,6-dichloropyridin-4-yl)benzoic acid (2.00 g, 1 eq, 6.82 mmol) and 4-methyl-3-thiosemicarbazide (1.08 g. 1.5 eq, 10.3 mmol) in DMF (20 mL) were added T₃P in DMF (8.68 g. 50% Wt, 4 Eq, 27.3 mmol) and DIPEA (7.06 g. 8 Eq. 54.6 mmol) at room temperature. The resulting mixture was stirred for 2 h at room temperature. The crude product was purified by reverse flash column chromatography with the following conditions: Column, C18; mobile phase, Water (0.1% NH₄HCO₃) and ACN (0% ACN up to 50% in 30 min); Detector, UV 254/220 nm. The product-containing fractions were combined and concentrated under reduced pressure to afford A-4. (1.8 g, 4.73 mmol, 69%, LCMS purity 89%) as a yellow solid. MS (ES): m/z 380.2/382.2 [M+H]⁺.

### Synthesis of 3-(2,6-dichloropyridin-4-yl)-4-(4-methyl-5-sulfanyl-1,2,4-triazol-3-yl)benzonitrile (A-5)

A solution of 4-cyano-2-(2,6-dichloropyridin-4-yl)-N-[(methylcarbamothioyl)amino]benzamide (2.00 g, 1 eq, 5.26 mmol) in aq, of NaHCO₃ (40 mL, 1 M) was stirred for 4 h at 80° C. The mixture was cooled to room temperature and concentrated under reduced pressure. The crude product was purified by reverse flash column chromatography with the following conditions: Column, C18; mobile phase, Water (0.1% FA) and ACN (0% ACN up to 40% in 30 min); Detector, UV 254/220 nm. To afford a yellow solid A-5 (950 mg, 2.63 mmol, 54%, LCMS purity 92%). MS (ES): m/z 362.2/364.2 [M+H]⁻.

### Synthesis of 3-(2,6-dichloropyridin-4-yl)-4-(4-methyl-1,2,4-triazol-3-yl)benzonitrile (A-6)

To a stirred solution of 3-(2,6-dichloropyridin-4-yl)-4-(4-methyl-5-sulfanyl-1,2,4-triazol-3-yl)benzonitrile (20 mg, 1 Eq, 0.06 mmol) and AcOH (7 mg. 2 Eq. 0.11 mmol) in DCM (8 mL) was added H₂O₂ (31 mg, 30% Wt, 5 Eq. 0.28 mmol) at 0° C. The resulting mixture was stirred for 2 h at room temperature. The resulting mixture was concentrated under reduced pressure. The crude product was purified by reverse flash column chromatography with the following conditions: Column, C18; mobile phase, Water (0.1% NH₄HCO₃) and ACN (10% ACN up to 50% in 20 min); Detector, UV 254/220 nm. The crude product was purified by Prep-HPLC with the following conditions (Column: YMC-Actus Triart C18, 30* 150 mm, 5 m; Mobile Phase A: Water (0.1% NH₄HCO₃). Mobile Phase B: ACN; Flow rate: 60 mL/min: Gradient: 20% B to 50% B in 8 min: Wave Length: 254/210 nm; RT: 7.3). The fraction was collected and concentrated under vacuum, the residue was re-dissolved in CH₃CN and H₂O, and then was lyophilized to afford A-6 (14.4 mg, 43 μmol, 79%, LCMS purity 98%) as a white solid. MS (ES): m/z 330.0/332.0 [M+H]⁺.

### Synthesis of 3-{2-chloro-6-[(2-cyanoethyl)amino]pyridin-4-yl}-4-(4-methyl-1,2,4-triazol-3-yl)benzonitrile (A-7)

To a stirred solution of 3-(2,6-dichloropyridin-4-yl)-4-(4-methyl-1,2,4-triazol-3-yl)benzonitrile (2.00 g, 1 Eq, 6.06 mmol) and β aminopropionitrile (4.25 g, 10 Eq, 60.6 mmol) in NMP (20 mL) was added K₂CO₃ (8.37 g, 10 Eq, 60.6 mmol) at room temperature. The resulting mixture was stirred for overnight at 100° C. The mixture was allowed to cool down to room temperature. The crude product was purified by reverse flash column chromatography with the following conditions: Column, C18; mobile phase, Water (0.1% NH4HCO3) and ACN (10% ACN up to 60% in 25 min): Detector. UV 254/220 nm. To afford an off-white solid A-7 (1.8 g, 4.96 mmol, 82%, LCMS purity 93%). MS (ES): m/z 364.1/366.1 [M+H]⁺

### Synthesis of 4-[5-cyano-2-(4-methyl-1,2,4-triazol-3-yl)phenyl]-6-[(2-cyanoethyl)amino]pyridine-2-carboxylic acid (A-8)

To a stirred mixture of 3-{2-chloro-6-[(2-cyanoethyl)amino]pyridin-4-yl}-4-(4-methyl-1,2,4-triazol-3-yl)benzonitrile (60 mg, 1 Eq, 0.16 mmol) and oxalic acid (18 mg, 1.2 Eq, 0.20 mmol) in DMF (3 mL) were added Ac₂O (25 mg, 1.5 Eq, 0.25 mmol), DIPEA (32 mg, 1.5 Eq, 0.25 mmol), XantPhos (19 mg, 0.2 Eq, 0.03 mmol) and Pd(OAc)₂ (4 mg, 0.1 Eq, 0.02 mmol) at room temperature under nitrogen atmosphere. The resulting mixture was stirred for overnight at 100° C. under nitrogen atmosphere. The mixture was allowed to cool down to room temperature. The crude product was purified by reverse flash column chromatography with the following conditions: Column. C18; mobile phase, Water (0.1% FA) and ACN (10% ACN up to 50% in 30 min); Detector, UV 254/220 nm. The crude product was purified by Prep-HPLC with the following conditions (Column: SunFire Prep C18 OBD Column, 19*150 mm, 5 m; Mobile Phase A: Water (0.1% FA), Mobile Phase B: ACN; Flow rate: 25 mL/min; Gradient: 25% B to 50% B in 9 min: Wave Length: 254/210 nm; RT: 7.7). The fraction was collected and concentrated under vacuum, the residue was re-dissolved in CH₃CN and H₂O, and then was lyophilized to afford A-8 (3.1 mg, 8.3 μmol, 5.0%, LCMS purity 99%) as a white solid. MS (ES): m/z 374.1 [M+H]. ¹H NMR (400 MHz, DMSO-d₆) δ 12.66 (s, 1H), 8.52 (s, 1H), 8.21-8.08 (m, 2H), 7.83 (d, 1H), 7.31 (t, 1H), 6.97 (d, 1H), 6.54-6.50 (m, 1H), 3.59-3.49 (m, 2H), 3.26 (s, 3H), 2.77 (t, 2H).

### Synthesis of 4-[5-cyano-2-(4-methyl-1,2,4-triazol-3-yl)phenyl]-6-[(2-cyanoethyl)amino]-N-methoxy-N-methylpyridine-2-carboxamide (A-9)

To a stirred mixture of 4-[5-cyano-2-(4-methyl-1.2.4-triazol-3-yl)phenyl]-6-[(2-cyanoethyl)amino]pyridine-2-carboxylic acid (70 mg, 1 Eq, 0.19 mmol) and N,O-dimethylhydroxylamine (14 mg, 1.2 Eq, 0.22 mmol) in Pyridine (3 mL) was added EDCI (72 mg, 2 Eq, 0.37 mmol) at room temperature. The resulting mixture was stirred for overnight at 60° C. The mixture was allowed to cool down to room temperature. The resulting mixture was concentrated under reduced pressure. The crude product was purified by reverse flash column chromatography with the following conditions: Column, C18; mobile phase. Water (0.1% NH₄HCO₃) and ACN (0% ACN up to 30% in 25 min): Detector. UV 254/220 nm. The crude product was purified by Prep-HPLC with the following conditions (Column: XBridge Prep OBD C18 Column, 30*150 mm, 5 μm; Mobile Phase A: Water (0.1% NH₄HCO₃), Mobile Phase B: ACN; Flow rate: 60 mL/min; Gradient: 16% B to 20% B in 7 min; Wave Length: 254 nm; RT: 7.37). The fraction was collected and concentrated under vacuum, the residue was re-dissolved in CH₃CN and H₂O, and then was lyophilized to afford A-9 (4.1 mg. 9.8 μmol, 5.2%, LCMS purity 99%) as a white solid. MS (ES): m/z 417.3 [M+H]⁺. ¹H NMR (400 MHz, DMSO-d₆) δ 8.52 (s, 1H), 8.13 (d. 1H), 8.11-8.07 (m, 1H), 7.81 (d, 1H), 7.25 (t, 1H), 6.46 (d, 1H), 6.37 (d, 1H), 3.62 (s, 3H), 3.52-3.45 (m, 2H), 3.22 (d, 6H), 2.73 (t, 2H).

### Synthesis of 3-{2-[(2-cyanoethyl)amino]-6-formylpyridin-4-yl}-4-(4-methyl-1,2,4-triazol-3-yl)benzonitrile (A-10)

To a stirred mixture of 4-[5-cyano-2-(4-methyl-1,2,4-triazol-3-yl)phenyl]-6-[(2-cyanoethyl)amino]-N-methoxy-N-methylpyridine-2-carboxamide (2.00 g, 1 Eq, 4.80 mmol) in THF (100 mL) was added DIBAl-H (5.46 g, 1 M, 8 Eq, 38.4 mmol) in portions at 0° C. under nitrogen atmosphere. The resulting mixture was stirred for 10 min at 0° C. under nitrogen atmosphere. The reaction was then quenched by the addition of 10 mL of ice water at 0° C. The resulting mixture was concentrated under reduced pressure. The residue was purified by Prep-TLC with dichloromethane/methyl alcohol (10/1) to afford A-10 (1 g, 2.80 mmol, 58%. LCMS purity 93%) as a white solid. MS (ES): m/z 358.1 [M+H]⁺.

### Synthesis of methyl 4-hydroxy-3-nitro-5-(trifluoromethyl)benzoate (A-11)

Into a 250 mL round-bottom flask were added methyl 4-hydroxy-3-(trifluoromethyl)benzoate (1.93 g, 1 Eq, 8.77 mmol) in AcOH (40 mL), then HNO₃ (2.76 g, 5 Eq, 43.8 mmol) and HNO₃ fuming (5.52 g, 10 Eq, 87.7 mmol) were added at −10° C. The resulting mixture was stirred for overnight at room temperature. The reaction was then quenched by the addition of 5 mL of ice water at 0° C. The resulting mixture was diluted with water and extracted with ethyl acetate (3×100 mL). The combined organic layers were washed with brine (2×100 mL), dried over anhydrous sodium sulfate. After filtration, the filtrate was concentrated under reduced pressure. The crude product was purified by reverse flash column chromatography with the following conditions: Column, C18; mobile phase, Water (0.1% FA) and ACN (30% ACN up to 60% in 7 min); Detector, UV 254/220 nm to afford A-11 (1.94 g, 7.29 mmol, 83%, LCMS purity 89%) as a yellow solid. MS (ES): m/z 266.0 [M+H]⁺.

### Synthesis of methyl 3-amino-4-hydroxy-5-(trifluoromethyl)benzoate (A-12)

Into an 8 mL sealed tube were added methyl 4-hydroxy-3-nitro-5-(trifluoromethyl)benzoate (370 mg, 1 Eq, 1.40 mmol) and NH₄Cl (746 mg, 10 Eq, 14.0 mmol) in MeOH (10 mL) at room temperature. To the above mixture was added Zn powder (913 mg, 10 Eq. 14.0 mmol) over 2 min at room temperature. The resulting mixture was stirred for 2 h at 80° C. The mixture was allowed to cool down to room temperature. The resulting mixture was filtered: the filter cake was washed with ethanol (3×3 mL). The filtrate was concentrated under reduced pressure. To afford a yellow solid A-12 (200 mg, 0.85 mmol, 61%, 85% Purity). MS (ES): m/z 236.0 [M+H]⁺.

### Synthesis of methyl 2-{4-[5-cyano-2-(4-methyl-1,2,4-triazol-3-yl)phenyl]-6-[(2-cyanoethyl)amino]pyridin-2-yl}-7-(trifluoromethyl)-1,3-benzoxazole-5-carboxylate (I-11)

To a stirred mixture of 3-{2-[(2-cyanoethyl)amino]-6-formylpyridin-4-yl}-4-(4-methyl-1,2,4-triazol-3-yl)benzonitrile (70 mg, 1 eq, 0.20 mmol) and methyl 3-amino-4-hydroxy-5-(trifluoromethyl)benzoate (55 mg, 1.2 Eq, 0.24 mmol) in DCM (3 mL) the resulting mixture was stirred for 1 h at 60° C. The mixture was allowed to cool down to room temperature. To the above mixture was added DDQ (89 mg, 2 eq, 0.39 mmol) in portions at room temperature. The resulting mixture was stirred for 1 h at room temperature. The resulting mixture was concentrated under reduced pressure. The crude product was purified by reverse flash column chromatography with the following conditions: Column, C18: mobile phase, Water (0.1% FA) and ACN (10% ACN up to 60% in 25 min); Detector, UV 254/220 nm. The crude product was purified by Prep-HPLC with the following conditions (Column: XBridge Shield RP18 OBD Column, 30* 150 mm, 5 μm; Mobile Phase A: Water (0.1% NH₄HCO₃), Mobile Phase B: ACN; Flow rate: 60 mL/min; Gradient: 51% B to 56% B in 9 min; Wave Length: 254/210 nm; RT: 8.75). The fraction was collected and concentrated under vacuum, the residue was re-dissolved in CH₃CN and H₂O, and then was lyophilized to afford I-11 (3.2 mg, 5.6 μmol, 2.9%, 99.8% Purity) as a white solid. MS (ES): m/z 573.1 [M+H]. ¹H NMR (400 MHz, DMSO-d₆) δ 8.65 (d, 1H), 8.52 (s, 1H), 8.30-8.24 (m, 2H), 8.19-8.12 (m, 1H), 7.88 (d, 1H), 7.56 (t, 1H), 7.32 (d, 1H), 6.62 (d, 1H), 3.95 (s, 3H), 3.63-3.49 (m, 2H), 3.34 (s, 3H), 2.88 (t, 2H).

### Example 2. 3-{2-[(2-Cyanoethyl)amino]-6-(5-{[(2-hydroxy-2-methylpropyl)amino]methyl}-7-(trifluoromethyl)-1,3-benzoxazol-2-yl)pyridin-4-yl}-4-(4-methyl-1,2,4-triazol-3-yl)benzonitrile (I-15)

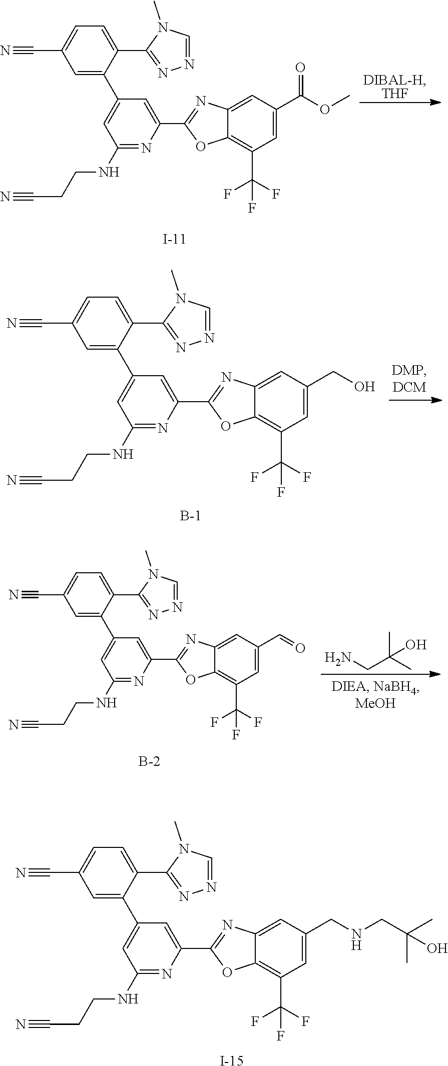

### Synthesis of 3-{2-[(2-cyanoethyl)amino]-6-[5-(hydroxymethyl)-7-(trifluoromethyl)-1,3-benzoxazol-2-yl]pyridin-4-yl}-4-(4-methyl-1,2,4-triazol-3-yl)benzonitrile (B-1)

To a stirred mixture of I-11 (300 mg, 1 eq, 0.52 mmol) in THF (10 mL) was added DIBAL-H (745 mg, 1 M, 10 eq, 5.24 mmol) at 0° C. The resulting mixture was stirred for 15 min at 0° C. The resulting mixture was concentrated under reduced pressure. The residue was purified by Prep-TLC with dichloromethane/methyl alcohol (10/1) to afford B-1 (60 mg, 0.11 mmol, 21%, LCMS purity 95%) as a white solid. MS (ES): m/z 545.2 [M+H]⁺.

### Synthesis of 3-{2-[(2-cyanoethyl)amino]-6-[5-formyl-7-(trifluoromethyl)-1,3-benzoxazol-2-yl]pyridin-4-yl}-4-(4-methyl-1,2,4-triazol-3-yl)benzonitrile (B-2)

To a stirred mixture of B-1 (60 mg, 1 eq, 0.09 mmol) in DCM (2 mL) was added DMP (58 mg. 1.5 eq. 0.14 mmol) at room temperature. The resulting mixture was stirred for 1 h at room temperature. The residue was purified by Prep-TLC with dichloromethane/methyl alcohol (10/1) to afford B-2 (50 mg, 0.09 mmol, 83%, LCMS purity 97%) as a white solid. MS (ES): m/z 543.1 [M+H]⁺.

### Synthesis of 3-{2-[(2-cyanoethyl)amino]-6-(5-{[(2-hydroxy-2-methylpropyl)amino]methyl}-7-(trifluoromethyl)-1,3-benzoxazol-2-yl)pyridin-4-yl}-4-(4-methyl-1,2,4-triazol-3-yl)benzonitrile (I-15)

To a stirred mixture of B-2 (60 mg, 1 Eq, 0.11 mmol) and 1-amino-2-methylpropan-2-ol (20 mg, 2 eq, 0.22 mmol) in MeOH (5 mL) was added DIPEA (42.89 mg. 0.333 mmol, 3 eq) at room temperature. The resulting mixture was stirred for overnight at 60° C. The mixture was allowed to cool down to room temperature. To the above mixture was added NaBH₄ (13 mg, 3 Eq, 0.33 mmol) at 0° C. The resulting mixture was stirred for additional 1 h at room temperature. The reaction was then quenched by the addition of 2 mL of methyl alcohol at 0° C. The resulting mixture was concentrated under reduced pressure. The crude product was purified by Prep-HPLC with the following conditions (Column: YMC-Actus Triart C18 150*30 mm: Mobile Phase A: Water (0.1% NH₄HCO₃), Mobile Phase B: CAN: Flow rate: 60 mL/min; Gradient: 42% B to 52% B in 10 min: Wave Length: 254 nm; RT: 8). The fraction was collected and concentrated under vacuum, the residue was re-dissolved in CH₃CN and H₂O, and then was lyophilized to afford I-15 (4.2 mg, 6.8 μmol, 6.1%, LCMS purity 98.6%) as a white solid. MS (ES): m/z 616.3 [M+H]⁺. ¹H NMR (400 MHz, DMSO-d₆) δ 8.51 (s, 1H), 8.26 (d, 1H), 8.17-8.12 (m, 1H), 8.10 (s, 1H), 7.87 (d, 1H), 7.81 (s, 1H), 7.50 (t, 1H), 7.27 (d, 1H), 6.59 (d, 1H), 4.22 (s, 1H), 3.94 (s, 2H), 3.62-3.53 (m, 2H), 3.30 (s, 3H), 2.87 (t, 2H), 2.39 (s, 2H), 1.11 (s, 6H).

### Example 3. Isopropyl[(2-{3-[(1r,3s)-3-methyl-1-(4-methyl-1,2,4-triazol-3-yl)cyclobutyl]phenyl}-7-(trifluoromethyl)-1,3-benzoxazol-5-yl)methyl]amine (1-5)

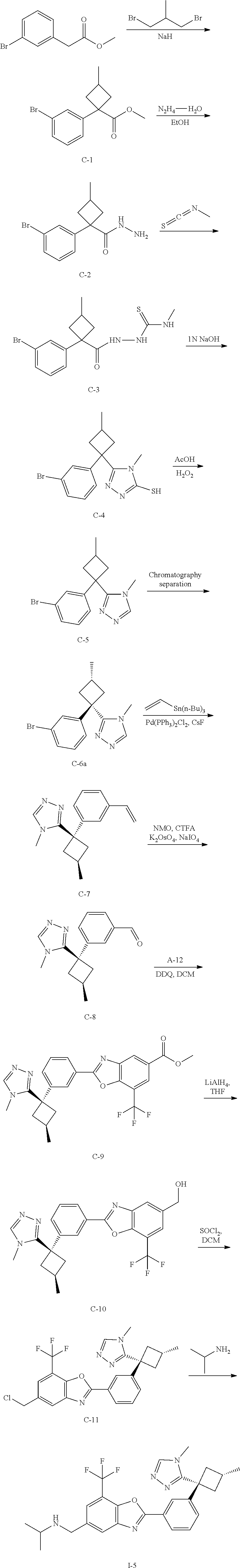

### Synthesis of Methyl 1-(3-bromophenyl)-3-methylcyclobutane-1-carboxylate (C-1)

Into a 500 mL round-bottom flask were added methyl 2-(3-bromophenyl) acetate (4.00 g, 1 eq, 17.5 mmol) and 1,3-dibromo-2-methylpropane (3.80 g, 1 eq, 17.5 mmol) in N, N-dimethyl formamide (60 mL) at room temperature. To the above mixture was added sodium hydride (1.40 g, 60% Wt, 2 eq. 34.9 mmol) over 3 min at 0° C. The resulting mixture was stirred for additional overnight at room temperature. The reaction was quenched with sat, ammonium chloride (aq.) at 0° C. The resulting mixture was diluted with water and extracted with dichloromethane (3×200 mL). The combined organic layers were washed with brine (2×200 mL), dried over anhydrous sodium sulfate. After filtration, the filtrate was concentrated under reduced pressure. The residue was applied on a silica gel column chromatography with petroleum ether/ethyl acetate (8/1) to afford C-1. (3.8 g, 13.5 mmol, 77%, LCMS purity 90%) as a yellow solid. MS (ES): m/z 283.0/285.0 [M+H]⁺.

### Synthesis of 1-(3-bromophenyl)-3-methylcyclobutane-1-carbohydrazide (C-2)

Into a 250 mL round-bottom flask were added Methyl 1-(3-bromophenyl)-3-methylcyclobutane-1-carboxylate (1.70 g, 1 eq, 6.00 mmol) in ethyl alcohol (25 mL) at room temperature. To the above mixture was added hydrazine (20 mL) dropwise over 5 min at room temperature. The resulting mixture was stirred for additional overnight at 80 degrees C. The mixture was cooled to room temperature. The resulting mixture was diluted with water and extracted with ethyl acetate (3×200 mL). The combined organic layers were washed with brine (2×200 mL), dried over anhydrous sodium sulfate. After filtration, the filtrate was concentrated under reduced pressure to afford C-2 (1.6 g, 5.65 mmol, 94%, 92% Purity) as a yellow oil. MS (ES): m/z 283.0/285.0 [M+H]⁺.

### Synthesis of 1-(3-bromophenyl)-3-methyl-N-[(methylcarbamothioyl)amino]cyclobutane-1-carboxamide (C-3)

Into a 100 mL round-bottom flask were added 1-(3-bromophenyl)-3-methylcyclobutane-1-carbohydrazide (1.60 g, 1 eq, 5.65 mmol) and methyl isothiocyanate (1.24 g, 3 eq. 17.0 mmol) in tetrahydrofuran (30 mL) at room temperature. The resulting mixture was stirred for 3 h at 80 degrees C. The mixture was allowed to cool down to room temperature. The resulting mixture was concentrated under vacuum to afford C-3. (1.4 g, 3.94 mmol, 70%, LCMS purity 80%) as a colorless oil. MS (ES): m z 356.0/368.0 [M+H]⁺.

### Synthesis of 5-[1-(3-bromophenyl)-3-methylcyclobutyl]-4-methyl-1,2,4-triazole-3-thiol (C-4)

Into a 250 mL round-bottom flask were added 1-(3-bromophenyl)-3-methyl-N-[(methylcarbamothioyl) amino]cyclobutane-1-carboxamide (1.60 g, 1 eq, 4.49 mmol) in tetrahydrofuran (30 mL) at room temperature. To the above mixture was added sodium hydroxide (690 mg, 3.8 eq, 17.2 mmol) in water (5 mL) dropwise at room temperature. The resulting mixture was stirred for additional overnight at 60 degrees C. The mixture was allowed to cool down to room temperature. The resulting mixture was diluted with water (100 mL). The mixture was acidified to pH 5 with hydrochloric acid (aq., 1 molar). The resulting mixture was diluted with water and extracted with ethyl acetate (3×100 mL). The combined organic layers were washed with brine (2×100 mL), dried over anhydrous sodium sulfate. After filtration, the filtrate was concentrated under reduced pressure to afford C-4 (1.3 g, 3.86 mmol, 86%, LCMS purity 90%) as a yellow oil. MS (ES): m/z 338.0/340.0 [M+H]⁺.

### Synthesis of 3-[1-(3-bromophenyl)-3-methylcyclobutyl]-4-methyl-1,2,4-triazole (C-5)

Into a 20 mL sealed tube were added-[1-(3-bromophenyl)-3-methylcyclobutyl]-4-methyl-1,2,4-triazole-3-thiol (500 mg. 1 Eq, 1.48 mmol) and acetic acid (178 mg, 2 eq, 2.96 mmol) in dichloromethane (8 mL) at room temperature. To the above mixture was added hydrogen peroxide (251 mg, 30% Wt, 5 eq, 7.39 mmol) dropwise over 3 min at 0 degrees C. The resulting mixture was stirred for additional 3 h at room temperature. The resulting mixture was concentrated under reduced pressure. The residue was applied on a silica gel column chromatography with petroleum ether/ethyl acetate (5/1) to afford C-5 (300 mg, 983 μmol, 66%, LCMS purity 95%) as a yellow oil. MS (ES): m/z 306.1/308.1 [M+H]⁺.

### Synthesis of 4-methyl-3-[(1s,3s)-1-(3-bromophenyl)-3-methylcyclobutyl]-1,2,4-triazole (C-6a)

The mix if isomers C-5 (300 mg, 1 eq, 983 μmol) was purified by reverse flash column chromatography with the following conditions: Column, C18; mobile phase, Water (0.05% TFA) and ACN (10% ACN up to 40% in 30 min); Detector, UV 254/220 nm. Two fractions were isolated and concentrated under reduced pressure. Fraction-A affords C-6a (170 mg, 557 mol, 57%, LCMS purity 98%) as a yellow oil. MS (ES): m/z 306.1/308.1 [M+H]+ and Fraction B affords C-6b.

C-6a: ¹H NMR (400 MHz, Chloroform-d) δ 8.23 (s, 1H), 7.55 (d, 1H), 7.42 (dt, 1H), 7.24 (m, 2H), 3.24 (s, 3H), 2.90-2.79 (m, 2H), 2.73-2.61 (m, 3H), 1.16 (d, 3H).

C-6b: ¹H NMR (400 MHz, Chloroform-d) δ 8.18 (s, 1H), 7.41-7.35 (m, 2H), 7.21 (t, 1H), 7.11 (d, 1H), 3.25 (s, 3H), 3.14 (td, 2H), 2.64-2.52 (m, 1H), 2.34-2.22 (m, 2H), 1.14 (d, 3H).

### Synthesis of 4-methyl-3-[(1s,3s)-1-(3-ethenylphenyl)-3-methylcyclobutyl]-1,2,4-triazole (C-7)

To a solution of C-6a (500 mg, 1 eq, 1.63 mmol) and tributyl(ethenyl)stannane (1.04 g, 2 eq, 3.27 mmol) in 1,4-dioxane (10 mL) was added CsF (744 mg, 3 eq, 4.90 mmol) at room temperature under nitrogen atmosphere. Then Pd(PPh₃)₂Cl₂ (229 mg, 0.2 Eq, 0.33 mmol) was added at room temperature under nitrogen atmosphere. The resulting mixture was stirred for 2 h at 100° C. under nitrogen atmosphere. The mixture was cooled to room temperature, diluted with water and extracted with ethyl acetate (3×80 mL). The combined organic layers were washed with brine (2×80 mL), dried over anhydrous sodium sulfate. After filtration, the filtrate was concentrated under reduced pressure. The residue was purified by Prep-TLC with dichloromethane/methyl alcohol (15/1) to afford C-7 (400 mg, 1.57 mmol, 97%, LCMS purity 92%) as a yellow oil. MS (ES): m/z 254.2 [M+H]⁺.

### Synthesis of 3-[(1s,3s)-3-methyl-1-(4-methyl-1,2,4-triazol-3-yl)cyclobutyl]benzaldehyde (C-8)

To a stirred solution of C-7 (400 mg, 1 eq, 1.58 mmol), citric acid (406 mg. 1.34 eq, 2.12 mmol) and NMO (248 mg, 1.34 eq, 2.12 mmol) in tertiary butanol (8 mL) and water (8 mL) were added K₂OsO_{4.2}H₂O (31 mg, 0.06 Eq, 0.09 mmol) at room temperature. After 2 h, to the above mixture was added sodium periodate (676 mg, 2 eq, 3.16 mmol) at room temperature. The resulting mixture was stirred for 1 h at room temperature. The crude product was purified by reverse flash column chromatography with the following conditions: Column, C18: mobile phase, Water (0.1% NH₄HCO₃) and ACN (30% ACN up to 40% in 10 min): Detector. UV 254/220 nm. The product-containing fractions were combined and concentrated under reduced pressure to afford C-8 (240 mg, 0.94 mmol, 60%. LCMS purity 90%) as a yellow solid. MS (ES): m/z 256.1 [M+H]⁺.

### Synthesis of methyl 2-{3-[(1s,3s)-3-methyl-1-(4-methyl-1,2,4-triazol-3-yl)cyclobutyl]phenyl}-7-(trifluoromethyl)-1,3-benzoxazole-5-carboxylate (C-9)

A solution of C-8 (20 mg. 1 eq, 78 mol) and A-12 (37 mg. 2 eq, 0.16 mmol) in DCM (2 mL) was stirred for 1 h at 60° C. The mixture was allowed to cool down to room temperature. To the above mixture was added DDQ (26 mg, 2 Eq, 0.16 mmol) at room temperature. The resulting mixture was stirred for additional 2 h at room temperature. The mixture was diluted with water and extracted with ethyl acetate (3×20 mL). The combined organic layers were washed with brine (2×20 mL), dried over anhdrous sodium sulfate. After filtration, the filtrate was concentrated under reduced pressure. The crude product was purified by Prep-HPLC with the following conditions (Column: XBridge Prep OBD C18 Column, 30* 150 mm, 5 μm: Mobile Phase A: Water (0.1% NH₄HCO₃+0.1% NH₃·H₂O), Mobile Phase B: ACN; Flow rate: 60 mL/min; Gradient: 48% B to 62% B in 8 min; Wave Length: 254/220 nm; RT: 7.68). The fraction was collected and concentrated under vacuum, the residue was re-dissolved in CH₃CN and H₂O, and then was lyophilized to afford methyl C-9 (11.2 mg, 24 μmol, 31%, LCMS purity 99%) as a white solid. MS (ES): m/z 471.3 [M+H]⁺. ¹H NMR (300 MHz, Methanol-d₄) δ 8.60 (s, 1H), 8.33-8.20 (m, 3H), 8.22-8.17 (m, 1H), 7.73-7.62 (m, 2H), 3.99 (d, 3H), 3.30 (s, 3H), 3.02-2.97 (m, 2H), 2.73-2.62 (m, 3H), 1.20-1.17 (m, 3H).

### Synthesis of (2-{3-[(1s,3s)-3-methyl-1-(4-methyl-1,2,4-triazol-3-yl)cyclobutyl]phenyl}-7-(trifluoromethyl)-1,3-benzoxazol-5-yl)methanol (C-10)

To a stirred solution of methyl C-9 (20 mg, 1 eq, 37 μmol) in THF (2 mL) was added DIBAL-H in THF (0.39 mL, 1.1M, 3 eq, 0.11 mmol) dropwise at 0° C. under nitrogen atmosphere. The resulting mixture was stirred for 1 h at 0° C. under nitrogen atmosphere. The reaction was then quenched by the addition of 5 mL of ice water at 0° C. The resulting mixture was diluted with water and extracted with dichloromethane/methanol: 10:1 (3×20 mL). The combined organic layers were washed with brine (2×20 mL), dried over anhydrous sodium sulfate. After filtration, the filtrate was concentrated under reduced pressure. The crude product was purified by Prep-HPLC with the following conditions (Column: XBridge Prep OBD C18 Column, 30*150 mm, 5 μm: Mobile Phase A: Water (0.1% NH₄HCO₃+0.1% NH₃·H₂O), Mobile Phase B: ACN; Flow rate: 60 mL/min; Gradient: 40% B to 50% B in 9 min; Wave Length: 254/220 nm; RT: 6.30). The fraction was collected and concentrated under vacuum, the residue was re-dissolved in CH₃CN and H₂O, and then was lyophilized to afford C-10 (7.9 mg, 18 μmol, 48%, LCMS purity 99%) as an off-white solid. MS (ES): m/z 443.1 [M+H]⁺. ¹H NMR (300 MHz, Methanol-d₄) δ 8.33-8.28 (m, 2H), 8.20-8.15 (m, 1H), 7.98 (s, 1H), 7.71-7.61 (m, 3H), 4.79 (s, 2H), 3.30 (s, 3H), 3.05-2.95 (m, 2H), 2.77-2.61 (m, 3H), 1.20-1.16 (m, 3H).

### Synthesis of 5-(chloromethyl)-2-{3-[(1s,3s)-3-methyl-1-(4-methyl-1,2,4-triazol-3-yl)cyclobutyl]phenyl}-7-(trifluoromethyl)-1,3-benzoxazole (C-11)

Into a 8 mL vial were added C-10 (30 mg, 1 eq, 0.068 mmol), SOCl₂ (0.2 mL) and DCM (1.5 mL) at 0° C. The resulting mixture was stirred for additional 1 h at room temperature. The resulting mixture was filtered the filter cake was washed with ethyl acetate (3×2 mL). The filtrate was concentrated under reduced pressure to afford C-11 (25 mg, 0.05 mmol, 80%, LCMS purity 95%) as a white solid. MS (ES): m/z 461.1/463.1 [M+H]⁺.

### Synthesis of isopropyl[(2-{3-[(1s,3s)-3-methyl-1-(4-methyl-1,2,4-triazol-3-yl)cyclobutyl]phenyl}-7-(trifluoromethyl)-1,3-benzoxazol-5-yl)methyl]amine (I-5)

Into a 8 mL vial were added C-11 (25 mg, 1 eq, 0.05 mmol), isopropylamine (6.41 mg, 2 eq, 0.11 mmol), TEA (27 mg, 5 eq, 0.27 mmol) and DCM (1.5 mL) at room temperature. The resulting mixture was stirred for additional overnight at 60° C. The mixture was cooled to room temperature. The resulting mixture was diluted with water and extracted with dichloromethane (3×10 mL). The combined organic layers were washed with brine (2×10 mL), dried over anhydrous sodium sulfate. After filtration, the filtrate was concentrated under reduced pressure. The crude product was purified by Prep-HPLC with the following conditions: Column: XBridge Prep OBD C18 Column, 30×150 mm, 5 μm: Mobile Phase A: Water (0.1% NH₄HCO₃+0.1% NH₃·H₂O) Mobile Phase B: ACN: Flow rate: 60 mL/min; Gradient: 40% B to 50% B in 8 min: Detector, UV 254/210 nm: RT: 7.45. The fraction was collected and concentrated under vacuum, the residue was re-dissolved in CH₃CN and H₂O, and then was lyophilized to afford I-5 (4.4 mg, 9.1 μmol, 17%, LCMS purity 99%) as a white solid. MS (ES): m/z 484.4 [M+H]⁺. ¹H NMR (400 MHz, Methanol-d4) δ 8.33 (s, 1H), 8.27 (t, 1H), 8.20-8.16 (m, 1H), 8.07-8.00 (m, 1H), 7.78 (s, 1H), 7.72-7.62 (m, 2H), 4.62 (s, 2H), 4.03 (s, 2H), 3.04-2.90 (m, 3H), 2.77-2.60 (m, 3H), 1.25-1.10 (m, 9H).

### Example 4. 5-{1[(3S)-3-Methoxypiperidin-1-yl]methyl}-2-{3-[(1r,3s)-3-methyl-1-(4-methyl-1,2,4-triazol-3-yl)cyclobutyl]phenyl}-7-(trifluoromethyl)-1,3-benzoxazole (I-6)

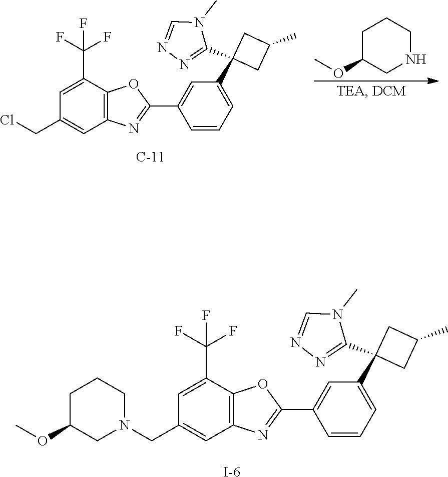

To a stirred solution of 5-(chloromethyl)-2-{3-[(1r,3s)-3-methyl-1-(4-methyl-1,2,4-triazol-3-yl)cyclobutyl]phenyl}-7-(trifluoromethyl)-1,3-benzoxazole (30 mg. 1 eq. 0.065 mmol) and (3S)-3-methoxypiperidine (23 mg, 3 eq, 0.20 mmol) in DCM (2 mL) was added TEA (33 mg, 5 eq, 0.33 mmol) at room temperature under nitrogen atmosphere. The resulting mixture was stirred for 16 h at 60° C. under nitrogen atmosphere. The mixture was cooled to room temperature. The resulting mixture was diluted with water and extracted with dichloromethane (3×10 mL). The combined organic layers were washed with brine (2×10 mL), dried over anhydrous sodium sulfate. After filtration, the filtrate was concentrated under reduced pressure. The crude product was purified by Prep-HPLC with the following conditions (Column: XBridge Shield RP18 OBD Column, 30*150 mm, 5 m; Mobile Phase A: Water (0.1% NH₄HCO₃+0.1% NH₃·H₂O), Mobile Phase B: ACN; Flow rate: 60 mL/min; Gradient: 33% B to 63% B in 7 min; Wave Length: 210/254 nm; RT: 6.13). The fraction was collected and concentrated under vacuum, the residue was re-dissolved in CH₃CN and H₂O, and then was lyophilized to afford I-6 (13.9 mg, 26 μmol. 39%, 98.9% Purity) as a white solid. MS (ES): m/z 541.0 [M+H]⁺. ¹H NMR (400 MHz, Methanol-d₄) δ 8.36-8.28 (m, 2H), 8.22-8.18 (m, 1H), 8.00 (s, 1H), 7.75 (s, 1H), 7.72-7.65 (m, 2H), 3.80-3.69 (m, 2H), 3.43-3.37 (m, 1H), 3.34 (s, 3H), 3.32 (s, 3H), 3.06-2.98 (m, 2H), 2.88 (d, 1H), 2.78-2.62 (m, 4H), 2.29-2.12 (m, 2H), 2.00-1.91 (m, 1H), 1.85-1.77 (m, 1H), 1.64-1.51 (m, 1H), 1.41-1.32 (m, 1H), 1.21 (d, 3H).

### Example 5. 5-{5-Azaspiro[2.4]heptan-5-ylmethyl}-2-{3-[(1r,3s)-3-methyl-1-(4-methyl-1,2,4-triazol-3-yl)cyclobutyl]phenyl}-7-(trifluoromethyl)-1,3-benzoxazole (I-9)

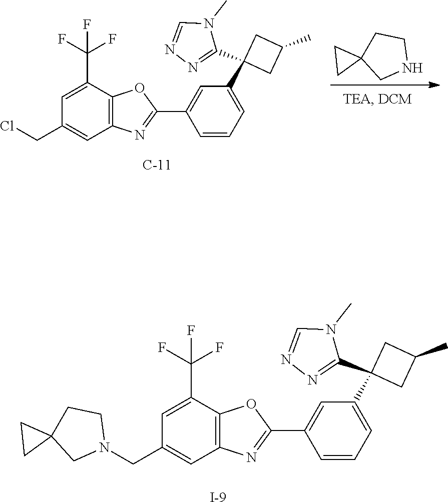

Into a 8 mL vial were added 5-(chloromethyl)-2-{3-[(1r,3s)-3-methyl-1-(4-methyl-1,2,4-triazol-3-yl)cyclobutyl]phenyl}-7-(trifluoromethyl)-1,3-benzoxazole (17 mg, 1 eq, 0.04 mmol), 5-azaspiro [2.4]heptane (7 mg, 2 eq, 0.07 mmol), TEA (19 mg, 5 eq, 0.19 mmol) and DCM (1.5 mL) at room temperature. The resulting mixture was stirred for additional overnight at 60° C. The mixture was cooled to room temperature. The resulting mixture was diluted with water and extracted with dichloromethane (3×10 mL). The combined organic layers were washed with brine (2×10 mL), dried over anhydrous sodium sulfate. After filtration, the filtrate was concentrated under reduced pressure. The crude product was purified by Prep-HPLC with the following conditions: Column: XBridge Prep OBD C18 Column, 30×150 mm, 5 μm; Mobile Phase A: Water (0.1% NH₄HCO₃+0.1% NH₃·H₂O) Mobile Phase B: ACN; Flow rate: 60 mL/min; Gradient: 40% B to 50% B in 8 min; Detector, UV 254/210 nm; RT: 7.45. The fraction was collected and concentrated under vacuum, the residue was re-dissolved in CH₃CN and H₂O, and then was lyophilized to afford I-9 (6 mg, 12 μmol, 31%, 99.4% Purity) as a white solid. MS (ES): m/z 522.5 [M+H]⁺. H NMR (400 MHz, Methanol-d₄) δ 8.33 (s, 1H), 8.30-8.26 (m, 1H), 8.20-8.15 (m, 1H), 8.02-7.96 (m, 1H), 7.74 (s, 1H), 7.69-7.62 (m, 2H), 3.85 (s, 2H), 3.30 (s, 3H), 3.04-2.94 (m, 2H), 2.82 (t, 2H), 2.71-2.61 (m, 3H), 2.56 (s, 2H), 1.87 (t, 2H), 1.18 (d, 3H), 0.61-0.53 (m, 4H).

### Example 6. 1-Cyclobutyl-N-methyl-N-((2-(3-((1s,3s)-3-methyl-1-(4-methyl-4H-1,2,4-triazol-3-yl)cyclobutyl)phenyl)-7-(trifluoromethyl)benzo[d]oxazol-5-yl)methyl)methanamine (1-10)

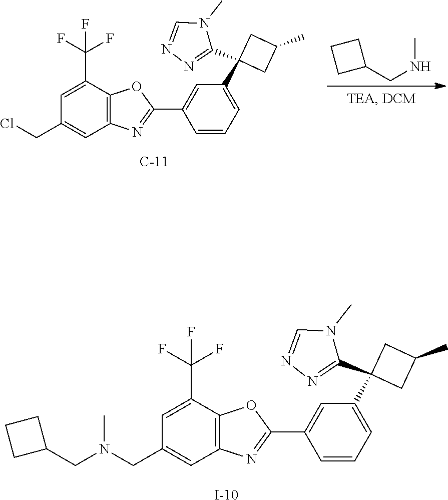

Into a 8 mL vial were added 5-(chloromethyl)-2-{3-[(1r,3s)-3-methyl-1-(4-methyl-1,2,4-triazol-3-yl)cyclobutyl]phenyl}-7-(trifluoromethyl)-1,3-benzoxazole (20 mg, 1 eq, 0.04 mmol), (cyclobutylmethyl) (methyl)amine (5 mg, 1.2 eq, 0.05 mmol), TEA (22 mg, 5 Eq, 0.22 mmol) and DCM (1.5 mL) at room temperature. The resulting mixture was stirred for additional overnight at 60° C. The mixture was cooled to room temperature. The resulting mixture was diluted with water and extracted with dichloromethane (3×10 mL). The combined organic layers were washed with brine (2×10 mL), dried over anhydrous sodium sulfate. After filtration, the filtrate was concentrated under reduced pressure. The crude product was purified by Prep-HPLC with the following conditions: Column: XBridge Prep OBD C18 Column, 30×150 mm, 5 μm; Mobile Phase A: Water (0.1% NH₄HCO₃+0.1% NH₃·H₂O) Mobile Phase B: ACN; Flow rate: 60 mL/min; Gradient: 40% B to 50% B in 8 min: Detector. UV 254/210 nm: RT: 7.45. The fraction was collected and concentrated under vacuum, the residue was re-dissolved in CH₃CN and H₂O, and then was lyophilized to afford I-10 (17 mg, 0.03 mmol, 75%, LCMS purity 99%) as a white solid. MS (ES): m/z 524.1 [M+H]⁻. ¹H NMR (400 MHz, Methanol-d₄) δ 8.37 (s, 1H), 8.32 (d, 1H), 8.24-8.19 (m, 1H), 8.02 (s, 1H), 7.77-7.66 (m, 3H), 3.81 (s, 2H), 3.30 (s, 3H), 3.08-2.99 (m, 2H), 2.80-2.64 (m, 4H), 2.61 (d, 2H), 2.32 (s, 3H), 2.20-2.10 (m, 2H), 2.06-1.92 (m, 1H), 1.89-1.71 (m, 3H), 1.22 (d, 3H).

### Example 7. 3-{[6-(5-{5-azaspiro[2.4]heptan-5-ylmethyl}-7-(trifluoromethyl)-1,3-benzoxazol-2-yl)-4-[4-fluoro-2-(4-methyl-1,2,4-triazol-3-yl) phenyl]pyridin-2-yl]amino}propanenitrile (1-12)

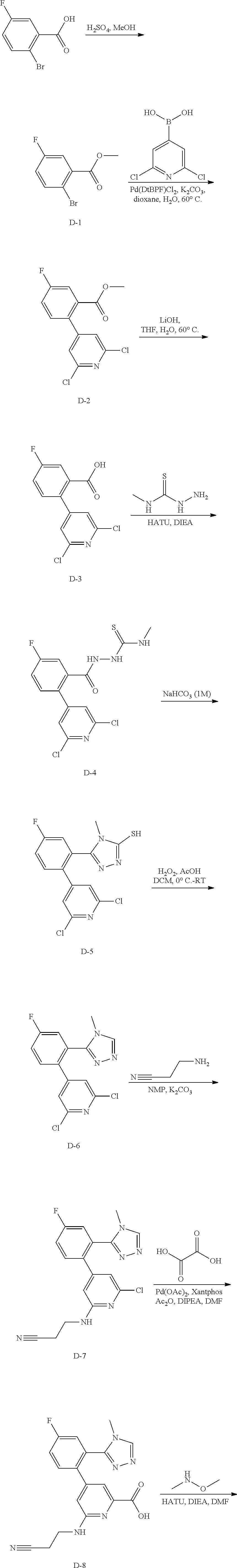

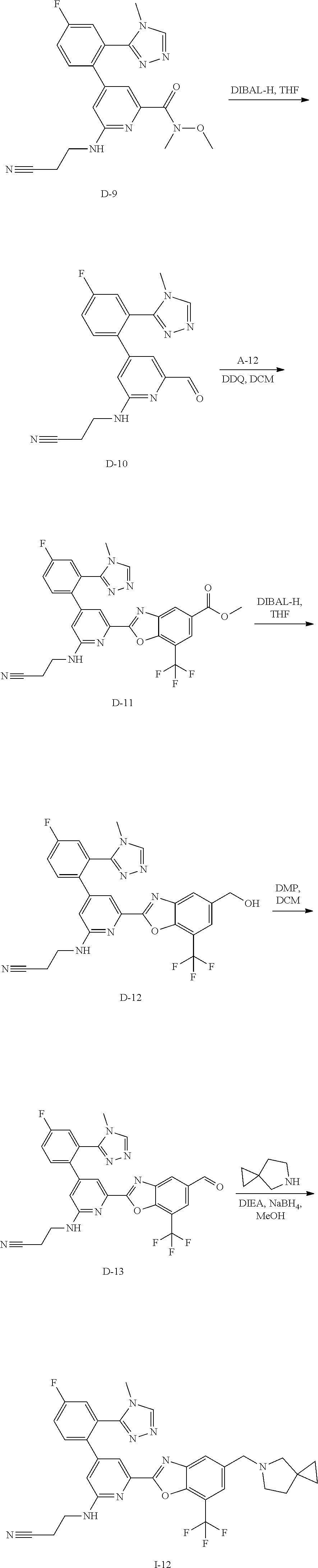

### Synthesis of methyl 2-bromo-5-fluorobenzoate (D-1)

Into a 1-L round-bottom flask purged and maintained with an inert atmosphere of nitrogen, was placed 2-bromo-5-fluorobenzoic acid (5.00 g, 1 eq, 22.8 mmol) in methanol (400 mL), then sulfuric acid (4.48 g, 2 eq, 45.7 mmol) was added at room temperature. The resulting solution was stirred overnight at 70 degrees C. The resulting mixture was diluted with water and extracted with ethyl acetate (3×500 mL). The combined organic layers were washed with brine (2×500 mL), dried over anhydrous sodium sulfate. After filtration, the filtrate was concentrated under reduced pressure to afford D-1 (4.2 g, 18.0 mmol, crude) as a yellow oil. MS (ES): m/z 233.0/235.0 [M+H]⁺.

### Synthesis of methyl 2-(2,6-dichloropyridin-4-yl)-5-fluorobenzoate (D-2)

Into a 500-mL round-bottom flask purged and maintained with an inert atmosphere of nitrogen, was placed methyl 2-bromo-5-fluorobenzoate (4.20 g, 1 eq. 18.0 mmol) and 2,6-dichloropyridin-4-ylboronic acid (3.46 g, 1 eq, 18.0 mmol) in 1,4-dioxane (150 mL) and water (30 mL), then Pd(DtBPF)Cl₂ (1.17 g, 0.1 eq, 1.80 mmol) and potassium carbonate (7.47 g, 3 Eq. 54.1 mmol) were added at room temperature. The resulting solution was stirred for overnight at 60 degrees C. The mixture was cooled to room temperature, diluted with water and extracted with ethyl acetate (3×300 mL). The combined organic layers were washed with brine (2×300 mL), dried over anhydrous sodium sulfate. After filtration, the filtrate was concentrated under reduced pressure. The crude product was purified by reverse flash column chromatography with the following conditions: Column. C18; mobile phase, Water (0.1% NH₄HCO₃) and ACN (26% ACN up to 52% in 15 min); Detector, UV 254/220 nm to afford D-2 (2.5 g, 8.36 mmol, 46%, 90% Purity) as a yellow solid. MS (ES): m/z 300.0/302.0 [M+H]⁺.

### Synthesis of 2-(2,6-dichloropyridin-4-yl)-5-fluorobenzoic acid (D-3)

Into a 250-mL round-bottom flask, was placed methyl 2-(2,6-dichloropyridin-4-yl)-5-fluorobenzoate (2.50 g, 1 eq, 8.33 mmol) in tetrahydrofuran (100 mL) and water (25 mL), then lithium hydroxide (600 mg. 3 eq. 25.0 mmol) was added at room temperature. The resulting solution was stirred for 16 h at 60 degrees C. The mixture was cooled to room temperature, diluted with water and extracted with ethyl acetate (3×150 mL). The combined organic layers were washed with brine (2×150 mL), dried over anhydrous sodium sulfate. After filtration, the filtrate was concentrated under reduced pressure to afford D-3 (2.2 g, 7.72 mmol, 92%, LCMS purity 92%) as a brown yellow solid. MS (ES): m/z 286.0/288.0 [M+H]⁺.

### Synthesis of 2-(2-(2,6-dichloropyridin-4-yl)-5-fluorobenzoyl)-N-methylhydrazine-1-carbothioamide (D-4)

Into a 100-mL round-bottom flask purged and maintained with an inert atmosphere of nitrogen, was placed 2-(2,6-dichloropyridin-4-yl)-5-fluorobenzoic acid (1.60 g, 1 eq, 5.59 mmol) and 1-amino-3-methylthiourea (710 mg, 1.2 eq, 6.71 mmol) in N, N-dimethyl formamide (50 mL) at room temperature. Then 1-propyl three-ring phosphate acid anhydride (7.12 g, 4 eq, 22.4 mmol) and N, N-diisopropylethylamine (2.17 g, 3 eq, 16.8 mmol) were added at room temperature. The resulting solution was stirred for overnight at 60 degrees C. The mixture was cooled to room temperature, purified by reverse flash column chromatography with the following conditions: Column, C18; mobile phase, Water (0.1% FA) and ACN (23% ACN up to 46% in 15 min); Detector, UV 254/220 nm. The product-containing fractions were combined and concentrated under reduced pressure to afford D-4 (810 g, 2.18 mmol, 39%, LCMS purity 88%) as brown yellow solid. MS (ES): m/z 373.0/375.0 [M+H]⁺.

### Synthesis of 5-(2-(2,6-dichloropyridin-4-yl)-5-fluorophenyl)-4-methyl-4H-1,2,4-triazole-3-thiol D-5)

Into a 100-mL round-bottom flask, was placed 2-(2-(2,6-dichloropyridin-4-yl)-5-fluorobenzoyl)-N-methylhydrazine-1-carbothioamide (800 mg, 1 eq, 2.14 mmol) in sodium bicarbonate solution (20 mL, 1 molar) at room temperature at room temperature. The resulting solution was stirred for overnight at 60 degrees C. The mixture was cooled to room temperature, diluted with water and extracted with ethyl acetate (3×80 mL). The combined organic layers were washed with brine (2×80 mL), dried over anhydrous sodium sulfate. After filtration, the filtrate was concentrated under reduced pressure to afford D-5 (700 mg, 1.97 mmol, crude) as a brown solid. MS (ES): m/z 355.0/357.0 [M+H]⁺.

### Synthesis of 2,6-dichloro-4-(4-fluoro-2-(4-methyl-4H-1,2,4-triazol-3-yl) phenyl) pyridine (D-6)

Into a 100-mL round-bottom flask purged and maintained with an inert atmosphere of nitrogen, was placed 5-(2-(2,6-dichloropyridin-4-yl)-5-fluorophenyl)-4-methyl-4H-1,2,4-triazole-3-thiol (700 mg, 1 eq. 1.97 mmol) in dichloromethane (20 mL), then hydrogen peroxide (335 mg, 30% Wt, 5 eq, 9.90 mmol) and acetic acid (237 mg, 2 eq, 3.94 mmol) were added at room temperature. The resulting solution was stirred for 3 h at room temperature. The resulting mixture was diluted with water and extracted with dichloromethane (3×80 mL). The combined organic layers were washed with brine (2×80 mL), dried over anhydrous sodium sulfate. After filtration, the filtrate was concentrated under reduced pressure to afford D-6 (650 mg. 2.02 mmol, crude) as brown solid. MS (ES): m/z 323.0/325.0 [M+H]⁺.

### Synthesis of 3-({6-chloro-4-[4-fluoro-2-(4-methyl-1,2,4-triazol-3-yl)phenyl]pyridin-2-yl}amino) propanenitrile (D-7)

To a stirred solution of 2,6-dichloro-4-[4-fluoro-2-(4-methyl-1,2,4-triazol-3-yl)phenyl]pyridine (500 mg, 1 Eq, 1.56 mmol) and β aminopropionitrile (3.25 g, 30 eq, 46.4 mmol) in NMP (15 mL) was added K₂CO₃ (2.14 g. 10 eq, 15.5 mmol) at room temperature. The resulting mixture was stirred for 12 h at 100° C. The mixture was allowed to cool down to room temperature. The crude product was purified by reverse flash column chromatography with the following conditions: Column, C18: mobile phase, Water (0.1% NH₄HCO₃) and ACN (40% ACN up to 50% in 10 min); Detector, UV 254/220 nm to afford D-7 (390 mg, 1.10 mmol, 71%, LCMS purity 94%) as a white solid. MS (ES): m z 357.1/359.1 [M+H]⁺.

### Synthesis of 6-[(2-cyanoethyl) amino]-4-[4-fluoro-2-(4-methyl-1,2,4-triazol-3-yl) phenyl]pyridine-2-carboxylic acid (D-8)

To a stirred solution of 3-({6-chloro-4-[4-fluoro-2-(4-methyl-1,2,4-triazol-3-yl) phenyl]pyridin-2-yl}amino) propanenitrile (100 mg, 1 eq, 0.28 mmol) and oxalic acid (30 mg, 1.2 eq, 0.34 mmol) in DMF (15 mL) were added Ac₂O (43 mg. 1.5 eq, 0.42 mmol) and DIPEA (54 mg, 1.5 eq, 0.42 mmol) at room temperature under nitrogen atmosphere. To the above mixture were added Xantphos (32 mg, 0.2 eq, 0.06 mmol) and Pd(OAc)₂ (6.3 mg, 0.1 eq, 0.03 mmol) at room temperature under nitrogen atmosphere. The resulting mixture was stirred for 12 hours at 100° C. under nitrogen atmosphere. The mixture was allowed to cool down to room temperature. The crude product was purified by reverse flash column chromatography with the following conditions: Column, C18: mobile phase, Water (0.1% FA) and ACN (50% ACN up to 60% in 10 min); Detector, UV 254/220 nm. The crude product was purified by Prep-HPLC with the following conditions (Column: X-Select Prep OBD C18 Column, 30*150 mm, 5 M; Mobile Phase A: Water (0.05% FA), Mobile Phase B: ACN: Flow rate: 60 mL/min mL/min; Gradient: 30% B to 50% B in 7 min; Wave Length: 254 nm/220 nm; RT: 6.56). The fraction was collected and concentrated under vacuum, the residue was re-dissolved in CH₃CN and H₂O, and then was lyophilized to afford D-8 (7.3 mg, 20.0 μmol, 7.1%, LCMS purity 99%) as a white solid. MS (ES): m/z 367.1 [M+H]⁺.

### Synthesis of 6-[(2-cyanoethyl)amino]-4-[4-fluoro-2-(4-methyl-1,2,4-triazol-3-yl)phenyl]-N-methoxy-N-methylpyridine-2-carboxamide (D-9)

To a stirred solution of 6-[(2-cyanoethyl)amino]-4-[4-fluoro-2-(4-methyl-1,2,4-triazol-3-yl)phenyl]pyridine-2-carboxylic acid (200 mg, 1 eq, 0.55 mmol) and N,O-dimethylhydroxylamine (40 mg, 1.2 eq, 0.66 mmol) in THF (20 mL) were added DIPEA (212 mg, 3 eq, 1.64 mmol) and HATU (270 mg, 1.3 Eq, 0.71 mmol) at room temperature. The resulting mixture was stirred for 1 h at room temperature. The resulting mixture was concentrated under vacuum. The residue was purified by Prep-TLC with dichloromethane/methyl alcohol (8/1). The crude product was purified by Prep-HPLC with the following conditions (Column: XBridge Prep OBD C18 Column, 30*150 mm, 5 μm; Mobile Phase A: Water (0.1% NH₄HCO₃), Mobile Phase B: ACN; Flow rate: 60 mL/min; Gradient: 19% B to 23% B in 7 min: Wave Length: 254 nm/220 nm: RT: 6.47). The fraction was collected and concentrated under vacuum, the residue was re-dissolved in CH₃CN and H₂O, and then was lyophilized to afford D-9 (3.7 mg, 9.0 mol. 1.6%. LCMS purity 97%) as a white solid. MS (ES): m/z 410.1 [M+H]⁺.

### Synthesis of 3-({4-14-fluoro-2-(4-methyl-1,2,4-triazol-3-yl) phenyl]-6-formylpyridin-2-yl}amino) propanenitrile (D-10)

To a stirred solution of 6-[(2-cyanoethyl) amino]-4-[4-fluoro-2-(4-methyl-1,2,4-triazol-3-yl) phenyl]-N-methoxy-N-methylpyridine-2-carboxamide (400 mg, 1 eq, 0.98 mmol) in THF (20 mL) was added DIBAl-H (695 mg, 1 M, 5 eq, 4.89 mmol) in portions at 0° C. under nitrogen atmosphere. The resulting mixture was stirred for 15 minutes at 0° C. under nitrogen atmosphere. The reaction was then quenched by the addition of 2 mL of methyl alcohol at 0° C., and then concentrated under vacuum. The residue was purified by TLC with dichloromethane/methanol (8/1) to afford D-10 (170 mg, 0.49 mmol, 50%, LCMS purity 97%) as a yellow solid. MS (ES): m/z 351.1 [M+H]⁺. LC-MS-PH-NIMB-1621-10 (ES, m/z):351.1 (M+H+).

### Synthesis of methyl 2-{6-[(2-cyanoethyl) amino]-4-[4-fluoro-2-(4-methyl-1,2,4-triazol-3-yl) phenyl]pyridin-2-yl}-7-(trifluoromethyl)-1,3-benzoxazole-5-carboxylate (D-11)

A solution of 3-({4-[4-fluoro-2-(4-methyl-1,2,4-triazol-3-yl) phenyl]-6-formylpyridin-2-yl}amino) propanenitrile (150 mg, 1 eq, 0.43 mmol) and methyl 3-amino-4-hydroxy-5-(trifluoromethyl) benzoate (101 mg, 1 eq, 0.43 mmol) in DCM (15 mL) was stirred for 1 h at room temperature. To the above mixture was added DDQ (194 mg, 2 eq, 0.86 mmol) at room temperature. The resulting mixture was stirred for additional 1 h at room temperature. The resulting mixture was concentrated under vacuum. The residue was purified by TLC with dichloromethane/methanol (9/1). The crude product was purified by Prep-HPLC with the following conditions (Column: XBridge Prep OBD C18 Column, 30*150 mm, 5 μm; Mobile Phase A: Water (0.1% NH₄HCO₃), Mobile Phase B: ACN; Flow rate: 60 mL/min: Gradient: 41% B to 51% B in 7 min; Wave Length: 254 nm/220 nm; RT: 7.03). The fraction was collected and concentrated under vacuum, the residue was re-dissolved in CH₃CN and H₂O, and then was lyophilized to D-11 (7.9 mg, 14 mol, 3.3%, LCMS purity 99%) as a white solid. MS (ES): m/z 566.1 [M+H]⁺.

### Synthesis of 3-({4-[4-fluoro-2-(4-methyl-1,2,4-triazol-3-yl) phenyl]-6-[5-(hydroxymethyl)-7-(trifluoromethyl)-1,3-benzoxazol-2-yl]pyridin-2-yl}amino) propanenitrile (D-12)

To a stirred solution of methyl 2-{6-[(2-cyanoethyl) amino]-4-[4-fluoro-2-(4-methyl-1,2,4-triazol-3-yl) phenyl]pyridin-2-yl}-7-(trifluoromethyl)-1,3-benzoxazole-5-carboxylate (60 mg, 1 eq, 0.11 mmol) in THF (10 mL) was added DIBAL-H (76 mg, 1 M, 5 eq, 0.53 mmol) at 0° C. under nitrogen atmosphere. The resulting mixture was stirred for 15 minutes at 0° C. under nitrogen atmosphere. The reaction was then quenched by the addition of 2 mL of methyl alcohol at 0° C., and then concentrated under reduced pressure. The residue was purified by TLC with dichloromethane/methanol (8/1). The crude product was purified by Prep-HPLC with the following conditions (Column: XBridge Prep OBD C18 Column, 30* 150 mm, 5 m; Mobile Phase A: Water (0.1% NH₄HCO₃), Mobile Phase B; ACN; Flow rate: 60 mL/min; Gradient: 28% B to 38% B in 7 min: Wave Length: 254 nm/220 nm; RT; 5.5). The fraction was collected and concentrated under vacuum, the residue was re-dissolved in CH₃CN and H₂O, and then was lyophilized to afford D-12 (6.1 mg, 11 μmol, 11%, LCMS purity 99%) as a white solid. MS (ES): m/z 538.2 [M+H]⁻. LC-MS-PH-NIMB-1621-12 (ES, m/z): 538.2 (M+H+).

### Synthesis of 3-({4-[4-fluoro-2-(4-methyl-1,2,4-triazol-3-yl) phenyl]-6-[5-formyl-7-(trifluoromethyl)-1,3-benzoxazol-2-yl]pyridin-2-yl}amino) propanenitrile (D-13)

To a stirred solution of 3-({4-[4-fluoro-2-(4-methyl-1,2,4-triazol-3-yl) phenyl]-6-[5-(hydroxymethyl)-7-(trifluoromethyl)-1,3-benzoxazol-2-yl]pyridin-2-yl}amino) propanenitrile (27 mg, 1 eq, 0.05 mmol) in DCM (5 mL) was added DMP (28 mg, 1.3 eq, 0.07 mmol) at room temperature. The resulting mixture was stirred for 1 h at room temperature. The resulting mixture was filtered; the filter cake was washed with dichloromethane (3×3 mL). The filtrate was concentrated under reduced pressure. The crude product D-13 was used in the next step directly without further purification. MS (ES): m/z 536.1 [M+H]⁺.

### Synthesis of 3-{[6-(5-{5-azaspiro [2.4]heptan-5-ylmethyl}-7-(trifluoromethyl)-1,3-benzoxazol-2-yl)-4-[4-fluoro-2-(4-methyl-1,2,4-triazol-3-yl) phenyl]pyridin-2-yl]amino}propanenitrile (I-12)

To a stirred solution of 3-({4-[4-fluoro-2-(4-methyl-1,2,4-triazol-3-yl) phenyl]-6-[5-formyl-7-(trifluoromethyl)-1,3-benzoxazol-2-yl]pyridin-2-yl}amino) propanenitrile (30 mg. 1 eq. 0.06 mmol) and 5-azaspiro [2.4]heptane (8 mg, 1.5 eq, 0.08 mmol) in DCM (5 mL) were added DIPEA (9 mg. 1.2 eq, 0.07 mmol) and NaBH(OAc)₃ (24 mg, 2 eq, 0.11 mmol) at room temperature. The resulting mixture was stirred for 1 h at room temperature. The reaction was then quenched by the addition of 2 mL of methyl alcohol at 0° C., and then concentrated under reduced pressure. The residue was purified by TLC with dichloromethane/methanol (9/1). The crude product was purified by Prep-HPLC with the following conditions (Column: XBridge Prep OBD C18 Column, 30* 150 mm, 5 m; Mobile Phase A: Water (0.1% NH₄HCO₃), Mobile Phase B: ACN: Flow rate: 60 mL/min: Gradient: 49% B to 59% B in 7 min; Wave Length: 254 nm/220 nm; RT: 6.03). The fraction was collected and concentrated under vacuum, the residue was re-dissolved in CH₃CN and H₂O, and then was lyophilized to afford I-12 (6.9 mg, 11 μmol, 20%, LCMS purity 99%) as a white solid. MS (ES): m/z 617.3 [M+H]⁺. ¹H NMR (400 MHz, DMSO-d₆) δ 8.48 (s, 1H), 8.06 (d, 1H), 7.81-7.73 (m, 2H), 7.68-7.58 (m, 2H), 7.47 (t, 1H), 7.20 (d. 1H), 6.52 (d, 1H), 3.78 (s, 2H), 3.57 (t, 2H), 3.34 (s, 3H), 2.86 (t, 2H), 2.70 (t, 2H), 2.46 (s, 2H), 1.76 (t, 2H), 0.56-0.48 (m, 4H).

### Example 8. 3-[2-(5-{5-azaspiro[2.4]heptan-5-ylmethyl}-7-(trifluoromethyl)-1,3-benzoxazol-2-yl)-6-[(2-cyanoethyl)amino]pyridin-4-yl]-4-(4-methyl-1,2,4-triazol-3-yl)benzonitrile (I-16)

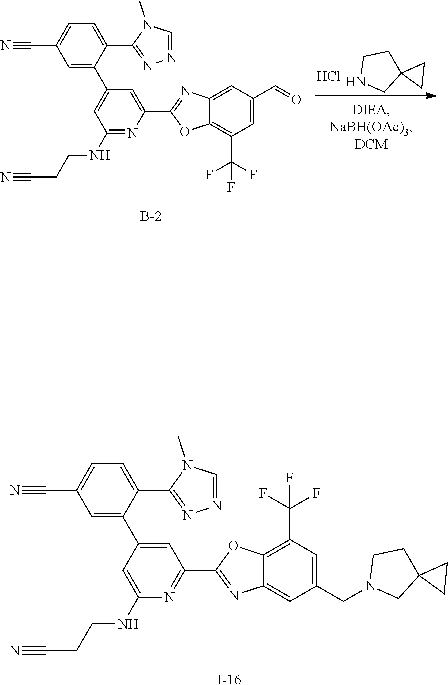

To a stirred mixture of B-2 (80 mg, 1 Eq. 0.15 mmol) and 5-azaspiro[2.4]heptane hydrochloride (23 mg, 1.2 Eq, 0.18 mmol) in DCM (3 mL) were added DIPEA (114 mg, 6 Eq, 0.88 mmol) and NaBH(OAc)₃ (188 mg, 6 Eq, 0.88 mmol) at room temperature. The resulting mixture was stirred for overnight at room temperature. The resulting mixture was concentrated under reduced pressure. The crude product was purified by Prep-HPLC with the following conditions (Column: XBridge Prep OBD C18 Column, 30* 150 mm, 54m; Mobile Phase A: Water (0.1% NH₄HCO₃), Mobile Phase B: ACN; Flow rate: 60 mL/min mL/min: Gradient: 50% B to 51% B in 7 min; Wave Length: 254 nm/220 nm; RT: 5.9). The fraction was collected and concentrated under vacuum, the residue was re-dissolved in CH₃CN and H₂O, and then was lyophilized to afford I-16 (13.8 mg, 0.02 mmol, 15%, LCMS purity 98.7%) as a white solid. MS (ES): m/z 624.4 [M+H]⁺. ¹H NMR (400 MHz, DMSO-d₆) δ 8.51 (s, 1H), 8.26 (d, 1H), 8.18-8.11 (m, 1H), 8.06 (s, 1H), 7.87 (d, 1H), 7.75 (s, 1H), 7.51 (t, 1H), 7.26 (d. 1H), 6.60 (s, 1H), 3.78 (s, 2H), 3.62-3.53 (m, 2H), 3.33 (s, 3H), 2.87 (t, 2H), 2.70 (t, 2H), 2.46 (s, 2H), 1.77 (t, 2H), 0.50 (d, 4H).

### Example 9. Synthesis of 3-{2-[(2-cyclopropylethyl) amino]-6-(5-{[(3S)-3-methylpiperidin-1-yl]methyl}-7-(trifluoromethyl)-1,3-benzoxazol-2-yl) pyridin-4-yl}-4-(4-methyl-1,2,4-triazol-3-yl) benzonitrile (I-17)

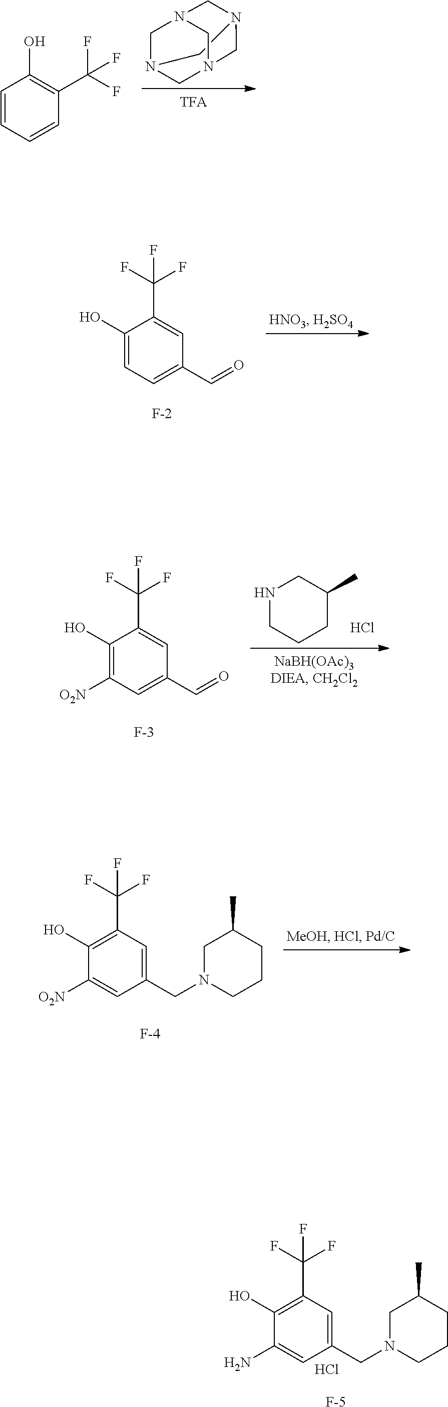

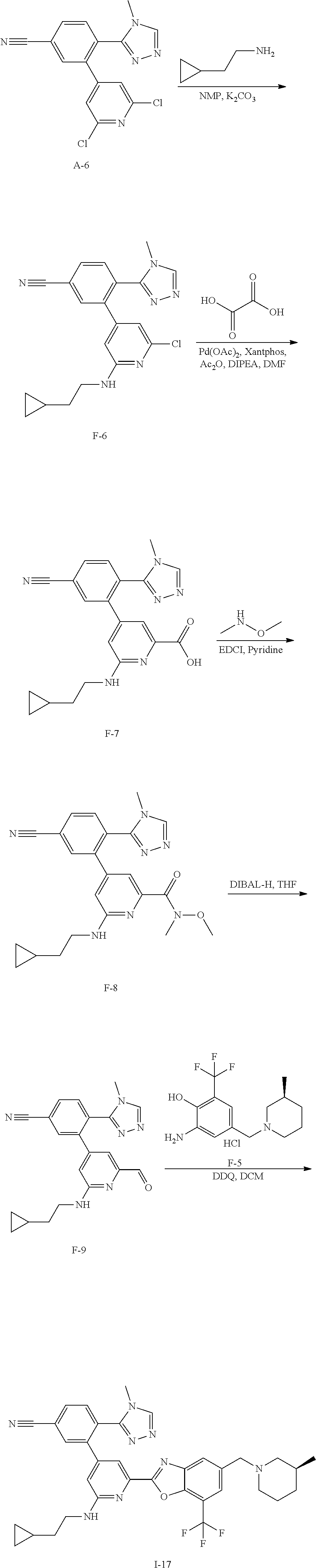

### Synthesis of 4-hydroxy-3-(trifluoromethyl) benzaldehyde (F-2)

To a stirred solution of O-trifluoromethylphenol (6 g. 37.012 mmol, 1 equiv) and 1,3,5,7-tetraazatricyclo [3.3.1.1\hat{ }{3,7}]decane (10.376 g, 74.024 mmol, 2 equiv) in trifluoroacetaldehyde (50 mL) at room temperature. The resulting mixture was stirred for overnight at 65 degrees C. The mixture was allowed to cool down to room temperature. The resulting mixture was extracted with EtOAc (3×200 mL). The combined organic layers were washed with brine (2×200 mL), dried over anhydrous Na₂SO₄. After filtration, the filtrate was concentrated under reduced pressure. The residue was purified by silica gel column chromatography, eluted with PE/EA (1:1) to afford F-2 (2.4 g, 30.70%) as a light brown solid, m/z 191.0 (M+H)⁺ (ES+).

### Synthesis of 4-hydroxy-3-nitro-5-(trifluoromethyl) benzaldehyde (F-3)

To a stirred solution of F-2 (2 g, 10.520 mmol, 1.00 equiv) in H₂SO₄ (20 mL) was added HNO₃ (1 mL) dropwise at 0 degrees C. under air atmosphere. The resulting mixture was stirred for 0.5 h at room temperature under air atmosphere. Desired product could be detected by LCMS. The aqueous layer was extracted with EtOAc (3×50 mL). The resulting mixture was concentrated under vacuum. The residue was purified by silica gel column chromatography, eluted with PE/EA (1:1) to afford F-3 (1.5 g, 60.65%) as a yellow solid, m/z 236.0 (M+H)⁺ (ES+).

### Synthesis of 4-{[(3S)-3-methylpiperidin-1-yl]methyl}-2-nitro-6-(trifluoromethyl) phenol (F-4)

To a stirred solution of F-3 (1.0 g, 4.253 mmol, 1 equiv) and (3S)-3-methylpiperidine hydrochloride (865.35 mg, 6.380 mmol, 1.5 equiv) in DCM (20 mL) were added DIEA (2.75 g, 21.265 mmol. 5 equiv) and NaBH(OAc)₃ (4.51 g, 21.265 mmol, 5 equiv) at room temperature under nitrogen atmosphere. The resulting mixture was stirred for overnight at 40° C. under nitrogen atmosphere. The mixture was allowed to cool down to room temperature. The reaction was quenched by the addition of MeOH (10 mL) at 0° C. The resulting mixture was concentrated under reduced pressure. The residue was purified by silica gel column chromatography, eluted with CH₂Cl₂/MeOH (15:1) to afford F-4 (750 mg, 45.43%) as a yellow solid, m/z 319.1 (M+H)⁺ (ES+).

### Synthesis of 2-amino-4-{[(3S)-3-methylpiperidin-1-yl]methyl}-6-(trifluoromethyl) phenol (F-5)

To a stirred solution of F-4 (500 mg, 1.571 mmol, 1 equiv) in MeOH (15 mL) was added HCl (15 mL) at 0° C. under nitrogen atmosphere. To the above mixture was added Pd/C (83.59 mg, 0.785 mmol, 0.50 equiv) at room temperature under nitrogen atmosphere. The resulting mixture was stirred for overnight at room temperature under hydrogen atmosphere. The resulting mixture was filtered, the filter cake was washed with MeOH (3×3 mL). The filtrate was concentrated under reduced pressure. The crude product was purified by Prep-HPLC with the following conditions (Column: Xselect CSH PrepC18 Column, 19*250 mm. 5 μm; Mobile Phase A: Water (0.1% HCL). Mobile Phase B: ACN: Flow rate: 25 mL/min: Gradient: 6% B to 16% B in 8 min; Wave Length: 254 nm/220 nm; RT: 7.35) to afford F-5 (360 mg, 77.90%) as a brown solid, m/z 288.2 (M+H)⁺ (ES+).

### Synthesis of 3-{2-chloro-6-[(2-cyclopropylethyl) amino]pyridin-4-yl}-4-(4-methyl-1,2,4-triazol-3-yl) benzonitrile (F-6)

To a stirred solution of A-6 (3.00 g, 1 eq, 9.09 mmol) and 2-cyclopropylmethanamine (15.5 g, 20 eq. 181.7 mmol) in NMP (15 mL) was added K₂CO₃ (37.7 g, 30 eq. 272.6 mmol) at room temperature under nitrogen atmosphere. The resulting mixture was stirred for overnight at 100° C. under nitrogen atmosphere. The mixture was allowed to cool down to room temperature. The crude product was purified by reverse flash column chromatography with the following conditions: Column, C18; mobile phase, Water (0.1% NH₄HCO₃) and ACN (30% ACN up to 60% in 15 min); Detector, UV 254/220 nm. This resulted in F-6 (2.1 g, 5.41 mmol, 58%, 92% purity) as an off-white solid, m/z 379.1/381.1 (M+H)⁻ (ES+).

### Synthesis of 4-(5-cyano-2-(4-methyl-4H-1,2,4-triazol-3-yl)phenyl)-6-((2-cyclopropylethyl)amino)picolinic acid (F-7)

To a stirred solution of F-6 (50 mg, 1 eq, 0.13 mmol) and oxalic acid (18 mg, 1.5 eq, 0.20 mmol) in DMF (4 mL) were added Ac₂O (20 mg. 1.5 eq. 0.20 mmol) and DIPEA (34 mg, 2 eq, 0.26 mmol) at room temperature under nitrogen atmosphere. To the above mixture were added XantPhos (15 mg, 0.2 eq, 0.03 mmol) and Pd(OAc)₂ (3 mg, 0.1 eq, 0.013 mmol) at room temperature under nitrogen atmosphere. The resulting mixture was stirred for overnight at 100° C. under nitrogen atmosphere. The mixture was allowed to cool down to room temperature. The crude product was purified by reverse flash column chromatography with the following conditions: Column, C18: mobile phase, Water (0.1% FA) and ACN (0% ACN up to 30% in 30 min); Detector, UV 254/220 nm. The crude product was purified by Prep-HPLC with the following conditions (Column: XSelect CSH Prep C18 OBD Column. 19*250 mm. 5 m: Mobile Phase A: CAN (0.1% FA), Mobile Phase B: ACN: Gradient: 40% B to 50% B in 7 min; Wave Length: 254 nm; RT: 7). The fraction was collected and concentrated under vacuum, the residue was re-dissolved in CH₃CN and H₂O, and then was lyophilized to afford F-7 (7.4 mg, 0.02 mmol, 14%, 98.1% purity) as a white solid, m/z 389.3 (M+H)⁺ (ES+). ¹H NMR (400 MHz, DMSO-d₆) δ 12.62 (s, 1H), 8.52 (s, 1H), 8.14 (d. 1H), 8.11-8.07 (m, 1H), 7.81 (d, 1H), 6.96-6.87 (m, 2H), 6.38 (d. 1H), 3.29-3.19 (m, 5H), 1.43-1.34 (m, 2H), 0.78-0.67 (m, 1H), 0.45-0.35 (m, 2H), 0.09-0.04 (m, 2H).

### Synthesis of 4-[5-cyano-2-(4-methyl-1,2,4-triazol-3-yl) phenyl]-6-[(2-cyclopropylethyl) amino]-N-methoxy-N-methylpyridine-2-carboxamide (F-8)

To a stirred solution of F-7 (50 mg, 1 eq, 0.13 mmol) and N,O-dimethylhydroxylamine hydrochloride (19 mg, 1.5 eq, 0.19 mmol) in Pyridine (5 mL) was added EDCI (49 mg, 2 eq. 0.25 mmol) at room temperature under nitrogen atmosphere. The resulting mixture was stirred for 1 h at 60° C. under nitrogen atmosphere. The mixture was allowed to cool down to room temperature. The resulting mixture was concentrated under reduced pressure. The residue was purified by Prep-TLC with dichloromethane/methyl alcohol (20/1). The crude product was purified by Prep-HPLC with the following conditions (Column: X-Select Prep OBD C18 Column, 30*150 mm, 5 m; Mobile Phase A: Water(0.1% FA), Mobile Phase B: ACN; Flow rate: 60 mL/min: Gradient: 30% B to 50% B in 7 min; Wave Length: 254 nm/220 nm; RT: 6.56). The fraction was collected and concentrated under vacuum, the residue was re-dissolved in CH3CN and H2O, and then was lyophilized to afford F-8 (11.7 mg, 0.03 mmol, 21%, 98.8% purity) as a white solid, m/z 432.1 (M+H)⁺ (ES+), ¹H NMR (400 MHz, DMSO-d₆) δ 8.52 (s, 1H), 8.13-8.06 (m, 2H), 7.79 (d, 1H), 6.84 (t, 1H), 6.40 (s, 1H), 6.23 (d, 1H), 3.63 (s, 3H), 3.20 (s, 8H), 1.42-1.33 (m, 2H), 0.77-0.66 (m, 1H), 0.44-0.32 (m, 2H), 0.06 (d, 2H).

### Synthesis of 3-{2-[(2-cyclopropylethyl) amino]-6-formylpyridin-4-yl}-4-(4-methyl-1,2,4-triazol-3-yl) benzonitrile (F-9)

To a stirred solution of F-8 (120 mg. 0.278 mmol, 1 equiv) in THF (10 mL, 12.343 mmol) was added DIBAl-H (1.39 mL, 1.390 mmol, 5 equiv) at 0° C. under nitrogen atmosphere. The resulting mixture was stirred for 10 min at 0° C. under nitrogen atmosphere. The reaction was quenched by the addition of MeOH (0.5 mL) at 0° C. The residue was purified by Prep-TLC (CH₂Cl₂/MeOH 10:1) to afford F-9 (70 mg. 64.21%) as a off-white solid, m/z 373.2.2 (M+H)⁺ (ES+).

### Synthesis of 3-{2-[(2-cyclopropylethyl) amino]-6-(5-{[(3S)-3-methylpiperidin-1-yl]methyl}-7-(trifluoromethyl)-1,3-benzoxazol-2-yl) pyridin-4-yl}-4-(4-methyl-1,2,4-triazol-3-yl) benzonitrile (I-17)

To a stirred solution of F-9 (70 mg, 0.188 mmol, 1 equiv) in DCM (5 mL) was added F-5 (81.28 mg, 0.282 mmol. 1.5 equiv) at room temperature under nitrogen atmosphere. The resulting mixture was stirred for 1 h at 60° C. under nitrogen atmosphere. The mixture was allowed to cool down to room temperature. To the above mixture was added DDQ (85.33 mg, 0.376 mmol, 2 equiv) in portions at room temperature under nitrogen atmosphere. The resulting mixture was stirred for 1 h at room temperature under nitrogen atmosphere. The resulting mixture was extracted with CH₂Cl₂ (3×15 mL). The combined organic layers were washed with brine (2×15 mL), dried over anhydrous Na₂SO₄. After filtration, the filtrate was concentrated under reduced pressure. The residue was purified by Prep-TLC (CH₂Cl₂/MeOH 10:1). The crude product was purified by Prep-HPLC with the following conditions (Column: XBridge Prep OBD C18 Column, 30*150 mm, 5 m; Mobile Phase A: Water(10 mmol/L NH₄HCO₃), Mobile Phase B: 20 mm NaOH+10% ACN; Flow rate: 60 mL/min mL/min: Gradient: 69% B to 73% B in 7 min; Wave Length: 254 nm/220 nm; RT1 (min): 6.2) to afford I-17 (14.3 mg, 11.85%) as a white solid, m/z 641.2 (M+H)⁺ (ES+). ¹H NMR (400 MHz, DMSO-d₆) δ 8.51 (s, 1H), 8.23 (d, J=1.7 Hz, 1H), 8.16-8.11 (m, 1H), 8.03 (s, 1H), 7.86 (d, 1H), 7.72 (s, 1H), 7.23 (d, 1H), 7.15 (t, 1H), 6.43 (d, 1H), 3.64 (s, 2H), 3.35-3.31 (m, 2H), 3.29 (s, 3H), 2.73 (d, 2H), 1.99-1.89 (m, 1H), 1.70-1.56 (m, 4H), 1.56-1.41 (m, 3H), 0.91-0.71 (m, 5H), 0.45-0.38 (m, 2H), 0.14-0.07 (m, 2H).

### Example 10. 3-(2-(6-((5-azaspiro[2.4]heptan-5-yl)methyl)-3-oxo-8-(trifluoromethyl)imidazo[1,5-a]pyridin-2(3H)-yl)-6-((1-(cyanomethyl)cyclopropyl)amino)pyridin-4-yl)-4-(4-methyl-4H-1,2,4-triazol-3-yl)benzonitrile (I-18)

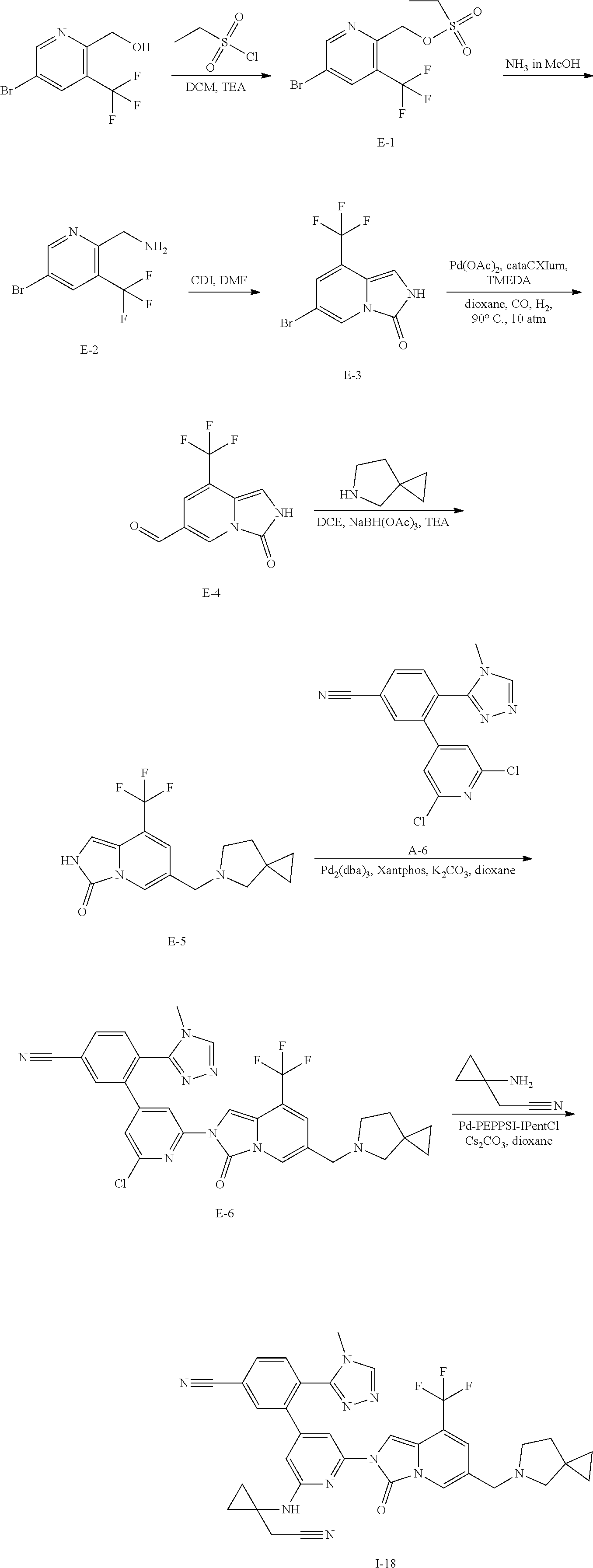

### Synthesis of [5-bromo-3-(trifluoromethyl) pyridin-2-yl]methyl ethanesulfonate (E-1)

To a stirred solution of [5-bromo-3-(trifluoromethyl) pyridin-2-yl]methanol (1.16 g, 4.53 mmol, 1 equiv) in dichloromethane (20 mL) were added TEA (1.38 g, 13.59 mmol, 3 equiv) and ethanesulfonyl chloride (0.78 g, 6.80 mmol, 1.5 equiv) dropwise at 0° C. The resulting mixture was stirred for 1 hour at 0° C. The reaction was quenched with water/ice at 0° C. The mixture was diluted with water and extracted with dichloromethane. The combined organic layer was washed with brine, dried over anhydrous sodium sulfate. The filtrate was concentrated under reduced pressure. The crude product E-1 was used into next step without further purification, m/z 347.9/349.9 (M+H)⁺ (ES+).

### Synthesis of 1-[5-bromo-3-(trifluoromethyl) pyridin-2-yl]methanamine (E-2)

To a solution of NH₃ (g) in MeOH (15 mL, 4.0 M) was added E-1 (1.67 g, 5.00 mmol, 1 equiv) and stirred for 2 hours at 60° C. The reaction was cooled to room temperature. The mixture was diluted with water and extracted with dichloromethane/methanol (10/1). The combined organic layer was washed with brine, dried over anhydrous sodium sulfate. The filtrate was concentrated under reduced pressure. The crude product E-2 was used into next step without further purification, m/z 255.0/257.0 (M+H)⁺ (ES+).

### Synthesis of 6-bromo-8-(trifluoromethyl)-2H-imidazo[1,5-a]pyridin-3-one (E-3)

To a stirred solution of L-2 (972 mg, 3.81 mmol, 1 equiv) in N, N-dimethylfomamide (15 mL) was added CDI (2.47 g, 15.24 mmol, 4 equiv) in portions at room temperature. The resulting mixture was stirred for 2 hours at room temperature. The mixture was diluted with water and extracted with ethyl acetate. The combined organic layer was washed with brine, dried over sodium sulfate. The filtrate was concentrated under reduced pressure. The residue was purified by reverse flash chromatography with the following conditions: column, C18 silica gel; mobile phase, ACN in water, 50% to 60% gradient in 20 min; detector, UV 254 nm. The fraction was concentrated under vacuum to afford E-3 (340 mg, 31.74%) as brown solid, m/z 280.9/282.9 (M+H)⁺ (ES+).

### Synthesis of 3-oxo-8-(trifluoromethyl)-2H-imidazo[1,5-a]pyridine-6-carbaldehyde (E-4)

To a stirred solution of E-3 (340 mg, 1.21 mmol, 1 equiv) in 1,4-dioxane (15 mL) were added TMEDA (421.8 mg, 3.63 mmol, 3 equiv), diadamantan-1-yl(butyl)phosphine (173.5 mg, 0.48 mmol, 0.4 equiv) and Pd(OAc)₂ (27.2 mg, 0.12 mmol, 0.1 equiv) at room temperature under nitrogen atmosphere. The mixture was purged with hydrogen and then was pressurized to 10 atm with carbon monoxide at 90° C. for 16 hours. The resulting mixture was cooled to room temperature and concentrated under reduced pressure. The residue was purified by reverse flash chromatography with the following conditions: column, C18 silica gel; mobile phase, ACN in water, 60% to 70% gradient in 15 min; detector, UV 254 nm. The fraction was concentrated under vacuum to afford E-4 (130 mg, 46.69%) as brown solid, m/z 231.0 (M+H)⁺ (ES+).

### Synthesis of 6-{5-azaspiro [2.4]heptan-5-ylmethyl}-8-(trifluoromethyl)-2H-imidazo[1,5-a]pyridin-3-one (E-5)

To a stirred solution of E-4 (130 mg, 0.56 mmol, 1 equiv) and 5-azaspiro[2.4]heptane (82.3 mg, 0.85 mmol, 1.5 equiv) in DCE (5 mL) were added TEA (342.9 mg. 3.39 mmol, 6 equiv) and NaBH(OAc)₃ (718.3 mg, 3.39 mmol, 6 equiv) at 0° C. The resulting mixture was stirred for 2 hours at room temperature. The resulting mixture was concentrated under reduced pressure. The residue was purified by reverse flash chromatography with the following conditions: column, C18 silica gel; mobile phase. ACN in water, 45% to 50% gradient in 15 min; detector, UV 254 nm. The fraction was concentrated under vacuum to afford E-5 (110 mg, 62.56%) as white solid, m/z 312.1 (M+H)⁺ (ES+).

### Synthesis of 3-[2-(6-{5-azaspiro [2.4]heptan-5-ylmethyl}-3-oxo-8-(trifluoromethyl) imidazo[1,5-a]pyridin-2-yl)-6-chloropyridin-4-yl]-4-(4-methyl-1,2,4-triazol-3-yl) benzonitrile (E-6)

To a stirred solution of E-5 (110 mg, 0.35 mmol, 1 equiv) and 3-(2,6-dichloropyridin-4-yl)-4-(4-methyl-1.2.4-triazol-3-yl) benzonitrile (116.7 mg, 0.35 mmol, 1 equiv) in 1,4-dioxane (3 mL) were added K₂CO₃ (146.5 mg, 1.06 mmol, 3 equiv), XantPhos (40.9 mg, 0.07 mmol, 0.2 equiv) and Pd₂(dba)₃ (32.4 mg, 0.04 mmol, 0.1 equiv) at room temperature under nitrogen atmosphere. The resulting mixture was stirred for 2 hours at 100° C. under nitrogen atmosphere. The resulting mixture was cooled to room temperature and concentrated under reduced pressure. The residue was purified by reverse flash chromatography with the following conditions: column, C18 silica gel; mobile phase, ACN in water, 50% to 55% gradient in 15 min; detector, UV 254 nm. The fraction was concentrated under vacuum to afford E-6 (90 mg, 42.10%) as yellow solid, m/z 605.2/607.2 (M+H)⁺ (ES+).

### Synthesis of 3-[2-(6-{5-azaspiro [2.4]heptan-5-ylmethyl}-3-oxo-8-(trifluoromethyl) imidazo[1,5-a]pyridin-2-yl)-6-{[1-(cyanomethyl) cyclopropyl]amino}pyridin-4-yl]-4-(4-methyl-1,2,4-triazol-3-yl) benzonitrile; formic acid (I-18)

To a stirred solution of E-6 (40 mg, 0.07 mmol, 1 equiv) and 2-(1-aminocyclopropyl)acetonitrile (9.5 mg, 0.10 mmol, 1.5 equiv) in 1,4-dioxane (2 mL) were added Cs₂CO₃ (64.6 mg, 0.20 mmol, 3 equiv) and Pd-PEPPSI-IPentCl 2-methylpyridine (o-picoline) (5.6 mg. 0.01 mmol, 0.1 equiv) at room temperature under nitrogen atmosphere. The resulting mixture was stirred for 2 hours at 100° C. under nitrogen atmosphere. The resulting mixture was cooled to room temperature and concentrated under reduced pressure. The residue was purified by prep-TLC with dichloromethane/methanol (10/1). Then the crude product was re-purified by prep-HPLC with the following conditions: Column: X-Select Prep OBD C18 Column. 30*150 mm. 5 m: Mobile Phase A: Water(0.1% FA), Mobile Phase B: 20 mm NaOH+10% ACN; Flow rate: 60 mL/min; Gradient: 30% B to 50% B in 7 min; Wave Length: 254 nm/220 nm: RT(min): 6.56. The fraction was collected and concentrated under vacuum, the residue was re-dissolved in CH₃CN and H₂O, and then was lyophilized to afford I-18 (5.7 mg, 12.40%) as white solid, m/z 665.6 (M+H)⁺ (ES+). ¹H NMR (400 MHz, DMSO-d₆) δ 8.53 (s, 1H), 8.19-8.09 (m, 2H), 7.86 (d, 1H), 7.69 (d, 2H), 7.44 (d, 2H), 7.06 (t, 1H), 6.28 (d, 1H), 3.39 (s, 5H), 2.86 (s, 2H), 2.68 (t, 2H), 2.45 (s, 2H), 1.75 (t, 2H), 0.90-0.71 (m, 4H), 0.56-0.44 (m, 4H).

### Example 11: Synthesis of 2-(6-(Ethylamino)-4-(2-(4-methyl-4H-1,2,4-triazol-3-yl) phenyl)pyridin-2-yl)-8-(trifluoromethyl)-[1,2,4]triazolo[4,3-a]pyridin-3(2H)-one (G-5)

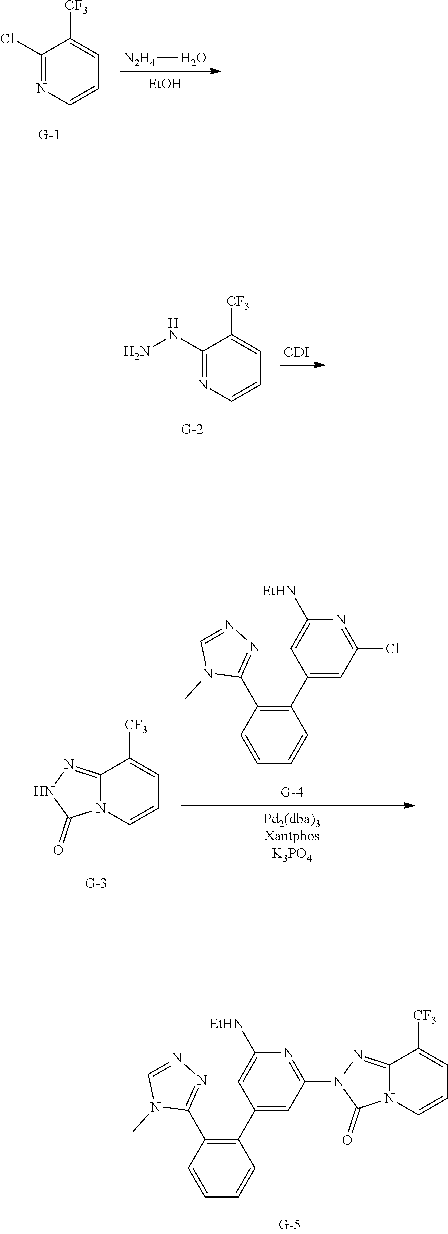

### Synthesis of 2-Hydrazineyl-3-(trifluoromethyl) pyridine (G-2)

To a stirred solution of 2-chloro-3-(trifluoromethyl)pyridine (362 mg. 1 Eq, 1.99 mmol) in EtOH (2 mL) was added hydrazine hydrate (1.50 g, 98% Wt, 15 Eq. 30.0 mmol) at rt under nitrogen atmosphere. The resulting mixture was stirred for 3 h at 80° C. under nitrogen atmosphere. The mixture was allowed to cool down to rt and concentrated in vacuo. The residue was purified by Prep-TLC with EtOAc/petroleum ether (1/1) to afford the sub-title compound G-2 (240 mg, 1.36 mmol, 68%) as a grey solid, m/z 178.1 (M+H)⁺ (ES+).

### Synthesis of 8-(Trifluoromethyl)-[1,2,4]triazolo[4,3-a]pyridin-3(2H)-one (G-3)

To a stirred solution of the product from step 1 above (130 mg, 1 Eq, 734 μmol) in DCM (6 mL) was added CDI (179 mg, 1.5 Eq, 1.10 mmol) at rt under nitrogen atmosphere. The resulting mixture was stirred for 3 h at rt under nitrogen atmosphere. The mixture was allowed to cool down to rt and concentrated in vacuo. The crude product was purified by reverse flash column chromatography with the following conditions: Column, C18; mobile phase, Water (0.1% FA) and MeCN (10% MeCN up to 80% in 20 min): Detector, UV 254/220 nm to afford the sub-title compound G-3 (80 mg, 394 mol, 54%) as a white solid, m/z 204.0 (M+H)⁺ (ES+).

### Synthesis of 2-(6-(Ethylamino)-4-(2-(4-methyl-4H-1,2,4-triazol-3-yl)phenyl)pyridin-2-yl)-8-(trifluoromethyl)-[1,2,4]triazolo[4,3-a]pyridin-3(2H)-one (G-5)

To a solution of intermediate G-4 (70 mg, 1 Eq, 223 μmol), potassium phosphate (118 mg, 2.5 Eq, 557 μmol) and the product from step 2 above (50 mg, 1.1 Eq, 245 μmol) in 1,4-dioxane (6 mL) were added Pd₂(dba)₃-CHCl₃ (46 mg, 0.2 Eq, 45 mol) and XantPhos (52 mg, 0.4 Eq, 89 μmol), under a nitrogen atmosphere. The resulting mixture was stirred for overnight at 100° C. under nitrogen atmosphere. The mixture was allowed to cool down to rt and concentrated. The residue was purified by Prep-TLC with DCM/MeOH (10/1) and Prep-HPLC with the following conditions: (Column: XBridge Prep OBD C18 Column, 30* 150 mm, 5 μm; Mobile Phase A: Water (0.1% NH₄HCO₃), Mobile Phase B: MeCN; Flow rate: 60 mL/min; Gradient: 33% B to 44% B in 8 min; Wave Length: 254/220 nm; RT: 7.7). The product-containing fractions were combined and evaporated partially in vacuo and lyophilized overnight to afford the sub-title compound G-5 (24.6 mg, 51 μmol. 23%) as a yellow solid, m/z 481.1 (M+H)⁺ (ES+). ¹H NMR (300 MHz, Methanol-d4) δ 8.46 (s, 1H), 8.09 (d, 1H), 7.84-7.72 (in. 3H), 7.72-7.59 (m, 2H), 7.05 (s, 1H), 6.77 (t, 1H), 6.18 (s, 1H), 3.38 (s, 3H), 3.27-3.16 (m, 2H), 1.21 (t, 3H).

Certain compounds of formula III are prepared according to the general procedures shown in Scheme V and Scheme VI, wherein X is —CH₂— or —O—.

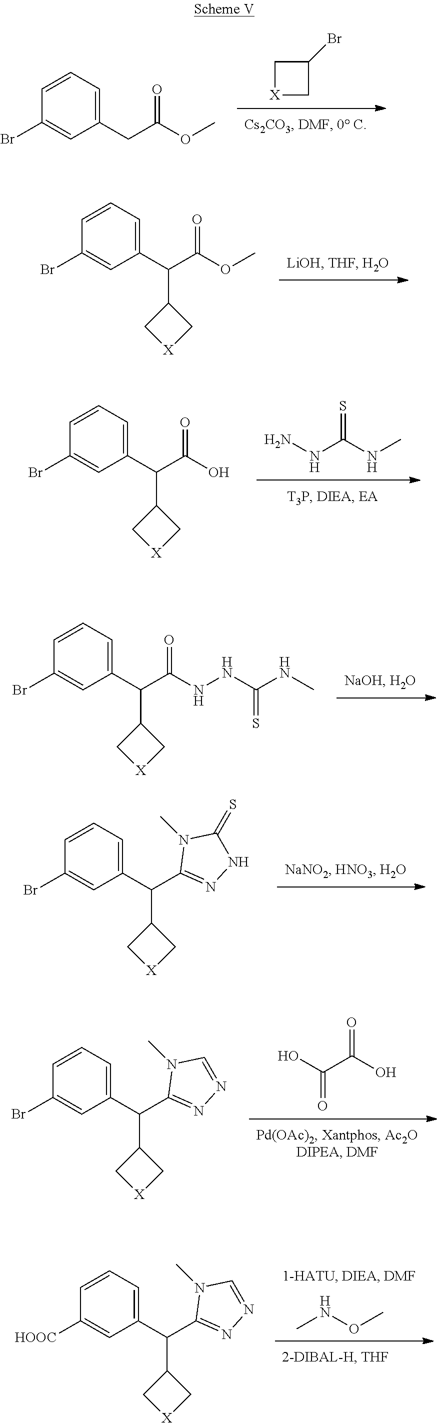

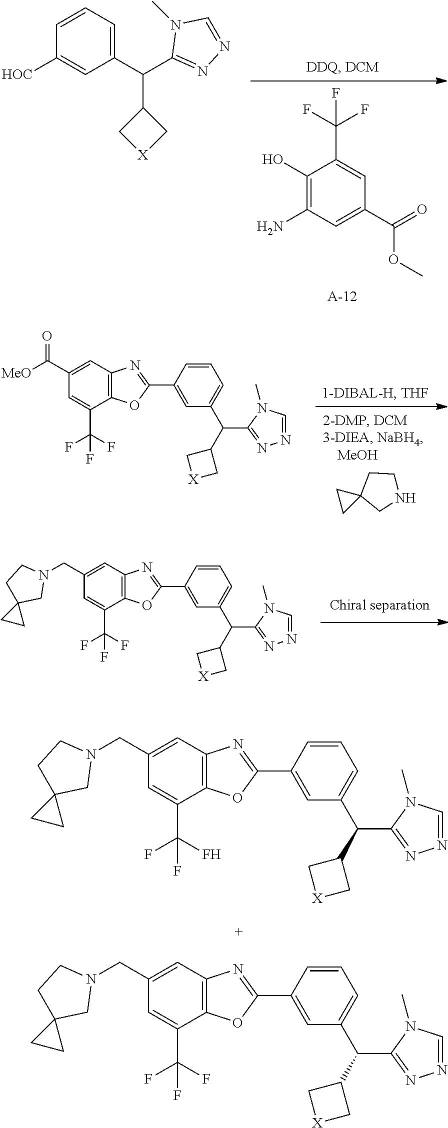

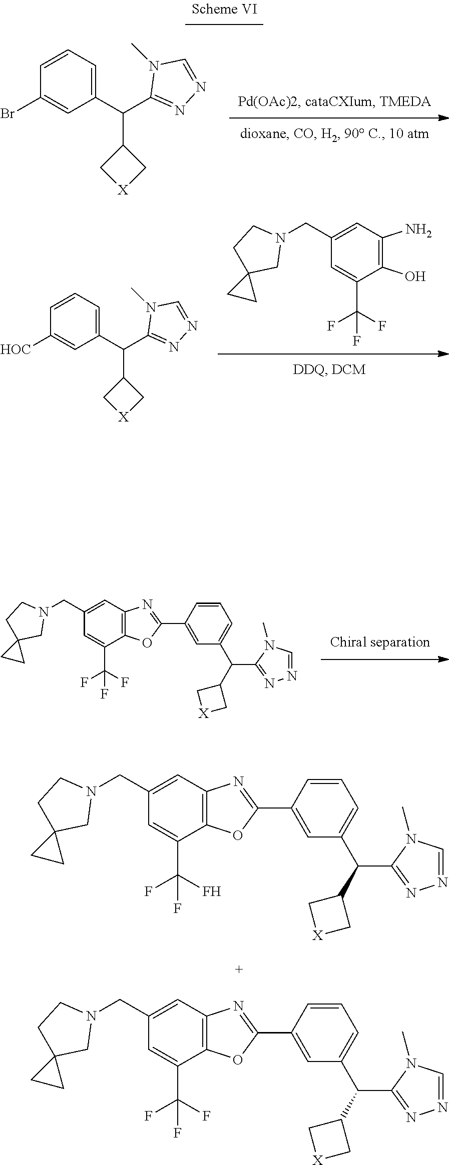

### Example 12. 4-((6-(5-(((2-Hydroxy-2-methylpropyl)amino)methyl)-7-(trifluoromethyl)benzo[d]oxazol-2-yl)-4-(2-(4-methyl-4H-1,2,4-triazol-3-yl)phenyl)pyridin-2-yl)amino)butanenitrile (I-55)

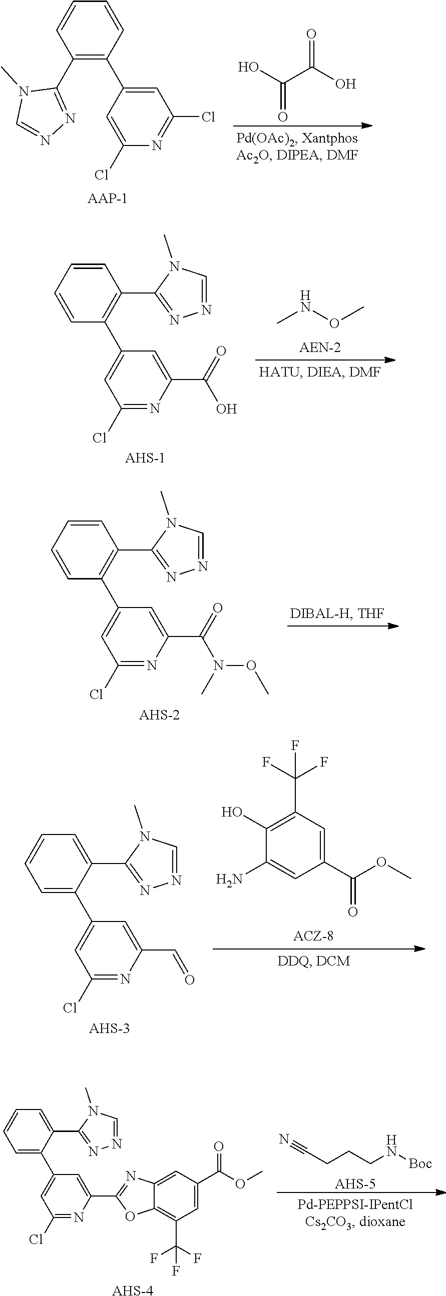

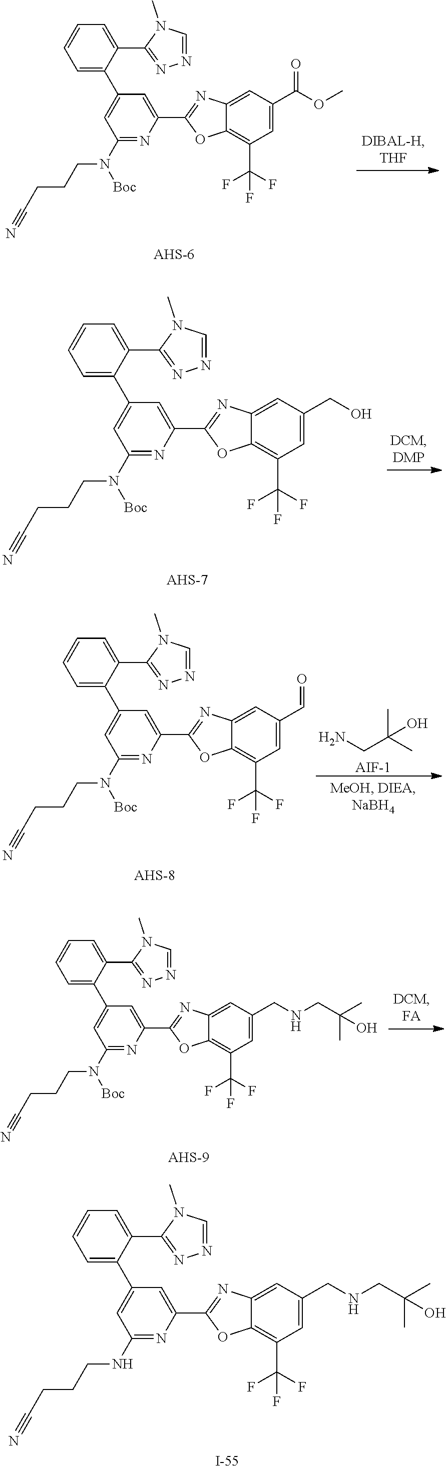

### Synthesis of 6-chloro-4-(2-(4-methyl-4H-1,2,4-triazol-3-yl)phenyl)picolinic acid (AHS-1)

To a stirred solution of 2,6-dichloro-4-[2-(4-methyl-1,2,4-triazol-3-yl)phenyl]pyridine (AAP-1) (300 mg, 1 eq, 0.98 mmol) and oxalic acid (133 mg, 1.5 eq, 1.47 mmol) in DMF (12 mL) were added Ac₂O (151 mg, 1.5 eq, 1.47 mmol), DIPEA (191 mg, 1.5 eq, 1.47 mmol), XantPhos (114 mg. 0.2 eq, 0.20 mmol) and Pd(OAc)₂ (22 mg, 0.1 eq, 0.10 mmol) at room temperature under nitrogen atmosphere. The resulting mixture was stirred for 3 h at 100° C. under nitrogen atmosphere. The mixture was allowed to cool down to room temperature, purified by reverse flash column chromatography with the following conditions: Column, C18; mobile phase, Water (0.1% FA) and ACN (30% ACN up to 50% in 15 min); Detector, UV 254/220 nm. The mixture was concentrated under vacuum to obtain the sub-title compound (AHS-1) (200 mg, 0.63 mmol, 65%, 90% Purity) as a white solid, m/z 315.1/317.1 (M+H)⁺ (ES+).

### Synthesis of 6-chloro-N-methoxy-N-methyl-4-(2-(4-methyl-4H-1,2,4-triazol-3-yl)phenyl) picolinamide (AHS-2)

To a stirred solution of the product from step 1 (AHS-1) (600 mg, 1 eq, 1.91 mmol) and N,O-dimethylhydroxylamine (AEN-2) (140 mg, 1.2 eq, 2.29 mmol) in THF (20 mL) were added DIPEA (739 mg, 3 eq, 5.72 mmol) and HATU (942 mg, 1.3 eq, 2.48 mmol) at 0° C. The resulting mixture was stirred for 1 h at room temperature. The crude product was purified by reverse flash column chromatography with the following conditions: Column, C18; mobile phase, Water (0.1% NH₄HCO₃) and ACN (20% ACN up to 50% in 15 min): Detector, UV 254/220 nm. The mixture was concentrated under vacuum to obtain the sub-title compound (AHS-2) (400 mg, 1.12 mmol, 59%, 92% Purity) as a light yellow solid, m/z 358.1/360.1 (M+H)⁺ (ES+)

### Synthesis of 6-chloro-4-(2-(4-methyl-4H-1,2,4-triazol-3-yl)phenyl)picolinaldehyde (AHS-3)

A solution of the product from step 2 (AHS-2) (100 mg, 1 eq, 0.28 mmol) and DIBAl-H in THF (1.23 mL, 2.5 M, 10 eq, 2.79 mmol) in THF (3 mL) was stirred for 15 min at 0° C. under nitrogen atmosphere. The reaction was then quenched by the addition of 1 mL of methyl alcohol at 0° C. The residue was purified by Prep-TLC with dichloromethane/methyl alcohol (10/1) to afford the sub-title compound (AHS-3) (40 mg, 0.13 mmol, 48%, 95% Purity) as an off-white solid, m/z 299.1/301.1 (M+H)⁺ (ES+).

### Synthesis of methyl 2-(6-chloro-4-(2-(4-methyl-4H-1,2,4-triazol-3-yl)phenyl)pyridin-2-yl)-7-(trifluoromethyl)benzo[d]oxazole-5-carboxylate (AHS-4)

A solution of the product from step 3 (AHS-3) (140 mg, 1 eq, 0.47 mmol) in DCM (5 mL) was treated with methyl 3-amino-4-hydroxy-5-(trifluoromethyl)benzoate (ACZ-8) (110 mg, 1 eq, 0.47 mmol) for 1 h at 60° C. The mixture was cooled to room temperature. To the above mixture was added DDQ (213 mg, 2 eq, 0.94 mmol) at 0° C. The resulting mixture was stirred for 1 h at 0° C. The resulting mixture was concentrated under vacuum. The crude product was purified by reverse flash column chromatography with the following conditions: Column, C18; mobile phase, Water (0.1% NH₄HCO₃) and ACN (40% ACN up to 80% in 20 min); Detector, UV 254/220 nm. The mixture was concentrated under vacuum to afford the sub-title compound (AHS-4) (130 mg, 0.25 mmol, 54%, 90% Purity) as a Brown yellow solid, m/z 514.1/516.1 (M+H)⁺ (ES+).

### Synthesis of methyl 2-(6-((tert-butoxycarbonyl)(3-cyanopropyl)amino)-4-(2-(4-methyl-4H-1,2,4-triazol-3-yl)phenyl)pyridin-2-yl)-7-(trifluoromethyl)benzo[d]oxazole-5-carboxylate (AHS-6)

To a stirred solution of the product from step 4 (AHS-4) (68 mg, 1 eq, 0.13 mmol) and tert-butyl N-(3-cyanopropyl)carbamate (AHS-5) (73 mg, 3 eq, 0.40 mmol) in dioxane (5 mL) were added Cs₂CO₃ (129 mg, 3 eq, 0.40 mmol) and Pd-PEPPSI-IPentCl 2-methylpyridine (o-picoline) (11 mg, 0.1 eq, 13 μmol) at room temperature under nitrogen atmosphere. The resulting mixture was stirred for overnight at 80° C. under nitrogen atmosphere. The mixture was cooled to room temperature and concentrated under reduced pressure. The crude product was purified by reverse flash column chromatography with the following conditions: Column, C18: mobile phase, Water (0.1% NH₄HCO₃) and ACN (60% ACN up to 80% in 15 min); Detector, UV 254/220 nm. The mixture was concentrated under vacuum to afford the sub-title compound (AHS-6) (22 mg, 0.03 mmol, 25%, 94% Purity) as a Brown yellow solid, m/z 662.2 (M+H)⁺ (ES+).

### Synthesis of tert-butyl (3-cyanopropyl)(6-(5-(hydroxymethyl)-7-(trifluoromethyl)benzo[d]oxazol-2-yl)-4-(2-(4-methyl-4H-1,2,4-triazol-3-yl)phenyl)pyridin-2-yl)carbamate (AHS-7)

To a stirred solution of the product from step 5 (AHS-6) (21 mg, 32 μmol, 1 eq) in THF (5 mL) were added and DIBAL-H in THF (0.13 mL, 2.5 M, 10 eq, 0.32 mmol) dropwise at 0° C. under nitrogen atmosphere. The resulting mixture was stirred for 15 min at 0° C. under nitrogen atmosphere. The reaction was then quenched by the addition of 2 mL of methyl alcohol at 0° C. The resulting mixture was concentrated under vacuum. The residue was purified by TLC with dichloromethane/methanol (8/1) to afford the sub-title compound (AHS-7) (17 mg, 0.03 mmol, 85%, 90% Purity) as a light yellow solid, m z 634.2 (M+H)⁺ (ES+).

### Synthesis of tert-butyl (3-cyanopropyl)(6-(5-formyl-7-(trifluoromethyl)benzo[d]oxazol-2-yl)-4-(2-(4-methyl-4H-1,2,4-triazol-3-yl)phenyl)pyridin-2-yl)carbamate (AHS-8)

To a stirred solution of the product from step 6 (AHS-7) (20 mg, 1 eq, 0.03 mmol) in DCM (4 mL) were added DMP (17 mg, 1.3 eq, 0.04 mmol) at room temperature. The resulting mixture was stirred for 1 h at room temperature. The resulting mixture was concentrated under vacuum. The crude product was used in the next step directly without further purification, in z 632.8 (M+H)⁺ (ES+).

### Synthesis of tert-butyl (3-cyanopropyl)(6-(5-(((2-hydroxy-2-methylpropyl)amino)methyl)-7-(trifluoromethyl)benzo[d]oxazol-2-yl)-4-(2-(4-methyl-4H-1,2,4-triazol-3-yl)phenyl)pyridin-2-yl)carbamate (AHS-9)

A solution of the product from step 7 (AHS-8) (20 mg, 1 eq, 0.03 mmol) and 1-amino-2-methylpropan-2-ol (AIF-1) (5.7 mg, 2 eq, 0.06 mmol) in MeOH (4 mL) was treated with DIPEA (12 mg, 3 eq, 0.10 mmol) for 1 h at 60° C. The mixture was cooled to room temperature. To the above mixture wad added NaBH₄ (2.4 mg, 0.06 mmol, 2 eq) at 0° C. The resulting mixture was stirred for 1 h at room temperature. The reaction was then quenched by the addition of 2 mL of methyl alcohol at 0° C. The resulting mixture was concentrated under vacuum. The residue was purified by TLC with dichloromethane/methanol (6/1) to afford the sub-title compound (AHS-9) (15 mg, 0.02 mmol, 67%, 92% Purity) as a light yellow solid, m/z 705.3 (M+H)⁺ (ES+).

### Synthesis of 4-((6-(5-(((2-hydroxy-2-methylpropyl)amino)methyl)-7-(trifluoromethyl)benzo[d]oxazol-2-yl)-4-(2-(4-methyl-4H-1,2,4-triazol-3-yl)phenyl)pyridin-2-yl)amino)butanenitrile (I-55)

To a stirred solution of the product from step 8 (AHS-9) (15 mg, 1 eq, 21 μmol) in DCM (3 mL) and FA (3 mL) at room temperature. The resulting mixture was stirred for overnight at room temperature. The resulting mixture was concentrated under vacuum. The crude product was purified by Prep-HPLC with the following conditions (Column: xBridge Prep Phenyl 5 m OBD 19*250 mm; Mobile Phase A: Water (0.1% NH₄HCO₃), Mobile Phase B: ACN; Flow rate: 20 mL/min; Gradient: isocratic 35% B-65% B IN 20 MIN; Wave Length: 220/254 nm; RT: 14). The fraction was collected and concentrated under vacuum, the residue was re-dissolved in CH₃CN and H₂O, and then was lyophilized to afford the title compound (I-55) (1.9 mg, 3.1 μmol, 14%, 97.4% Purity) as a white solid, m/z 605.5 (M+H)⁺ (ES+). ¹H NMR (400 MHz, DMSO-d6) δ 8.45 (s, 1H), 8.09 (s, 1H), 7.81 (s, 1H), 7.76-7.64 (m, 4H), 7.24-7.18 (m, 2H), 6.43 (s, 1H), 4.22 (s, 1H), 3.94 (s, 2H), 3.34 (s, 2H), 3.27 (s, 3H), 2.58 (t, 2H), 2.39 (s, 2H), 1.91-1.84 (m, 2H), 1.11 (s, 6H).

### Example 13. 4-{[6-(5-{[(Cyclopropylmethyl)amino]methyl}-7-(trifluoromethyl)-1,3-benzoxazol-2-yl)-4-12-(4-methyl-1,2,4-triazol-3-yl)-5-(trifluoromethyl)phenyl]pyridin-2-yl]amino}butanenitrile (I-54)

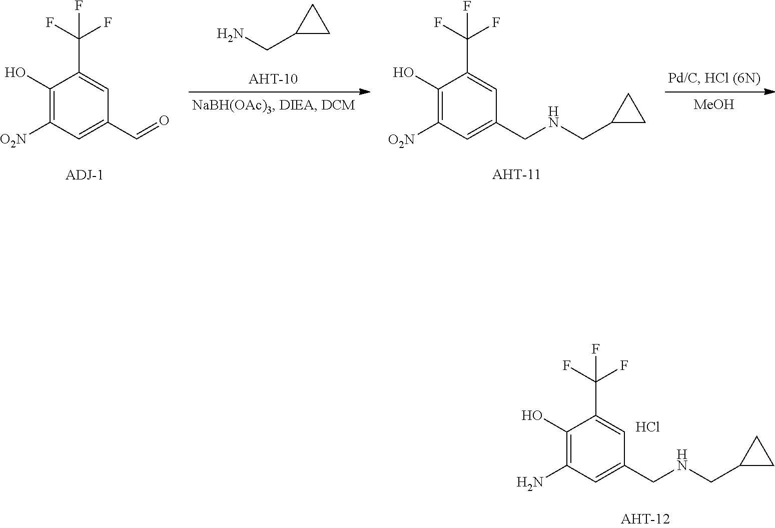

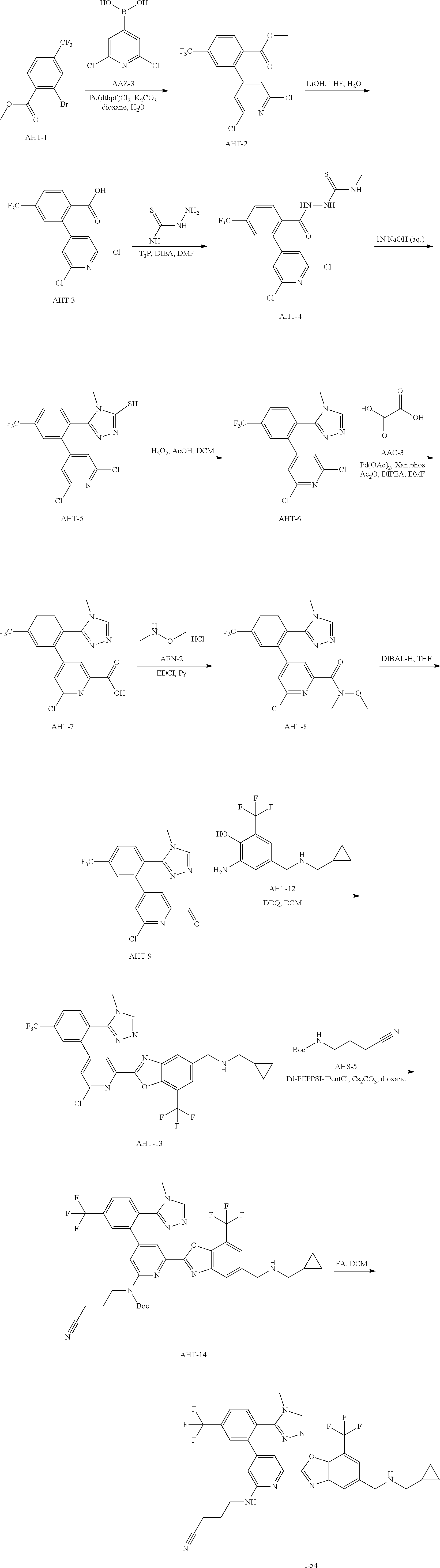

### Synthesis of methyl 2-(2,6-dichloropyridin-4-yl)-4-(trifluoromethyl)benzoate (AHT-2)

To a stirred mixture of methyl 2-bromo-4-(trifluoromethyl)benzoate (AHT-1) (25.00 g, 1 eq, 88.3 mmol) and 2,6-dichloropyridin-4-ylboronic acid (AAZ-3) (16.9 g, 1 eq, 88.3 mmol) in dioxane (200 mL) and H₂O (20 mL) were added K₂CO₃ (36.6 g, 3 eq, 264 mmol) and Pd(DtBPF)Cl₂ (5.76 g, 0.1 eq, 8.83 mmol) at room temperature under nitrogen atmosphere. The resulting mixture was stirred for overnight at 60° C. under nitrogen atmosphere. The mixture was allowed to cool down to room temperature. The resulting mixture was concentrated under reduced pressure. The residue was applied on a silica gel column chromatography with petroleum ether/ethyl acetate (1/1) to afford the sub-title compound (AHT-1) (10.0 g, 2.86 mmol, 32.3%, 92% Purity) as a white solid, m/z 350.0/352.0 (M+H) (ES+).

### Synthesis of 2-(2,6-dichloropyridin-4-yl)-4-(trifluoromethyl)benzoic acid (AHT-3)

To a stirred mixture of the product from step 1 (AHT-3) (5.00 g, 1 eq, 14.3 mmol) in THF (100 mL) and H₂O (30 mL) was added LiOH (1.03 g, 3 eq, 42.8 mmol) at room temperature. The resulting mixture was stirred for 3 h at 60° C. The mixture was cooled to room temperature, diluted with water and extracted with ethyl acetate (3×300 mL). The combined organic layers were washed with brine (2×300 mL), dried over anhydrous sodium sulfate. After filtration, the filtrate was concentrated under reduced pressure. This resulted in the sub-title compound (AHT-3) (4.00 g, 11.9 mmol, 83%, 85% Purity) as a white solid, n/z 336.0/338.0 (M+H)⁺ (ES+).

### Synthesis of 2-(2,6-dichloropyridin-4-yl)-N-[(methylcarbamothioyl)amino]-4-(trifluoromethyl) benzamide (AHT-4)

To a stirred mixture of the product from step 2 (AHT-3) (5.00 g, 1 eq, 14.9 mmol) and 4-methyl-3-thiosemicarbazide (1.56 g, 1 eq, 14.9 mmol) in DMF (10 mL) were added T₃P in EA (37.9 g, 50% Wt, 8 eq, 119 mmol) and DIPEA (11.5 g, 6 eq, 89.3 mmol) at room temperature. The resulting mixture was stirred for overnight at room temperature. The crude product was purified by reverse flash column chromatography with the following conditions: Column. C18: mobile phase. Water (0.1% NH₄HCO₃) and ACN (10% ACN up to 50% in 20 min); Detector, UV 254/220 nm. This resulted in the sub-title compound (AHT-4) (1.2 g, 2.84 mmol, 19%, 90% Purity) as a white solid, mi z 423.0/425.0 (M+H)⁺ (ES+).

### Synthesis of 5-[2-(2,6-dichloropyridin-4-yl)-4-(trifluoromethyl)phenyl]-4-methyl-1,2,4-triazole-3-thiol (AHT-5)

To a stirred mixture of the product from step 3 (AHT-4) (2.00 g, 1 eq, 4.73 mmol) in H₂O (10 mL) was added NaOH (0.04 g, 0.21 eq, 1.00 mmol) at room temperature. The resulting mixture was stirred for overnight at 80° C. The mixture was allowed to cool down to room temperature. The resulting mixture was concentrated under reduced pressure. The crude product was purified by reverse flash column chromatography with the following conditions: Column, C18; mobile phase, Water (0.1% NH₄HCO₃) and ACN (10% ACN up to 55% in 15 min): Detector, UV 254/220 nm. This resulted in the sub-title compound (AHT-5) (1.8 g, 4.46 mmol, 94%, 94% Purity) as a white solid, m/z 405.0/407.0 (M+H)⁺ (ES+).

### Synthesis of 2,6-dichloro-4-[2-(4-methyl-1,2,4-triazol-3-yl)-5-(trifluoromethyl)phenyl]pyridine (AHT-6)

To a stirred mixture of the product from step 4 (AHT-5) (3.90 g, 1 eq, 9.62 mmol) in DCM (40 mL) were added AcOH (1.16 g, 2 eq. 19.2 mmol) and H₂O₂ (1.64 g, 30% Wt. 5 eq. 48.1 mmol) at room temperature. The resulting mixture was stirred for 2 h at room temperature. The resulting mixture was concentrated under reduced pressure. The crude product was purified by reverse flash column chromatography with the following conditions: Column, C18; mobile phase, Water (0.1% NH₄HCO₃) and ACN (50% ACN up to 80% in 20 min); Detector, UV 254/220 nm. This resulted in the sub-title compound (AHT-6) (1.80 g, 4.84 mmol. 50%. 94% Purity) as a white solid, m/z 373.0/375.0 (M+H) (ES+).

### Synthesis of 6-chloro-4-[2-(4-methyl-1,2,4-triazol-3-yl)-5-(trifluoromethyl)phenyl]pyridine-2-carboxylic acid (AHT-7)

To a stirred solution of the product from step 5 (AHT-6) (300 mg, 1 eq, 0.80 mmol) and oxalic acid (87 mg, 1.2 eq, 0.97 mmol) in DMF (15 mL) were added DIPEA (156 mg, 1.5 eq. 1.21 mmol) and Ac₂O (123 mg. 1.5 eq. 1.21 mmol) at room temperature under nitrogen atmosphere. To the above mixture were added XantPhos (93 mg, 0.2 eq, 0.16 mmol) and Pd(OAc)₂ (18 mg, 0.1 eq, 0.08 mmol) at room temperature under nitrogen atmosphere. The resulting mixture was stirred for 3 h at 100° C. under nitrogen atmosphere. The mixture was allowed to cool down to room temperature. The crude product was purified by reverse flash column chromatography with the following conditions: Column, C18; mobile phase. Water (0.1% FA) and ACN (0% ACN up to 30% in 30 min): Detector, UV 254/220 nm. This resulted in the sub-title compound (AHT-7) (120 mg, 0.31 mmol, 36%, 92% Purity) as an off-white solid, m/z 383.0/385.0 (M+H)⁺ (ES+).

### Synthesis of 6-chloro-N-methoxy-N-methyl-4-[2-(4-methyl-1,2,4-triazol-3-yl)-5-(trifluoromethyl) phenyl]pyridine-2-carboxamide (AHT-8)

To a stirred solution of the product from step 6 (AHT-7) (110 mg, 1 eq, 0.29 mmol) and N, O-dimethylhydroxylamine hydrochloride (AEN-2) (42 mg, 1.5 eq, 0.43 mmol) in Pyridine (5 mL) was added EDCI (110 mg, 2 eq, 0.57 mmol) at room temperature under nitrogen atmosphere. The resulting mixture was stirred for 1 h at 60° C. under nitrogen atmosphere. The mixture was allowed to cool down to room temperature. The resulting mixture was concentrated under reduced pressure. The residue was purified by Prep-TLC with dichloromethane/methyl alcohol (20/1) to afford the sub-title compound (AHT-8) (85 mg, 0.20 mmol, 62%, 90% Purity) as a yellow solid, m/z 426.1/428.1 (M+H)⁺ (ES+).

### Synthesis of 6-chloro-4-[2-(4-methyl-1,2,4-triazol-3-yl)-5-(trifluoromethyl)phenyl]pyridine-2-carbaldehyde (AHT-9)

To a stirred solution of the product from step 7 (AHT-8) (30 mg, 1 eq, 0.07 mmol) in THF (5 mL) was added DIBALH in THF (0.35 mL, 1.0 M, 5 eq, 0.35 mmol) at 0° C. under nitrogen atmosphere. The resulting mixture was stirred for 10 min at 0° C. under nitrogen atmosphere. The reaction was then quenched by the addition of 0.5 mL of ice water at 0° C. The resulting mixture was concentrated under reduced pressure. The residue was purified by Prep-TLC with dichloromethane/methyl alcohol (10/1) to afford the sub-title compound (AHT-9) (20 mg, 0.05 mmol, 70%, 90% Purity) as a light yellow solid, m/z 367.1/369.1 (M+H)⁺ (ES+).

### Synthesis of 4-{[(cyclopropylmethyl)amino]methyl}-2-nitro-6-(trifluoromethyl)phenol (AHT-11)

To a stirred solution of 4-hydroxy-3-nitro-5-(trifluoromethyl)benzaldehyde (ADJ-1) (500 mg, 1 eq, 2.13 mmol) and 1-cyclopropylmethanamine (AHT-10) (227 mg. 1.5 eq. 3.19 mmol) in DCM (20 mL) were added DIPEA (825 mg, 3 eq, 6.38 mmol) and NaBH(OAc)₃ (1.35 g, 3 eq, 6.38 mmol) at room temperature under nitrogen atmosphere. The resulting mixture was stirred for overnight at room temperature under nitrogen atmosphere. T The reaction was then quenched by the addition of 2 mL of methyl alcohol at 0° C. The resulting mixture was concentrated under reduced pressure. The residue was purified by Prep-TLC with dichloromethane/methyl alcohol (20/1) to afford the sub-title compound (AHT-11) (550 mg. 1.90 mmol. 79%. 96% Purity) as a yellow solid, m/z 291.1 (M+H)⁺ (ES+).

### Synthesis of 2-amino-4-{[(cyclopropylmethyl)amino]methyl}-6-(trifluoromethyl)phenol (AHT-12)

To a stirred solution of the product from step 9 (AHT-11) (500 mg, 1 eq, 1.72 mmol) in MeOH (8 mL) was added HCl (8 mL) at 0° C. under nitrogen atmosphere. To the above mixture was added wet Pd/C (100 mg, 10% Wt) at room temperature under nitrogen atmosphere. The resulting mixture was stirred for overnight at room temperature under hydrogen atmosphere. The resulting mixture was filtered; the filter cake was washed with MeOH (3×5 mL). The filtrate was concentrated under reduced pressure. The crude product was purified by Prep-HPLC with the following conditions (Column: X-Select Prep OBD C18 Column, 30*150 mm, 5 μm; Mobile Phase A: Water (0.1% HCL), Mobile Phase B: ACN; Flow rate: 60 mL/min; Gradient: 30% B to 50% B in 7 min; Wave Length: 254 nm/220 nm; RT: 6.56) to afford the sub-title compound (AHT-12) (200 mg, 0.77 mmol, 40%, 99% Purity) as an off-white solid, m/z 261.1 (M+H)⁺ (ES+).

### Synthesis of [(2-{6-chloro-4-[2-(4-methyl-1,2,4-triazol-3-yl)-5-(trifluoromethyl)phenyl]pyridin-2-yl}-7-(trifluoromethyl)-1,3-benzoxazol-5-yl)methyl](cyclopropylmethyl)amine (AHT-13)

To a stirred solution of the product from step 8 (AHT-9) (50 mg, 1 eq, 0.14 mmol) and the product from step 10 (AHT-12) (71 mg, 2 eq, 0.27 mmol) in DCM (5 mL) and NMP (1 mL) at room temperature under nitrogen atmosphere. The resulting mixture was stirred for 1 h at 60° C. under nitrogen atmosphere. The mixture was allowed to cool down to room temperature. To the above mixture was added DDQ (62 mg, 0.27 mmol, 2 equiv) at room temperature under nitrogen atmosphere. The resulting mixture was stirred for 1 h at room temperature under nitrogen atmosphere. The resulting mixture was concentrated under reduced pressure. The crude product was purified by reverse flash column chromatography with the following conditions: Column, C18: mobile phase, Water (0.1% NH₄HCO₃) and ACN (50% ACN up to 70% in 20 min); Detector, UV 254/220 nm. This resulted in the sub-title compound (AHT-13) (32 mg, 0.05 mmol, 34%, 90% Purity) as a brown solid, m/z 607.1/609.1 (M+H) (ES+).

### Synthesis of tert-butyl N-(3-cyanopropyl)-N-[6-(5-{[(cyclopropylmethyl)amino]methyl}-7-(trifluoromethyl)-1,3-benzoxazol-2-yl)-4-12-(4-methyl-1,2,4-triazol-3-yl)-5-(trifluoromethyl)phenyl]pyridin-2-yl]carbamate (AHT-14)

To a stirred solution of the product from step 11 (AHT-13) (90 mg, 1 eq, 0.15 mmol) and tert-butyl N-(3-cyanopropyl)carbamate (AHS-5) (33 mg, 1.2 eq, 0.18 mmol) in dioxane (5 mL) was added Cs₂CO₃ (145 mg, 3 eq, 0.44 mmol) at room temperature under nitrogen atmosphere. To the above mixture was added Pd-PEPPSI-IPentCl 2-methylpyridine (o-picoline) (13 mg, 0.1 eq, 15 μmol) at room temperature under nitrogen atmosphere. The resulting mixture was stirred for 3 h at 90° C. under nitrogen atmosphere. The mixture was allowed to cool down to room temperature. The resulting mixture was concentrated under reduced pressure. The residue was purified by Prep-TLC with dichloromethane/methyl alcohol (5/1) to afford the sub-title compound (AHT-14) (48 mg, 0.06 mmol, 32%, 92% Purity) as an off-white solid, m/z 755.3 (M+H)⁺ (ES+).

### Synthesis of 4-{[6-(5-{[(cyclopropylmethyl)amino]methyl}-7-(trifluoromethyl)-1,3-benzoxazol-2-yl)-4-[2-(4-methyl-1,2,4-triazol-3-yl)-5-(trifluoromethyl)phenyl]pyridin-2-yl]amino}butanenitrile; formic acid (I-54)

To a stirred solution of the product from step 14 (AHT-14) (40 mg, 1 eq, 53 μmol) in DCM (2 mL) was added formic acid (2 mL) at room temperature under nitrogen atmosphere. The resulting mixture was stirred for overnight at room temperature under nitrogen atmosphere. The resulting mixture was concentrated under reduced pressure. The crude product was purified by Prep-HPLC with the following conditions (Column: XSelect Prep OBD C18 Column, 30*150 mm, 5 m; Mobile Phase A: Water (0.1% FA). Mobile Phase B: ACN; Flow rate: 60 mL/min; Gradient: 25% B to 55% B in 8 min; Wave Length: 254 nm/220 nm: RT: 6.6). The fraction was collected and concentrated under vacuum, the residue was re-dissolved in CH₃CN and H0, and then was lyophilized to afford the title compound (I-54) (1.9 mg, 2.9 μmol, 5.1%, 98.8% Purity) as a light yellow solid, m/z 655.3 (M+H)⁺ (ES+). ¹H NMR (400 MHz, DMSO-d6) δ 8.50 (s, 1H), 8.13-7.99 (m, 3H), 7.91 (d, 1H), 7.82 (s, 1H), 7.26 (d, 2H), 6.52 (s, 1H), 3.94 (s, 2H), 3.35 (s, 2H), 3.32 (s, 3H), 2.59 (s, 2H), 2.42 (d, 2H), 1.93-1.83 (n, 2H), 0.99-0.83 (m, 1H), 0.41 (d, 2H), 0.11 (d, 2H).

### Example 14. (S)-3-(2-(6-((6-Azaspiro[3.4]octan-6-yl)methyl)-3-oxo-8-(trifluoromethyl)imidazo[1,5-a]pyridin-2(3H)-yl)-6-((3-cyano-2-hydroxypropyl)amino)pyridin-4-yl)-4-(4-methyl-4H-1,2,4-triazol-3-yl)benzonitrile (I-50)

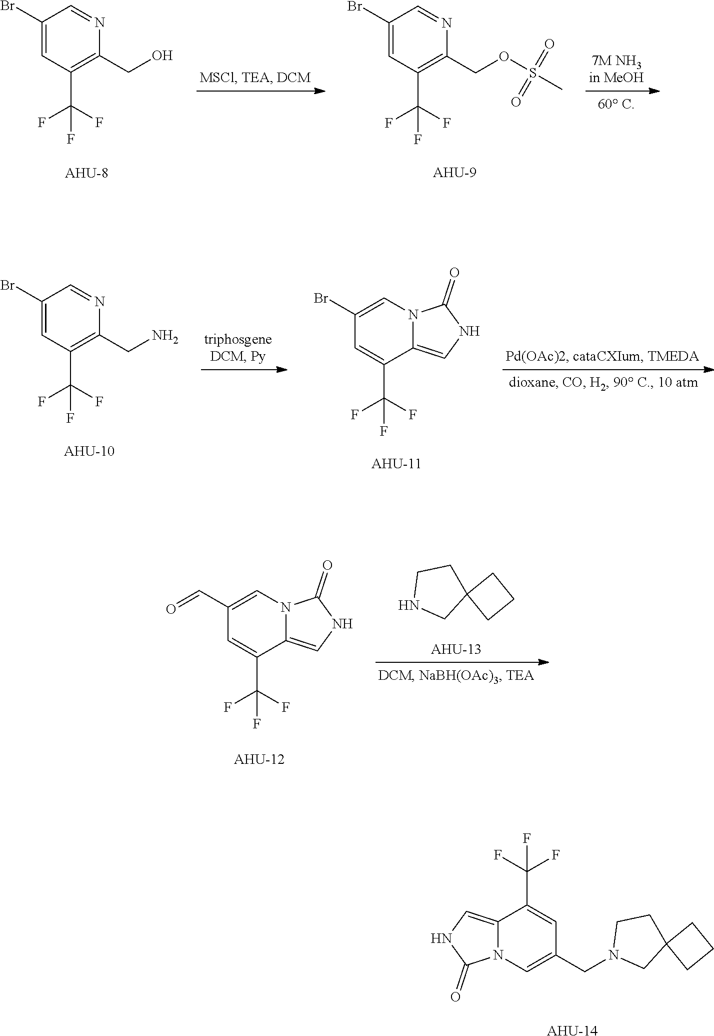

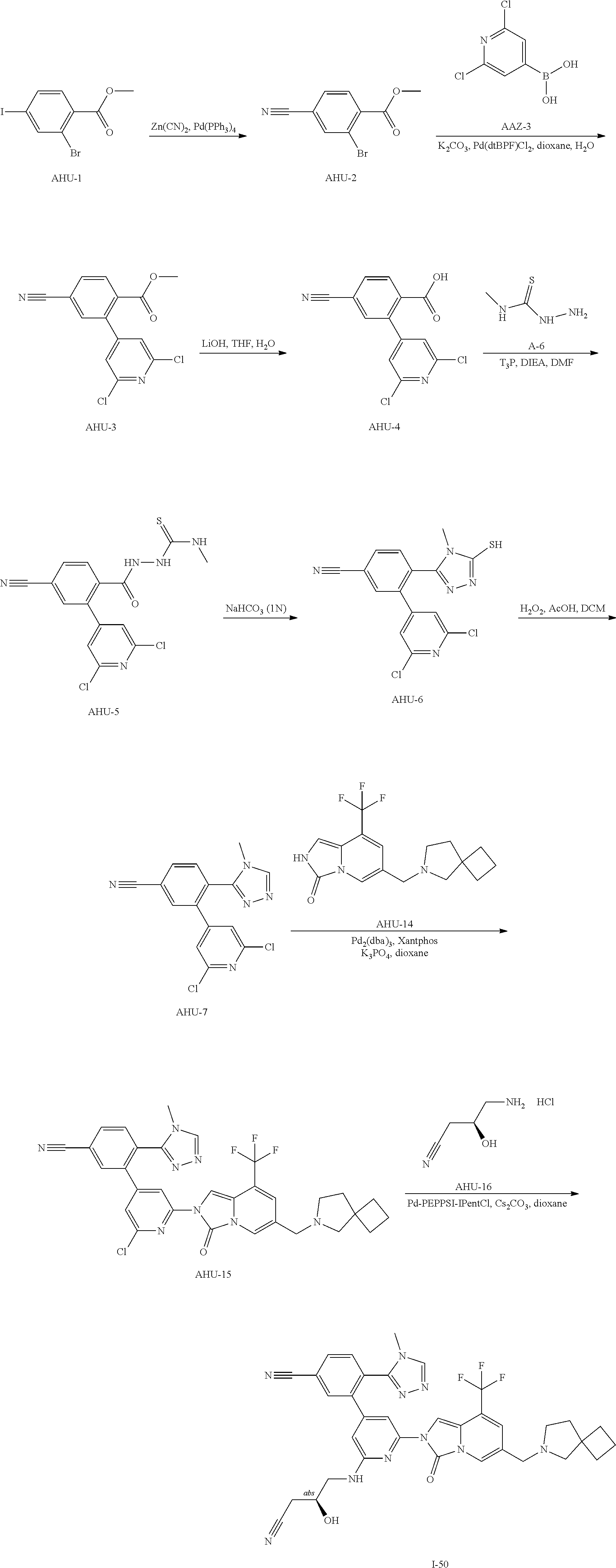

### Synthesis of methyl 2-bromo-4-cyanobenzoate (AHU-2)

To a stirred solution of methyl 2-bromo-4-iodobenzoate (AHU-1) (3.00 g, 1 eq, 8.80 mmol) and Zn(CN)₂ (1.34 g, 1.3 eq, 11.4 mmol) in DMF (20 mL) was added Pd(PPh₃)₄ (1.02 g, 0.1 eq, 0.88 mmol) at room temperature under nitrogen atmosphere. The resulting mixture was stirred for 4 h at 60° C. under nitrogen atmosphere. The mixture was cooled to room temperature, diluted with water and extracted with ethyl acetate (3×200 mL). The combined organic layers were washed with brine (2×200 mL), dried over anhydrous sodium sulfate. After filtration, the filtrate was concentrated under reduced pressure. The residue was applied on a silica gel column chromatography with petroleum ether/ethyl acetate (20/1) to afford the sub-title compound (AHU-2) (1.5 g, 6.27 mmol, 71%, 90% Purity) as a yellow solid, m/z 240.1/242.1 (M+H)⁺ (ES+).

### Synthesis of methyl 4-cyano-2-(2,6-dichloropyridin-4-yl)benzoate (AHU-4)

To a stirred solution of the product from step 1 above (AHU-2) (2.00 g. 1 eq. 8.33 mmol) 2,6-dichloropyridin-4-ylboronic acid (AAZ-3) (1.92 g, 1.2 eq, 10.0 mmol) and K₂CO₃ (3.45 g, 25.0 mmol, 3 eq) in dioxane (20 mL) and H₂O (2 mL) was added Pd(DtBPF)Cl₂ (540 g, 0.1 eq, 0.83 mmol) at room temperature under nitrogen atmosphere. The resulting mixture was stirred for 4 h at 60° C. under nitrogen atmosphere. The mixture was allowed to cool down to room temperature. The resulting mixture was concentrated under reduced pressure. The residue was applied on a silica gel column chromatography with petroleum ether/ethyl acetate (5/1) to afford the sub-title compound (AHU-3) (4.92 g. 16.0 mmol, crude, 52% Purity) as a brown solid, m/z 307.1/309.1 (M+H)⁺ (ES+).

### Synthesis of 4-cyano-2-(2,6-dichloropyridin-4-yl)benzoic acid (AHU-4)

To a stirred solution of the product from step 2 above (AHU-3) (3.00 g, 1 eq, 9.77 mmol) and LiOH (1.17 g, 5 eq, 48.8 mmol) in THF (60 ml) and H₂O (20 mL) at room temperature. The resulting mixture was stirred for 3 h at room temperature. The mixture was acidified to pH 2 with cone. HCl at 0° C. The resulting mixture was diluted with water and extracted with ethyl acetate (3×200 mL). The combined organic layers were washed with brine (2×200 mL), dried over anhydrous sodium sulfate. After filtration, the filtrate was concentrated under reduced pressure. This resulted in the sub-title compound (AHU-4) (7.1 g, crude) as a brown solid, m/z 293.1/295.1 (M+H)⁺ (ES+).

### Synthesis of 4-cyano-2-(2,6-dichloropyridin-4-yl)-N-[(methylcarbamothioyl)amino]benzamide (AHU-5)

To a stirred solution of the product from step 3 above (AHU-4) (2.00 g. 1 eq. 6.82 mmol) and 4-methyl-3-thiosemicarbazide (1.08 g, 1.5 eq, 10.3 mmol) in DMF (20 mL) were added T₃P (8.68 g, 4 eq, 27.3 mmol) and DIPEA (7.06 g, 8 eq, 54.6 mmol) at room temperature. The resulting mixture was stirred for 2 h at room temperature. The crude product was purified by reverse flash column chromatography with the following conditions: Column, C18; mobile phase, Water (0.1% NHCO₃) and ACN (0% ACN up to 50% in 30 min); Detector, UV 254/220 nm. The product-containing fractions were combined and concentrated under reduced pressure. This resulted in the sub-title compound (AHU-5) (1.8 g, 4.73 mmol, 69%, 89% Purity) as a yellow solid, m/z 380.2/382.2 (M+H)⁺ (ES+).

### Synthesis of 3-(2,6-dichloropyridin-4-yl)-4-(4-methyl-5-sulfanyl-1,2,4-triazol-3-yl)benzonitrile (AHU-6)

A solution of the product from step 4 above (AHU-5) (2.00 g. 1 eq. 5.26 mmol) in aq, of NaHCO₃ (40 mL, 1 M) was stirred for 4 h at 80° C. The mixture was cooled to room temperature and concentrated under reduced pressure. The crude product was purified by reverse flash column chromatography with the following conditions: Column, C18; mobile phase, Water (0.1% FA) and ACN (0% ACN up to 40% in 30 min); Detector, UV 254/220 nm. This resulted in the sub-title compound (AHU-6) (950 mg, 2.63 mmol, 54%, 92% Purity) as a yellow solid, m/z 362.2/364.2 (M+H)⁺ (ES+).

### Synthesis of 3-(2,6-dichloropyridin-4-yl)-4-(4-methyl-1,2,4-triazol-3-yl)benzonitrile (AHU-7)

To a stirred solution of the product from step 5 above (AHU-6) (20 mg. 1 eq, 0.06 mmol) and AcOH (7 mg, 2 eq, 0.11 mmol) in DCM (8 mL) was added H₂O₂ (31 mg, 30% Wt, 5 eq, 0.28 mmol) at 0° C. The resulting mixture was stirred for 2 h at room temperature. The resulting mixture was concentrated under reduced pressure. The crude product was purified by reverse flash column chromatography with the following conditions: Column, C18: mobile phase, Water (0.1% NH₄HCO₃) and ACN (10% ACN up to 50% in 20 min); Detector. UV 254/220 nm. The crude product was purified by Prep-HPLC with the following conditions (Column: YMC-Actus Triart C18, 30*150 mm, 5 m; Mobile Phase A: Water (0.1% NH₄HCO₃), Mobile Phase B: ACN: Flow rate: 60 mL/min: Gradient: 20% B to 50% B in 8 min; Wave Length: 254/210 nm; RT: 7.3). The product-containing fractions were combined and evaporated partially in vacuo and lyophilized overnight to afford the sub-title compound (AHU-7) (14.4 mg, 43 mol, 79%, 98% Purity) as a white solid, m/z 330.0/332.0 (M+H)⁺ (ES+).

### Synthesis of (5-bromo-3-(trifluoromethyl)pyridin-2-yl)methyl methanesulfonate (AHU-9)

To a stirred solution of [5-bromo-3-(trifluoromethyl)pyridin-2-yl]methanol (AHU-8) (1.16 g, 1 eq, 4.53 mmol) and TEA (1.38 g, 3 eq, 13.6 mmol) in DCM (15 mL) was added ethanesulfonyl chloride (870 mg, 1.5 eq, 6.80 mmol) dropwise at 0° C. under nitrogen atmosphere. The resulting mixture was stirred for 45 min at 0° C. under nitrogen atmosphere. The resulting mixture was diluted with water and extracted with dichloromethane (3×100 mL). The combined organic layers were washed with brine (2×100 mL), dried over anhydrous sodium sulfate. After filtration, the filtrate was concentrated under reduced pressure. This resulted in the sub-title compound (AHU-9) (1.63 g, crude) as a brown oil, m/z 333.9/335.9 (M+H)⁺ (ES+).

### Synthesis of 1-[5-bromo-3-(trifluoromethyl)pyridin-2-yl]methanamine (AHU-10)

To a stirred solution of the product from step 7 (AHU-9) (1.67 g, 1 eq, 5.01 mmol) and NH₃ (g) in MeOH (15 mL, 7 M) was stirred for 2 h at 60° C. The mixture was cooled to room temperature and concentrated under reduced pressure. The resulting mixture was diluted with water and extracted with dichloromethane/methyl alcohol (10/1) (3×100 mL). The combined organic layers were washed with brine (2×100 mL), dried over anhydrous sodium sulfate. After filtration, the filtrate was concentrated under reduced pressure. This resulted in the sub-title compound (AHU-10) (1.3 g, crude) as a brown oil, m % z 255.0/257.0 (M+H)⁺ (ES+).

### Synthesis of 6-bromo-8-(trifluoromethyl)-2H-imidazo[1,5-a]pyridin-3-one (AHU-11)

To a stirred solution of the product from step 8 (AHU-10) (1.20 g, 1 eq, 4.71 mmol) in DMF (17 mL) was added CDI (3.05 g, 4 eq, 18.8 mmol) at room temperature and stirred for overnight. The crude product was purified by reverse flash column chromatography with the following conditions: Column, C18: mobile phase, Water (0.1% NH4HCO3) and ACN (10% ACN up to 50% in 15 min); Detector, UV 254/220 nm. This resulted in the sub-title compound (AHU-11) (340 mg. 1.21 mmol, 26%, 90% Purity) as a yellow solid, m/z 280.9/282.9 (M+H)⁺ (ES+).

### Synthesis of 3-oxo-8-(trifluoromethyl)-2H-imidazo[1,5-a]pyridine-6-carbaldehyde (AHU-12)

To a solution of the product from step 9 (AHU-11) (340 mg, 1 eq, 1.21 mmol) in 1,4-dioxane (15 mL) were added TMEDA (422 mg, 3 eq, 3.63 mmol), butylbis[(3R,5S,7s)-adamantan-1-yl]phosphane (174 mg, 0.4 eq, 0.48 mmol) and Pd(OAc)₂ (27 mg, 0.1 eq, 0.12 mmol) in a pressure tank. The mixture was purged with nitrogen for 0.5 min and then was pressurized to 10 Mpa with carbon monoxide at 90° C. for overnight. The reaction mixture was cooled to room temperature. The resulting mixture was filtered the filter cake was washed with ethyl acetate (3×15 mL). The filtrate was concentrated under reduced pressure. The residue was purified by Prep-TLC with ethyl acetate/petroleum ether (1/1) to afford the sub-title compound (AHU-12) (130 mg. 0.57 mmol, 47%, 92% Purity) as a yellow solid, m/z 231.0 (M+H)⁻ (ES+).

### Synthesis of 6-{6-azaspiro[3.4]octan-6-ylmethyl}-8-(trifluoromethyl)-2H-imidazo[1,5-a]pyridin-3-one (AHU-14)

To a stirred solution of the product from step 10 (AHU-12) (90 mg, 1 eq, 0.39 mmol) and 6-azaspiro[3.4]octane (AHU-13) (65 mg, 1.5 eq, 0.59 mmol) in DCM (8 mL) were added TEA (237 mg, 6 eq, 2.35 mmol) and NaBH(OAc)₃ (497 mg, 6 eq, 2.35 mmol) at 0° C. under nitrogen atmosphere. The resulting mixture was stirred for 2 h at room temperature under nitrogen atmosphere. The reaction was then quenched by the addition of 2 mL of methyl alcohol at 0° C. The resulting mixture was concentrated under reduced pressure. The crude product was purified by reverse flash column chromatography with the following conditions: Column, C18; mobile phase, Water (0.1% NH₄HCO₃) and ACN (20% ACN up to 70% in 20 min); Detector, UV 254/220 nm. This resulted in the sub-title compound (AHU-14) (82 mg, 0.25 mmol, 64%, 95% Purity) as a yellow solid, m/z 326.1 (M+H)⁺ (ES+).

### Synthesis of 3-[2-(6-{6-azaspiro[3.4]octan-6-ylmethyl}-3-oxo-8-(trifluoromethyl)imidazo[1,5-a]pyridin-2-yl)-6-chloropyridin-4-yl]-4-(4-methyl-1,2,4-triazol-3-yl)benzonitrile (AHU-15)

To a stirred solution of the product from step 11 (AHU-14) (77 mg, 1 eq, 0.24 mmol) and the product from step 6 (AHU-7) (94 mg, 1.2 eq, 0.28 mmol) in 1,4-dioxane (4 mL) were added K₃PO₄ (151 mg, 3 eq, 0.71 mmol), Pd₂(dba)₃ (43 mg, 0.2 eq, 47 μmol) and XantPhos (55 mg, 0.4 eq, 95 mol) at room temperature under nitrogen atmosphere. The resulting mixture was stirred for 1 h at 100° C. under nitrogen atmosphere. The mixture was cooled to room temperature and concentrated under reduced pressure. The crude product was purified by reverse flash column chromatography with the following conditions: Column, C18; mobile phase, Water (0.1% NH₄HCO₃) and ACN (20% ACN up to 70% in 20 min); Detector, UV 254/220 nm. This resulted in the sub-title compound (AHU-15) (90 mg, 0.15 mmol, 61%, 92% Purity) as a yellow solid, m/z 619.2/621.2 (M+H)⁺ (ES+).

### Synthesis of (S)-3-(2-(6-((6-azaspiro[3.4]octan-6-yl)methyl)-3-oxo-8-(trifluoromethyl)imidazo [1,5-a]pyridin-2(3H)-yl)-6-((3-cyano-2-hydroxypropyl)amino)pyridin-4-yl)-4-(4-methyl-4H-1,2,4-triazol-3-yl)benzonitrile (I-50)

To a stirred solution of the product from step 12 (AHU-15) (40 mg, 1 eq, 65 μmol) and (3S)-4-amino-3-hydroxybutanenitrile (AHU-16) (9.7 mg, 1.5 eq, 98 mol) in 1,4-dioxane (3 mL) were added Cs₂CO₃ (105 mg, 5 eq, 0.33 mmol) and Pd-PEPPSI-IPentCl 2-methylpyridine (o-picoline) (16 mg, 0.3 eq, 19 μmol) at room temperature under nitrogen atmosphere. The resulting mixture was stirred for overnight at 100° C. under nitrogen atmosphere. The mixture was cooled to room temperature and concentrated under reduced pressure. The residue was purified by Prep-TLC with dichloromethane/methyl alcohol (8/1). The crude product was purified by Prep-HPLC with the following conditions (Column: XBridge Prep OBD C18 Column, 30* 150 mm, 5 μm; Mobile Phase A: Water (0.1% NH₄HCO₃), Mobile Phase B: ACN; Flow rate: 60 mL/min; Gradient: 50% B to 54% B in 7 min; Wave Length: 254 nm/220 nm; RT: 5.72). The fraction was collected and concentrated under vacuum, the residue was re-dissolved in CH3CN and H2O, and then was lyophilized to afford the title compound (1-50) (7.9 mg, 12 μmol, 18%, 99.4%) as a yellow solid, m/z 683.3 (M+H)⁺ (ES+). ¹H NMR (400 MHz, DMSO-d6) δ 8.52 (s, 1H), 8.12 (d, J=7.4 Hz, 2H), 7.89-7.82 (m, 1H), 7.63 (s, 1H), 7.43 (d, 1H), 7.31 (d, 1H), 7.12 (s, 1H), 7.02 (s, 1H), 6.30 (d, 1H), 5.60 (d, 1H), 4.00-3.91 (m, 1H), 3.40 (s, 3H), 3.36 (s, 2H), 3.31 (s, 2H), 2.77-2.65 (m, 1H), 2.71-2.67 (m, 1H), 2.53 (d, 2H), 2.53-2.50 (m, 2H), 1.95-1.87 (m, 4H), 1.87-1.74 (m, 4H).

### Example 15. 2-(3-((R)-Cyclobutyl(4-methyl-4H-1,2,4-triazol-3-yl) methyl)phenyl)-5-(((S)-3-methylpiperidin-1-yl)methyl)-7-(trifluoromethyl)benzo[d]oxazole (I-33)

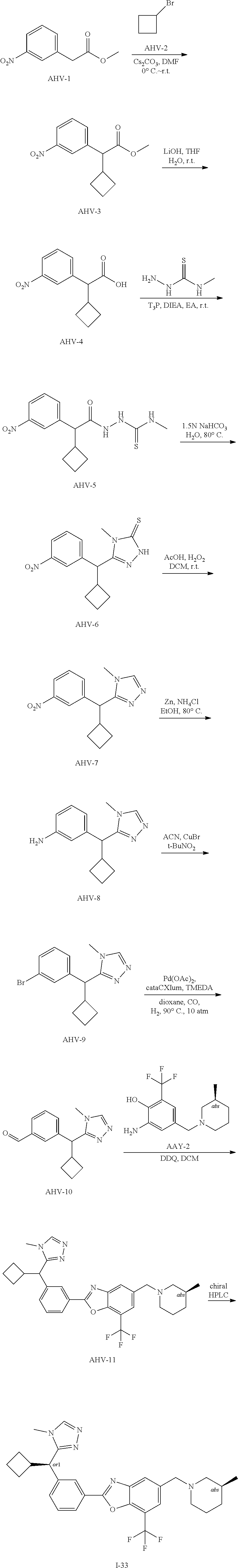

### Synthesis of methyl 2-cyclobutyl-2-(3-nitrophenyl)acetate (AHV-3)

A solution of methyl 2-(3-nitrophenyl)acetate (AHV-1) (10.0 g, 1 eq, 51.2 mmol) in DMF (100 mL) was treated with Cs₂CO₃ (83 g. 5 eq. 256 mmol) for 1 min at 0° C. under nitrogen atmosphere. The resulting mixture was stirred for 3 h at room temperature under nitrogen atmosphere. To the above mixture was added bromocyclobutane (AHV-2) (20.8 g, 3 eq, 154 mmol) dropwise over 1 min at room temperature. The resulting mixture was stirred for overnight at 60° C. The mixture was cooled to room temperature. The reaction was then quenched by the addition of 50 mL of sat, aq. NH4Cl at 0° C. The resulting mixture was diluted with water and extracted with ethyl acetate (3×500 mL). The combined organic layers were washed with brine (2×500 mL), dried over anhydrous sodium sulfate. After filtration, the filtrate was concentrated under reduced pressure. The residue was applied on a silica gel column chromatography with petroleum ether/ethyl acetate (2/1) to afford the sub-title compound (AHV-3) (8.50 g, 34.1 mmol, 63%, 83% Purity) as a yellow solid, m/z 250.1 (M+H)⁺ (ES+).

### Synthesis of 2-cyclobutyl-2-(3-nitrophenyl)acetic acid (AHV-4)

To a stirred solution of the product from step 1 (AHV-3) (7.30 g, 1 eq, 29.3 mmol) in THF (90 mL) and H₂O (30 mL) was added LiOH (3.51 g, 5 eq, 146 mmol) at room temperature under air atmosphere. The resulting mixture was stirred for 2 h at room temperature. The mixture was basified to pH 7 with citric acid at 0° C. The resulting mixture was diluted with water and extracted with ethyl acetate (3×500 mL). The combined organic layers were washed with brine (2×500 mL), dried over anhydrous sodium sulfate. After filtration, the filtrate was concentrated under reduced pressure. This resulted in the sub-title compound (AHV-4) (8.1 g, 34.5 mmol, crude, 65% Purity) as a yellow solid, m z 236.1 (M+H)⁺ (ES+).

### Synthesis of 2-(2-cyclobutyl-2-(3-nitrophenyl)acetyl)-N-methylhydrazine-1-carbothioamide (AHV-5)

To a stirred solution of the product from step 2 (AHV-4) (8.00 g, 1 eq, 34.0 mmol) and 4-methyl-3-thiosemicarbazide (7.15 g, 2 eq, 68.0 mmol) were added T₃P in EA (86.6 g, 50% Wt, 8 eq, 272 mmol) and DIPEA (26.4 g, 6 eq, 204 mmol) at room temperature. The resulting mixture was stirred for additional overnight at room temperature. The resulting mixture was concentrated under reduced pressure. The crude product was purified by reverse flash column chromatography with the following conditions: Column, C18: mobile phase. Water (0.1% NH₄HCO₃) and ACN (20% ACN up to 60% in 20 min); Detector, UV 254/220 nm. This resulted in the sub-title compound (AHV-5) (5.2 g, 16.1 mmol, 44%, 90% Purity) as a yellow solid, m/z 323.1 (M+H)⁺ (ES+).

### Synthesis of 5-(cyclobutyl(3-nitrophenyl)methyl)-4-methyl-2,4-dihydro-3H-1,2,4-triazole-3-thione (AHV-6)

To a stirred solution of the product from step 3 (AHV-5) (5.20 g, 1 eq, 16.1 mmol) in H₂O (120 mL) was added NaHCO₃ (36.5 g. 26.9 eq, 434 mmol) at room temperature. The resulting mixture was stirred for additional 3 h at 80° C. The mixture was cooled to room temperature and concentrated under reduced pressure. The resulting mixture was diluted with water and extracted with dichloromethane/methyl alcohol (10/1) (3×300 mL). The combined organic layers were washed with brine (2×300 mL), dried over anhydrous sodium sulfate. After filtration, the filtrate was concentrated under reduced pressure. This resulted in the sub-title compound (AHV-6) (8.80 g. 28.9 mmol, crude, 70% Purity) as a yellow solid, m/z 305.1 (M+H)⁺ (ES+).

### Synthesis of 3-(cyclobutyl(3-nitrophenyl)methyl)-4-methyl-4H-1,2,4-triazole (AHV-7)

To a stirred solution of the product from step 4 (AHV-6) (8.80 g, 1 eq, 28.9 mmol) in DCM (100 mL) were added AcOH (3.47 g, 2 eq, 57.8 mmol) and H₂O₂ (4.92 g, 30% Wt, 5 eq, 145 mmol) dropwise at 0° C. The resulting mixture was stirred for additional 2 h at room temperature. The resulting mixture was diluted with water and extracted with dichloromethane/methyl alcohol (10/1) (3×500 mL). The combined organic layers were washed with brine (2×500 mL), dried over anhydrous sodium sulfate. After filtration, the filtrate was concentrated under reduced pressure. This resulted in the sub-title compound (AHV-7) (8.1 g, 29.8 mmol, 95%, 80% Purity) as a yellow solid, m/z 273.1 (M+H)⁺ (ES+).

### Synthesis of 3-(cyclobutyl(4-methyl-4H-1,2,4-triazol-3-yl)methyl)aniline (AHV-8)

To a stirred solution of the product from step 7 (AHV-7) (600 mg, 1 eq, 2.20 mmol) and NH₄Cl (1.18 g, 10 eq, 22.0 mmol) in EtOH (20 mL) were added Zn (1.44 g, 10 eq, 22.0 mmol) in portions at room temperature. The resulting mixture was stirred for additional 4 h at 80° C. The mixture was cooled to room temperature. The resulting mixture was filtered; the filter cake was washed with MeOH (3×5 mL). The filtrate was concentrated under reduced pressure. The crude product was purified by reverse flash column chromatography with the following conditions: Column, C18; mobile phase, Water (0.1% NH₄HCO₃) and ACN (20% ACN up to 70% in 20 min); Detector, UV 254/220 nm. This resulted in the sub-title compound (AHV-8) (348 mg, 1.44 mmol, 59%, 92% Purity) as a yellow solid, m/z 243.2 (M+H)⁺ (ES+).

### Synthesis of 3-[(3-bromophenyl)(cyclobutyl)methyl]-4-methyl-1,2,4-triazole (AHV-9)

A solution of CuBr (107 mg, 1.5 eq, 0.74 mmol) and t-BuNO₂ (306 mg, 6 eq, 2.97 mmol) in ACN (8 mL) was stirred for 1 h at 0° C. under nitrogen atmosphere. To the above mixture was added the product from step 6 (AHV-8) (120 mg, 1 eq, 0.50 mmol) at 0° C. under nitrogen atmosphere. The resulting mixture was stirred for additional 1.5 h at 50° C. under nitrogen atmosphere. The mixture was allowed to cool down to room temperature. The resulting mixture was concentrated under reduced pressure. The crude product was purified by reverse flash column chromatography with the following conditions: Column. C18; mobile phase, Water (0.1% NH₄HCO₃) and ACN (30% ACN up to 80% in 30 min); Detector, UV 254/220 nm to afford the sub-title compound (AHV-9) (80 mg, 0.26 mmol, 53%, 92% Purity) as a light yellow solid, m/z 306.1/308.1 (M+H)⁺ (ES+).

### Synthesis of 3-[cyclobutyl(4-methyl-1,2,4-triazol-3-yl)methyl]benzaldehyde (AHV-10)

To a stirred mixture of the product from step 7 (AHV-9) (600 mg, 1 eq, 1.96 mmol) and TMEDA (683 mg, 3 eq, 5.88 mmol) in dioxane (25 mL) were added Pd(OAc)₂ (44 mg, 0.1 eq, 0.20 mmol) and cataCXium (281 mg, 0.4 eq, 0.78 mmol) at room temperature under nitrogen atmosphere. The resulting mixture was stirred for 1 day at 90° C. under carbon monoxide and hydrogen atmosphere. The mixture was allowed to cool down to room temperature. The resulting mixture was concentrated under vacuum. The crude product was purified by reverse flash column chromatography with the following conditions: Column, C18; mobile phase, Water (0.1% NH₄HCO₃) and ACN (10% ACN up to 50% in 15 min); Detector, UV 254/220 nm. This resulted in the sub-title compound (AHV-10) (70 mg, 0.27 mmol, 13%, 93% Purity) as a white solid, m/z 256.1 (M+H)⁺ (ES+).

### Synthesis of 2-{3-[cyclobutyl(4-methyl-1,2,4-triazol-3-yl)methyl]phenyl}-5-{[(3S)-3-methylpiperidin-1-yl]methyl}-7-(trifluoromethyl)-1,3-benzoxazole (AHV-11)

A solution of the product from step 8 (AHV-10) (110 mg, 1 eq, 0.43 mmol) in DCM (3 mL) was treated with 2-amino-4-{[(3S)-3-methylpiperidin-1-yl]methyl}-6-(trifluoromethyl) phenol (AAY-2) (137 mg, 1.1 eq, 0.47 mmol) for 1 h at 60° C. under nitrogen atmosphere. The mixture was cooled to room temperature. To the above mixture was added DDQ (196 mg, 2 eq, 0.86 mmol) at 0° C. under nitrogen atmosphere. The resulting mixture was stirred for 1 h at room temperature under nitrogen atmosphere. The resulting mixture was concentrated under vacuum. The crude product was purified by Prep-HPLC with the following conditions (Column: XBridge Prep OBD C18 Column, 30*150 mm, 5 μm: Mobile Phase A: Water (0.1% NH₄HCO₃), Mobile Phase B: ACN; Flow rate: 60 mL/min; Gradient: 69% B to 74% B in 8 min; Wave Length: 254 nm/220 nm; RT: 7.23) to afford the sub-title compound (AHV-11) (5.4 mg. 0.01 mmol. 2.4%, 99.4% Purity) as a white solid, m % z 524.3 (M+H)⁺ (ES+).

### Synthesis of 2-(3-((R)-cyclobutyl(4-methyl-4H-1,2,4-triazol-3-yl) methyl)phenyl)-5-(((S)-3-methylpiperidin-1-yl)methyl)-7-(trifluoromethyl)benzo[d]oxazole (I-33)

The product from step 9 (AHV-11) (30 mg, 1 eq, 0.06 mmol) was purified by Prep-CHIRAL-HPLC with the following conditions (Column: JW-CHIRAL ART Cellulose-SB, 20*250 mm, 5 m; Mobile Phase A: IPA-HPLC, Mobile Phase B: Hex (0.5% 2M NH3-MeOH)-HPLC; Flow rate: 20 mL/min; Gradient: 70% B to 70% B in 14 min; Wave Length: 220/254 nm; RT2 (min): 11.68). The second eluting isomer fraction was collected and concentrated under vacuum, the residue was re-dissolved in CH₃CN and H₂O, and then was lyophilized to afford the title compound (I-33) (12.4 mg, 24 μmol, 41%, 99.9% Purity) as a white solid with stereochemistry arbitrarily assigned, m/z 524.3 (M+H)⁻ (ES+). ¹H NMR (400 MHz, DMSO-d6) δ 8.36 (s, 1H), 8.11 (d, 1H), 8.08-8.03 (m, 1H), 8.02 (s, 1H), 7.69 (s, 1H), 7.65-7.55 (m, 2H), 4.43 (d, 1H), 3.63 (s, 2H), 3.45 (s, 3H), 3.27-3.13 (m, 1H), 2.73 (t, 2H), 2.17-2.03 (m, 1H), 1.98-1.87 (m, 1H), 1.87-1.70 (m, 5H), 1.69-1.55 (m, 4H), 1.54-1.43 (m, 1H), 0.93-0.77 (m, 4H). Column: CHIRALPAK IA-3, 4.6*50 mm, 3 μm; Mobile Phase: Hex(0.1% FA): EtOH=70:30, Flow rate: 1 mL/min; RT2:4.097.

### Example 16. 3-[2-(6-{5-Azaspiro[2.4]heptan-5-ylmethyl}-3-oxo-8-(trifluoromethyl)imidazo[1,5-a]pyridin-2-yl)-6-(but-3-yn-1-ylamino)pyridin-4-yl]-4-(4-methyl-1,2,4-triazol-3-yl)benzonitrile (I-22)

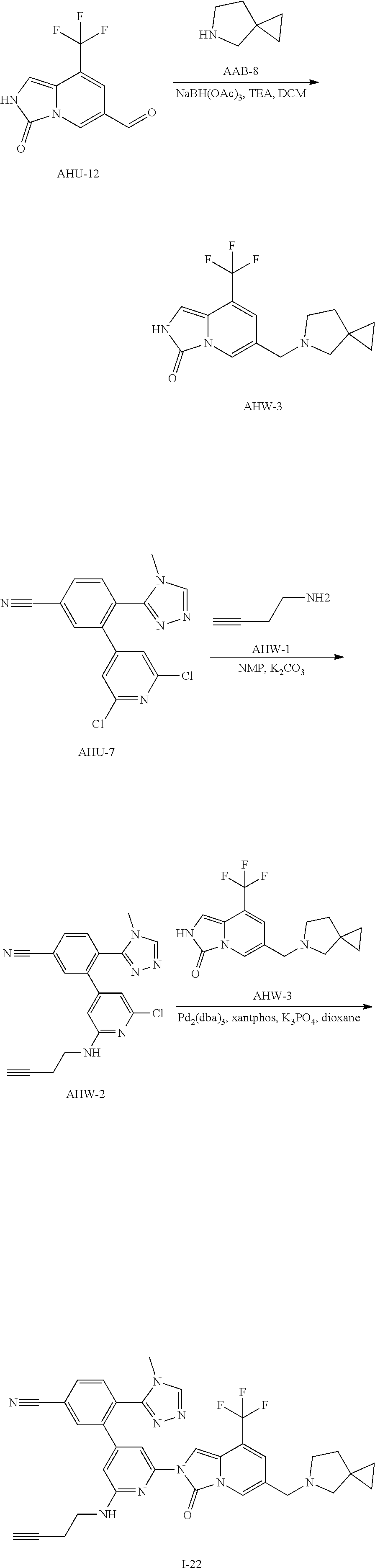

### Synthesis of 3-[2-(but-3-yn-1-ylamino)-6-chloropyridin-4-yl]-4-(4-methyl-1,2,4-triazol-3-yl)benzonitrile (AHW-2)

To a stirred mixture of 3-(2,6-dichloropyridin-4-yl)-4-(4-methyl-1,2,4-triazol-3-yl)benzonitrile (AHU-7) (200 mg. 1 eq. 0.61 mmol) and but-3-yn-1-amine (AHW-1) (209 mg, 5 eq, 3.03 mmol) in NMP (10 ml) was added K₂CO₃ (837 mg, 10 eq. 6.06 mmol) at room temperature. The resulting mixture was stirred for overnight at 100° C. The mixture was allowed to cool down to room temperature. The crude product was purified by reverse flash column chromatography with the following conditions: Column, C18; mobile phase, Water (0.1% NH₄HCO₃) and ACN (30% ACN up to 80% in 20 min); Detector, UV 254/220 nm. This resulted in the sub-title compound (AHW-2) (60 mg, 0.17 mmol, 26%, 95% Purity) as a white solid, m/z 363.1/365.1 (M+H)⁺ (ES+).

### Synthesis of 6-{5-azaspiro[2.4]heptan-5-ylmethyl}-8-(trifluoromethyl)-2H-imidazo[1,5-a]pyridin-3-one (AHW-3)

To a stirred solution of 3-oxo-8-(trifluoromethyl)-2,3-dihydroimidazo[1,5-a]pyridine-6-carbaldehyde (AHU-12) (116 mg, 1 eq, 0.50 mmol) and 5-azaspiro[2.4]heptane (AAB-8) (73 mg, 1.5 eq, 0.76 mmol) in DCM (8 mL) were added TEA (306 mg, 6 eq, 3.02 mmol) and NaBH(OAc)₃ (641 mg, 6 eq, 3.02 mmol) at 0° C. The resulting mixture was stirred for additional 1.5 h at room temperature. The reaction was then quenched by the addition of 2 mL of methyl alcohol at 0° C. The resulting mixture was concentrated under vacuum. The residue was purified by Prep-TLC with dichloromethane/methyl alcohol (10/1) to afford the sub-title compound (AHW-3) (136 mg. 0.44 mmol, 87%. 92% Purity) as a yellow oil, m/z 312.1 (M+H)⁺ (ES+).

### Synthesis of 3-[2-(6-{5-azaspiro[2.4]heptan-5-ylmethyl}-3-oxo-8-(trifluoromethyl)imidazo[1,5-a]pyridin-2-yl)-6-(but-3-yn-1-ylamino)pyridin-4-yl]-4-(4-methyl-1,2,4-triazol-3-yl)benzonitrile (I-22)

To a stirred mixture of the product from step 1 (AHW-2) (50 mg. 1 eq. 0.14 mmol) and the product from step 2 (AHW-3) (51 mg. 1.2 eq, 0.17 mmol) in 1,4-dioxane (3 mL) were added K₃PO₄ (88 mg, 3 eq, 0.41 mmol), Pd₂(dba)₃ (13 mg. 0.1 eq, 14 μmol) and XantPhos (16 mg, 0.2 eq, 28 mol) at room temperature under nitrogen atmosphere. The resulting mixture was stirred for overnight at 100° C. under nitrogen atmosphere. The mixture was allowed to cool down to room temperature. The resulting mixture was concentrated under reduced pressure. The residue was purified by Prep-TLC with dichloromethane/methyl alcohol (10/1). The crude product was purified by Prep-HPLC with the following conditions (Column: xBridge Prep Phenyl 5 tam OBD 19*250 mm: Mobile Phase A: Water (0.1% NH₄HCO₃), Mobile Phase B: ACN; Flow rate: 20 mL/min; Gradient: 45% B to 55% B in 9 min; Wave Length: 220/254 nm: RT: 7.6). The fraction was collected and concentrated under vacuum, the residue was re-dissolved in CH₃CN and H₂O, and then was lyophilized to afford the title compound (1-22) (4.1 mg, 6.4 μmol, 4.6%. 98.0% Purity) as a yellow solid, m/z 638.4 (M+H)⁺ (ES+). ¹H NMR (400 MHz, DMSO-d6) δ 8.52 (s, 1H), 8.12 (d, 2H), 7.85 (d, 1H), 7.65 (s, 1H), 7.42 (s, 1H), 7.29 (s, 1H), 7.23 (t, J=5.8 Hz, 1H), 7.06 (s, 1H), 6.27 (s, 1H), 3.44-3.34 (m, 7H), 2.85 (d, 1H), 2.68 (t, 2H), 2.47-2.39 (m, 4H), 1.75 (t, 2H), 0.51 (d, 4H).

### Example 17. 3-(2-(6-((5-Azaspiro[2.4]heptan-5-yl)methyl)-3-oxo-8-(trifluoromethyl)imidazo[1,5-a]pyridin-2(3H)-yl)-6-(((1-(cyanomethyl)cyclopropyl)methyl)amino)pyridin-4-yl)-4-(4-methyl-4H-1,2,4-triazol-3-yl)benzonitrile (1-19)

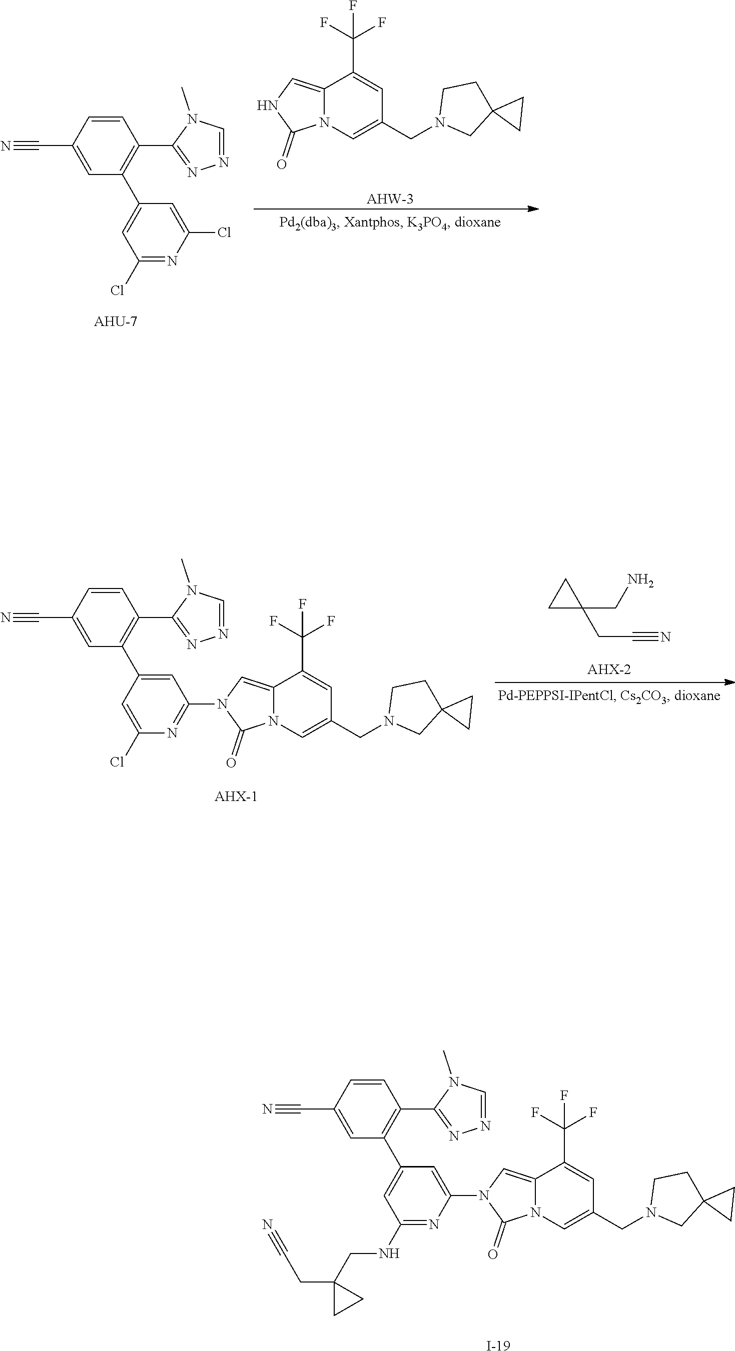

### Synthesis of 3-[2-(6-{5-azaspiro[2.4]heptan-5-ylmethyl}-3-oxo-8-(trifluoromethyl)imidazo[1,5-a]pyridin-2-yl)-6-chloropyridin-4-yl]-4-(4-methyl-1,2,4-triazol-3-yl)benzonitrile (AHX-1)

To a stirred solution of 6-{5-azaspiro[2.4]heptan-5-ylmethyl}-8-(trifluoromethyl)-2H-imidazo[1,5-a]pyridin-3-one (AHW-3) (89 mg, 1 eq, 0.29 mmol) and 3-(2,6-dichloropyridin-4-yl)-4-(4-methyl-1,2,4-triazol-3-yl)benzonitrile (AHU-7) (94 mg, 1 eq, 0.29 mmol) in dioxane (3.5 mL) were added K₃PO₄ (182 mg, 3 eq, 0.86 mmol), Pd₂(dba)₃ (26 mg, 0.1 eq, 29 μmol) and XantPhos (33 mg, 0.2 eq, 57 μmol) at room temperature under nitrogen atmosphere. The final reaction mixture was stirred for 1 h at 100° C. under nitrogen atmosphere. The mixture was cooled to room temperature and concentrated under reduced pressure. The crude product was purified by reverse flash column chromatography with the following conditions: Column, C18; mobile phase, Water (0.1% NH₄HCO₃) and ACN (30% ACN up to 80% in 20 min); Detector, UV 254/220 nm. This resulted in the sub-title compound (AHX-1) (93 mg, 0.15 mmol, 48%, 92% Purity) as a yellow solid, m/z 605.2/607.2 (M+H)⁺ (ES+).

### Synthesis of 3-(2-(6-((5-azaspiro[2.4]heptan-5-yl)methyl)-3-oxo-8-(trifluoromethyl)imidazo[1,5-a]pyridin-2(3H)-yl)-6-(((1-(cyanomethyl)cyclopropyl)methyl)amino)pyridin-4-yl)-4-(4-methyl-4H-1,2,4-triazol-3-yl)benzonitrile (I-19)

To a stirred solution of the product from step 1 (AHX-1) (40 mg, 0.066 mmol, 1 equiv) and 2-[1-(aminomethyl)cyclopropyl]acetonitrile hydrochloride (AHX-2) (29 mg, 3 eq, 0.20 mmol) in 1,4-dioxane (2 mL) and DMF (1 mL) were added Cs₂CO₃ (108 mg, 5 eq, 0.33 nmol) and Pd-PEPPSI-IPentCl 2-methylpyridine (o-picoline) (17 mg, 0.3 eq, 0.02 mmol) at room temperature under nitrogen atmosphere. The resulting mixture was stirred for 1 h at 100° C. under nitrogen atmosphere. The mixture was cooled to room temperature and concentrated under reduced pressure. The residue was purified by Prep-TLC with dichloromethane/methyl alcohol (10/1). The crude product was purified by Prep-HPLC with the following conditions (Column: XSelect CSH Prep C18 OBD Column, 19*250 mm, 5 μm; Mobile Phase A: Water (0.1% NH₄HCO₃), Mobile Phase B: MeOH; Flow rate: 25 mL/min; Gradient: 45% B to 50% B in 7 min; Wave Length: 254 nm; RT: 7). The fraction was collected and concentrated under vacuum, the residue was re-dissolved in CH₃CN and H₂O, and then was lyophilized to afford the title compound (1-19) (11.9 mg, 18 mol, 26%, 97.7% Purity) as a yellow solid, m/z 679.6 (M+H)⁻ (ES+). ¹H NMR (400 MHz, DMSO-d6) δ 8.51 (s, 1H), 8.14-8.08 (m, 2H), 7.84 (d, 1H), 7.64 (s, 1H), 7.45 (s, 1H), 7.30 (d, 1H), 7.19 (t, 1H), 7.04 (s, 1H), 6.26 (d, 1H), 3.38 (s, 3H), 3.31 (s, 2H), 2.67 (t, 2H), 2.61 (s, 2H), 2.50-2.49 (m, 2H), 2.45 (s, 2H), 1.74 (t, 2H), 0.65-0.59 (m, 2H), 0.56-0.39 (m, 6H).

### Example 18. 2-(3-((R)-(4-Methyl-4H-1,2,4-triazol-3-yl)((1s,3S)-3-(trifluoromethyl)cyclobutyl)methyl)phenyl)-5-(((S)-3-methyl piperidin-1-yl)methyl)-7-(trifluoromethyl)benzo[d]oxazole (1-59)

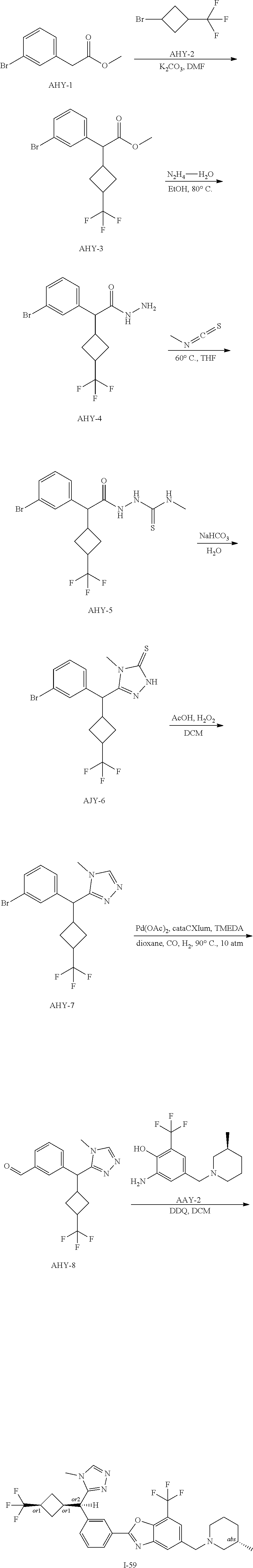

### Synthesis of methyl 2-(3-bromophenyl)-2-[3-(trifluoromethyl)cyclobutyl]acetate (AHY-3)

To a stirred solution of methyl 2-(3-bromophenyl)acetate (AHY-1) (200 mg, 1 eq, 0.87 mmol) and 1-bromo-3-(trifluoromethyl)cyclobutane (AHY-2) (266 mg, 1.5 eq, 1.31 mmol) in DMF (10 mL) was added Cs₂CO₃ (1.42 g, 5 eq, 4.37 mmol) at room temperature under nitrogen atmosphere. The resulting mixture was stirred for 12 h at 60° C. under nitrogen atmosphere. The mixture was allowed to cool down to room temperature. The resulting mixture was diluted with water and extracted with ethyl acetate (3×50 mL). The combined organic layers were washed with brine (2×50 mL), dried over anhydrous sodium sulfate. After filtration, the filtrate was concentrated under reduced pressure. The crude product was purified by reverse flash column chromatography with the following conditions: Column, C18; mobile phase, Water (0.1% NH₄HCO₃) and ACN (80% ACN up to 90% in 10 min); Detector, UV 254/220 nm. This resulted in the sub-title compound (AHY-3) (200 mg, 0.57 mmol, 65%, 92% Purity) as a colorless oil, m/z 351.0/353.0 (M+H)⁺ (ES+).

### Synthesis of 2-(3-bromophenyl)-2-(3-(trifluoromethyl)cyclobutyl)acetohydrazide (AHY-4)

To a stirred solution of the product from step 1 (AHY-3) (3.03 g, 1 eq, 8.64 mmol) in EtOH (50 mL) was added hydrazine hydrate (64% hydrazine) (21.6 g, 64% Wt) dropwise at 0° C., at room temperature. The resulting mixture was stirred for 3 h at 80° C. The mixture was cooled to room temperature and concentrated under reduced pressure. The crude product used directly in next step without any further purification, m/z 351.0/353.0 (M+H)⁺ (ES+).

### Synthesis of 2-(2-(3-bromophenyl)-2-(3-(trifluoromethyl)cyclobutyl)acetyl)-N-methylhydrazine-1-carbothioamide (AHY-5)

To a stirred solution of the product from step 2 (AHY-4) (3.04 g, 1 eq, 8.66 mmol) in THF (50 mL) was added methyl isothiocyanate (1.58 g, 2.5 eq, 21.6 mmol) at room temperature and stirred at 60° C. for overnight. The mixture was cooled to room temperature, diluted with water and extracted with ethyl acetate (3×200 mL). The combined organic layers were washed with brine (2×200 mL), dried over anhydrous sodium sulfate. After filtration, the filtrate was concentrated under reduced pressure, m/z 424.0/426.0 (M+H)⁺ (ES+).

### Synthesis of 5-((3-bromophenyl)(3-(trifluoromethyl)cyclobutyl)methyl)-4-methyl-2,4-dihydro-3H-1,2,4-triazole-3-thione (AHY-6)

A solution of the product from step 3 (AHY-5) (3.04 g, 1 eq. 7.17 mmol) and NaHCO₃ (4.20 g, 7 eq. 50.0 mmol) in H₂O (50 mL) was stirred for 2 h at 80° C. The mixture was cooled to room temperature, diluted with water and extracted with ethyl acetate (3×200 mL). The combined organic layers were washed with brine (2×200 mL), dried over anhydrous sodium sulfate. After filtration, the filtrate was concentrated under reduced pressure. The crude product used directly in next step without any further purification, m/z 406.0/408.0 (M+H)⁺ (ES+).

### Synthesis of 3-((3-bromophenyl)(3-(trifluoromethyl)cyclobutyl)methyl)-4-methyl-4H-1,2,4-triazole (AHY-7)

To a stirred solution of the product from step 4 (AHY-6) (3.80 g, 1 eq, 9.35 mmol) and AcOH (1.69 g, 3 eq, 28.1 mmol) in DCM (50 mL) was added H₂O₂ (3.18 g, 30% Wt, 5 eq, 46.8 mmol) dropwise at 0° C., and stirred for 10 min, then the mixture was stirred at room temperature for 2 h. The resulting mixture was diluted with water and extracted with ethyl acetate (3×200 mL). The combined organic layers were washed with brine (2×200 mL), dried over anhydrous sodium sulfate. After filtration, the filtrate was concentrated under reduced pressure. The residue was applied on a silica gel column chromatography with dichloromethane/methyl (10/1) to afford the sub-title compound (AHY-7) (2.65 g, 7.10 mmol, 76%, 90% Purity) as a yellow solid, m/z 374.0/376.0 (M+H)⁺ (ES+).

### Synthesis of 3-((4-methyl-4H-1,2,4-triazol-3-yl)(3-(trifluoromethyl)cyclobutyl)methyl) benzaldehyde (AHY-8)

To a stirred solution of the product from step 5 (AHY-7) (500 mg, 1 eq, 1.34 mmol) and TMEDA (465 mg, 3 eq, 4.01 mmol) in 1,4-dioxane (10 mL) were added bis(adamantan-1-yl)(butyl)phosphane (192 mg, 0.4 eq, 0.53 mmol) and Pd(OAc)₂ (30 mg, 0.1 eq, 0.13 mmol) at room temperature and stirred at 90° C. for overnight under carbon monoxide atmosphere and hydrogen atmosphere. The mixture was cooled to room temperature and concentrated under reduced pressure. The crude product was purified by reverse flash column chromatography with the following conditions: Column, C18; mobile phase, Water (0.1% NH₄HCO₃) and ACN (10% ACN up to 60% in 10 min); Detector, UV 254/220 nm to afford the sub-title compound (AHY-8) (116 mg, 0.36 mmol, 27%, 92% Purity) as a yellow solid, m/z 324.1 (M+H)⁺ (ES+).

### Synthesis of 2-(3-((R)-(4-methyl-4H-1,2,4-triazol-3-yl)((1s,3S)-3-(trifluoromethyl)cyclobutyl) methyl)phenyl)-5-(((S)-3-methylpiperidin-1-yl)methyl)-7-(trifluoromethyl)benzo[d]oxazole (I-59)

To a stirred solution of the product from step 6 (AHY-8) (75 mg, 1 eq, 0.23 mmol) in DCM (5 mL) was added 2-amino-4-{[(3S)-3-methylpiperidin-1-yl]methyl}-6-(trifluoromethyl)phenol (AAY-2) (134 mg, 2 eq, 0.46 mmol) at room temperature and stirred at 60° C. for 1 h. The mixture was allowed to cool down to 0° C., then the DDQ (105 mg, 2 eq, 0.46 mmol) was added into the mixture in portions and stirred at room temperature for 1 h. The resulting mixture was concentrated under reduced pressure. The crude product was purified by reverse flash column chromatography with the following conditions: Column, C18: mobile phase. Water (0.1% NH₄HCO₃) and ACN (50% ACN up to 75% in 20 min); Detector, UV 254/220 nm to afford a crude product. The crude product was re-purified by Prep-HPLC and Chiral-Prep-HPLC with the following conditions (Column: JW-CHIRAL ART Amylose-SB 20*250 mm, 5 μm; Mobile Phase A: IPA: DCM=1: 1-HPLC, Mobile Phase B: Hex (0.5% 2M NH3-MeOH)-HPLC; Flow rate: 20 mL/min; Gradient: 80% B to 80% B in 29 min; Wave Length: 220/254 nm; RT2 (min): 12.34). The second eluting isomer fraction was collected and concentrated under vacuum, the residue was re-dissolved in CH₃CN and H₂O, and then was lyophilized to afford the title compound (1-59) (13.6 mg, 23 mol, 9.8%, 99.3% Purity) as a white solid with stereochemistry arbitrarily assigned, m/z 592.2 (M+H)⁺ (ES+). ¹H NMR (400 MHz, DMSO-d6) δ 8.39 (s, 1H), 8.12 (d, 1H), 8.10-8.06 (m, 1H), 8.02 (s, 1H), 7.70 (s, 1H), 7.66-7.57 (m, 2H), 4.60 (d, 1H), 3.64 (s, 2H), 3.45 (s, 3H), 3.31-3.13 (m, 2H), 2.74 (s, 2H), 2.28 (d, 1H), 2.16-1.88 (m, 4H), 1.63 (t, 4H), 1.50 (t, 1H), 0.82 (d, 4H).

The following compounds in Table 3 were prepared according to methods analogous to those mentioned above.

TABLE 3
Characterization Data of Further Compounds of the Invention

| | | LCMS | |
|---|---|---|---|
| I-# | Structure | Data | ¹H NMR Data (400 MHz) |
| I-1 | 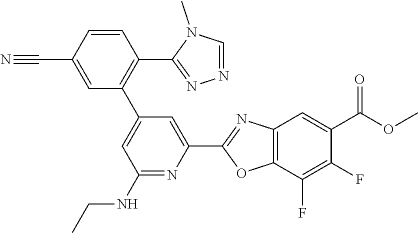 [SMILES: CCNc1cc(-c2cc(C#N)ccc2-c2nncn2C)cc(-c2nc3cc(C(=O)OC)c(F)c(F)c3o2)n1] | m/z 516.0 (M + H)⁺ (ES+) | (Methanol-d4) δ 8.48 (s, 1H), 8.17-8.01 (m, 2H), 7.85 (d, 1H), 7.69-7.63 (m, 1H), 7.34 (d, 1H), 6.43 (d, , 1H), 4.00 (d, 3H), 3.38-3.33 (m, 5H), 1.24- 1.22 (m, 3H). |
| I-2 | 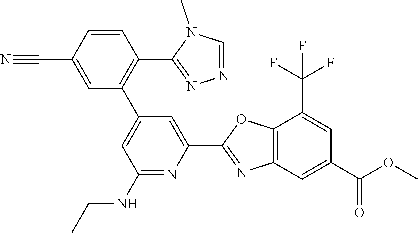 [SMILES: CCNc1cc(-c2cc(C#N)ccc2-c2nncn2C)cc(-c2nc3cc(C(=O)OC)cc(C(F)(F)F)c3o2)n1] | m/z 548.0 (M + H)⁺ (ES+) | (DMSO-d₆) δ 8.66-8.63 (m, 1H), 8.52 (s, 1H), 8.30-8.23 (m, 2H), 8.17-8.12 (m, 1H), 7.87 (d, 1H), 7.28 (d, 1H), 7.16 (t, 1H), 6.45 (d, 1H), 3.96 (s, 3H), 3.32 (s, 3H), 3.30-3.24 (m, 2H), 1.16 (t, 3H). |
| I-3 | 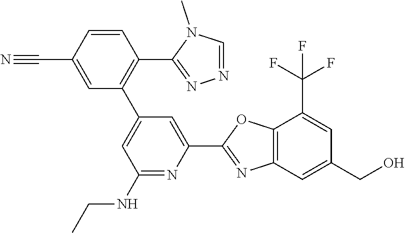 [SMILES: CCNc1cc(-c2cc(C#N)ccc2-c2nncn2C)cc(-c2nc3cc(CO)cc(C(F)(F)F)c3o2)n1] | m/z 520.1 (M + H)⁺ (ES+) | (DMSO-d₆) δ 8.52 (s, 1H), 8.24 (d, 1H), 8.16-8.11 (m, 1H), 8.05 (s, 1H), 7.86 (d, 1H), 7.76 (s, 1H), 7.26 (d, 1H), 7.10 (t, 1H), 6.41 (d, 1H), 5.55 (t, 1H), 4.70 (d, 2H), 3.31-3.26 (m, 5H), 1.16 (t, 3H). |
| I-4 | 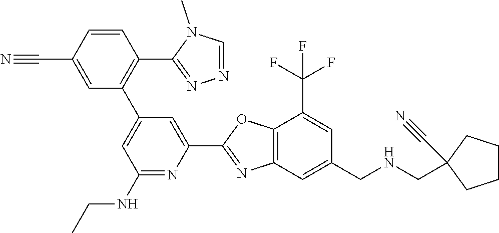 [SMILES: CCNc1cc(-c2cc(C#N)ccc2-c2nncn2C)cc(-c2nc3cc(CNCC4(C#N)CCCC4)cc(C(F)(F)F)c3o2)n1] | m/z 626.1 (M + H)⁺ (ES+) | (DMSO-d₆) δ 8.51 (s, 1H), 8.24 (d, 1H), 8.16-8.10 (m, 2H), 7.88-7.83 (m, 2H), 7.24 (d, 1H), 7.10 (t, 1H), 6.41 (d, 1H), 3.97 (s, 2H), 3.30 (s, 3H), 3.28- 3.22 (m, 2H), 2.64 (s, 2H), 2.00-1.91 (m, 2H), 1.77-1.59 (m, 6H), 1.15 (t, 3H). |
| I-7 | 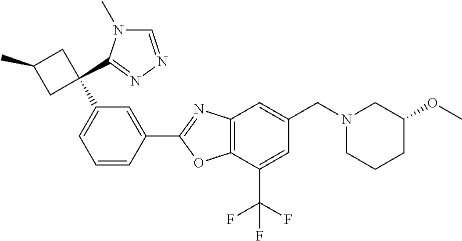 | m/z 540.5 (M + H)⁺ (ES+) | (Chloroform-d) δ 8.45 (t, 1H), 8.21-8.15 (m, 1H), 8.00 (s, 1H), 7.98-7.92 (m, 1H), 7.62 (s, 1H), 7.54 (t, 1H), 7.45 (d, 1H), 3.71 (s, 2H), 3.36 (d, 4H), 3.23 (s, 3H), 2.97 (t, 3H), 2.73 (t, 4H), 2.08 (d, 3H), 1.78 (s, 2H), 1.28 (s, 1H), 1.20 (d, 3H). |
| I-8 | 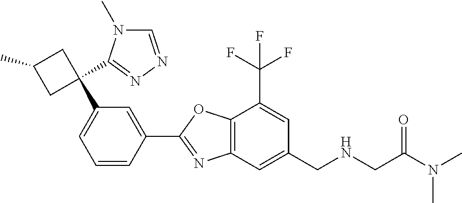 | m/z 527.4 (M + H)⁺ (ES+) | (Methanol-d4) δ 8.35 (d, 1H), 8.31 (d, 1H), 8.23-8.18 (m, 1H), 8.04 (s, 1H), 7.78 (s, 1H), 7.71-7.65 (m, 2H), 3.98 (s, 2H), 3.53 (s, 2H), 3.34 (s, 3H), 3.06-2.99 (m, 5H), 2.97 (d, 3H), 2.77-2.64 (m, 3H), 1.25- 1.15 (m, 3H). |
| I-13 | 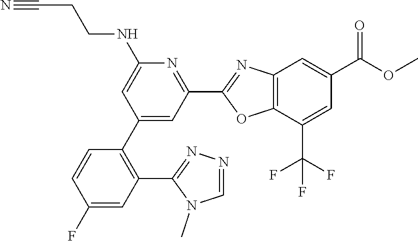 [SMILES: COC(=O)c1cc(C(F)(F)F)c2oc(-c3cc(-c4ccc(F)cc4-c4nncn4C)cc(NCCC#N)n3)nc2c1] | m/z 566.2 (M + H)⁺ (ES+) | (DMSO-d6) δ 8.65 (d, 1H), 8.49 (s, 1H), 8.27 (s, 1H), 7.81- 7.74 (m, 1H), 7.67-7.56 (m, 2H), 7.51 (t, 1H), 7.25 (d, 1H), 6.54 (d, 1H), 3.95 (s, 3H), 3.62- 3.53 (m, 2H), 3.35 (s, 3H), 2.87 (t, 2H). |
| I-14 | 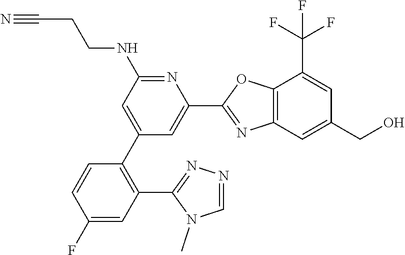 [SMILES: Cn1cnnc1-c1cc(F)ccc1-c1cc(NCCC#N)nc(-c2nc3cc(CO)cc(C(F)(F)F)c3o2)c1] | m/z 538.2 (M + H)⁺ (ES+) | (DMSO-d6) δ 8.49 (s, 1H), 8.06 (s, 1H), 7.82-7.74 (m, 2H), 7.71-7.54 (m, 2H), 7.47 (d, 1H), 7.23 (d, 1H), 6.51 (d, 1H), 5.54 (t, 1H), 4.70 (d, 2H), 3.64- 3.49 (m, 2H), 3.34 (s, 3H), 2.87 (t, 2H). |
| I-20 | 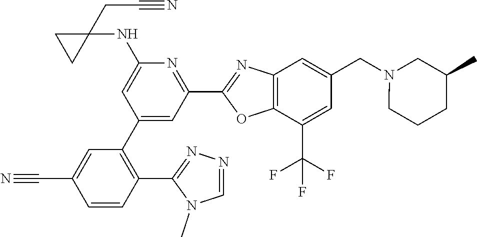 [SMILES: C[C@H]1CCCN(Cc2cc(C(F)(F)F)c3oc(-c4cc(-c5cc(C#N)ccc5-c5nncn5C)cc(NC5(CC#N)CC5)n4)nc3c2)C1] | m/z 652.3 (M + H)⁺ (ES+) | (DMSO-d₆) δ 8.54 (s, 1H), 8.29 (d, 1H), 8.19-8.13 (m, 1H), 8.05 (s, 1H), 7.93-7.86 (m, 2H), 7.74 (s, 1H), 7.44 (s, 1H), 6.57 (s, 1H), 3.65 (s, 2H), 3.31 (s, 3H), 2.93 (s, 2H), 2.74 (t, , 2H), 2.00-1.89 (m, 1H), 1.72- 1.56 (m, 4H), 1.55-1.43 (m, 1H), 0.90-0.85 (m, 3H), 0.83 (d, 3H), 0.77 (d, 2H). |
| I-21 | 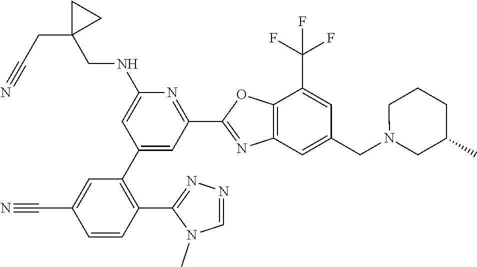 [SMILES: C[C@H]1CCCN(Cc2cc(C(F)(F)F)c3oc(-c4cc(-c5cc(C#N)ccc5-c5nncn5C)cc(NCC5(CC#N)CC5)n4)nc3c2)C1] | m/z 666.3 (M + H)⁺ (ES+) | (DMSO-d₆) δ 8.51 (s, 1H), 8.23 (d, 1H), 8.18-8.12 (m, 1H), 8.03 (s, 1H), 7.87 (d, 1H), 7.72 (s, 1H), 7.38 (t, 1H), 7.25 (d, 1H), 6.52 (d, 1H), 3.64 (s, 2H), 3.36 (s, 2H), 3.31 (s, 3H), 2.73 (d, 2H), 2.68 (s, 2H), 1.98- 1.90 (m, 1H), 1.72-1.56 (m, 4H), 1.55-1.43 (m, 1H), 0.92- 0.81 (m, 4H), 0.80-0.74 (m, 2H), 0.58-0.51 (m, 2H). |
| I-24 | 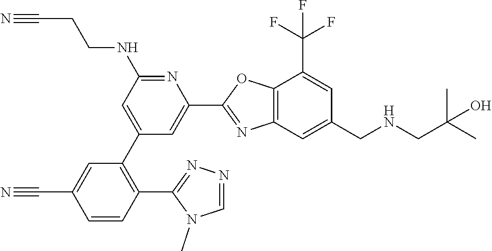 | m/z 589.3 (M − H)− (ES−) | (400 MHz, DMSO-d6) δ 8.45 (s, 1H), 8.10 (s, 1H), 7.81 (s, 1H), 7.75-7.64 (m, 4H), 7.46 (t, J = 5.6 Hz, 1H), 7.24 (d, 1H), 6.54 (s, 1H), 4.22 (s, 1H), 3.93 (s, 2H), 3.59-3.54 (m, 2H), 3.28 (s, 3H), 2.86 (t, 2H), 2.38 (s, 2H), 1.11 (s, 6H). |
| I-27 | 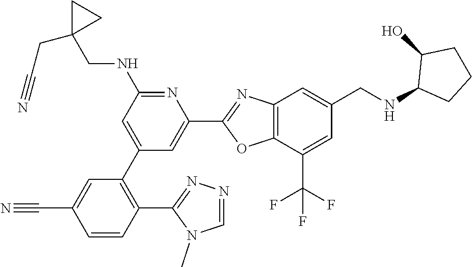 [SMILES: Cn1cnnc1-c1ccc(C#N)cc1-c1cc(NCC2(CC#N)CC2)nc(-c2nc3cc(CN[C@@H]4CCC[C@@H]4O)cc(C(F)(F)F)c3o2)c1] | m/z 668.3 (M + H)⁺ (ES+) | (400 MHz, DMSO-d6) δ 8.51 (s, 1H), 8.23 (s, 1H), 8.16- 8.07 (m, 2H), 7.87 (d, J = 8.0 Hz, 1H), 7.82 (s, 1H), 7.38 (t, 1H), 7.25 (s, 1H), 6.52 (s, 1H), 4.02-3.79 (m, 4H), 3.36 (d, 2H), 3.31 (s, 3H), 2.84-2.74 (m, 1H), 2.68 (s, 2H), 1.75- 1.54 (m, 4H), 1.48-1.36 (m, 2H), 0.77 (d, 2H), 0.54 (d, 2H). |
| I-29 | 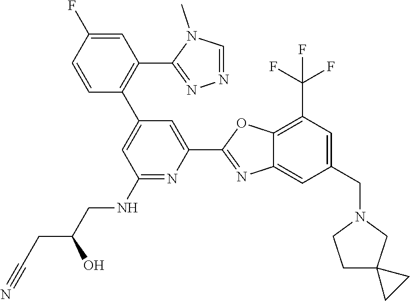 [SMILES: Cn1cnnc1-c1cc(F)ccc1-c1cc(NC[C@@H](O)CC#N)nc(-c2nc3cc(CN4CCC5(CC5)C4)cc(C(F)(F)F)c3o2)c1] | m/z 647.6 (M + H)⁺ (ES+) | (400 MHz, DMSO-d₆) δ 8.48 (s, 1H), 8.05 (s, 1H), 7.75 (d, 2H), 7.67-7.52 (m, 2H), 7.27- 7.12 (m, 2H), 6.53 (s, 1H), 5.68 (d, 1H), 4.05-3.95 (m, 1H), 3.78 (s, 2H), 3.35 (s, 5H), 2.80 -2.61 (m, 4H), 2.46 (s, 2H), 1.77 (t, J 2H), 0.51 (d, 4H). |
| I-34 | 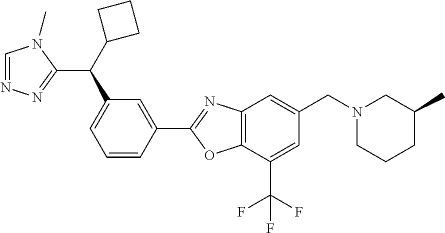 [SMILES: C[C@H]1CCCN(Cc2cc(C(F)(F)F)c3oc(-c4cccc([C@@H](c5nncn5C)C5CCC5)c4)nc3c2)C1] | m/z 524.3 (M + H)⁺ (ES+) | (400 MHz, DMSO-d₆) δ 8.36 (s, 1H), 8.11 (d, 1H), 8.08- 8.04 (m, 1H), 8.02 (s, 1H), 7.69 (s, 1H), 7.64-7.56 (m, 2H), 4.43 (d, 1H), 3.63 (s, 2H), 3.45 (s, 3H), 3.25-3.15 (m, 1H), 2.73 (t, 2H), 2.15-2.05 (m, 1H), 1.98-1.88 (m, 1H), 1.87- 1.70 (m, 5H), 1.70-1.56 (m, 4H), 1.54-1.42 (m, 1H), 0.91- 0.79 (m, 4H). |
| I-48 | 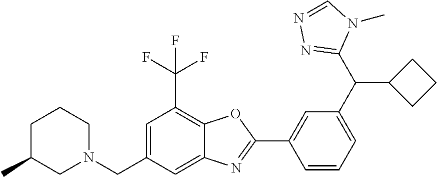 [SMILES: C[C@H]1CCCN(Cc2cc(C(F)(F)F)c3oc(-c4cccc(C(c5nncn5C)C5CCC5)c4)nc3c2)C1] | m/z 524.6 (M + H)⁺ (ES+) | (400 MHz, DMSO-d6) δ 8.36 (s, 1H), 8.10 (d, 1H), 8.08- 8.03 (m, 1H), 8.01 (s, 1H), 7.69 (s, 1H), 7.64-7.56 (m, 2H), 4.42 (d, 1H), 3.63 (s, 2H), 3.45 (s, 3H), 3.25-3.13 (m, 1H), 2.73 (t, 2H), 2.16-2.04 (m, 1H), 1.97-1.88 (m, 1H), 1.86- 1.55 (m, 9H), 1.53-1.43 (m, 1H), 0.90-0.78 (m, 4H). |
| I-49 | 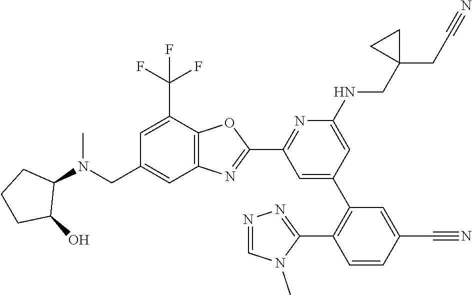 [SMILES: CN(Cc1cc(C(F)(F)F)c2oc(-c3cc(-c4cc(C#N)ccc4-c4nncn4C)cc(NCC4(CC#N)CC4)n3)nc2c1)[C@@H]1CCC[C@@H]1O] | m/z 682.2 (M + H)⁺ (ES+) | (400 MHz, DMSO-d6) δ 8.51 (s, 1H), 8.23 (d, 1H), 8.18- 8.11 (m, 2H), 7.87 (d, 1H), 7.81 (s, 1H), 7.39 (t, 1H), 7.23 (d, 1H), 6.54 (d, 1H), 4.13 (d, 1H), 4.02 (s, 1H), 3.82 (d, 1H), 3.69 (d, 1H), 3.36 (d, 2H), 3.31 (s, 3H), 2.68 (s, 2H), 2.50 (s, 1H), 2.12 (s, 3H), 1.92-1.49 (m, 6H), 0.78 (s, 2H), 0.58-0.51 (m, 2H). |
| I-50 | 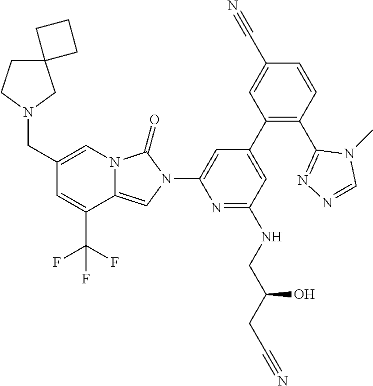 [SMILES: Cn1cnnc1-c1ccc(C#N)cc1-c1cc(NC[C@@H](O)CC#N)nc(-n2cc3c(C(F)(F)F)cc(CN4CCC5(CCC5)C4)cn3c2=O)c1] | m/z 683.3 (M + H)⁺ (ES+) | (400 MHz, DMSO-d6) δ 8.52 (s, 1H), 8.12 (d, 2H), 7.89- 7.82 (m, 1H), 7.63 (s, 1H), 7.43 (d, 1H), 7.31 (d, 1H), 7.12 (s, 1H), 7.02 (s, 1H), 6.30 (d, 1H), 5.60 (d, 1H), 4.00-3.91 (m, 1H), 3.40 (s, 3H), 3.36 (s, 2H), 3.31 (s, 2H), 2.77-2.65 (m, 1H), 2.71-2.67 (m, 1H), 2.53 (d, 2H), 2.53-2.50 (m, 2H), 1.95-1.87 (m, 4H), 1.87-1.74 (m, 4H). |
| I-51 | 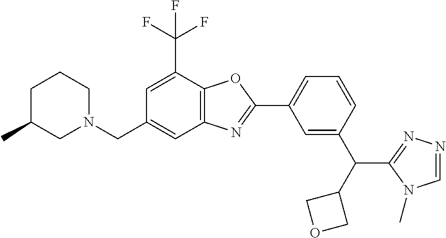 [SMILES: C[C@H]1CCCN(Cc2cc(C(F)(F)F)c3oc(-c4cccc(C(c5nncn5C)C5COC5)c4)nc3c2)C1] | m/z 526.2 (M + H)⁺ (ES+) | (400 MHz, DMSO-d6) δ 8.41 (s, 1H), 8.14-8.05 (m, 2H), 8.02 (s, 1H), 7.69 (s, 1H), 7.67- 7.54 (m, 2H), 4.99 (d, 1H), 4.79-4.73 (m, 1H), 4.55-4.43 (m, 2H), 4.31 (t, 1H), 3.97- 3.84 (m, 1H), 3.64 (s, 2H), 3.41 (s, 3H), 2.72 (d, 2H), 1.98- 1.88 (m, 1H), 1.70-1.54 (m, 4H), 1.54-1.42 (m, 1H), 0.92- 0.77 (m, 4H) |
| I-52 | 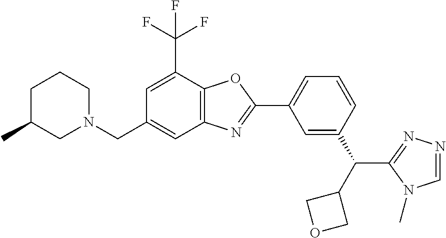 [SMILES: C[C@H]1CCCN(Cc2cc(C(F)(F)F)c3oc(-c4cccc([C@H](c5nncn5C)C5COC5)c4)nc3c2)C1] | m/z 526.3 (M + H)⁺ (ES+) | (400 MHz, DMSO-d6) δ 8.41 (s, 1H), 8.14-8.05 (m, 2H), 8.02 (s, 1H), 7.69 (s, 1H), 7.67- 7.54 (m, 2H), 4.99 (d, 1H), 4.79-4.73 (m, 1H), 4.55-4.43 (m, 2H), 4.31 (t, 1H), 3.97- 3.84 (m, 1H), 3.64 (s, 2H), 3.41 (s, 3H), 2.72 (d, 2H), 1.98- 1.88 (m, 1H), 1.71-1.42 (m, 5H), 0.92-0.77 (m, 4H). |
| I-53 | 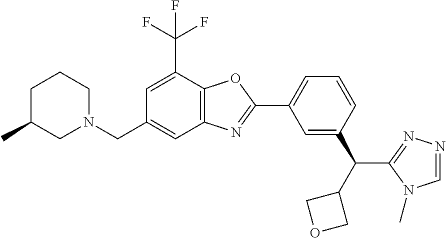 [SMILES: C[C@H]1CCCN(Cc2cc(C(F)(F)F)c3oc(-c4cccc([C@@H](c5nncn5C)C5COC5)c4)nc3c2)C1] | m/z 526.3 (M + H)⁺ (ES+) | (400 MHz, DMSO-d6) δ 8.41 (s, 1H), 8.14-8.05 (m, 2H), 8.03 (s, 1H), 7.70 (s, 1H), 7.68- 7.55 (m, 2H), 4.99 (d, 1H), 4.79-4.73 (m, 1H), 4.55-4.45 (m, 2H), 4.31 (t, 1H), 3.96- 3.84 (m, 1H), 3.64 (s, 2H), 3.41 (s, 3H), 2.74 (s, 2H), 1.94 (s, 1H), 1.72-1.43 (m, 5H), 0.82 (d, 4H). |
| I-56 | 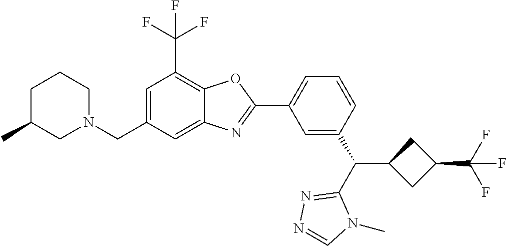 [SMILES: C[C@H]1CCCN(Cc2cc(C(F)(F)F)c3oc(-c4cccc([C@@H](c5nncn5C)[C@H]5C[C@@H](C(F)(F)F)C5)c4)nc3c2)C1] | m/z 592.4 (M + H)⁺ (ES+) | (400 MHz, DMSO-d6) δ 8.39 (s, 1H), 8.13-8.05 (m, 2H), 8.03 (s, 1H), 7.70 (s, 1H), 7.67- 7.55 (m, 2H), 4.45 (d, 1H), 3.64 (s, 2H), 3.42 (s, 3H), 3.26- 3.14 (m, 1H), 3.13-3.00 (m, 1H), 2.73 (d, 2H), 2.37-2.27 (m, 1H), 2.00-1.80 (m, 4H), 1.71-1.56 (m, 4H), 1.49 (d, 1H), 0.83 (d, 4H) |
| I-57 | 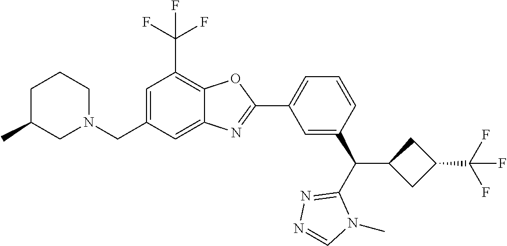 [SMILES: C[C@H]1CCCN(Cc2cc(C(F)(F)F)c3oc(-c4cccc([C@H](c5nncn5C)[C@H]5C[C@H](C(F)(F)F)C5)c4)nc3c2)C1] | m/z 592.3 (M + H)⁺ (ES+) | (400 MHz, DMSO-d6) δ 8.39 (s, 1H), 8.12 (t, 1H), 8.10-8.06 (m, 1H), 8.02 (s, 1H), 7.69 (s, 1H), 7.66-7.57 (m, 2H), 4.60 (d, 1H), 3.64 (s, 2H), 3.45 (s, 3H), 3.31-3.10 (m, 2H), 2.80- 2.69 (m, 2H), 2.28 (d, 1H), 2.17-1.87 (m, 4H), 1.75-1.54 (m, 4H), 1.50 (t, 1H), 0.82 (d, 4H). |
| I-58 | 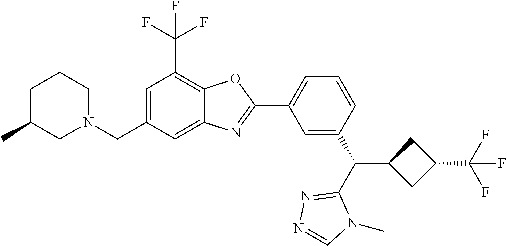 [SMILES: C[C@H]1CCCN(Cc2cc(C(F)(F)F)c3oc(-c4cccc([C@@H](c5nncn5C)[C@H]5C[C@H](C(F)(F)F)C5)c4)nc3c2)C1] | m/z 592.2 (M + H)⁺ (ES+) | (400 MHz, DMSO-d6) δ 8.39 (s, 1H), 8.17-7.97 (m, 3H), 7.76-7.53 (m, 3H), 4.45 (d, 1H), 3.64 (s, 2H), 3.42 (s, 3H), 3.25-3.01 (m, 2H), 2.74 (s, 2H), 2.39-2.26 (m, 1H), 2.04- 1.80 (m, 4H), 1.62 (d, 4H), 1.49 (d, 1H), 0.82 (d, 4H). |
| I-60 | 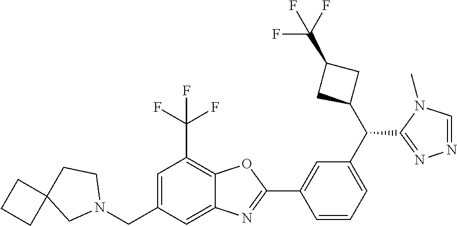 [SMILES: Cn1cnnc1[C@H](c1cccc(-c2nc3cc(CN4CCC5(CCC5)C4)cc(C(F)(F)F)c3o2)c1)[C@H]1C[C@@H](C(F)(F)F)C1] | m/z 604.3 (M + H)⁺ (ES+) | (400 MHz, DMSO-d6) δ 8.39 (s, 1H), 8.11-8.07 (m, 2H), 8.03 (s, 1H), 7.70 (s, 1H), 7.67- 7.55 (m, 2H), 4.45 (d, 1H), 3.75 (s, 2H), 3.42 (s, 3H), 3.25- 3.18 (m, 1H), 3.15-3.07 (m, 1H), 2.55 (d, 4H), 2.38-2.26 (m, 1H), 2.00-1.68 (m, 11H). |
| I-61 | 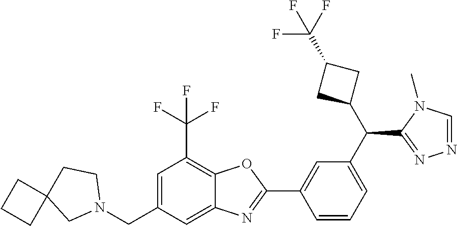 [SMILES: Cn1cnnc1[C@@H](c1cccc(-c2nc3cc(CN4CCC5(CCC5)C4)cc(C(F)(F)F)c3o2)c1)[C@H]1C[C@H](C(F)(F)F)C1] | m/z 604.4 (M + H)⁺ (ES+) | (400 MHz, DMSO-d6) δ 8.39 (s, 1H), 8.12 (d, 1H), 8.11- 8.07 (m, 1H), 8.02 (s, 1H), 7.70 (s, 1H), 7.66-7.57 (m, 2H), 4.60 (d, 1H), 3.75 (s, 2H), 3.45 (s, 3H), 3.31-3.11 (m, 2H), 2.55 (d, 4H), 2.30 (s, 1H), 2.16- 1.71 (m, 11H). |
| I-62 | 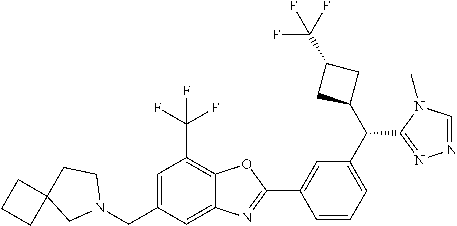 [SMILES: Cn1cnnc1[C@H](c1cccc(-c2nc3cc(CN4CCC5(CCC5)C4)cc(C(F)(F)F)c3o2)c1)[C@H]1C[C@H](C(F)(F)F)C1] | m/z 604.3 (M + H)⁺ (ES+) | (400 MHz, DMSO-d6) δ 8.39 (s, 1H), 8.16-8.05 (m, 2H), 8.03 (s, 1H), 7.70 (s, 1H), 7.67- 7.55 (m, 2H), 4.45 (d, 1H), 3.75 (s, 2H), 3.42 (s, 3H), 3.24- 3.13 (m, 1H), 3.11-3.01 (m, 1H), 2.55 (d, 4H), 2.38-2.26 (m, 1H), 2.01-1.67 (m, 11H). |
| I-63 | 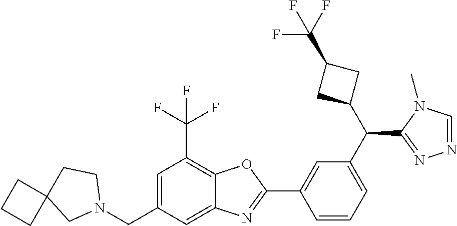 [SMILES: Cn1cnnc1[C@@H](c1cccc(-c2nc3cc(CN4CCC5(CCC5)C4)cc(C(F)(F)F)c3o2)c1)[C@H]1C[C@@H](C(F)(F)F)C1] | m/z 604.3 (M + H)⁺ (ES+) | (400 MHz, DMSO-d6) δ 8.39 (s, 1H), 8.13 (d, 1H), 8.10- 8.05 (m, 1H), 8.02 (s, 1H), 7.70 (s, 1H), 7.67-7.56 (m, 2H), 4.60 (d, 1H), 3.75 (s, 2H), 3.45 (s, 3H), 3.33-3.13 (m, 2H), 2.55 (d, 4H), 2.36-2.26 (m, 1H), 2.17-1.72 (m, 11H). |

### Example 19: Biochemical Assays

### Cbl-b Biochemical Assay (TR-FRET)

Recombinant human Cbl-b (aa 36-427) was expressed in E coli, purified and biotinylated in vitro. The protein was diluted to 12 nM in freshly prepared assay buffer consisting of 50 mM HEPES, pH 7.0, 100 mM NaCl, 5 mM MgCl2, 0.01% Triton-X 100, 0.01% BSA and 1 mM DTT. Recombinant human Src (aa 254-536)-GSSGSS-Zap-70 (aa 281-297) fusion protein was expressed in E coli and purified. Protein was diluted in assay buffer to 2-5 nM and supplemented with ATP to 1 mM. Fluorescein-BODIPY labeled UBED2D(C85K)-Ub was prepared by conjugating ubiquitin (Ub) labeled at its N-terminus with Fluorescein-BODIPY maleimide (ThermoFisher Catalog no B10250) to E coli expressed and purified UBED2D(C85K) [see Dou et al Nature Structural and Molecular Biology 8: 982-987, 2013]. Recombinant human UBE2D2(C85K) was expressed in E coli, purified and ubiquitinated and Bodipy labelled in vitro. Protein was diluted to 200 nM in assay buffer without MgCl₂ (or Cisbio PPI buffer). Streptavidin-Terbium was added to 2 nM and EDTA to 10 mM, to provide a binding assay mix. Compounds were dissolved in DMSO and diluted to prepare a ten-point dilution series. 100 nl of each compound concentration was dispensed in duplicate in a 384 well black assay plate using acoustic dispensing. Wells for maximum signal controls received 100 nl of DMSO only and wells for minimum signal controls received 100 nl of a reference inhibitor compound at a final assay concentration of 100 mM to produce 100% inhibition. 5 μl of diluted Cbl-b enzyme was added to all wells of the assay plate and incubated at RT for 30-60 min. The enzyme assay was initiated by addition of 5 μl of Src-Zap/ATP mix to all wells, and the plate incubated at RT for 60 min. The enzyme reaction was terminated and the binding reaction was initiated by adding 10p of binding assay mix to all wells and incubating the plate at RT for 60 min prior to assay read. Final assay conditions consisted of 6 nM Cbl-b, 1-2.5 nM Src-Zap70, 0.5 mM ATP, 1% (v/v) DMSO (enzyme reaction) and 100 nM UBE2D2(C85K)-Ub-FL-BODIPY, 5 mM EDTA, 1 nM Streptavidin-Tb (binding reaction). The HTRF assay signal was measured at 520 nm on an Envision plate reader, with reference signal at 485 or 620 nm. Data was normalized using maximum and minimum assay controls: % Inhibition=100−(100*((maximum control)−unknown)/(maximum control−minimum control)). A 4-parameter dose-response equation was used to fit the normalized dose-response data and derive an IC50 for test compounds.

### c-Cbl Biochemical Assay (TR-FRET)

Recombinant human c-Cbl (aa 47-435) was expressed in E coli, purified and biotinylated in vitro. The protein was diluted to 12 nM in freshly prepared assay buffer consisting of 50 mM HEPES, pH 7.0, 100 mM NaCl, 5 mM MgCl₂, 0.01% Triton-X 100, 0.01% BSA and 1 mM DTT. Recombinant human Src (aa 254-536)-GSSGSS-Zap-70 (aa 281-297) fusion protein was expressed in E coli and purified. Protein was diluted in assay buffer to 5-20 nM and ATP was added to 1 mM. Recombinant human UBE2D2(C85K) was expressed in E coli, purified and ubiquitinated and Bodipy labelled in vitro. Protein was diluted to 200 nM in assay buffer without MgCl₂ (or Cisbio PPI buffer). Streptavidin-Terbium was added to 2 nM and EDTA to 10 mM, to provide a binding assay mix. Compounds were dissolved in DMSO and diluted to prepare a ten-point half log dilution series. 100 nl of each compound concentration was dispensed in duplicate in a 384 well black assay plate using acoustic dispensing. Wells for maximum signal controls received 100 nl of DMSO only and wells for minimum controls received 100 nl of a reference inhibitor compound at a final assay concentration of 100 mM to produce 100% inhibition. 5 μl of diluted c-Cbl enzyme was added to all wells of the assay plate and incubated at RT for 30 min. The enzyme assay was initiated by addition of 5 d of Src-Zap/ATP mix to all wells, and the plate incubated at RT for 60-90 min. The enzyme reaction was terminated and the binding reaction was initiated by adding 10p of binding assay mix to all wells and incubating the plate at RT for 60 min prior to assay read. Final assay conditions consisted of 6 nM c-Cbl. 2.5-10 nM Src-Zap70, 0.5 mM ATP. 1% (v/v) DMSO (enzyme reaction) and 100 nM UBE2D2(C85K)-Ub-FL-BODIPY, 5 mM EDTA, 1 nM Streptavidin-Tb (binding reaction). The HTRF assay signal was measured at 520 nm on an Envision plate reader, with reference signal at 485 or 620 nm. Data was normalized using maximum and minimum assay controls: % Inhibition=100−(100*((maximum control)−unknown)/(maximum control−minimum control)). A 4-parameter dose-response equation was used to fit the normalized dose-response data and derive an IC₅₀ for test compounds.

Table 4 shows the ICₒ of selected compounds of this invention in the TR-FRET biochemical assays. Compound activity designations have the following values: A (<0.2 μM); B (0.2 to 5 μM); and C (>5 μM).

TABLE 4
Cbl-b and c-Cbl Biochemical Assay (TR-FRET) Results

| I-# | TR-FRET Cbl-b IC₅₀ | Cbl-b/c-Cbl Selectivity |
|---|---|---|
| I-4 | B | 8 |
| I-5 | B | 12 |
| I-6 | B | 10 |
| I-7 | B | 12 |
| I-8 | B | 9 |
| I-9 | A | 18 |
| I-10 | A | 10 |
| I-12 | A | 7 |
| I-14 | B | 7 |
| I-15 | A | 7 |
| I-16 | B | 7 |
| I-24 | B | 6 |
| I-27 | A | 1 |
| I-29 | A | 14 |
| I-33 | A | 10 |
| I-34 | C | 4 |
| I-48 | A | 9 |
| I-49 | A | 3 |
| I-50 | A | 21 |
| I-51 | B | 7 |
| I-52 | B | 7 |
| I-53 | C | 1 |
| I-54 | C | 7 |
| I-55 | A | 11 |
| I-56 | C | 5 |
| I-57 | C | 2 |
| I-58 | C | 4 |
| I-59 | A | 19 |
| I-60 | C | 1 |
| I-61 | C | 0 |
| I-62 | C | 2 |
| 1-63 | B | 13 |

While we have described a number of embodiments of this invention, it is apparent that our examples may be altered to provide other embodiments that utilize the compounds and methods of this invention. Therefore, it will be appreciated that the scope of this invention is to be defined by the appended claims rather than by the specific embodiments that have been represented by way of example.

1. A compound of formula I: 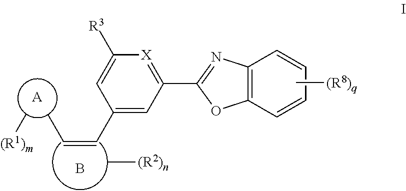
or a pharmaceutically acceptable salt thereof, wherein:
Ring A is a 5 membered heteroaryl ring having 1-3 nitrogen and 0-1 oxygen or sulfur;
R¹ is hydrogen, halogen, —CN, —OR, or an optionally substituted C₁₋₆ aliphatic:
Ring B is a divalent phenyl, or a divalent 5-6 membered heteroaryl ring having 1-4 heteroatoms independently selected from nitrogen, oxygen, and sulfur;
each R² is independently hydrogen, halogen, —CN, —CH₂OR, —CH(OR)R, —CRF₂, —CF₃, —OR, —SR, —NR₂, —SO₂R, —SO₂NR₂, —S(O)R, —C(O)R, —C(O)OR, —C(O)NR₂, —OC(O)R, —OC(O)NR₂, —NRC(O)OR, —NRC(O)R, —NRSO₂R; or an optionally substituted C₁₋₆ aliphatic or C4-6 heterocycloalkyl;
X is CH or N;
R³ is fluoro, bromo, iodo, —CN, —OR^{3D}, —SR^{3A}, —N(R^{3A})(R^{3E}), —S(O)₂R^{3A}, —S(O)₂N(R^{3A})₂, —S(O)R^{3A}, —S(O)N(R^{3A})₂, —C(O)R^{3A}, —C(O)OR^{3A}, —C(O)N(R^{3A})₂, —C(O)N(R^{3A})OR^{3A}, —OC(O)RWA, —OC(O)N(R^{3A})₂, —N(R^{3A})C(O)OR^{3A}, —N(R^{3A})C(O)R^{3A}, —N(R^{3A})C(O)N(R^{3A})₂, —N(R^{3A})C(NR^{3A})R^{3A}, —N(R^{3A})C(NR^{3A})N(R^{3A})₂, —N(R^{3A})N(R^{3A})₂, —N(R^{3A})S(O)₂N(R^{3A})₂, —N(R^{3A})S(O)₂R^{3A}, —N═S(O)(R^{3A})₂, —S(NR^{3A})(O)R^{3A}, —N(R^{3A})S(O)R^{3A}, —N(R^{3A})CN, —P(O)(OR^{3A})₂, —P(O)(R^{3A})₂, or an optionally substituted group selected from C₁₋₆ alkyl, alkenyl, or alkynyl; phenyl; a 4-7 membered saturated or partially unsaturated monocyclic carbocyclic ring; a 5-6 membered monocyclic heteroaryl ring having 1-4 heteroatoms independently selected from nitrogen, oxygen, and sulfur; a 4-8 membered saturated or partially unsaturated monocyclic heterocyclic ring having 1-2 heteroatoms independently selected from nitrogen, oxygen, and sulfur; and a 6-10 membered saturated or partially unsaturated spirocyclic ring having 0-3 heteroatoms independently selected from nitrogen, oxygen, and sulfur;
each R^{3A} are independently hydrogen, —CN, halogen, or an optionally substituted group selected from C₁₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic carbocyclic ring having 1-2 heteroatoms independently selected from nitrogen, oxygen, and sulfur; or:
two R^{3A} groups on the same atom are optionally taken together with the atom to form an optionally substituted ring selected from a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 7-10 membered saturated or partially unsaturated bicyclic heterocyclic ring having 1-4 heteroatoms independently selected from nitrogen, oxygen, phosphorus, and sulfur; a 5-8 membered saturated or partially unsaturated bridged bicyclic ring having 0-3 heteroatoms independently selected from nitrogen, oxygen, phosphorus, and sulfur; and a 6-10 membered saturated or partially unsaturated spirocyclic ring having 0-3 heteroatoms independently selected from nitrogen, oxygen, phosphorus, and sulfur;
R^{3B} is hydrogen, halogen, —CN, —OR^{3C}, —SR, —N(R^{3A})(R^{3A}), —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —NRC(O)OR, —NRC(O)R, —NRC(O)NR₂·—NRC(NR)NR₂, —NRNR₂, —NRS(O)₂NR₂, —NRS(O)₂R, —N═S(O)R₂, —S(NR)(O)R, —NRS(O)R, —NRCN, —P(O)R₂, —P(O)(OR)₂; or an optionally substituted group selected from C₁₋₆ aliphatic; a phenyl ring; a 4-7 membered saturated or partially unsaturated monocyclic carbocyclic ring; a 5-6 membered monocyclic heteroaryl ring having 1-4 heteroatoms independently selected from nitrogen, oxygen, and sulfur; a 4-8 membered saturated or partially unsaturated monocyclic heterocyclic ring having 1-2 heteroatoms independently selected from nitrogen, oxygen, and sulfur; and a 7-10 membered partially unsaturated or heteroaromatic bicyclic heterocyclic ring having 1-4 heteroatoms independently selected from nitrogen, oxygen, and sulfur;
each R^{3C} are independently an optionally substituted group selected from C₁₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic carbocyclic ring having 1-2 heteroatoms independently selected from nitrogen, oxygen, and sulfur;
R^{3D} is hydrogen, a substituted ethyl, or an optionally substituted group selected from methyl or C₃₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic carbocyclic ring having 1-2 heteroatoms independently selected from nitrogen, oxygen, and sulfur;
R^{3E} is a C₂ aliphatic substituted with v instances of R^{3B}, a substituted 2-hydroxyethyl, or an optionally substituted group selected from methyl or C₃₋₆ aliphatic: phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; and a 6-10 membered saturated or partially unsaturated spirocyclic ring having 0-3 heteroatoms independently selected from nitrogen, oxygen, and sulfur; a 6-11 membered saturated or partially unsaturated bicyclic carbocyclic ring having 1-2 heteroatoms independently selected from nitrogen, oxygen, and sulfur; or:
R^{3A} and R^{3E} groups on the same nitrogen are optionally taken together with the nitrogen to form an optionally substituted 3-7 membered saturated or partially unsaturated monocyclic heterocyclic ring having 0-2 heteroatoms, in addition to the nitrogen from which R^{3A} and R^{3E} are attached, independently selected from nitrogen, oxygen, and sulfur; a 5-8 membered saturated or partially unsaturated bridged bicyclic ring having 0-2 heteroatoms, in addition to the nitrogen from which R^{3A} and R^{3E} are attached, independently selected from nitrogen, oxygen, and sulfur; and a 6-10 membered saturated or partially unsaturated spirocyclic ring having 0-2 heteroatoms, in addition to the nitrogen from which R^{3A} and R^{3E} are attached, independently selected from nitrogen, oxygen, and sulfur;
each R⁸ is independently hydrogen, oxo, halogen, —CN, —NO₂, —CHF₂, —CF₃, —OR, —(OCH₂CH₂)₁₋₁₀NR₂, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —NRC(O)OR, —NRC(O)R, —NRC(O)NR₂, —NRC(NR)NR₂, —NRNR₂, —NRS(O)₂NR₂, —NRS(O)₂R, —N═S(O)R₂, —S(NR)(O)R, —NRS(O)R, —NRCN, —P(O)R₂, —P(O)(OR)₂; —CH₂NR(CH₂CH₂O)₁₋₁₀CH₂CH₂NR₂; or an optionally substituted C₁₋₆aliphatic;
each R is independently hydrogen, or an optionally substituted group selected from C₁₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic carbocyclic ring having 1-2 heteroatoms independently selected from nitrogen, oxygen, and sulfur; or:
two R groups on the same atom are optionally taken together with the atom to form an optionally substituted 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorus, and sulfur;
m is 0, 1, or 2;
n is 0, 1, 2, or 3;
q is 0, 1, 2, or 3; and
each instance of v is independently 0, 1, 2, 3, 4, or 5.

2. The compound of claim 1, wherein Ring A is

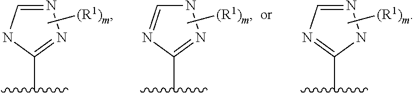

3. The compound of claim 1 or 2, wherein R¹ is an optionally substituted C₁₋₆ aliphatic.

4. The compound of any one of claims 1-3, wherein Ring A and its R⁸ substituents is

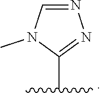 [SMILES: *c1nncn1C]

5. The compound of any one of claims 1-4, wherein Ring B is selected from

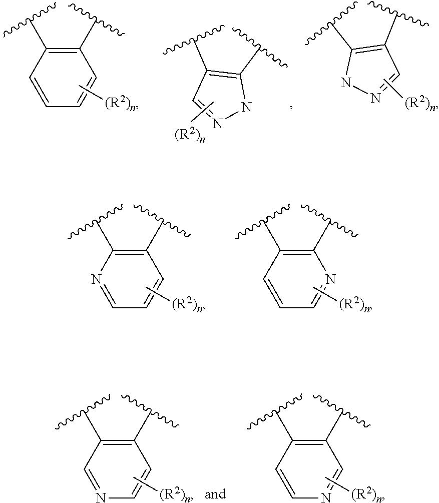

6. The compound of any one of claims 1-5, wherein R² is selected from halogen, —CN, —CH₂OR, —CH(OR)R, —OR, —NR₂, —C(O)R, —C(O)OR, —C(O)NR₂, —NRSO₂R; or an optionally substituted C₁₋₄ aliphatic.

7. The compound of any one of claims 1-6, wherein n is 1 and R² is choro, —CN, methyl, —CH₂OH, —CH₂OMe, —OMe, —CONH₂, —C(O)Me, —CH(OH)Me, —CO₂Me, or —NHSO₂Me.

8. The compound of any one of claims 1-7, wherein Ring B and its R² substituents are selected from

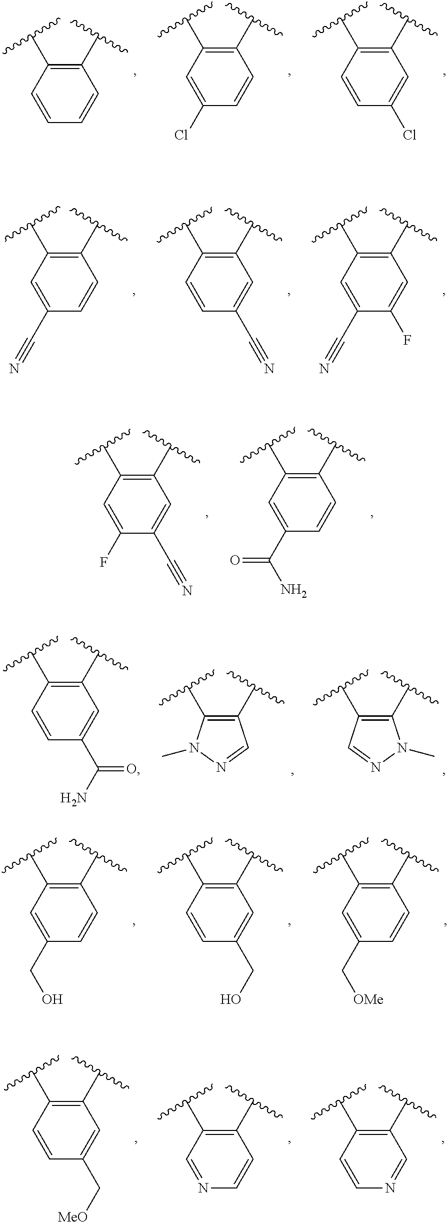

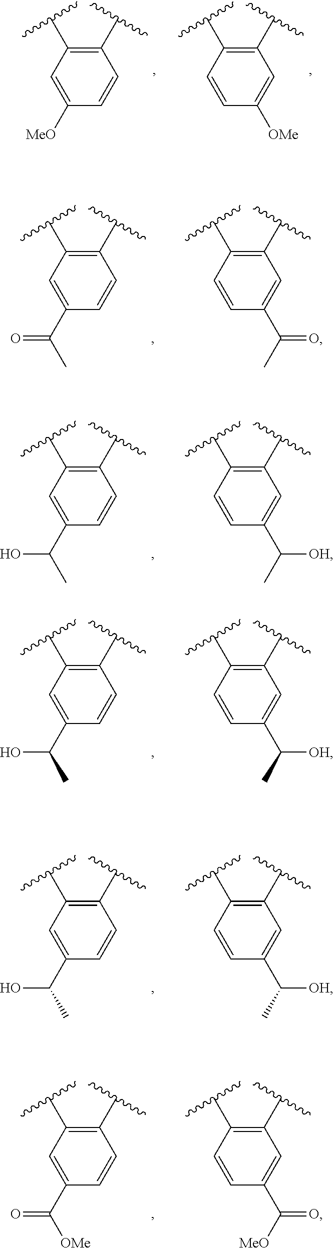

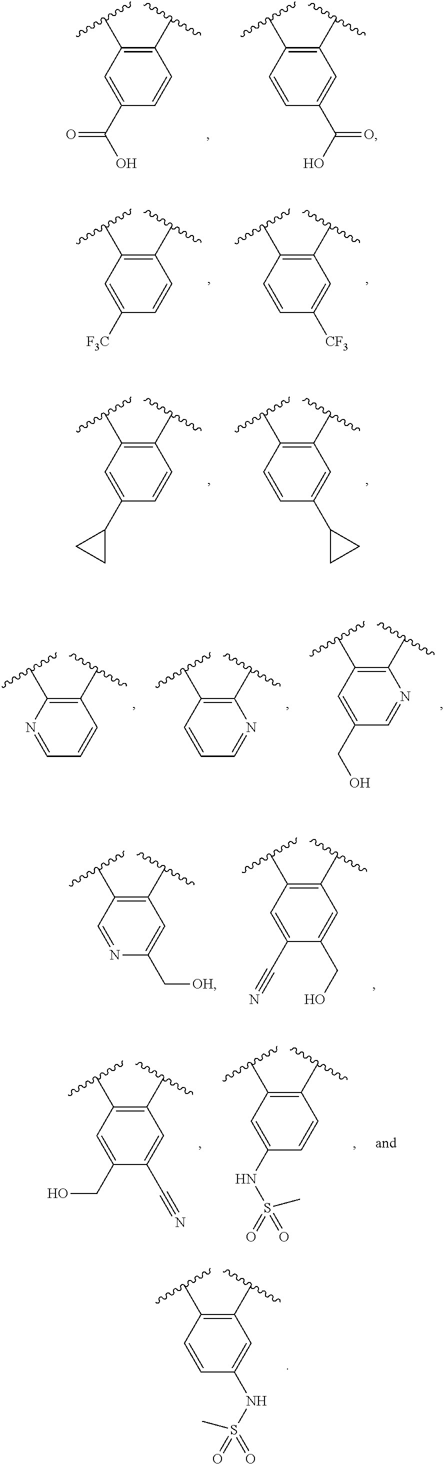

9. The compound of any one of claims 1-8, wherein Ring B and its R² substituents are selected from

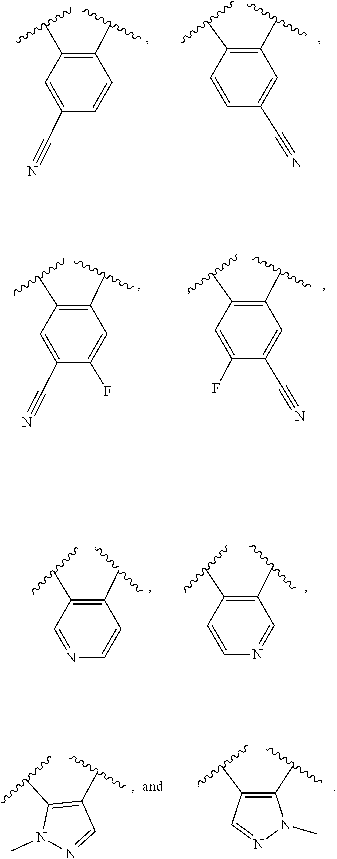

10. The compound of any one of claim 1-9, wherein R³ is —CN, —OR^{3A}, —N(R^{3A})(R^{3E}), —C(O)OR^{3A}, —C(O)N(R^{3A})OR^{3A}, —N(R^{3A})C(O)OR^{3A}, —N(R^{3A})C(O)R^{3A}, —N(R^{3A})C(O)N(R^{3A})₂, —N(R^{3A})S(O)₂R^{3A}, or an optionally substituted group selected from C₁₋₆ aliphatic: a 4-8 membered saturated or partially unsaturated monocyclic heterocyclic ring having 1-2 heteroatoms independently selected from nitrogen, oxygen, and sulfur; and a 6-10 membered saturated or partially unsaturated spirocyclic ring having 0-3 heteroatoms independently selected from nitrogen, oxygen, and sulfur.

11. The compound of any one of claims 1-10, wherein R³ is —CN, isopentyl, 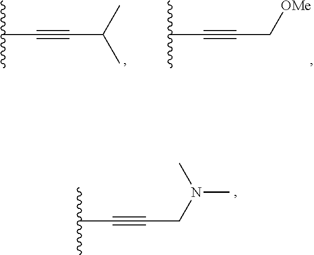
—CH₂OMe, —CH₂NHCOMe, —CH₂NHSO₂Me, 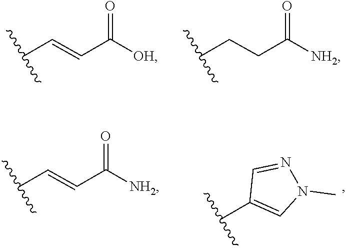
—CO₂H, —CO₂Me, —CON(Me)OMe, 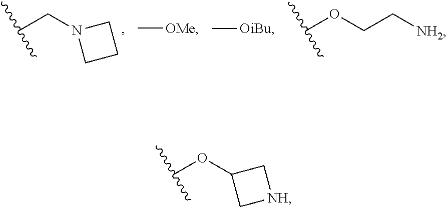
—NHnPr, —NHiBu, —NHCOiPr, —NHC(NH)iPr, —NHCO₂Me, —NHCONHMe, —NHCONMe₂, —NHSO₂Et, 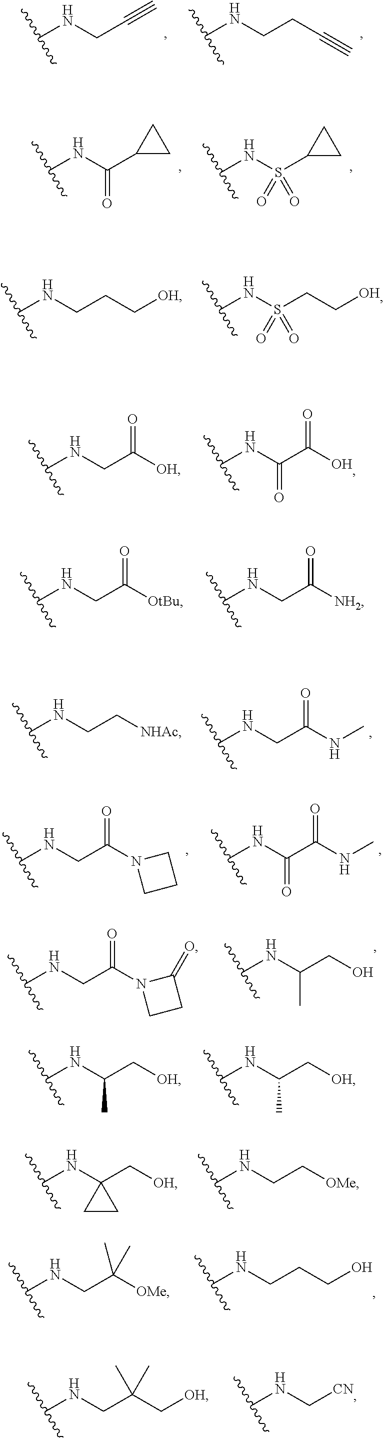 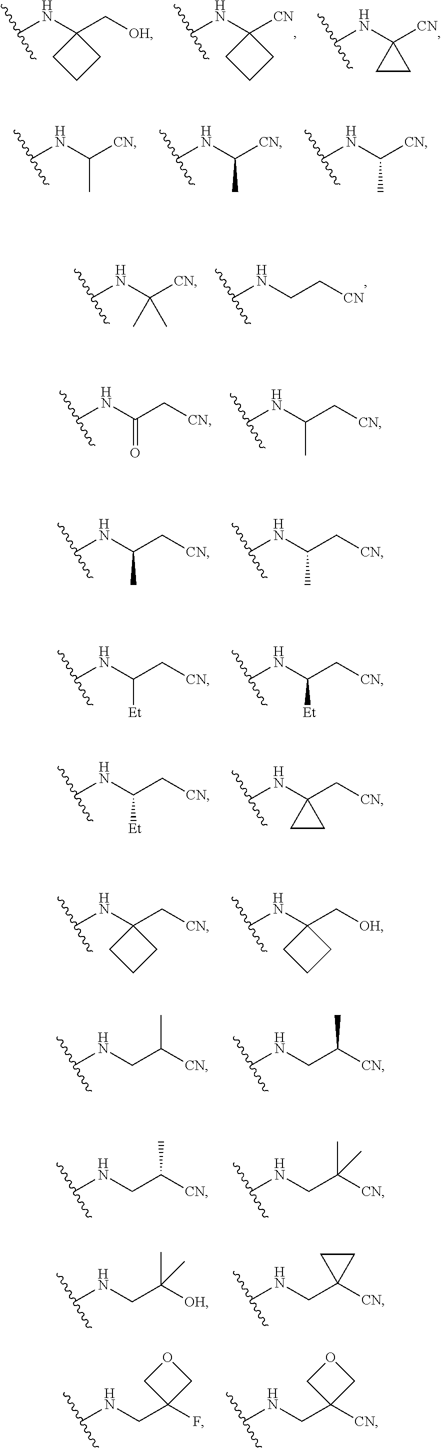 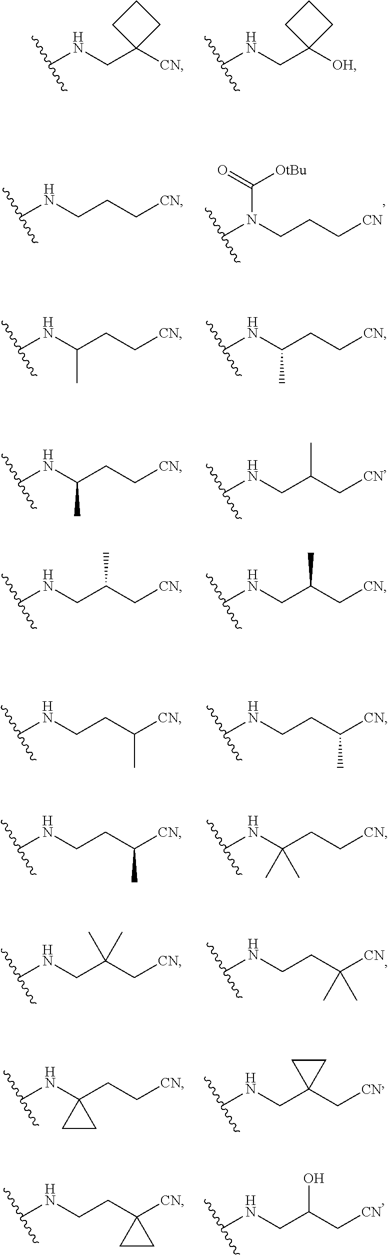 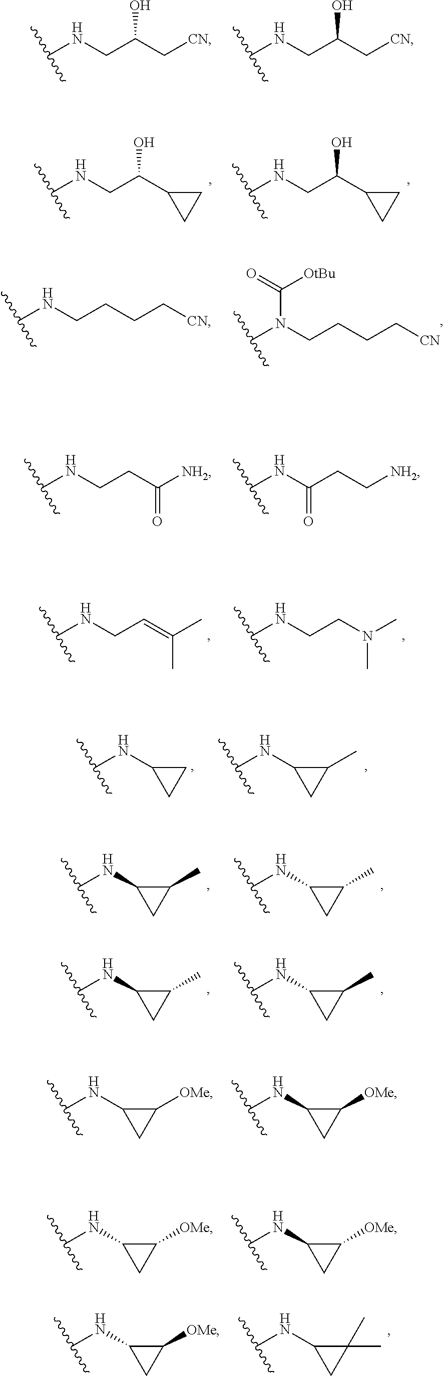 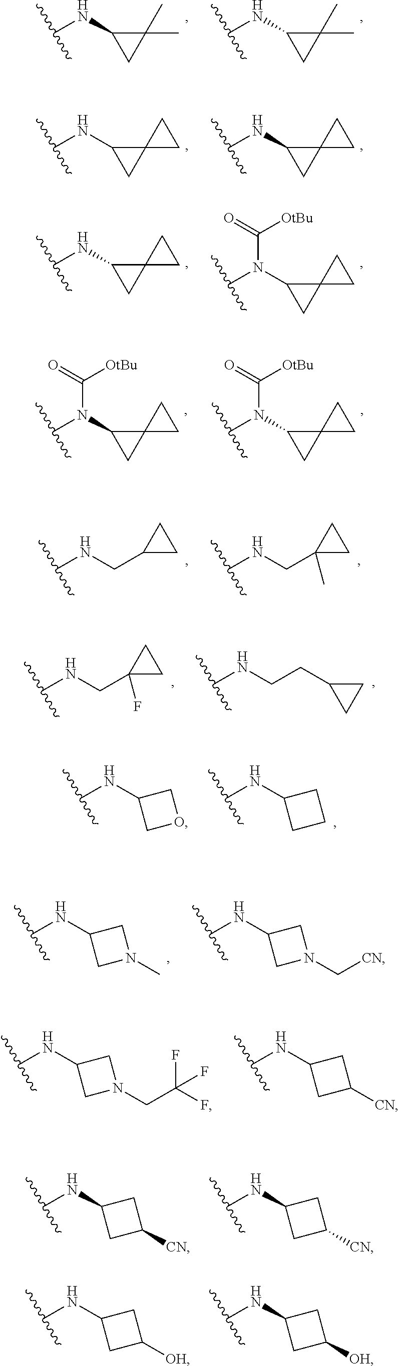 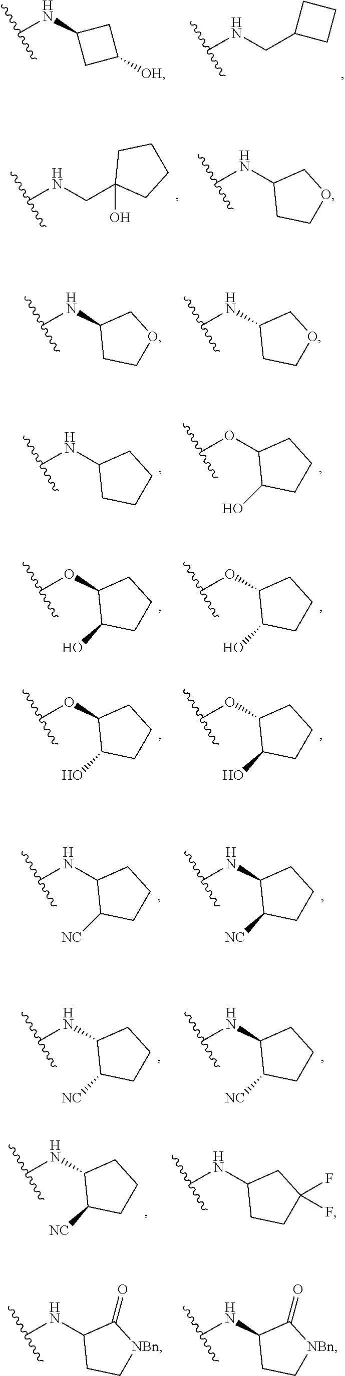 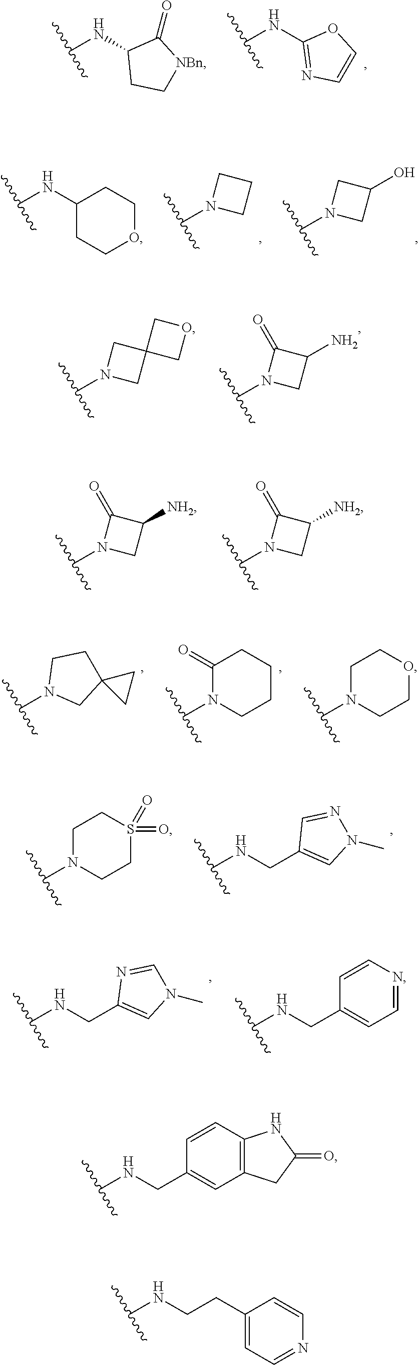
an optionally substituted phenyl, or an optionally substituted 5-6 membered heteroaryl having 1-3 ring heteroatoms selected from nitrogen, oxygen, and sulfur.

12. The compound of any one of claims 1-11, wherein R⁸ is selected from halogen, —CF₃, —OR, —CH₂NR(CH₂CH₂O)₁₋₁₀CH₂CH₂NR₂, and an optionally substituted C₁ aliphatic.

13. The compound of any one of claims 1-12, wherein R⁸ is selected from fluoro, chloro, methyl, —CF₃, —OH

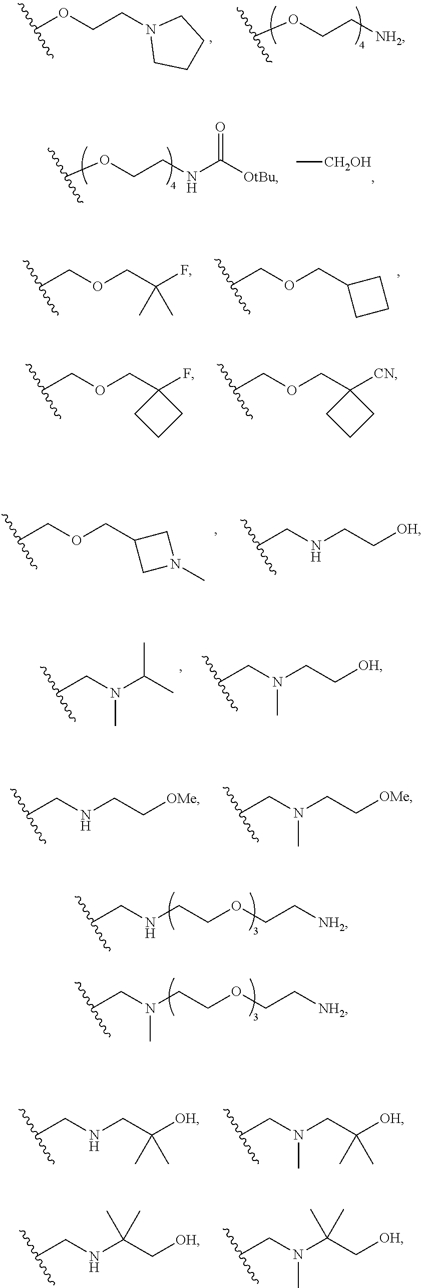

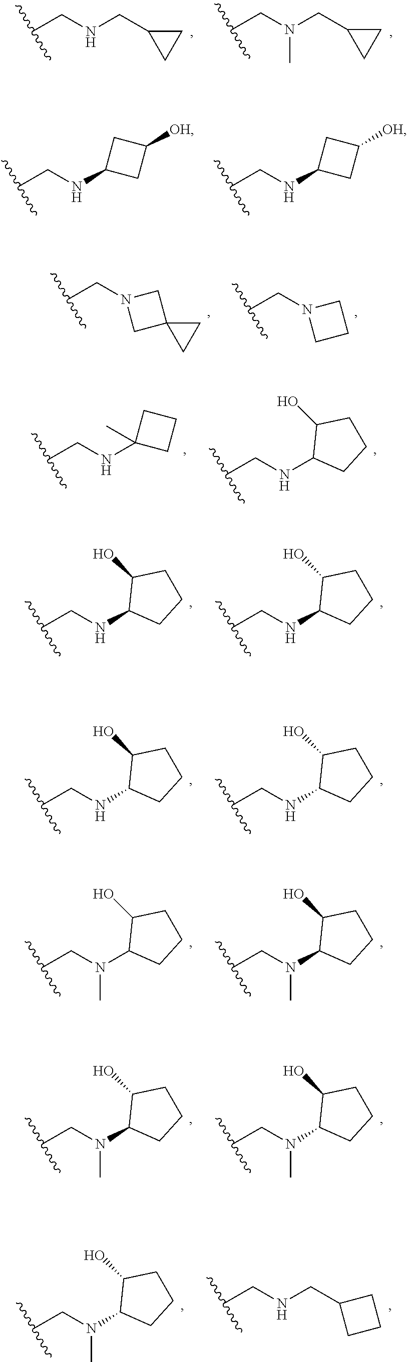

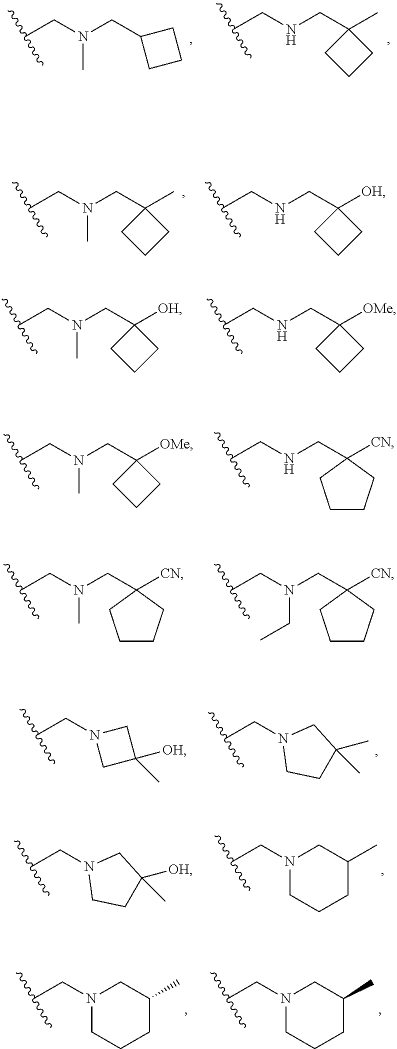

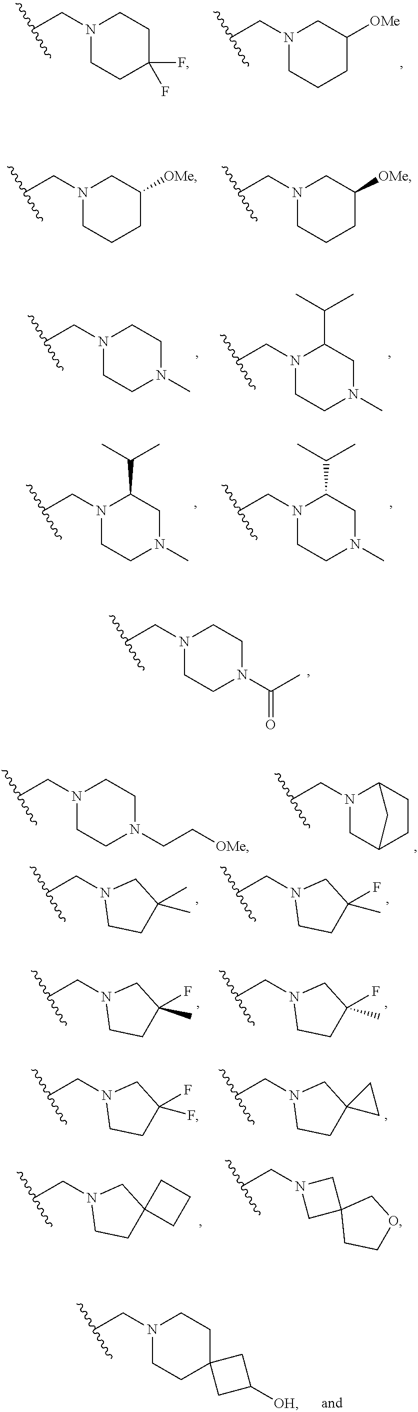

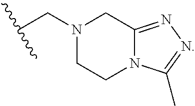 [SMILES: *CN1CCn2c(C)nnc2C1]

14. The compound of claims 1, 2, or 5, represented by any one of the following formulae: 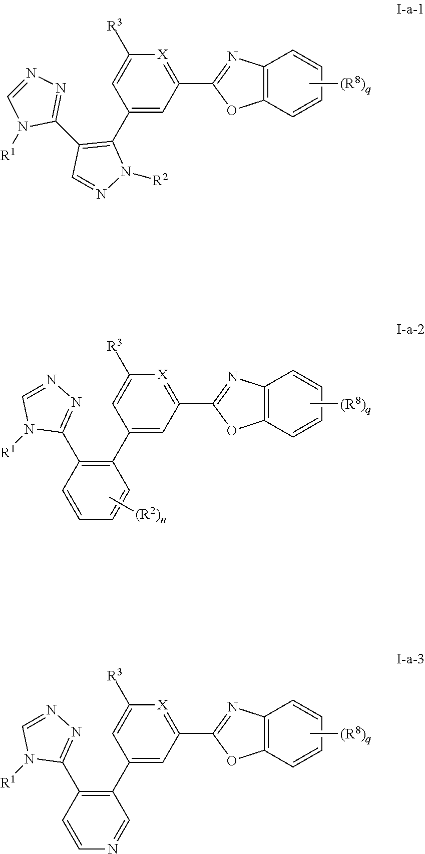
or a pharmaceutically acceptable salt thereof.

15. The compound of claim 14, wherein R² is halogen, —CN, —CH₂OR, —CH(OR)R, —OR, —NR₂, —C(O)R, —C(O)OR, —C(O)NR₂, —NRSO₂R, or an optionally substituted C₁₋₆aliphatic.

16. The compound of any one of claims 1-9 or claim 11, represented by any one of the following formulae: 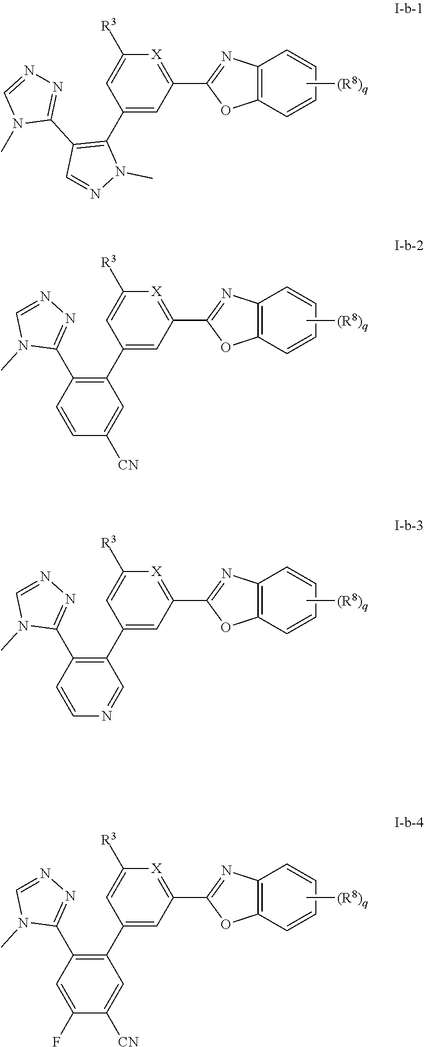
or a pharmaceutically acceptable salt thereof.

17. The compound of claim 16, represented by any one of the following formulae: 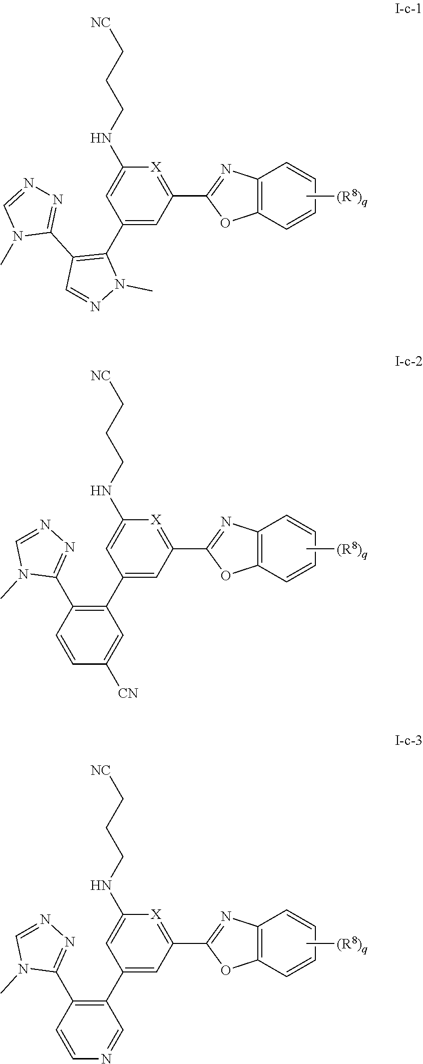 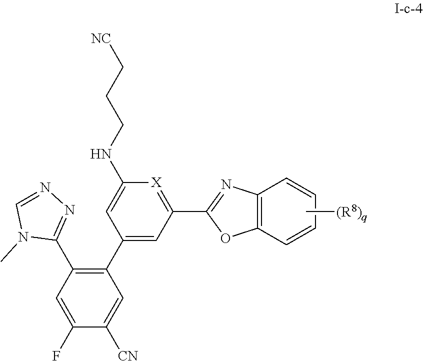
or a pharmaceutically acceptable salt thereof.

18. The compound of any one of claims 14-17, wherein R⁸ is selected from halogen, —CF₃, —OR, —NR₂, and an optionally substituted C₁₋₆ aliphatic.

19. The compound of any one of claims 1, 2, or 5, represented by any one of the following formulae: 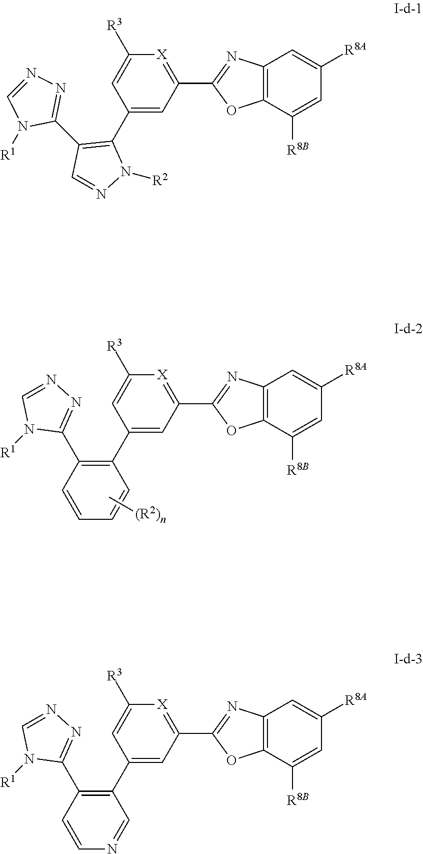
or a pharmaceutically acceptable salt thereof, wherein
R^{8A} and R^{8B} are independently selected from hydrogen, halogen, —CF₃, —OR, —NR₂, and an optionally substituted C₁₋₆ aliphatic.

20. The compound of any one of claims 1-19, wherein X is CH.

21. The compound of any one of claims 1-19, wherein X is N.

22. The compound of any one of claims 1-9 or claim 11, represented by any one of the following formulae: 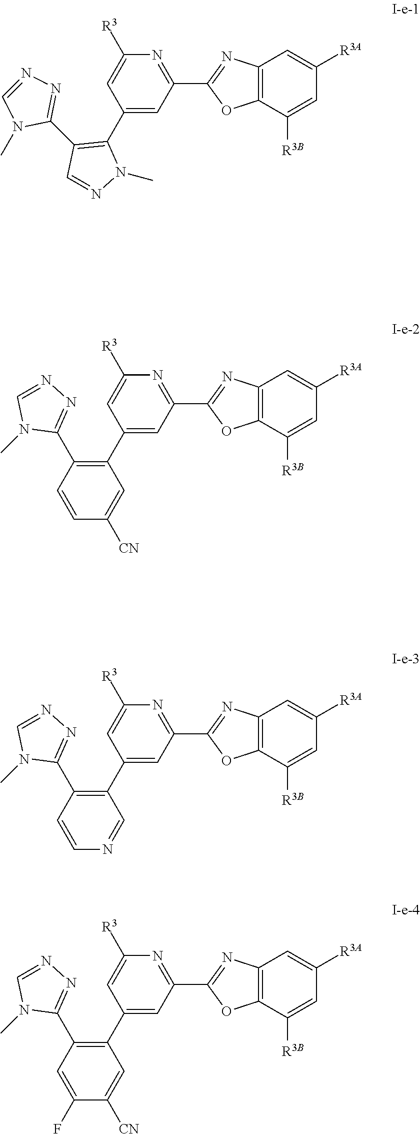 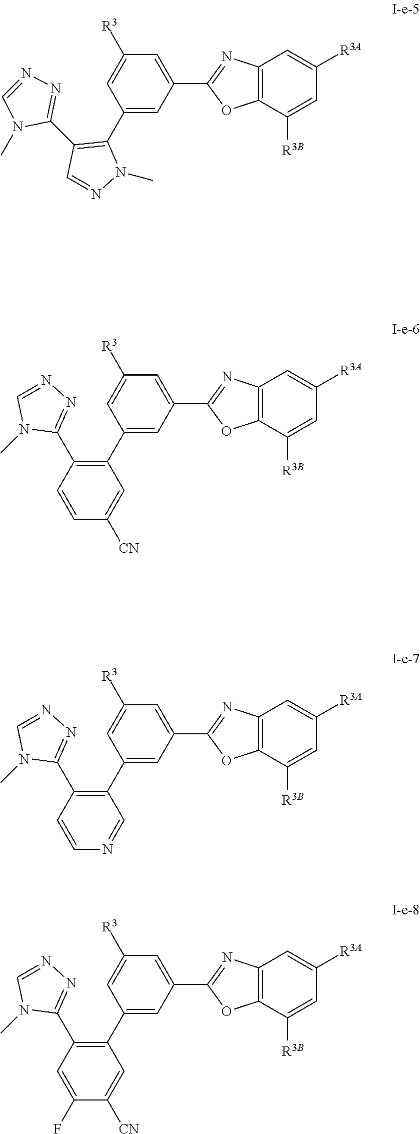
or a pharmaceutically acceptable salt thereof, wherein
R^{8A} and R^{8B} are independently selected from hydrogen, halogen, —CF₃, —OR, —NR₂, and an optionally substituted C₁_aliphatic.

23. The compound of claim 20 or 21, represented by any one of the following formulae: 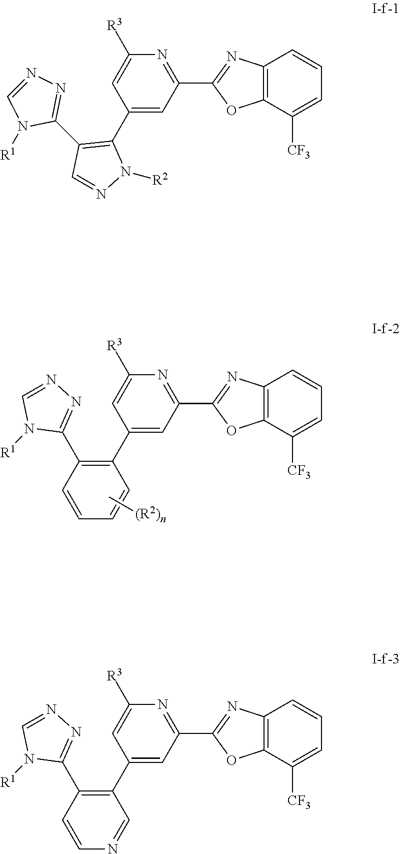 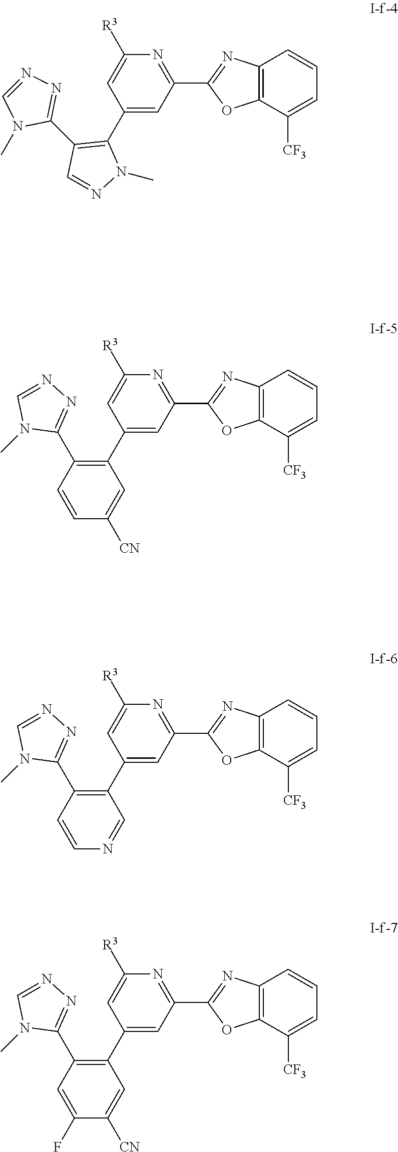 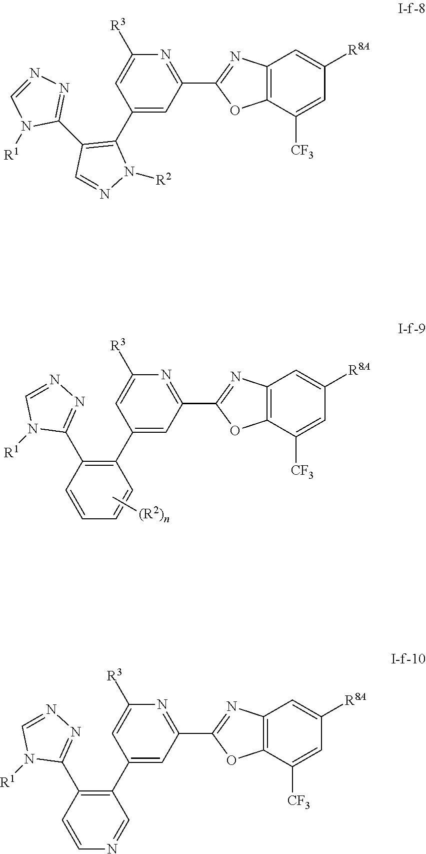 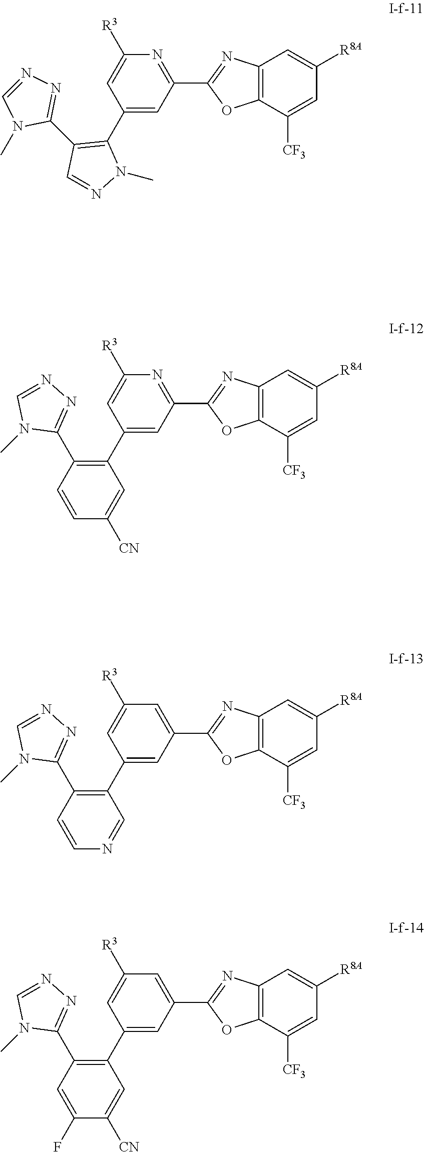 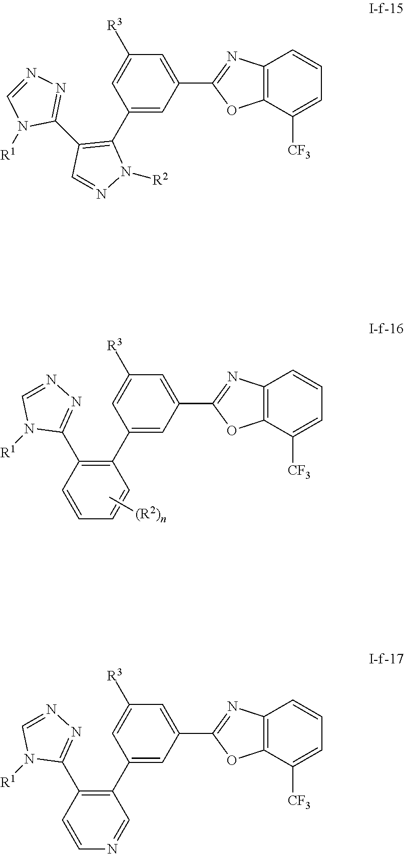 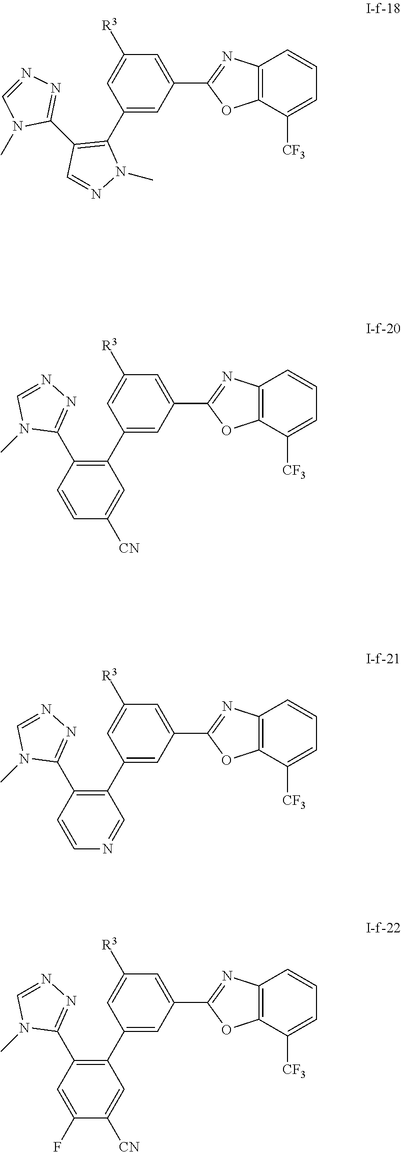 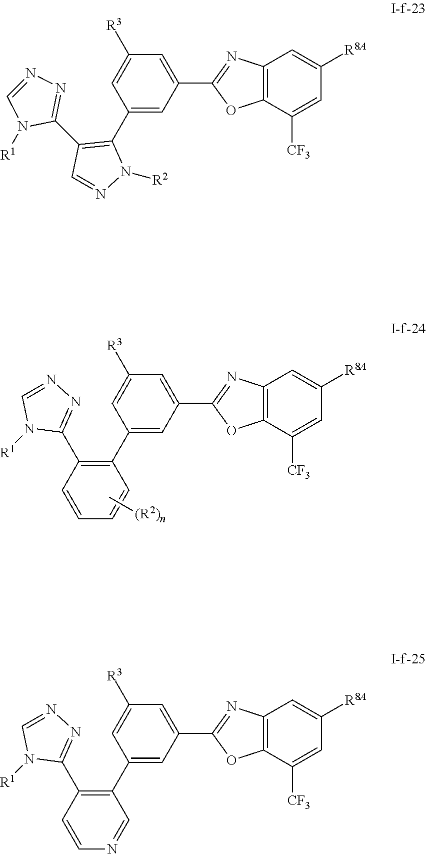 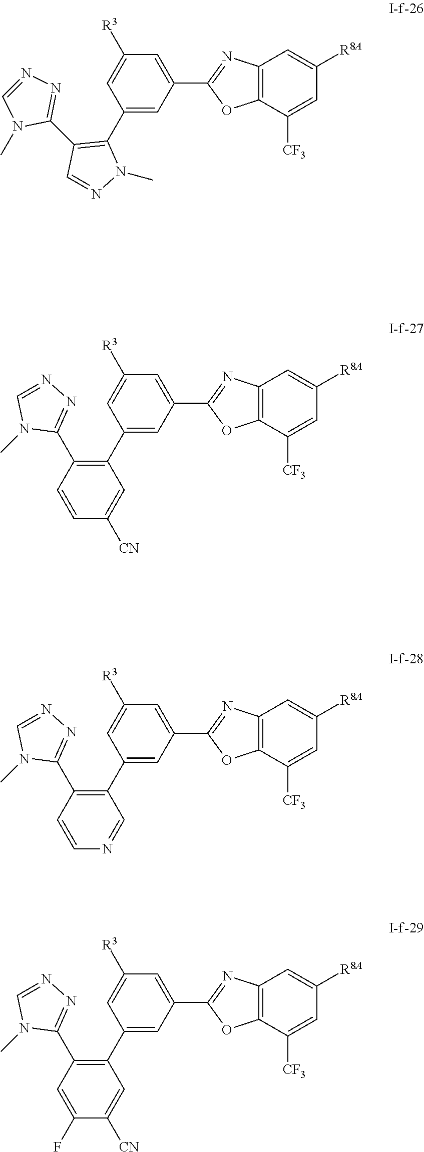
or a pharmaceutically acceptable salt thereof.

24. The compound of any one claims 16-23, wherein R³ is —CN, —CH(R^{3A})(R^{3B}), —OR^{3D}, —N(R^{3A})(R^{3E}), —C(O)OR^{3A}, —C(O)N(R^{3A})OR^{3A}, —N(R^{3A})C(O)OR^{3A}, —N(R^{3A})C(O)R^{3A}, —N(R^{3A})C(O)N(R^{3A})₂, —N(R^{3A})C(NR^{3A})R^{3A}, —N(R^{3A})S(O)₂R^{3A}, a substituted straight-chain C₂₋₃ aliphatic, or an optionally substituted group selected from C₄₋₆ aliphatic; a 4 or 6-8 membered saturated or partially unsaturated monocyclic heterocyclic ring having 1-2 heteroatoms independently selected from nitrogen, oxygen, and sulfur; and a 6-10 membered saturated or partially unsaturated spirocyclic ring having 0-3 heteroatoms independently selected from nitrogen, oxygen, and sulfur.

25. The compound of claim 20, represented by any one of the following formulae: 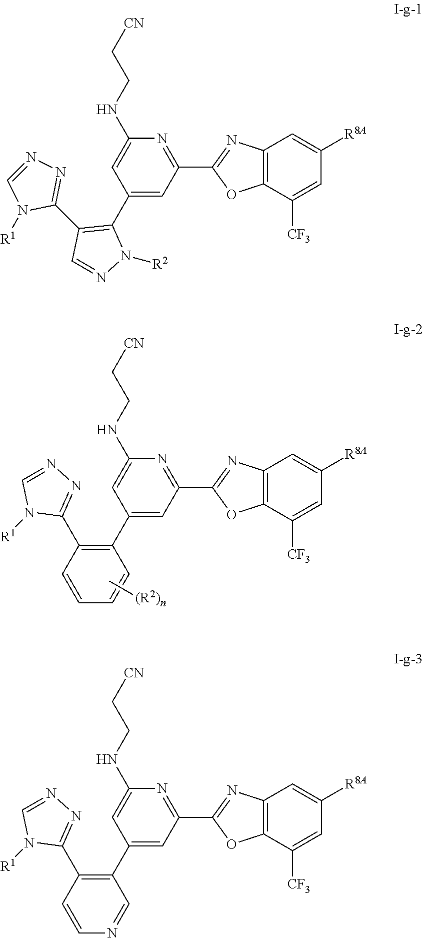 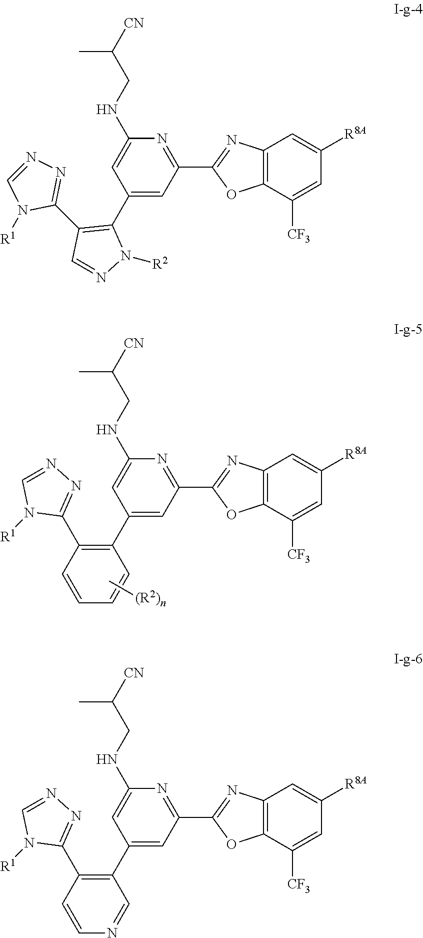 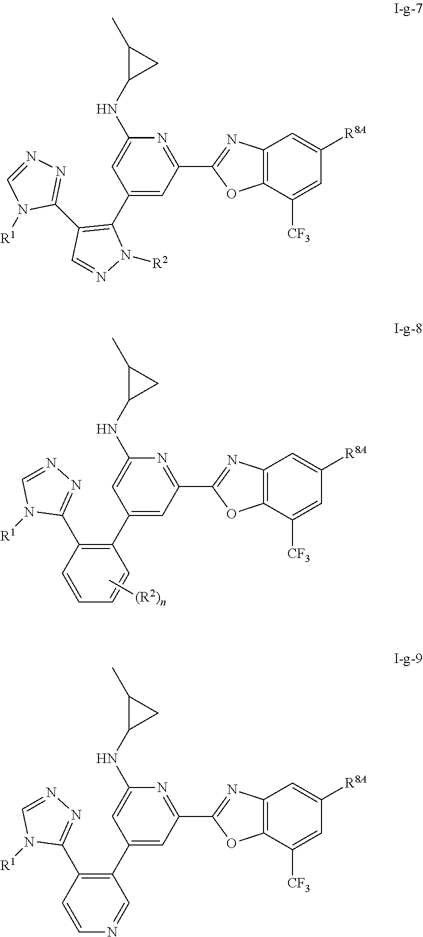 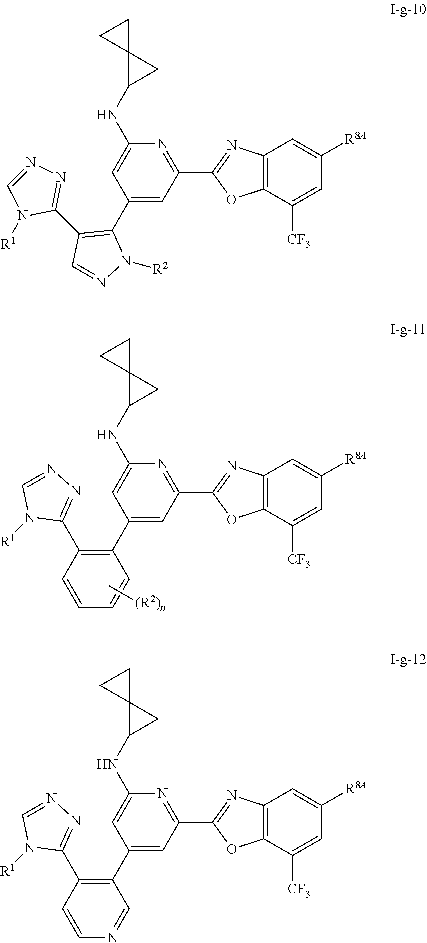 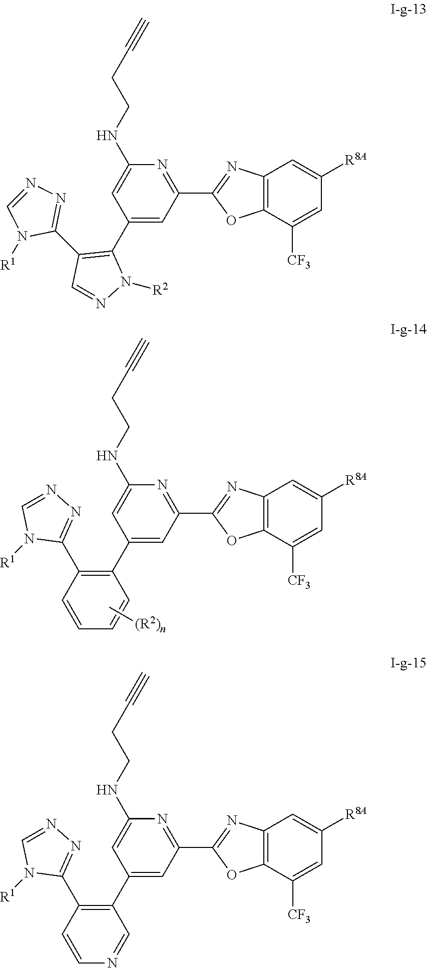 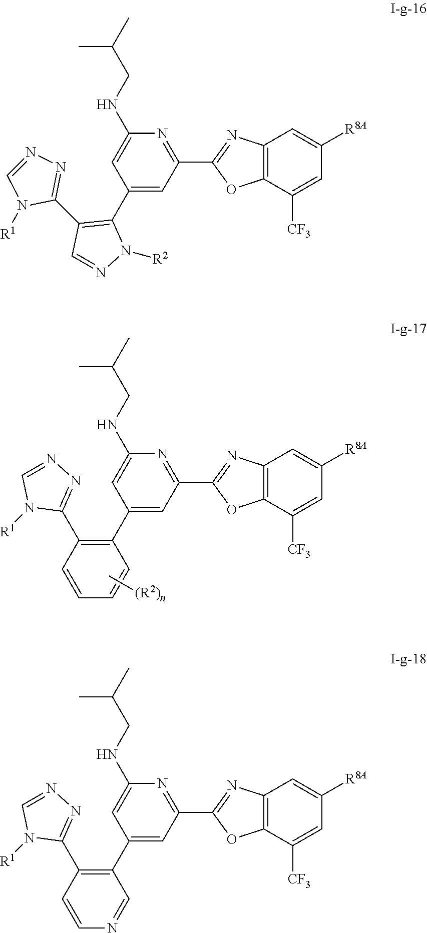 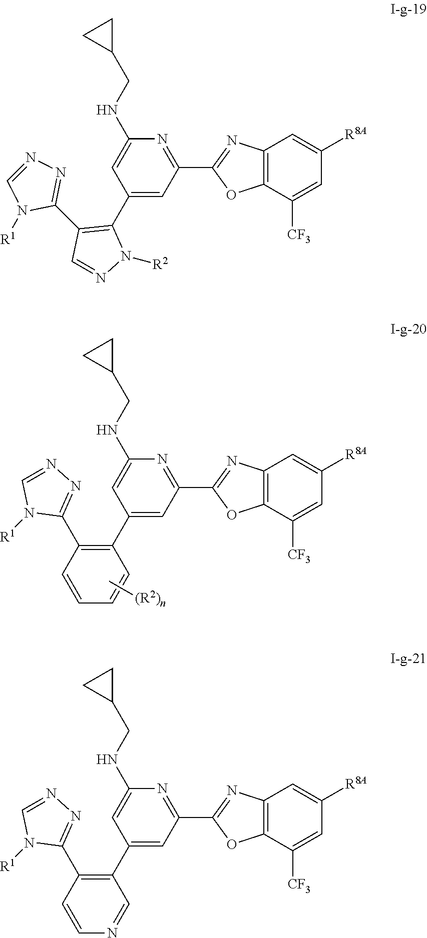 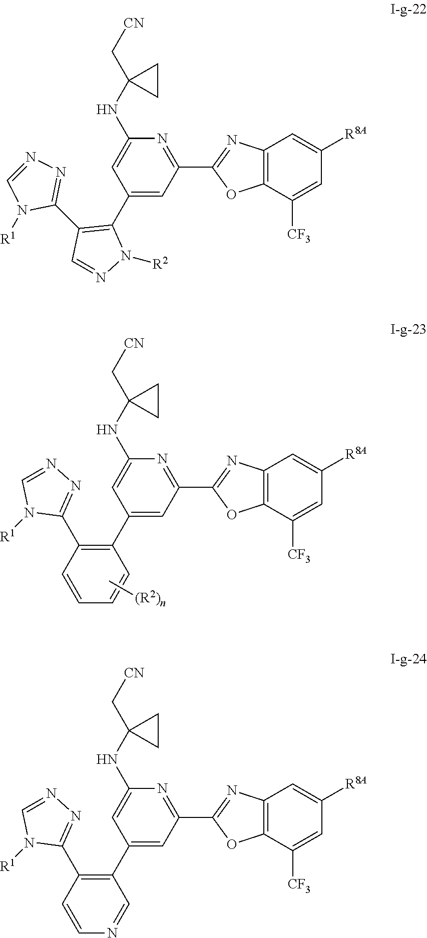 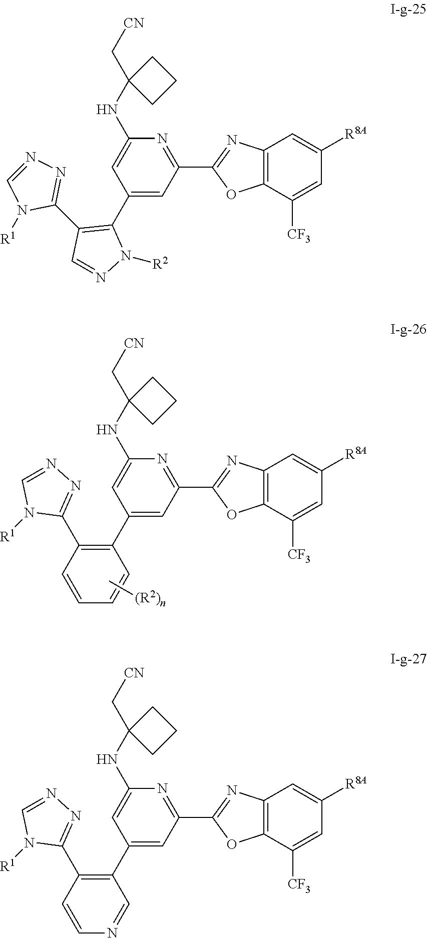 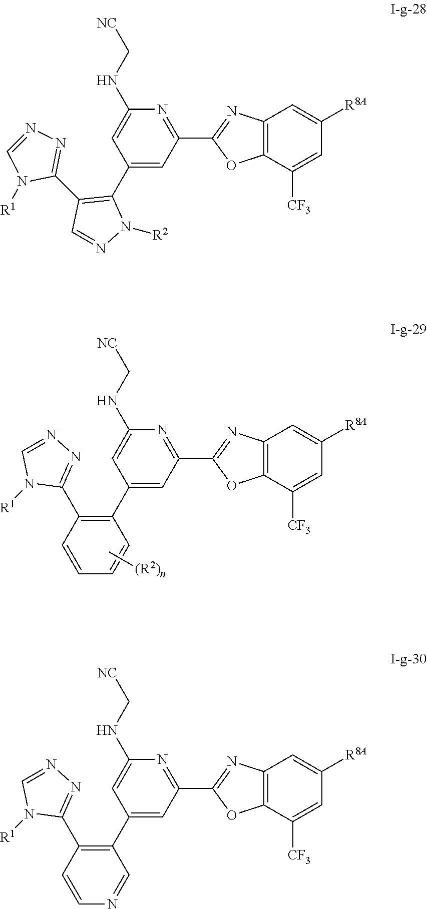 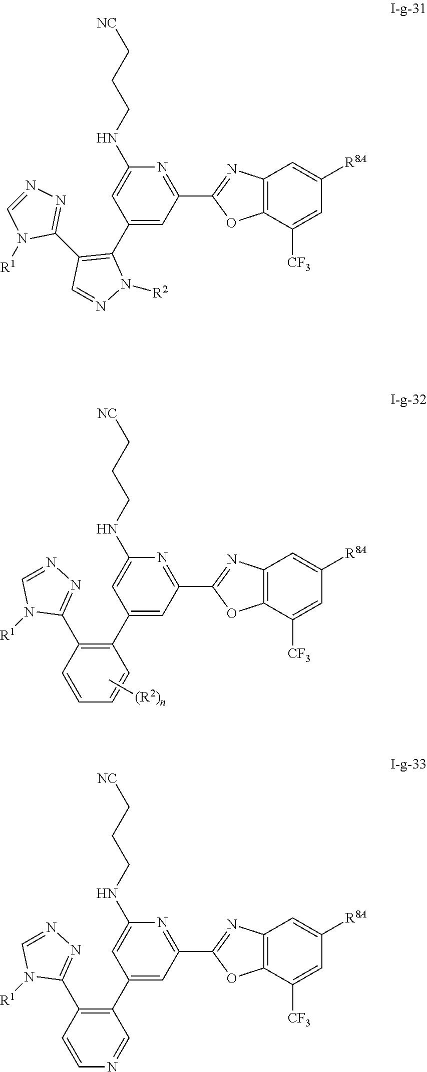 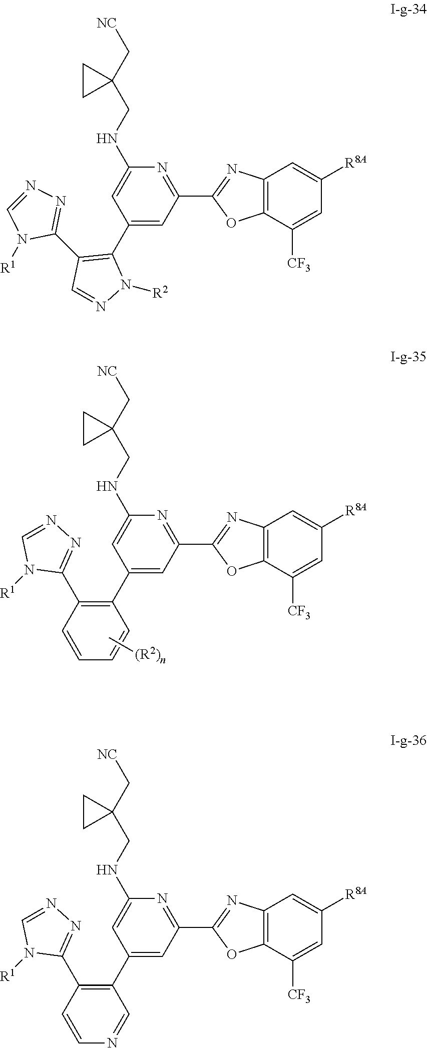 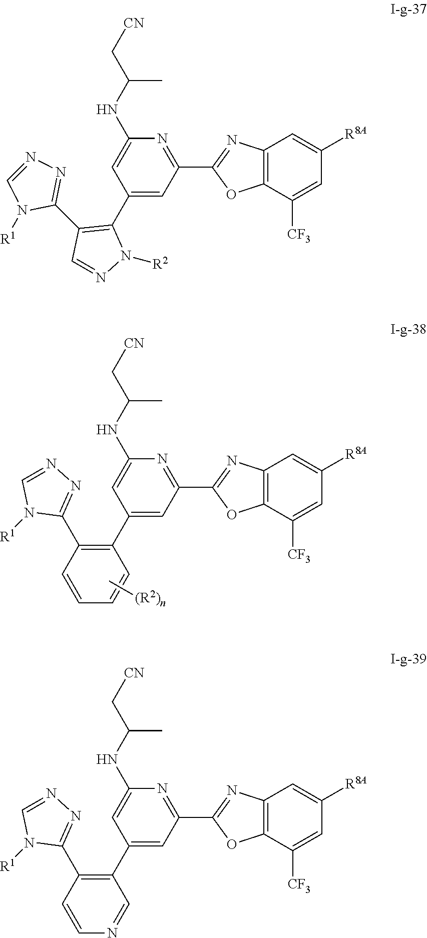 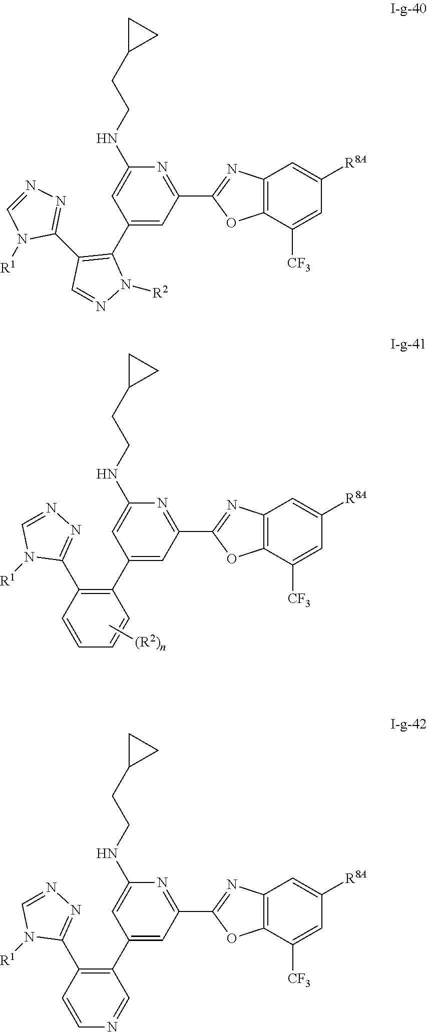 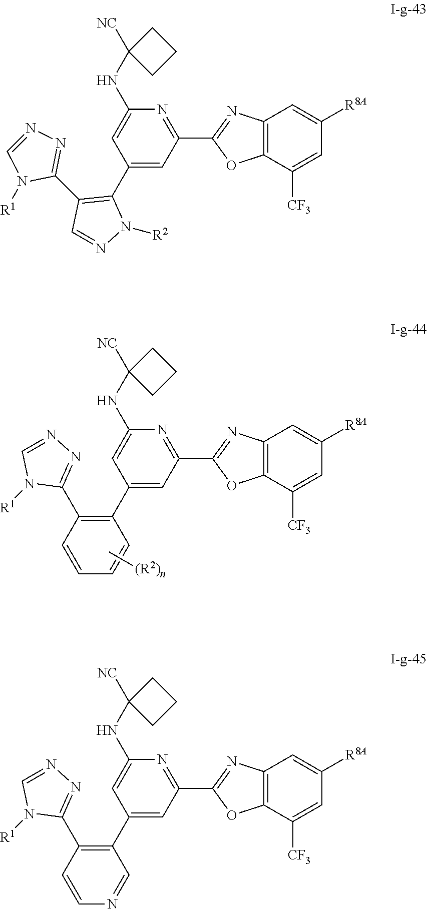 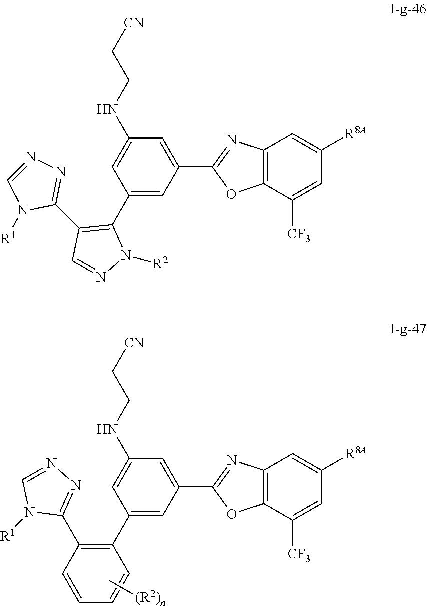 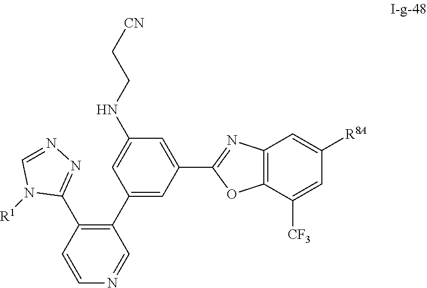 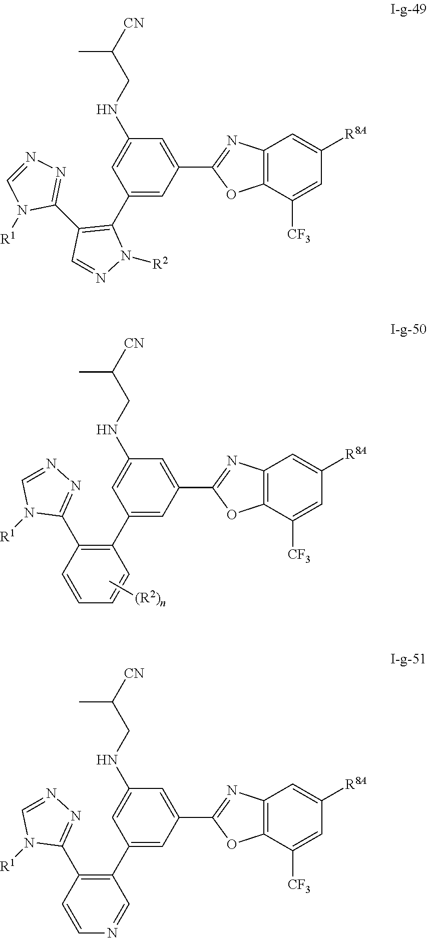 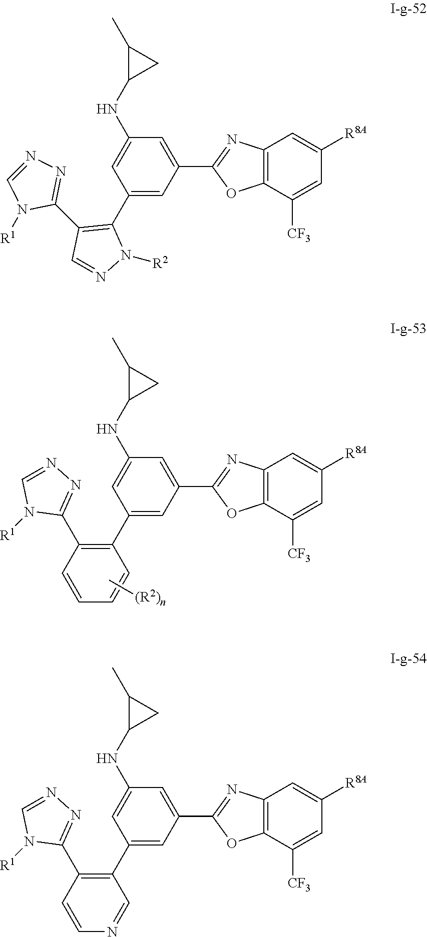 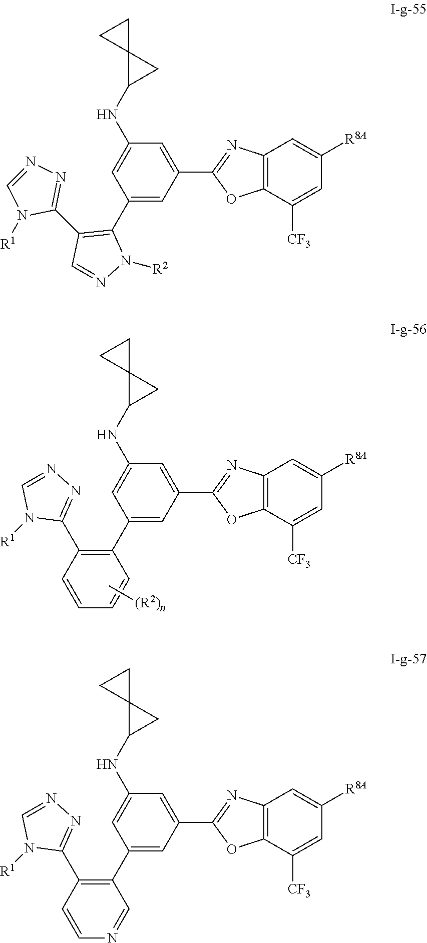 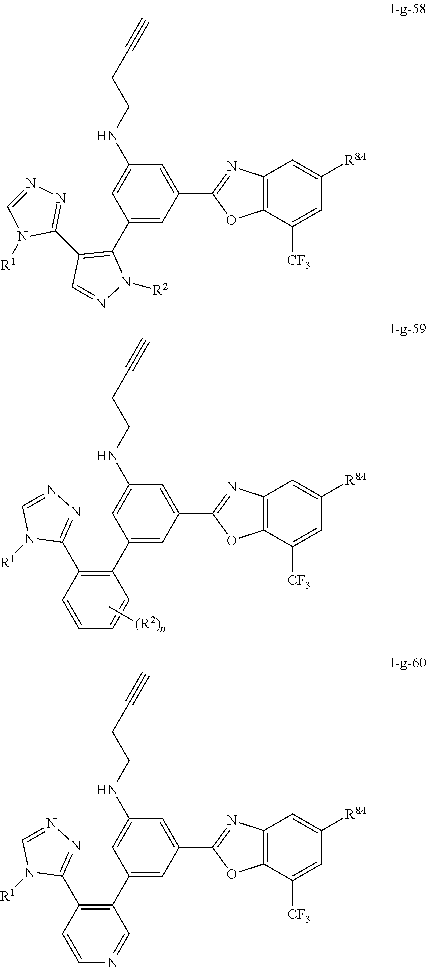 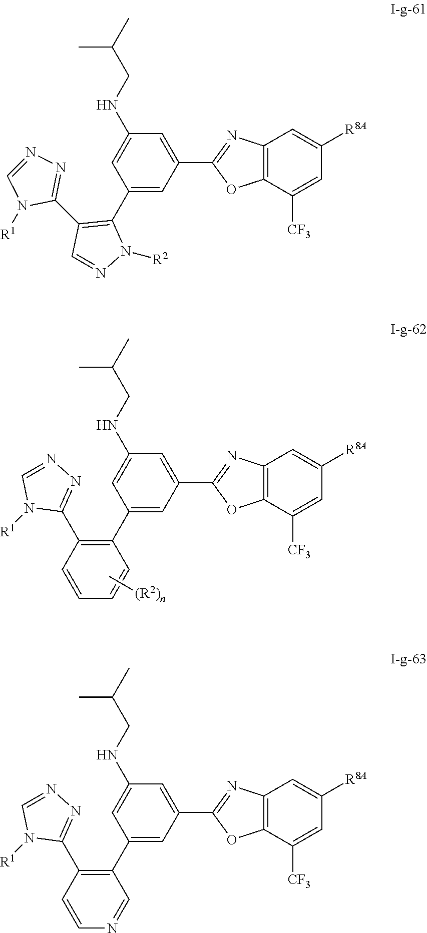 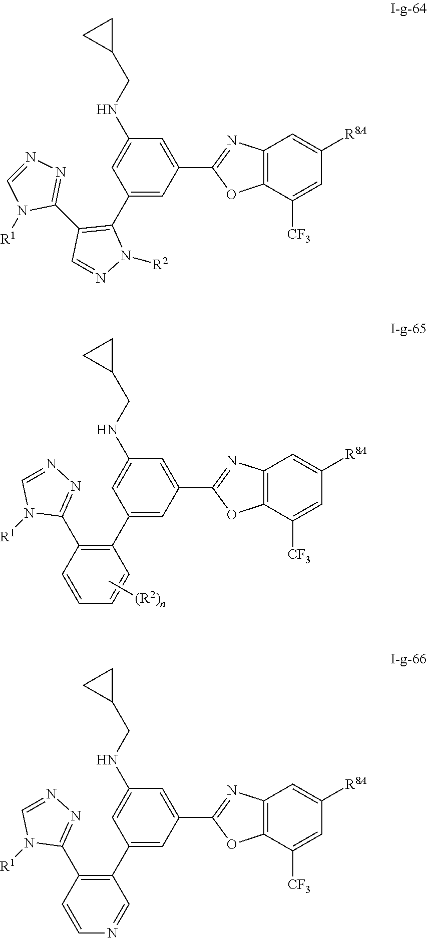 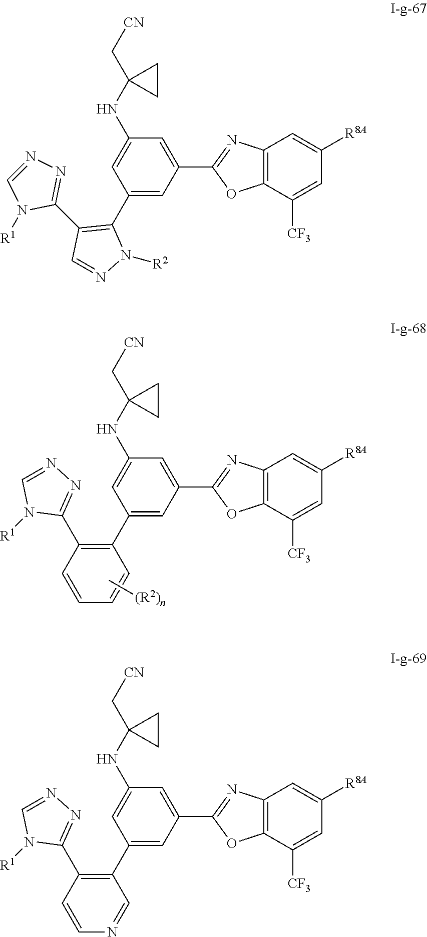 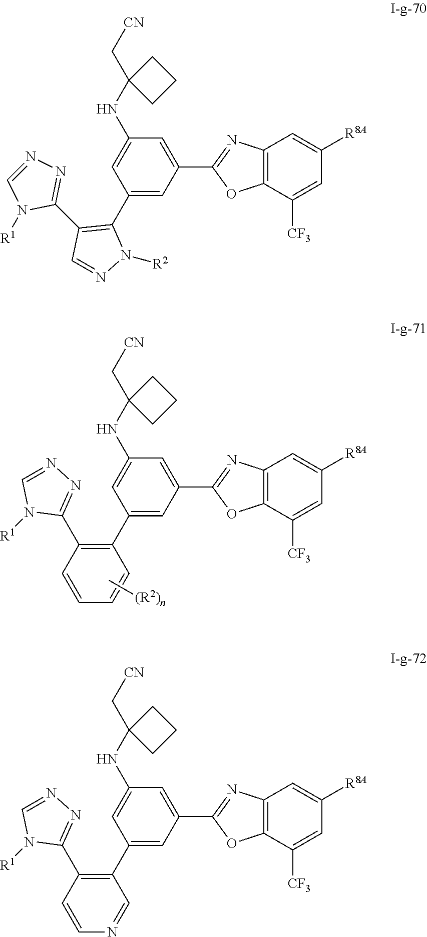 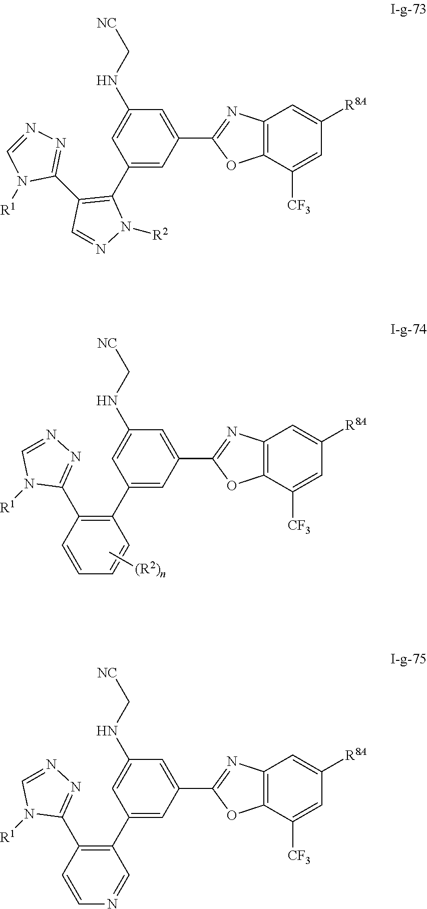 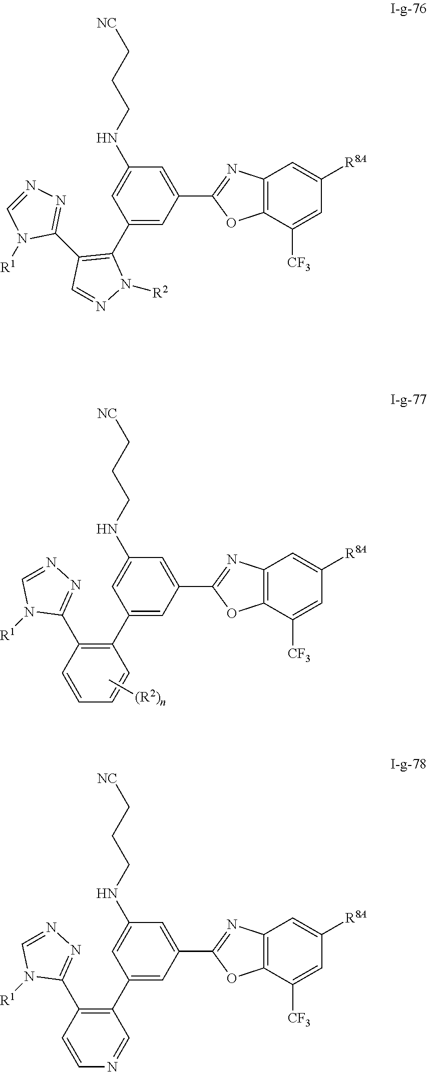 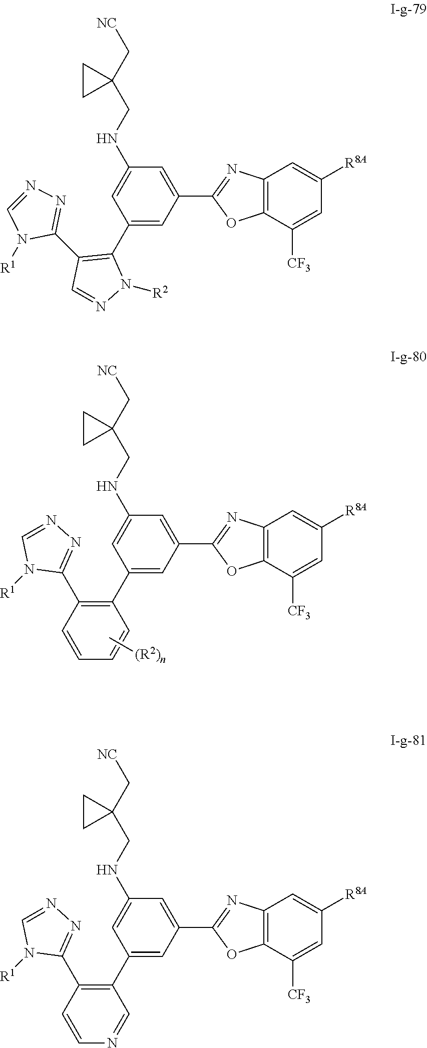 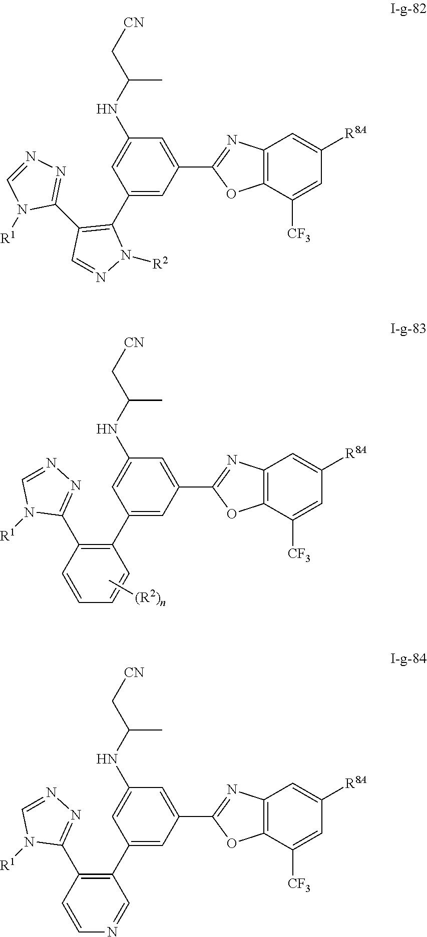 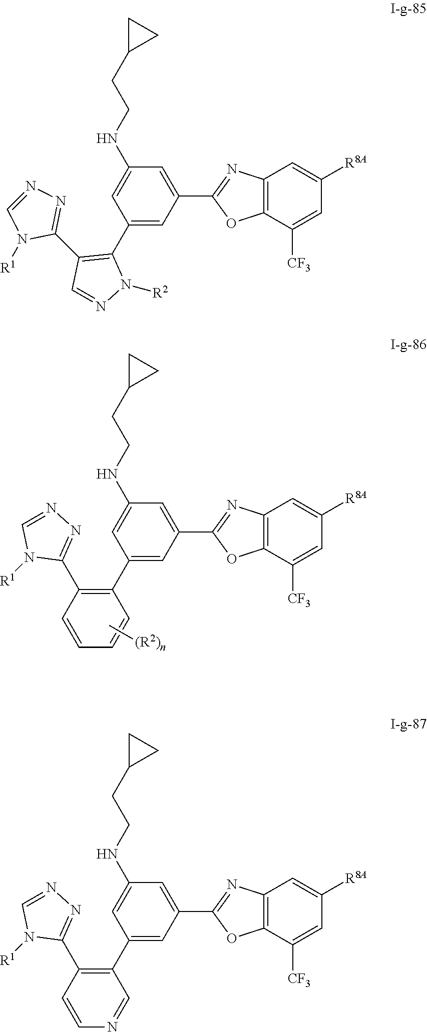 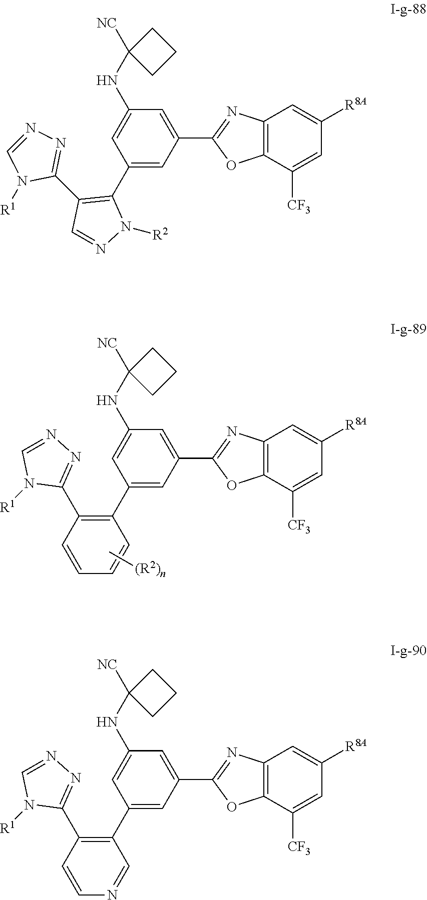
or a pharmaceutically acceptable salt thereof.

26. A compound of formula II: 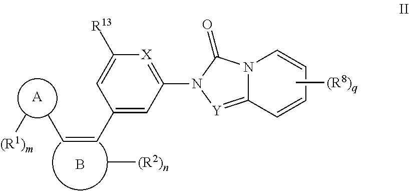
or a pharmaceutically acceptable salt thereof, wherein:
Ring A is a 5 membered heteroaryl ring having 1-3 nitrogen and 0-1 oxygen or sulfur;
R¹ is hydrogen, halogen, —CN, —OR, or an optionally substituted C₁₋₆ aliphatic;
Ring B is a divalent phenyl, or a divalent 5-6 membered heteroaryl ring having 1-4 heteroatoms independently selected from nitrogen, oxygen, and sulfur;
each R² is independently hydrogen, halogen, —CN, —CH₂OR, —CH(OR)R, —CRF₂, —CF₃, —OR, —SR, —NR₂, —SO₂R, —SO₂NR₂, —S(O)R, —C(O)R, —C(O)OR, —C(O)NR₂, —OC(O)R, —OC(O)NR₂, —NRC(O)OR, —NRC(O)R, —NRSO₂R; or an optionally substituted C₁₋₆ aliphatic or C₄₋₆ heterocycloalkyl;
X is CH or N;
Y is CH or N;
R¹³ is hydrogen, halogen, —CN, —OR^{3A}, —SR^{3A}, —N(R^{3A})(R^{3A}), —S(O)₂R^{3A}, —S(O)₂N(R^{3A})₂, —S(O)R^{3A}, —S(O)N(R^{3A})₂, —C(O)R^{3A}, —C(O)OR^{3A}, —C(O)N(R^{3A})₂, —C(O)N(R^{3A})OR^{3A}, —OC(O)R^{3A}, —OC(O)N(R^{3A})₂, —N(R^{3A})C(O)OR^{3A}, —N(R^{3A})C(O)R^{3A}, —N(R^{3A})C(O)N(R^{3A})₂, —N(R^{3A})C(NR^{3A})R^{3A}, —N(R^{3A})C(NR^{3A})N(R^{3A})₂, —N(R^{3A})N(R^{3A})₂, —N(R^{3A})S(O)₂N(R^{3A})₂, —N(R^{3A})S(O)₂R^{3A}, —N═S(O)(R^{3A}), —S(NR^{3A})(O)R^{3A}, —N(R^{3A})S(O)R^{3A}, —N(R^{3A})CN, —P(O)(R^{3A})OR^{3A}, —P(O)(R^{3A})₂, or an optionally substituted group selected from C₁₋₆ aliphatic: a phenyl ring; a 4-7 membered saturated or partially unsaturated monocyclic carbocyclic ring; a 5-6 membered monocyclic heteroaryl ring having 1-4 heteroatoms independently selected from nitrogen, oxygen, and sulfur; a 4-8 membered saturated or partially unsaturated monocyclic heterocyclic ring having 1-2 heteroatoms independently selected from nitrogen, oxygen, and sulfur; and a 6-10 membered saturated or partially unsaturated spirocyclic ring having 0-3 heteroatoms independently selected from nitrogen, oxygen, and sulfur;
each R^{3A} are independently hydrogen, —CN, halogen, or an optionally substituted group selected from C₁₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic carbocyclic ring having 1-2 heteroatoms independently selected from nitrogen, oxygen, and sulfur; or:
two R^{3A} groups on the same atom are optionally taken together with the atom to form an optionally substituted ring selected from a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 7-10 membered saturated or partially unsaturated bicyclic heterocyclic ring having 1-4 heteroatoms independently selected from nitrogen, oxygen, phosphorus, and sulfur; a 5-8 membered saturated or partially unsaturated bridged bicyclic ring having 0-3 heteroatoms independently selected from nitrogen, oxygen, phosphorus, and sulfur; and a 6-10 membered saturated or partially unsaturated spirocyclic ring having 0-3 heteroatoms independently selected from nitrogen, oxygen, phosphorus, and sulfur;
each R⁸ is independently hydrogen, oxo, halogen, —CN, —NO₂, —CHF₂, —CF₃, —OR, —(OCH₂CH₂)₁₋₁₀NR₂, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —NRC(O)OR, —NRC(O)R, —NRC(O)NR₂, —NRC(NR)NR₂, —NRNR₂, —NRS(O)₂NR₂, —NRS(O)₂R, —N═S(O)R₂, —S(NR)(O)R, —NRS(O)R, —NRCN, —P(O)R₂, —P(O)(OR)₂—CH₂NR(CH₂CH₂O)₁₋₁₀CH₂CH₂NR₂; or an optionally substituted C₁₋₆aliphatic;
each R is independently hydrogen, or an optionally substituted group selected from C_{1-_}aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic carbocyclic ring having 1-2 heteroatoms independently selected from nitrogen, oxygen, and sulfur; or:
two R groups on the same atom are optionally taken together with the atom to form an optionally substituted 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorus, and sulfur;
m is 0, 1, or 2;
n is 0, 1, 2, or 3; and
q is 0, 1, 2, or 3.

27. The compound of claim 26, represented by any one of the following formulae: 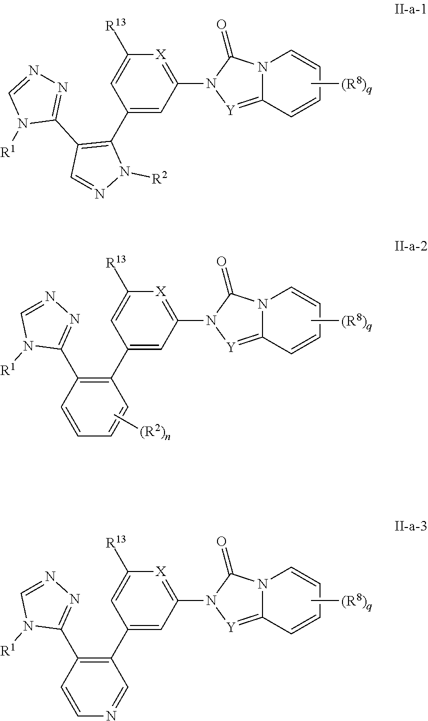
or a pharmaceutically acceptable salt thereof

28. A compound of formula III: 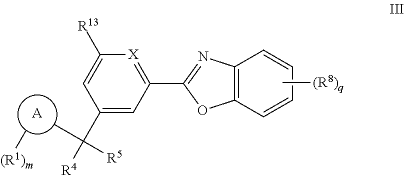
or a pharmaceutically acceptable salt thereof, wherein:
Ring A is a 5 membered heteroaryl ring having 1-3 nitrogen and 0-1 oxygen or sulfur;
R¹ is hydrogen, halogen, —CN, —OR, or an optionally substituted C₁₋₆ aliphatic;
R⁴ and R⁵ are each independently hydrogen or an optionally substituted group selected from C₁₋₆ aliphatic, 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; or:
R⁴ and R⁵ are optionally taken together with the carbon they are attached to form 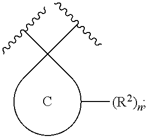
Ring C is a divalent spiro-fused 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur;
each R² is independently hydrogen, halogen, —CN, —CH₂OR, —CH(OR)R, —CRF₂, —CF₃, —OR, —SR, —NR₂, —SO₂R, —SO₂NR₂, —S(O)R, —C(O)R, —C(O)OR, —C(O)NR₂, —OC(O)R, —OC(O)NR₂, —NRC(O)OR, —NRC(O)R, —NRSO₂R; or an optionally substituted C₁₋₆ aliphatic or C₄₋₆ heterocycloalkyl;
X is CH or N;
R¹³ is hydrogen, halogen, —CN, —OR^{3A}, —SR^{3A}, —N(R^{3A})(R^{3A}), —S(O)₂R^{3A}, —S(O)₂N(R^{3A})₂, —S(O)R^{3A}, —S(O)N(R^{3A})₂, —C(O)R^{3A}, —C(O)OR^{3A}, —C(O)N(R^{3A})₂, —C(O)N(R^{3A})OR^{3A}, —OC(O)R^{3A}, —OC(O)N(R^{3A})₂, —N(R^{3A})C(O)OR^{3A}, —N(R^{3A})C(O)R^{3A}, —N(R^{3A})C(O)N(R^{3A})₂, —N(R^{3A})C(NR^{3A})R^{3A}, —N(R^{3A})C(NR^{3A})N(R^{3A})₂, —N(R^{3A})N(R^{3A})₂, —N(R^{3A})S(O)₂N(R^{3A})₂, —N(R^{3A})S(O)₂R^{3A}, —N═S(O)(R^{3A})₂, —S(NR^{3A})(O)R^{3A}, —N(R^{3A})S(O)R^{3A}, —N(R^{3A})CN, —P(O)(R^{3A})OR^{3A}, —P(O)(R^{3A})₂, or an optionally substituted group selected from C₁₋₆ aliphatic; a phenyl ring; a 4-7 membered saturated or partially unsaturated monocyclic carbocyclic ring; a 5-6 membered monocyclic heteroaryl ring having 1-4 heteroatoms independently selected from nitrogen, oxygen, and sulfur; a 4-8 membered saturated or partially unsaturated monocyclic heterocyclic ring having 1-2 heteroatoms independently selected from nitrogen, oxygen, and sulfur; and a 6-10 membered saturated or partially unsaturated spirocyclic ring having 0-3 heteroatoms independently selected from nitrogen, oxygen, and sulfur;
each R^{3A} are independently hydrogen, —CN, halogen, or an optionally substituted group selected from C₁₋₆ aliphatic; phenyl: naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic carbocyclic ring having 1-2 heteroatoms independently selected from nitrogen, oxygen, and sulfur; or:
two R^{3A} groups on the same atom are optionally taken together with the atom to form an optionally substituted ring selected from a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 7-10 membered saturated or partially unsaturated bicyclic heterocyclic ring having 1-4 heteroatoms independently selected from nitrogen, oxygen, phosphorus, and sulfur; a 5-8 membered saturated or partially unsaturated bridged bicyclic ring having 0-3 heteroatoms independently selected from nitrogen, oxygen, phosphorus, and sulfur; and a 6-10 membered saturated or partially unsaturated spirocyclic ring having 0-3 heteroatoms independently selected from nitrogen, oxygen, phosphorus, and sulfur;
each R⁸ is independently hydrogen, oxo, halogen, —CN, —NO₂, —CHF₂, —CF₃, —OR, —(OCH₂CH₂)₁₋₁₀NR₂, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —NRC(O)OR, —NRC(O)R, —NRC(O)NR₂, —NRC(NR)NR₂, —NRNR₂, —NRS(O)₂NR₂, —NRS(O)₂R, —N═S(O)R₂, —S(NR)(O)R, —NRS(O)R, —NRCN, —P(O)R₂, —P(O)(OR)₂—CH₂NR(CH₂CH₂O)₁₋₁₀CH₂CH₂NR₂; or an optionally substituted C₁₋₆ aliphatic;
each R is independently hydrogen, or an optionally substituted group selected from C₁₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic carbocyclic ring having 1-2 heteroatoms independently selected from nitrogen, oxygen, and sulfur; or:
two R groups on the same atom are optionally taken together with the atom to form an optionally substituted 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorus, and sulfur;
m is 0, 1, or 2;
n is 0, 1, 2, or 3; and
q is 0, 1, 2, or 3,
wherein the compound of formula III is not:

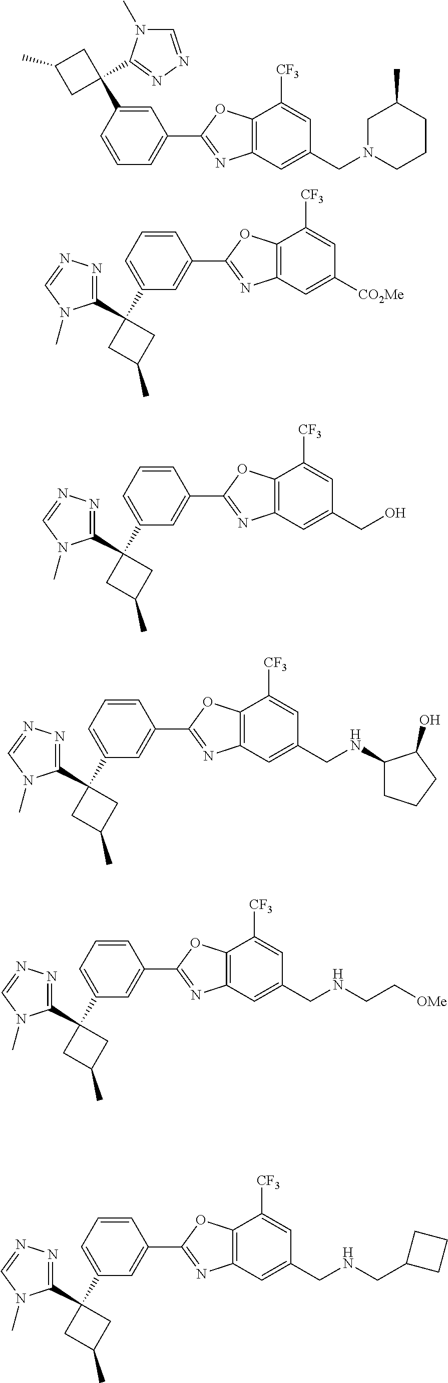

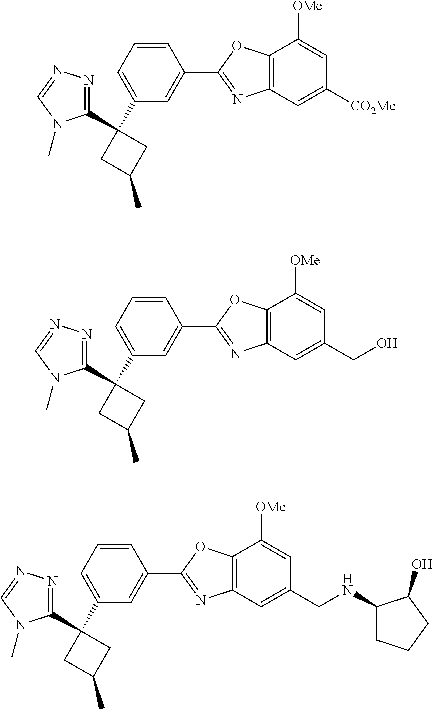 [SMILES: COC(=O)c1cc(OC)c2oc(-c3cccc([C@]4(c5nncn5C)C[C@@H](C)C4)c3)nc2c1 ; COc1cc(CO)cc2nc(-c3cccc([C@]4(c5nncn5C)C[C@@H](C)C4)c3)oc12 ; COc1cc(CN[C@@H]2CCC[C@@H]2O)cc2nc(-c3cccc([C@]4(c5nncn5C)C[C@@H](C)C4)c3)oc12]

29. A compound of formula III-a: 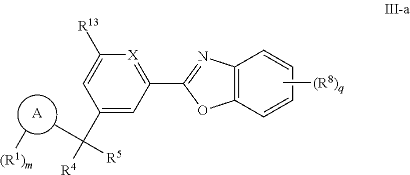
or a pharmaceutically acceptable salt thereof, wherein:
Ring A is a 5 membered heteroaryl ring having 1-3 nitrogen and 0-1 oxygen or sulfur;
R¹ is hydrogen, halogen, —CN, —OR, or an optionally substituted C₁₋₆ aliphatic:
R⁴ and R⁵ are each independently hydrogen or an optionally substituted group selected from C₁₋₆ aliphatic, 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; or:
R⁴ and R⁵ are optionally taken together with the carbon they are attached to form 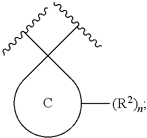
Ring C is a divalent spiro-fused 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur;
each R² is independently hydrogen, halogen, —CN, —CH₂OR, —CH(OR)R, —CRF₂, —CF₃, —OR, —SR, —NR₂, —SO₂R, —SO₂NR₂, —S(O)R, —C(O)R, —C(O)OR, —C(O)NR₂, —OC(O)R, —OC(O)NR₂, —NRC(O)OR, —NRC(O)R, —NRSO₂R; or an optionally substituted C₁₋₆ aliphatic or C₄₋₆ heterocycloalkyl;
R¹³ is hydrogen, halogen, —CN, —OR³, —SR^{3A}, —N(R^{3A})(R^{3A})—S(O)₂R^{3A}, —S(O)₂N(R^{3A})₂, —S(O)R^{3A}, —S(O)N(R^{3A})₂, —C(O)RWA, —C(O)OR^{3A}, —C(O)N(R^{3A})₂, —C(O)N(R^{3A})OR^{3A}, —OC(O)R^{3A}, —OC(O)N(R^{3A})₂, —N(R^{3A})C(O)OR^{3A}, —N(R^{3A})C(O)R^{3A}, —N(R^{3A})C(O)N(R^{3A})₂, —N(R^{3A})C(NR^{3A})R^{3A}, —N(R^{3A})C(NR^{3A})N(R^{3A})₂, —N(R^{3A})N(R^{3A})₂, —N(R^{3A})S(O)₂N(R^{3A})₂, —N(R^{3A})S(O)₂R^{3A}, —N═S(O)(R^{3A})₂, —S(NR^{3A})(O)R^{3A}, —N(R^{3A})S(O)R^{3A}, —N(R^{3A})CN, —P(O)(R^{3A})OR^{3A}, —P(O)(R^{3A})₂, or an optionally substituted group selected from C₁₋₆ aliphatic; a phenyl ring; a 4-7 membered saturated or partially unsaturated monocyclic carbocyclic ring; a 5-6 membered monocyclic heteroaryl ring having 1-4 heteroatoms independently selected from nitrogen, oxygen, and sulfur; a 4-8 membered saturated or partially unsaturated monocyclic heterocyclic ring having 1-2 heteroatoms independently selected from nitrogen, oxygen, and sulfur; and a 6-10 membered saturated or partially unsaturated spirocyclic ring having 0-3 heteroatoms independently selected from nitrogen, oxygen, and sulfur;
each R^{3A} are independently hydrogen, —CN, halogen, or an optionally substituted group selected from C₁₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic carbocyclic ring having 1-2 heteroatoms independently selected from nitrogen, oxygen, and sulfur; or:
two R^{3A} groups on the same atom are optionally taken together with the atom to form an optionally substituted ring selected from a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 7-10 membered saturated or partially unsaturated bicyclic heterocyclic ring having 1-4 heteroatoms independently selected from nitrogen, oxygen, phosphorus, and sulfur; a 5-8 membered saturated or partially unsaturated bridged bicyclic ring having 0-3 heteroatoms independently selected from nitrogen, oxygen, phosphorus, and sulfur; and a 6-10 membered saturated or partially unsaturated spirocyclic ring having 0-3 heteroatoms independently selected from nitrogen, oxygen, phosphorus, and sulfur;
each R⁸ is independently hydrogen, oxo, halogen, —CN, —NO₂, —CHF₂, —CF₃, —OR, —(OCH₂CH₂)₁₋₁₀NR₂, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —NRC(O)OR, —NRC(O)R, —NRC(O)NR₂, —NRC(NR)NR₂, —NRNR₂, —NRS(O)₂NR₂, —NRS(O)₂R, —N═S(O)R₂, —S(NR)(O)R, —NRS(O)R, —NRCN, —P(O)R₂, —P(O)(OR)₂—CH₂NR(CH₂CH₂O)₁₋₁₀CH₂CH₂NR₂; or an optionally substituted C₁₋₆aliphatic;
each R is independently hydrogen, or an optionally substituted group selected from C₁₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic carbocyclic ring having 1-2 heteroatoms independently selected from nitrogen, oxygen, and sulfur; or:
two R groups on the same atom are optionally taken together with the atom to form an optionally substituted 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorus, and sulfur;
m is 0, 1, or 2;
n is 0, 1, 2, or 3; and
q is 0, 1, 2, or 3.

30. The compound of claim 28 or claim 29, represented by the following formula: 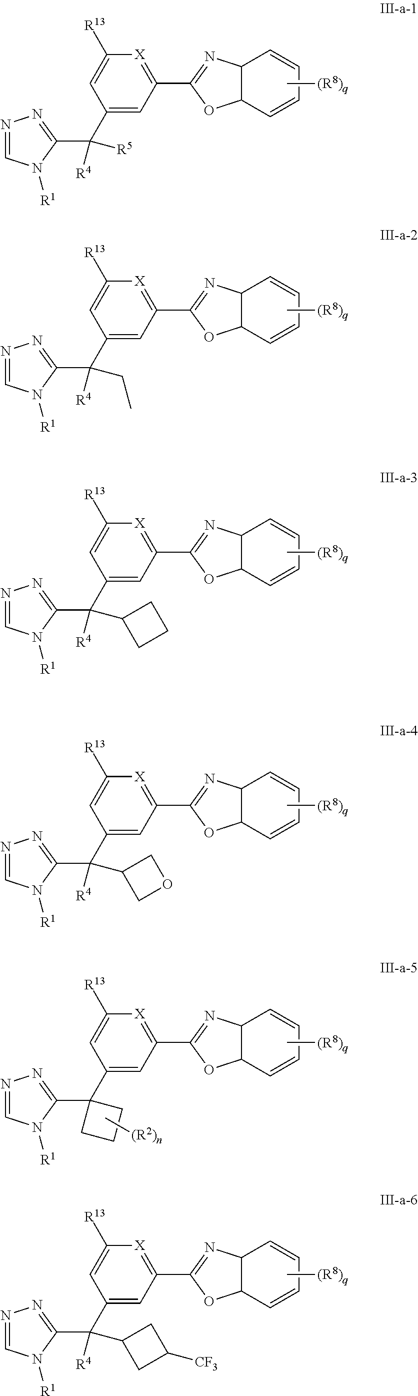
or a pharmaceutically acceptable salt thereof.

31. The compound of any one of claims 26-30, wherein R¹³ is —CN, —OR^{3A}, —N(R^{3A})(R^{3A}), —C(O)OR^{3A}, —C(O)N(R^{3A})OR^{3A}A, —N(R^{3A})C(O)OR^{3A}, —N(R^{3A})C(O)R^{3A}, —N(R^{3A})C(O)N(R^{3A})₂, —N(R^{3A})C(NR^{3A})R^{3A}, —N(R^{3A})S(O)₂R^{3A}, or an optionally substituted group selected from C₁₋₆ aliphatic: a 4-8 membered saturated or partially unsaturated monocyclic heterocyclic ring having 1-2 heteroatoms independently selected from nitrogen, oxygen, and sulfur; and a 6-10 membered saturated or partially unsaturated spirocyclic ring having 0-3 heteroatoms independently selected from nitrogen, oxygen, and sulfur.

32. The compound of any one of claims 26-31, wherein R¹³ is Cl, —CN, cyclopropyl, isopentyl, 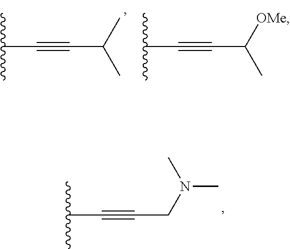
—CF₃, —CH₂OH, —CH₂OMe, —CH₂NHCOMe, — 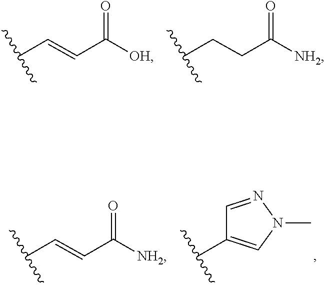
—CO₂H, —CO₂Me, —CON(Me)OMe, 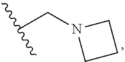 [SMILES: *CN1CCC1]
—OH, —OMe, —OEt, —OiBu, 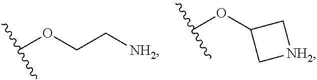
—NHEt, —NHnPr, —NHiPr, —NHiBu, —NHCOiPr, —NHC(NH)iPr, —NHCO₂Me, —NHCONHMe, —NHCONMe₂, —NHSO₂Me, —NHSO₂Et, 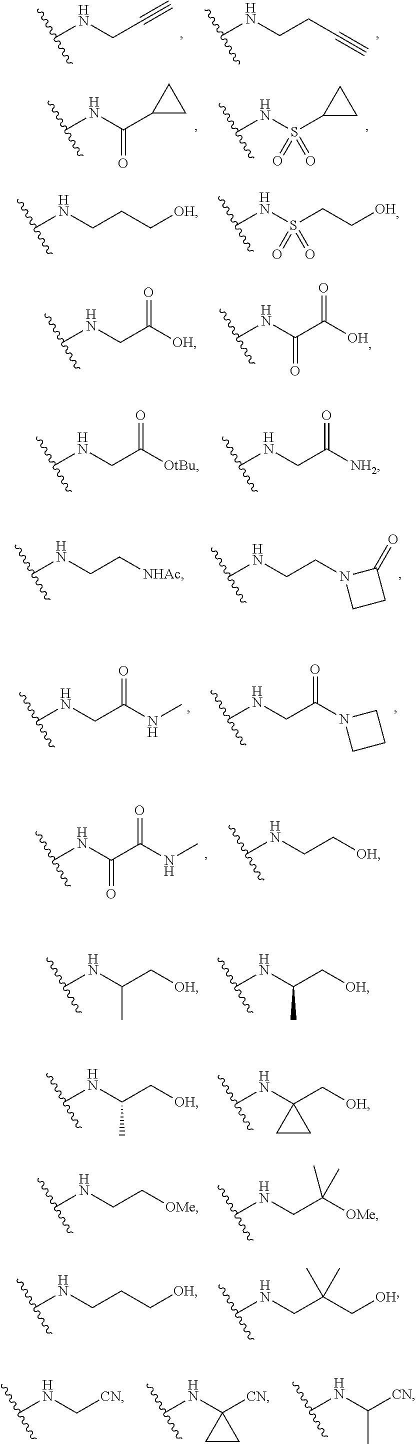 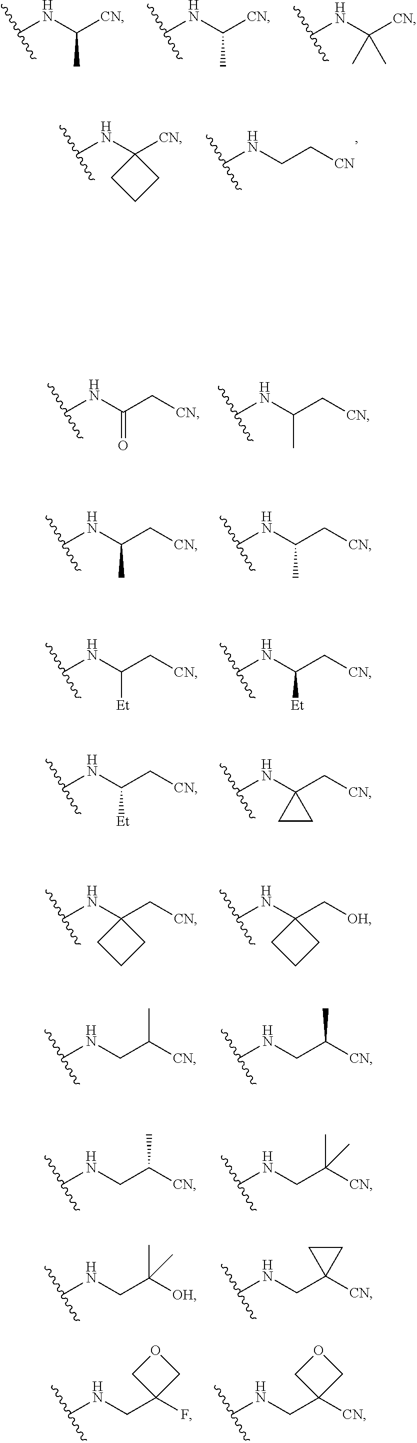 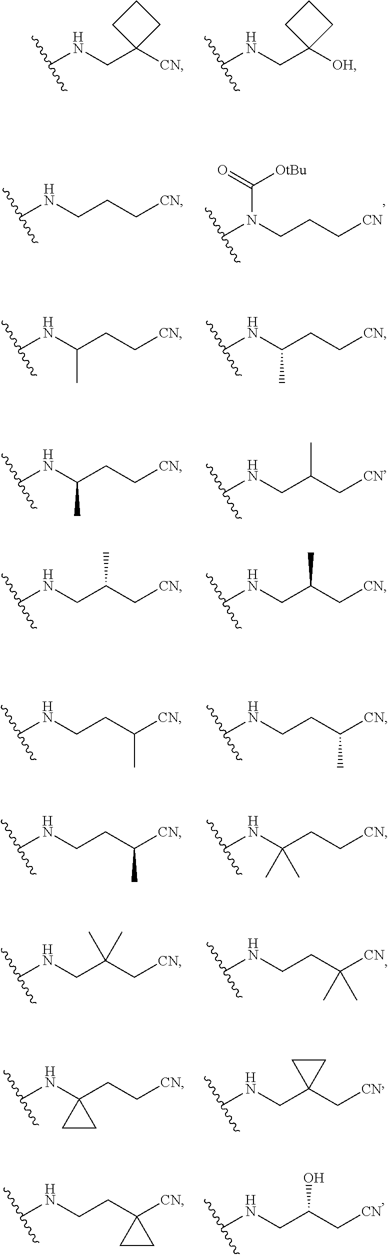 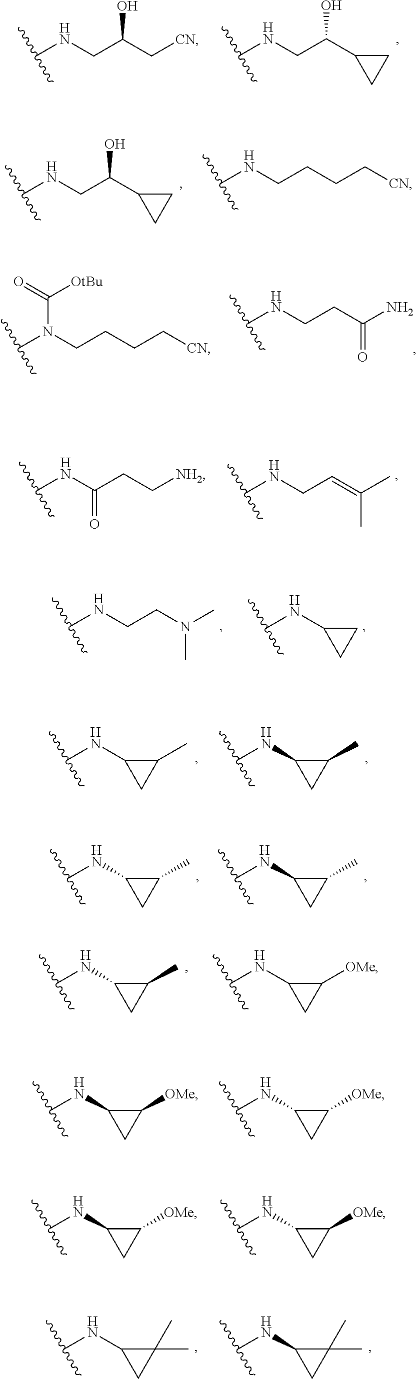 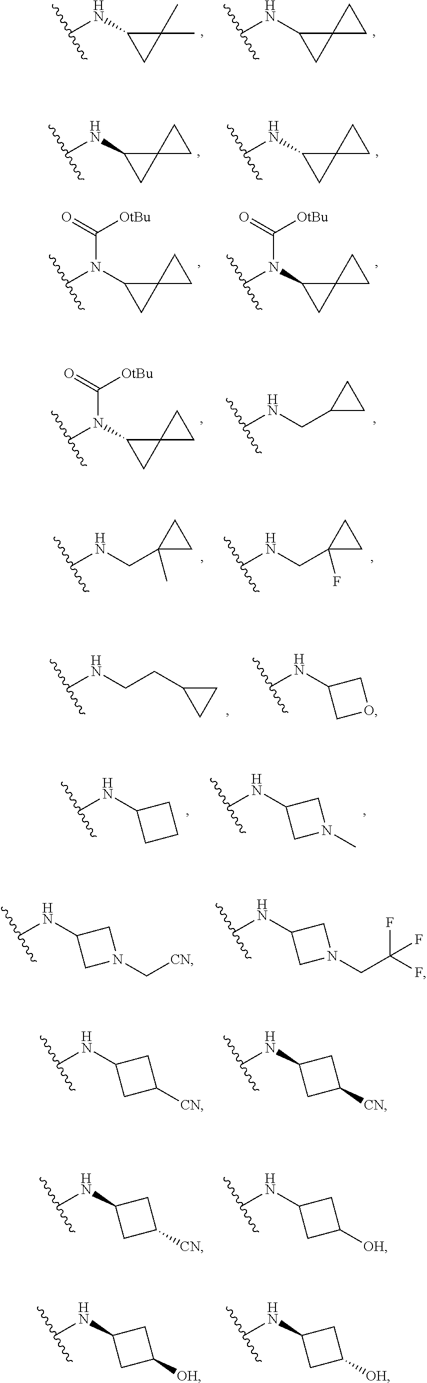 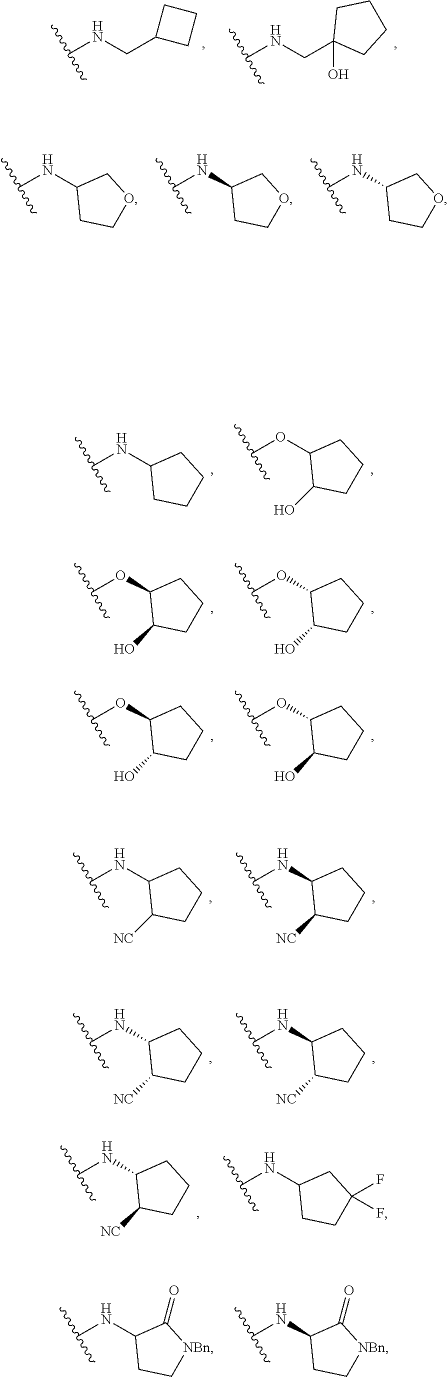 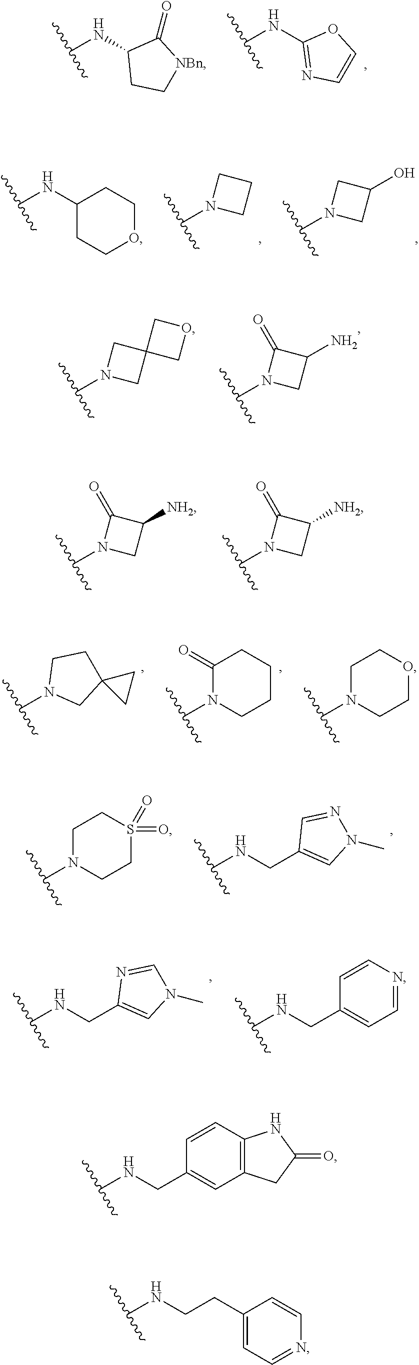
an optionally substituted phenyl, or an optionally substituted 5-6 membered heteroaryl having 1-3 ring heteroatoms selected from nitrogen, oxygen, and sulfur.

33. The compound of any one of claims 26-32, wherein R⁸ is selected from halogen, —CF₃, —OR, —NR₂, —CH₂NR(CH₂CH₂O)₁₋₄CH₂CH₂NR₂, and an optionally substituted C₁₋₆ aliphatic.

34. The compound of any one of claims 26-33, wherein R⁸ is selected from fluoro, chloro, methyl, —CF₃, —OH,

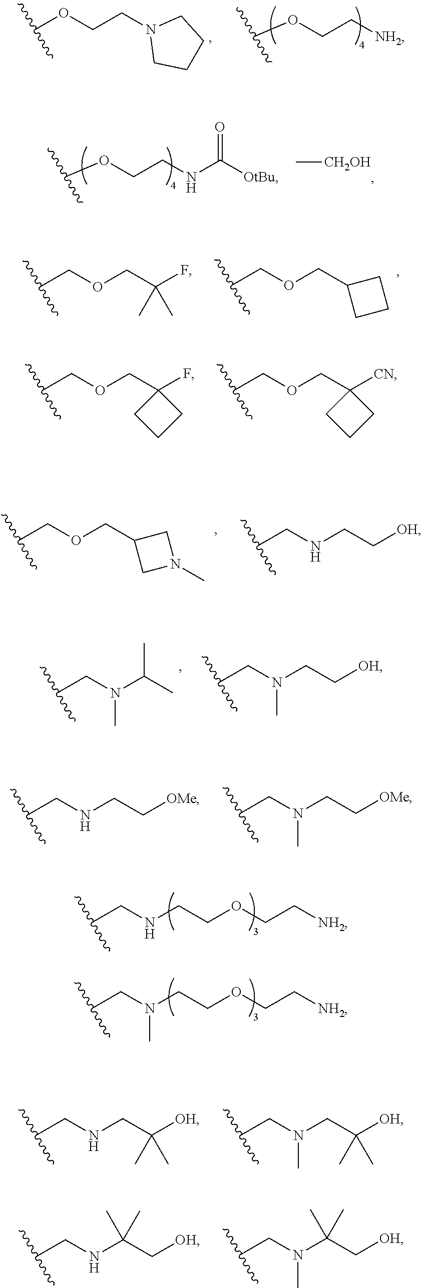

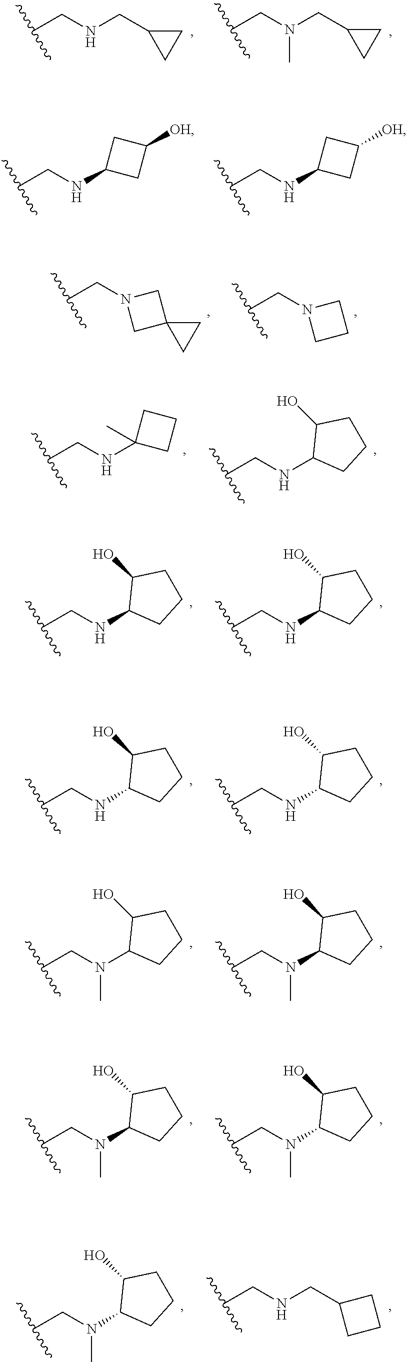

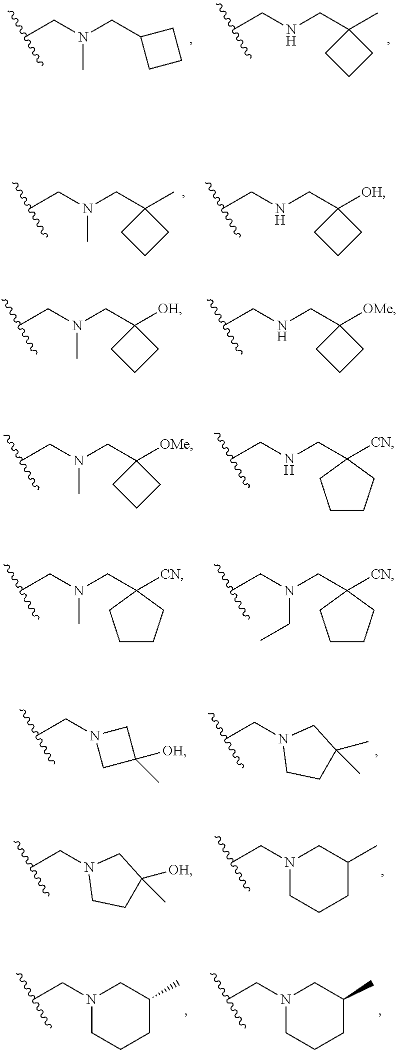

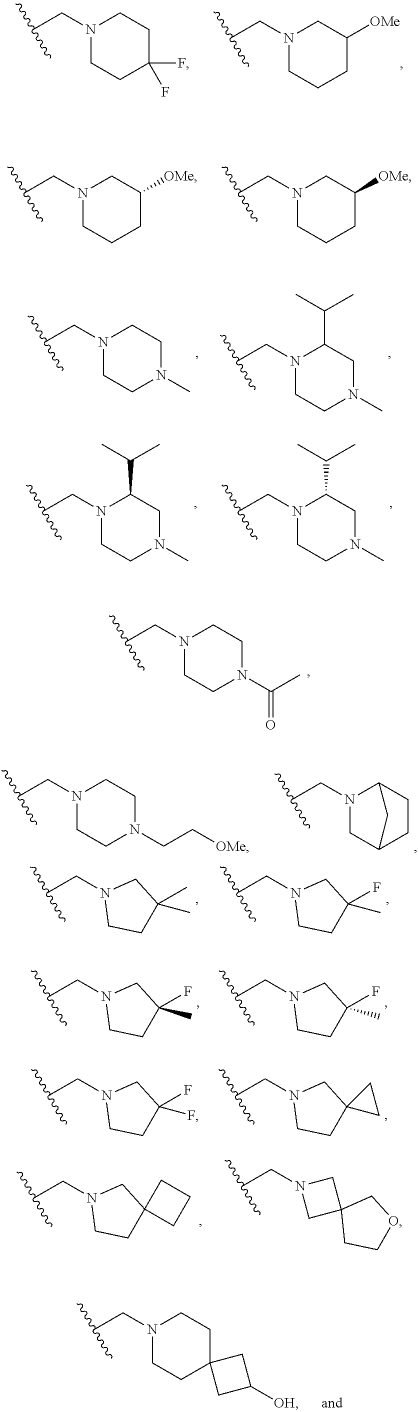

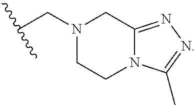 [SMILES: *CN1CCn2c(C)nnc2C1]

35. The compound of any one of the claims 1-34, wherein the compound has a Cbl-b/c-Cbl selectivity of greater than 20.

36. The compound of claim 35, wherein the compound has a Cbl-b/c-Cbl selectivity of greater than 50.

37. The compound of any one of the claims 1-36, wherein the compound is selected from those depicted in Table 1, or a pharmaceutically acceptable salt thereof.

38. A pharmaceutical composition comprising a compound according to any one of claims 1-37, or a pharmaceutically acceptable salt thereof, and a pharmaceutically acceptable carrier, adjuvant, or vehicle.

39. The compound of any one of claims 1-37, or the pharmaceutical composition of claim 38, for use as a medicament.

40. A method of inhibiting Cbl-b in a biological sample, comprising contacting the sample with the compound of any one of claims 1-37, or a pharmaceutically acceptable salt thereof, or the pharmaceutical composition of claim 38.

41. A method of treating an Cbl-b-mediated disorder, disease, or condition in a patient, comprising administering to said patient the compound of any one of claims 1-37, or a pharmaceutically acceptable salt thereof, or the pharmaceutical composition of claim 38.

42. The method of claim 41, wherein the Cbl-b-mediated disorder is a hematological cancer.

43. The method of claim 42, wherein the hematologic cancer is B-cell acute lymphoid leukemia (BALL), T-cell acute lymphoid leukemia (TALL), acute lymphoid leukemia (ALL), a chronic leukemia, a hematologic cancer or hematologic condition selected from B-cell prolymphocytic leukemia, blastic plasmacytoid dendritic cell neoplasm, Burkitt's lymphoma, diffuse large B-cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom's macroglobulinemia, and preleukemia.

44. The method of claim 41, wherein the Cbl-b-mediated disorder is bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, a chronic or acute leukemia selected from acute myeloid leukemia, chronic myeloid leukemia, acute lymphoblastic leukemia, and chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, an environmentally induced cancer, or a combination of said cancers.
